(19) United States
(12) Patent Application Publication
(10) Pub. No.: US 2024/0076675 A1
(43) Pub. Date: Mar. 7, 2024

(54) MODULATORS OF PNPLA3 EXPRESSION

(71) Applicant: Ionis Pharmaceuticals, Inc., Carlsbad, CA (US)

(72) Inventors: Susan M. FREIER, San Marcos, CA (US); Huynh-Hoa BUI, San Diego, CA (US)

(21) Appl. No.: 18/457,941

(22) Filed: Aug. 29, 2023

Related U.S. Application Data
(62) Division of application No. 16/936,060, filed on Jul. 22, 2020, now Pat. No. 11,781,143.
Application No. 16/936,060 is a continuation of application No. 16/574,407, filed on Sep. 18, 2019, now Pat. No. 10,774,333.
(60) Provisional application No. 62/733,152, filed on Sep. 19, 2018.

(51) Int. Cl. C12N 15/113; A61P 1/16

(52) U.S. Cl. CPC C12N 15/1137; A61P 1/16; C12N 2310/315; C12N 2310/32

(57) ABSTRACT

The present embodiments provide methods, compounds, and compositions useful for inhibiting PNPLA3 expression, which may be useful for treating, preventing, or ameliorating a disease associated with PNPLA3.

### SEQUENCE LISTING

The present application is being filed along with a Sequence Listing in electronic format. The Sequence Listing is provided as a file entitled 200797-BIOL0317SEQ.xml, created Aug. 25, 2023, which is 1,952 kb in size. The information in the electronic format of the sequence listing is incorporated herein by reference in its entirety.

### FIELD

The present embodiments provide methods, compounds, and compositions useful for inhibiting PNPLA3 (patatin like phospholipase domain containing 3; hypothetical protein dJ796I17.1; adiponutrin; DJ796I17.1) expression, and in certain instances, reducing the amount of PNPLA3 protein in a cell or animal, which can be useful for treating, preventing, or ameliorating a disease associated with PNPLA3.

### BACKGROUND

Non-alcoholic fatty liver disease (NAFLD) covers a spectrum of liver disease from steatosis to nonalcoholic steatohepatitis (NASH) and cirrhosis. NAFLD is defined as fat accumulation in the liver exceeding 5% by weight, in the absense of significant alcohol consumption, steatogenic medication, or hereditary disorders (Kotronen et al, Arterioscler Thromb. Vasc. Biol. 2008, 28: 27-38).

Non-alcoholic steatohepatitis (NASH) is NAFLD with signs of inflammation and hepatic injury. NASH is defined histologically by macrovesicular steatosis, hepatocellular ballooning, and lobular inflammatory infiltrates (Sanyal, Hepatol. Res. 2011. 41: 670-4). NASH is estimated to affect 2-3% of the general population. In the presence of other pathologies, such as obesity or diabetes, the estimated prevalence increases to 7% and 62% respectively (Hashimoto et al, J. Gastroenterol. 2011. 46(1): 63-69).

PNPLA3 is a 481 amino acid member of the patatin-like phospholipase domain-containing family that is expressed in the ER and on lipid droplets. In humans, PNPLA3 is highly expressed in the liver, whereas adipose tissue expression is five-fold less (Huang et al, Proc. Natl. Acad. Sci. USA 2010. 107: 7892-7).

### SUMMARY

Certain embodiments provided herein are compounds and methods for reducing the amount or activity of PNPLA3 mRNA, and in certain embodiments, reducing the amount of PNPLA3 protein in a cell or animal. In certain embodiments, the animal has a liver disease. In certain embodiments, the disease is NASH. In certain embodiments, the disease is NAFLD. In certain embodiments, the disease is hepatic steatosis. In certain embodiments, the disease is liver cirrhosis. In certain embodiments, the disease is hepatocellular carcinoma. In certain embodiments, the disease is alcoholic liver disease. In certain embodiments, the disease is alcoholic steatohepatitis (ASH). In certain embodiments, the disease is HCV hepatitis. In certain embodiments, the disease is chronic hepatitis. In certain embodiments, the disease is hereditary hemochromatosis. In certain embodiments, the disease is primary sclerosing cholangitis. Certain compounds provided herein are directed to compounds and compositions that reduce liver damage, steatosis, liver fibrosis, liver inflammation, liver scarring or cirrhosis, liver failure, liver enlargement, elevated transaminases, or hepatic fat accumulation in an animal.

Certain embodiments provided herein are directed to potent and tolerable compounds and compositions useful for inhibiting PNPLA3 expression, which can be useful for treating, preventing, ameliorating, or slowing progression of liver diseases. Certain embodiments provided herein are directed to compounds and compositions that are more potent or have greater therapeutic value than compounds publicly disclosed.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ION number indicate a combination of nucleobase sequence, chemical modification, and motif.

### Definitions

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE) refers to a 2′-O(CH₂)₂-OCH₃) in the place of the 2′—OH group of a ribosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“About” means within +10% of a value. For example, if it is stated, “the compounds affected about 70% inhibition of PNPLA3”, it is implied that PNPLA3 levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a ribosyl bicyclic sugar moiety wherein the second ring of the bicyclic sugar is formed via a bridge connecting the 4′-carbon and the 2′-carbon, wherein the bridge has the formula: 4′-CH(CH₃)—O-2′, and wherein the methyl group of the bridge is in the S configuration.

“cEt nucleoside” means a nucleoside comprising a cEt modified sugar moiety.

“Chemical modification” in a compound describes the substitutions or changes through chemical reaction, of any of the units in the compound relative to the original state of such unit. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” means chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Double-stranded antisense compound” means an antisense compound comprising two oligomeric compounds that are complementary to each other and form a duplex, and wherein one of the two said oligomeric compounds comprises an oligonucleotide.

“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to, the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Individual” means a human or non-human animal selected for treatment or therapy.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Lengthened oligonucleotides” are those that have one or more additional nucleosides relative to an oligonucleotide disclosed herein, e.g. a parent oligonucleotide.

“Linked nucleosides” means adjacent nucleosides linked together by an internucleoside linkage.

“Linker-nucleoside” means a nucleoside that links an oligonucleotide to a conjugate moiety. Linker-nucleosides are located within the conjugate linker of a compound. Linker-nucleosides are not considered part of the oligonucleotide portion of a compound even if they are contiguous with the oligonucleotide.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating PNPLA3 RNA can mean to increase or decrease the level of PNPLA3 RNA and/or PNPLA3 protein in a cell, tissue, organ or organism. A “modulator” effects the change in the cell, tissue, organ or organism. For example, a PNPLA3 compound can be a modulator that decreases the amount of PNPLA3 RNA and/or PNPLA3 protein in a cell, tissue, organ or organism.

“MOE” means methoxyethyl.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutically acceptable carrier or diluent” means any substance suitable for use in administering to an individual. For example, a pharmaceutically acceptable carrier can be a sterile aqueous solution, such as PBS or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prevent” refers to delaying or forestalling the onset, development or progression of a disease, disorder, or condition for a period of time from minutes to indefinitely.

“Prodrug” means a compound in a form outside the body which, when administered to an individual, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Reduce” means to bring down to a smaller extent, size, amount, or number.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence_databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites” are defined as unique nucleobase positions within a target nucleic acid.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” with reference to a target nucleic acid means to reduce or block expression of the target nucleic acid while exhibiting fewer, minimal, or no effects on non-target nucleic acids. Reduction does not necessarily indicate a total elimination of the target nucleic acid's expression.

“Standard cell assay” means assay(s) described in the Examples and reasonable variations thereof.

“Standard in vivo experiment” means the procedure(s) described in the Example(s) and reasonable variations thereof.

“Stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

“Sugar moiety” means an unmodified sugar moiety or a modified sugar moiety. “Unmodified sugar moiety” or “unmodified sugar” means a 2′-OH(H) ribosyl moiety, as found in RNA (an “unmodified RNA sugar moiety”), or a 2′-H(H) moiety, as found in DNA (an “unmodified DNA sugar moiety”). “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen or hydroxyl of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary compounds or nucleic acids.

“Synergy” or “synergize” refers to an effect of a combination that is greater than additive of the effects of each component alone at the same doses.

“PNPLA3” means any nucleic acid or protein of PNPLA3. “PNPLA3 nucleic acid” means any nucleic acid encoding PNPLA3. For example, in certain embodiments, a PNPLA3 nucleic acid includes a DNA sequence encoding PNPLA3, an RNA sequence transcribed from DNA encoding PNPLA3 (including genomic DNA comprising introns and exons), and an mRNA sequence encoding PNPLA3. “PNPLA3 mRNA” means an mRNA encoding a PNPLA3 protein. The target may be referred to in either upper or lower case.

“PNPLA3 specific inhibitor” refers to any agent capable of specifically inhibiting PNPLA3 RNA and/or PNPLA3 protein expression or activity at the molecular level. For example, PNPLA3 specific inhibitors include nucleic acids (including antisense compounds), peptides, antibodies, small molecules, and other agents capable of inhibiting the expression of PNPLA3 RNA and/or PNPLA3 protein.

“Target gene” refers to a gene encoding a target.

“Targeting” means the specific hybridization of a compound to a target nucleic acid in order to induce a desired effect.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

“Target segment” means the sequence of nucleotides of a target nucleic acid to which a compound is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to an individual.

“Treat” refers to administering a compound or pharmaceutical composition to an animal in order to effect an alteration or improvement of a disease, disorder, or condition in the animal.

### Certain Embodiments

Certain embodiments provide methods, compounds and compositions for inhibiting PNPLA3 (PNPLA3) expression.

Certain embodiments provide compounds targeted to a PNPLA3 nucleic acid. In certain embodiments, the PNPLA3 nucleic acid has the sequence set forth in RefSeq or GENBANK Accession No. NM_025225.2 (incorporated by reference, disclosed herein as SEQ ID NO: 1); NC_000022.11 truncated from nucleotides 43921001 to U.S. Pat. No. 43,954,500 (incorporated by reference, disclosed herein as SEQ ID NO: 2); AK123806.1 (incorporated by reference, disclosed herein as SEQ ID NO: 3); BQ686328.1 (incorporated by reference, disclosed herein as SEQ ID NO: 4); BF762711.1 (incorporated by reference, disclosed herein as SEQ ID NO: 5); DA290491.1 (incorporated by reference, disclosed herein as SEQ ID NO: 6); and the sequences listed as SEQ ID Nos 7, 8, 9, and 10. In certain embodiments, the compound is an antisense compound or oligomeric compound. In certain embodiments, the compound is single-stranded. In certain embodiments, the compound is double-stranded.

In certain embodiments, the compound comprises a modified oligonucleotide 16 linked nucleosides in length. In certain embodiments, the compound is an antisense compound or oligomeric compound.

Certain embodiments provide a compound comprising a modified oligonucleotide 12 to 30 linked nucleosides in length and having a nucleobase sequence comprising the nucleobase sequence of any one of SEQ ID NOs: 17-2169. In certain embodiments, the compound is an antisense compound or oligomeric compound. In certain embodiments, the compound is single-stranded. In certain embodiments, the compound is double-stranded. In certain embodiments, the modified oligonucleotide is 16 to 30 linked nucleosides in length.

Certain embodiments provide a compound comprising a modified oligonucleotide consisting of the nucleobase sequence of any one of SEQ ID NOs: 17-2169. In certain embodiments, the compound is an antisense compound or oligomeric compound. In certain embodiments, the compound is single-stranded. In certain embodiments, the compound is double-stranded.

Certain embodiments provide a compound comprising a modified oligonucleotide 12 to 30 linked nucleosides in length and complementary within nucleobases 5567-5642, 5644-5731, 5567-5731, 5567-5620, 13697-13733, 20553-20676, 20664-20824, 20553-20824, and 25844-25912 of SEQ ID NO: 2, wherein said modified oligonucleotide is at least 85%, at least 90%, at least 95%, or 100% complementary to SEQ ID NO: 2. In certain embodiments, the compound is an antisense compound or oligomeric compound. In certain embodiments, the compound is single-stranded. In certain embodiments, the compound is double-stranded. In certain embodiments, the modified oligonucleotide is 16 to 30 linked nucleosides in length.

In certain embodiments, compounds target nucleotides 5567-5620 of a PNPLA3 nucleic acid. In certain embodiments, compounds target within nucleotides 5567-5642, 5644-5731, 5567-5731, 5567-5620 of a PNPLA3 nucleic acid having the nucleobase sequence of SEQ ID NO: 2. In certain embodiments, compounds have at least an 8, 9, 10, 11, 12, 13, 14, 15, or 16 contiguous nucleobase portion complementary to an equal length portion within nucleotides 5567-5642, 5644-5731, 5567-5731, 5567-5620 of a PNPLA3 nucleic acid having the nucleobase sequence of SEQ ID NO: 2. In certain embodiments, these compounds are antisense compounds, oligomeric compounds, or oligonucleotides.

In certain embodiments, a compound comprises a modified oligonucleotide 12 to 30 linked nucleosides in length and having a nucleobase sequence comprising at least an 8, 9, 10, 11, 12, 13, 14, 15, or 16 contiguous nucleobase portion any one of SEQ ID NOs: 1089, 1757, 141, 1982, 330, 1665, 408, 830, and 899. In certain embodiments, the modified oligonucleotide is 16 to 30 linked nucleosides in length.

In certain embodiments, a compound comprises a modified oligonucleotide 12 to 30 linked nucleosides in length and having a nucleobase sequence comprising any one of SEQ ID NOs: 1089, 1757, 141, 1982, 330, 1665, 408, 830, and 899. In certain embodiments, the modified oligonucleotide is 16 to 30 linked nucleosides in length.

In certain embodiments, a compound comprises a modified oligonucleotide having a nucleobase sequence consisting of any one of SEQ ID NOs: 1089, 1757, 141, 1982, 330, 1665, 408, 830, and 899.

In certain embodiments, compounds targeted to PNPLA3 is ION 975616, 994284, 975605, 994282, 975613, 975617, 975735, 975736, or 975612. Out of over 2,384 compounds that were screened as described in the Examples section below, ION 975616, 994284, 975605, 994282, 975613, 975617, 975735, 975736, and 975612 emerged as the top lead compounds.

In certain embodiments, any of the foregoing modified oligonucleotides comprises at least one modified internucleoside linkage, at least one modified sugar, and/or at least one modified nucleobase.

In certain embodiments, any of the foregoing modified oligonucleotides comprises at least one modified sugar. In certain embodiments, at least one modified sugar comprises a 2′-O-methoxyethyl group. In certain embodiments, at least one modified sugar is a bicyclic sugar, such as a 4′-CH(CH3)-O-2′ group, a 4′-CH2-O-2′ group, or a 4′-(CH2)₂-O-2′group.

In certain embodiments, the modified oligonucleotide comprises at least one modified internucleoside linkage, such as a phosphorothioate internucleoside linkage.

In certain embodiments, any of the foregoing modified oligonucleotides comprises at least one modified nucleobase, such as 5-methylcytosine.

In certain embodiments, any of the foregoing modified oligonucleotides comprises:
a gap segment consisting of linked deoxynucleosides;
a 5′ wing segment consisting of linked nucleosides; and
a 3′ wing segment consisting of linked nucleosides;
   wherein the gap segment is positioned between the 5′ wing segment and the 3′ wing segment and wherein each nucleoside of each wing segment comprises a modified sugar. In certain embodiments, the modified oligonucleotide is 12 to 30 linked nucleosides in length having a nucleobase sequence comprising the sequence recited in any one of SEQ ID NOs: 1089, 1757, 141, 1982, 330, 1665, 408, 830, and 899. In certain embodiments, the modified oligonucleotide is 16 to 30 linked nucleosides in length having a nucleobase sequence comprising the sequence recited in any one of SEQ ID NOs: 1089, 1757, 141, 1982, 330, 1665, 408, 830, and 899. In certain embodiments, the modified oligonucleotide is 16 linked nucleosides in length having a nucleobase sequence consisting of the sequence recited in any one of SEQ ID NOs: 1089, 1757, 141, 1982, 330, 1665, 408, 830, and 899.

In certain embodiments, a compound comprises or consists of a modified oligonucleotide 12-30 linked nucleobases in length having a nucleobase sequence comprising the sequence recited in any one of SEQ ID NOs: 1089, 1757, 141, 1982, 330, 1665, 408, 830, and 899, wherein the modified oligonucleotide comprises
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide, wherein the modified oligonucleotide is 16 linked nucleosides in length and consists of the sequence of SEQ ID NO: 1089, wherein the modified oligonucleotide comprises:
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment, wherein each nucleoside of each wing segment comprises a cEt sugar; wherein each internucleoside linkage is a phosphorothioate linkage; and wherein each cytosine is a 5-methylcytosine.

In certain embodiments, a compound consists of a modified oligonucleotide and a conjugate group, wherein the modified oligonucleotide is 16 linked nucleosides in length and consists of the sequence of SEQ ID NO: 1089, wherein the modified oligonucleotide comprises:
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment, wherein each nucleoside of each wing segment comprises a cEt sugar; wherein each internucleoside linkage is a phosphorothioate linkage; wherein each cytosine is a 5-methylcytosine; and wherein the conjugate group is positioned at the 5′end of the modified oligonucleotide and is

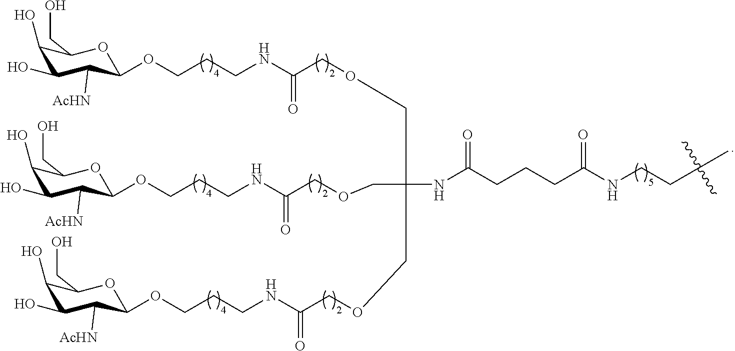

In certain embodiments, a compound comprises or consists of ION 916333 or salt thereof, having the following chemical structure (SEQ ID NO: 2173):

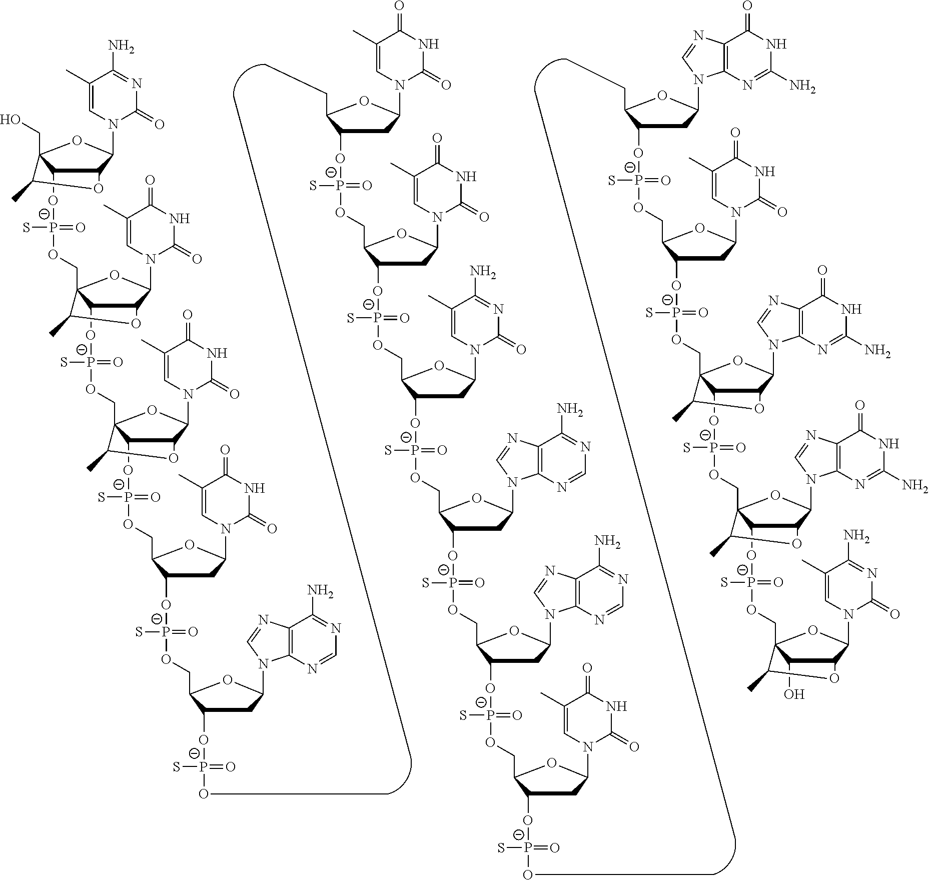

In certain embodiments, a compound comprises or consists of ION 975616 or salt thereof, having the following chemical structure (SEQ ID NO: 2174):

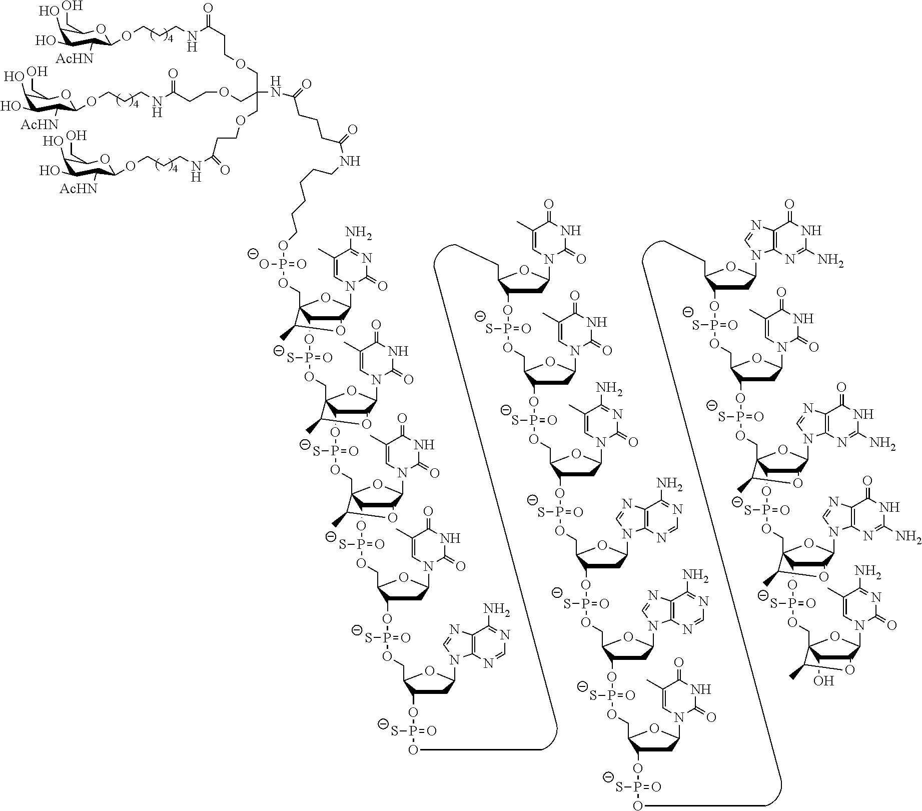

In certain embodiments, a compound comprises or consists of the sodium salt of 975616, having the following chemical structure (SEQ ID NO: 2174):

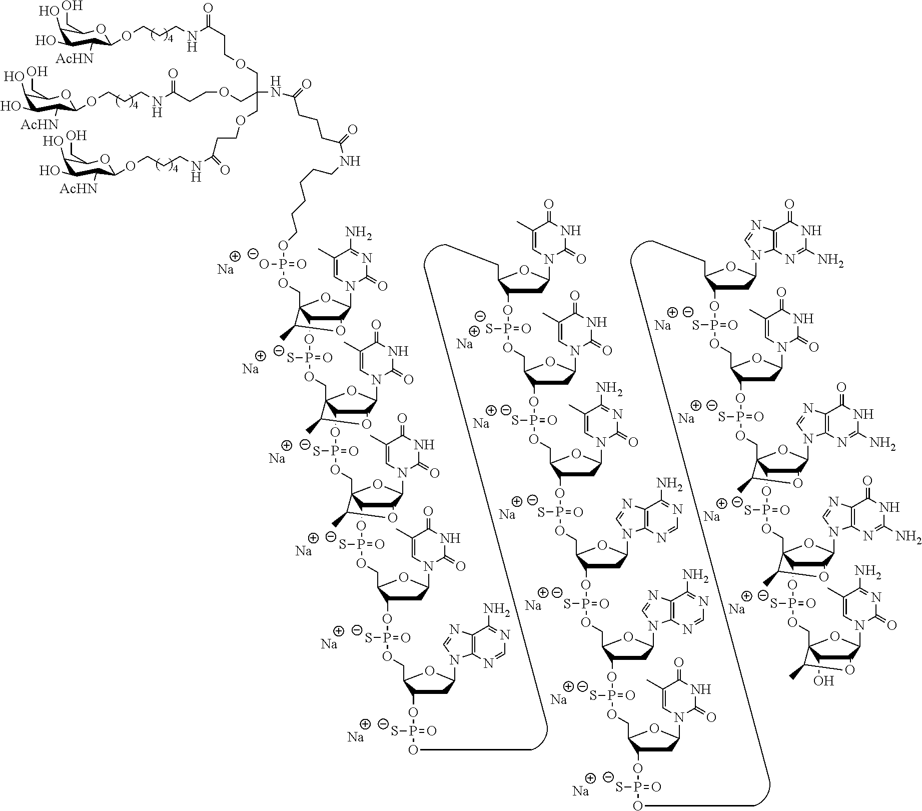

In certain embodiments, a compound comprises or consists of ION 975613 or salt thereof, having the following chemical structure (SEQ ID NO: 2176):

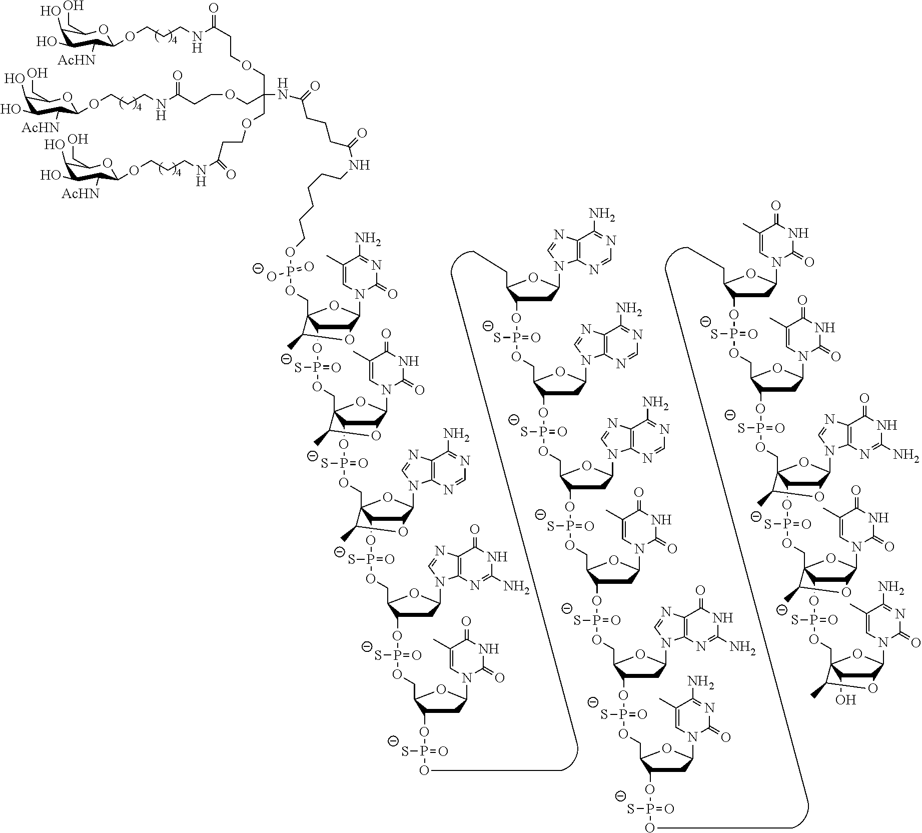

In certain embodiments, a compound comprises or consists of the sodium salt of 975613, having the following chemical structure (SEQ ID NO: 2176):

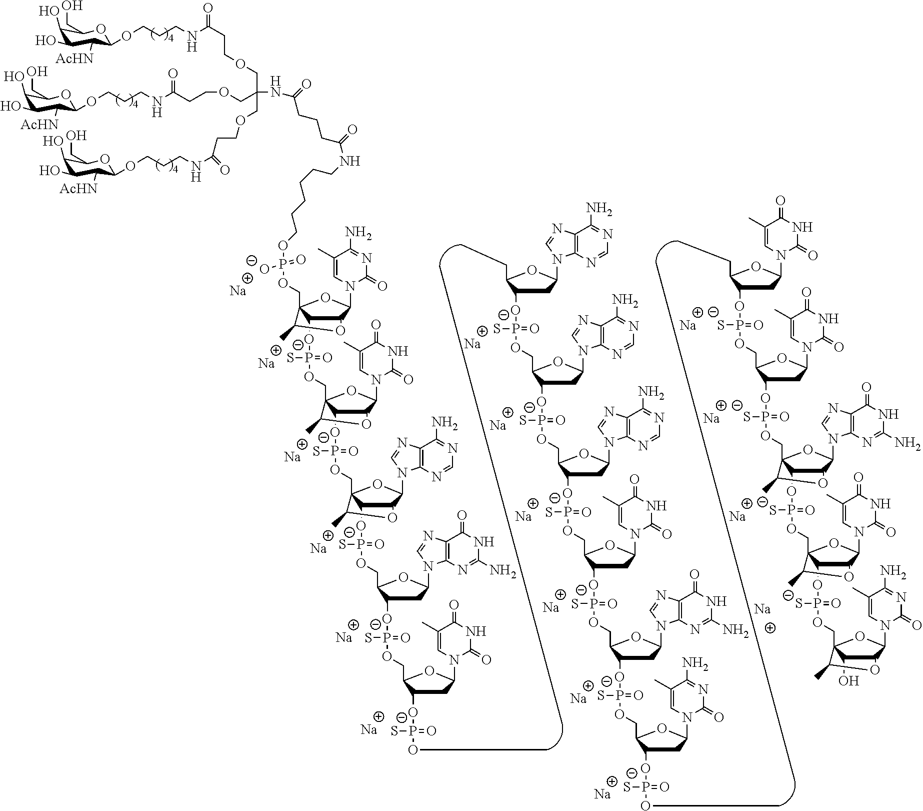

In certain embodiments, a compound comprises or consists of ION 975612 or salt thereof, having the following chemical structure (SEQ ID NO: 2178):

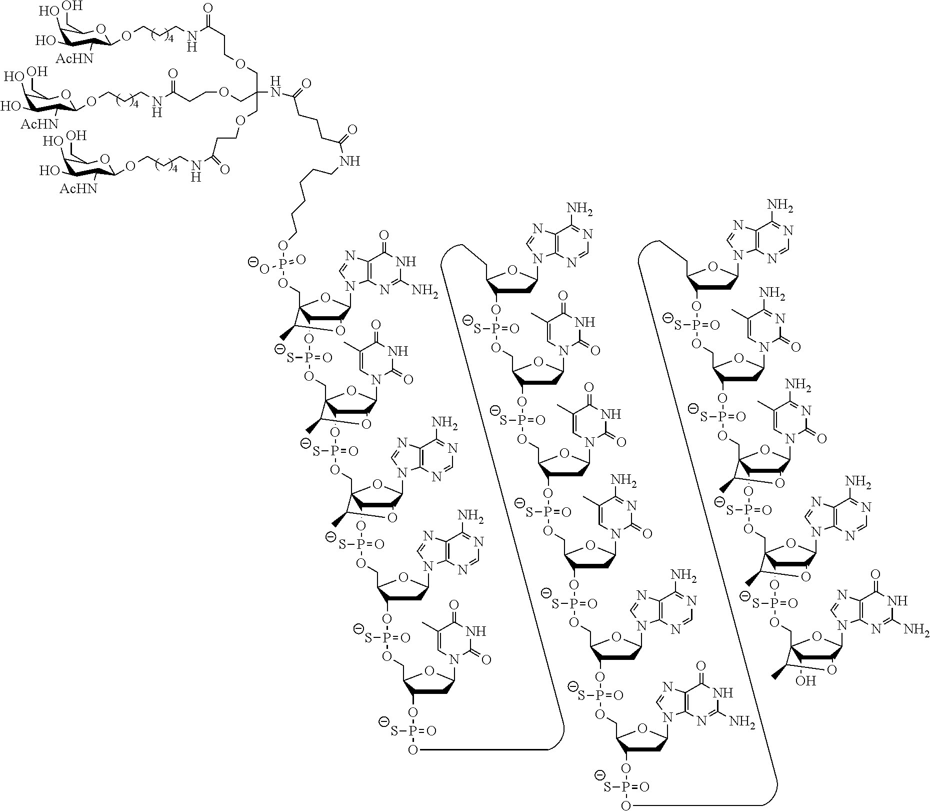

In certain embodiments, a compound comprises or consists of the sodium salt of 975612, having the following chemical structure (SEQ ID NO: 2178):

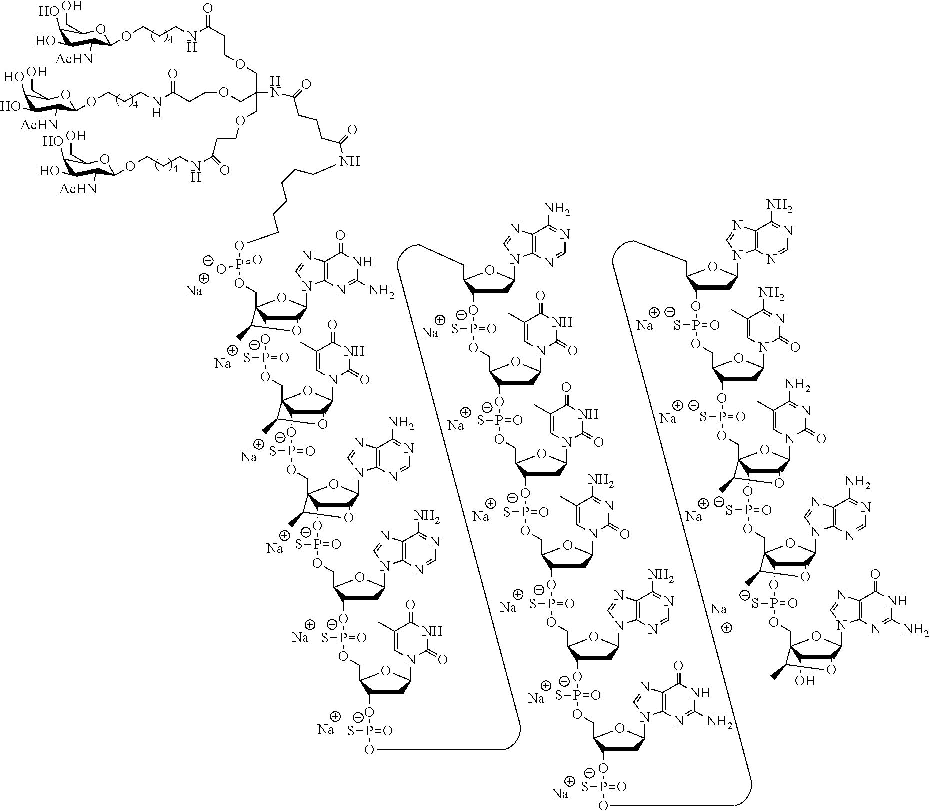

In certain embodiments, a compound comprises or consists of ION 916789 or salt thereof, having the following chemical structure (SEQ ID NO: 2177):

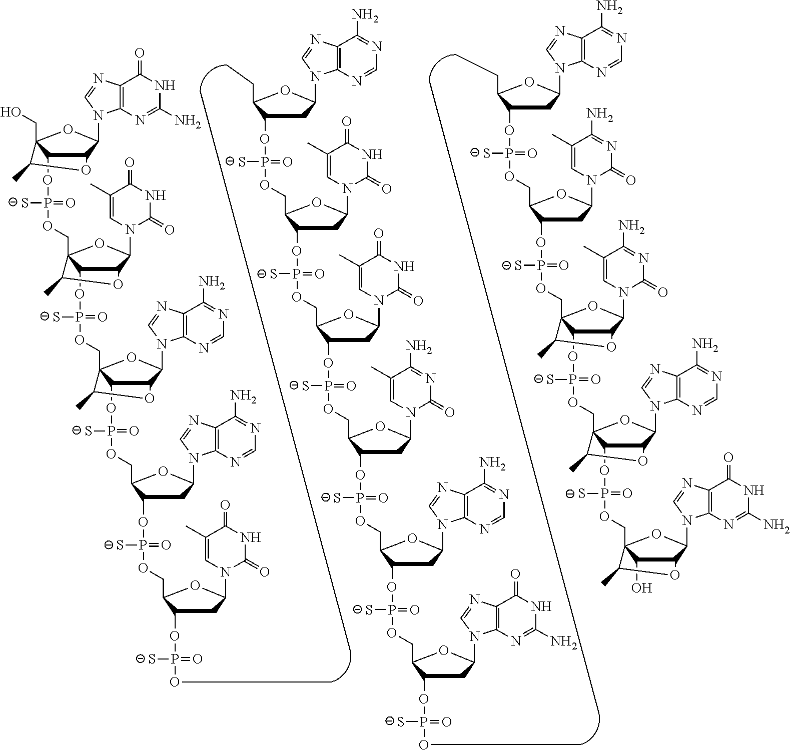

In certain embodiments, a compound comprises or consists of the sodium salt of 916789, having the following chemical structure (SEQ ID NO: 2177):

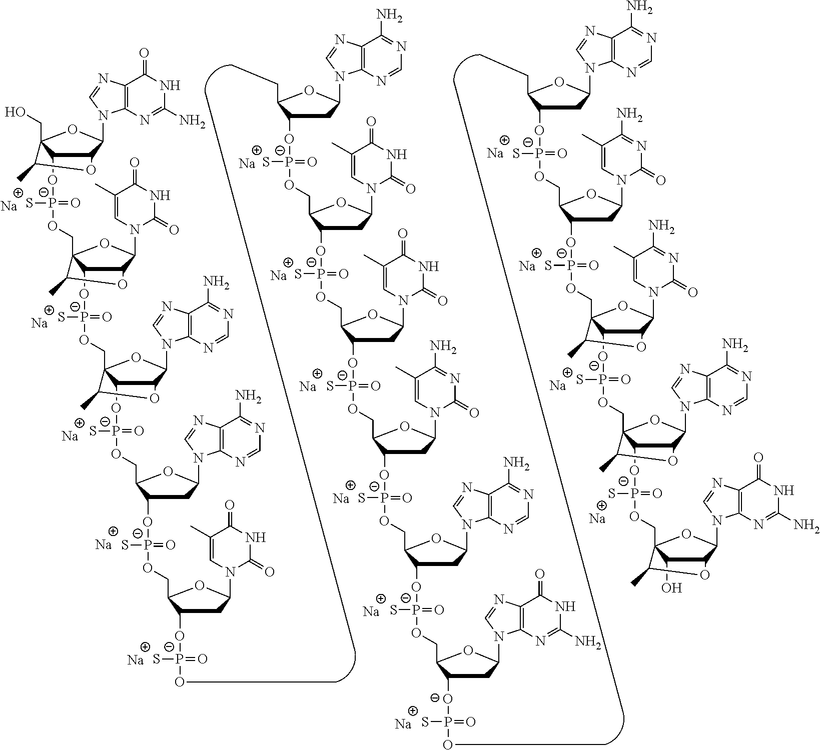

In certain embodiments, a compound comprises or consists of ION 916602 or salt thereof, having the following chemical structure (SEQ ID NO: 2175):

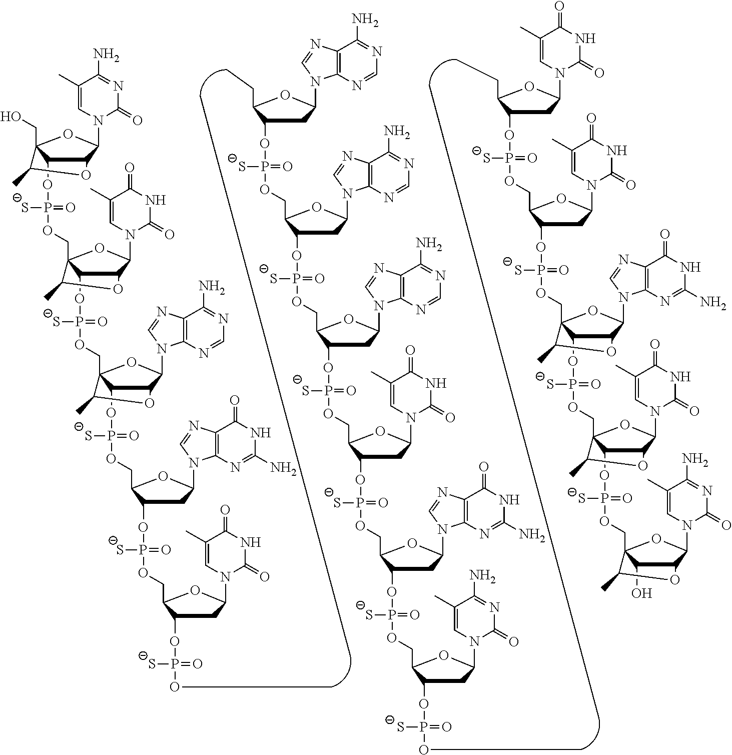

In certain embodiments, a compound comprises or consists of the sodium salt of 916602, having the following chemical structure (SEQ ID NO: 2175):

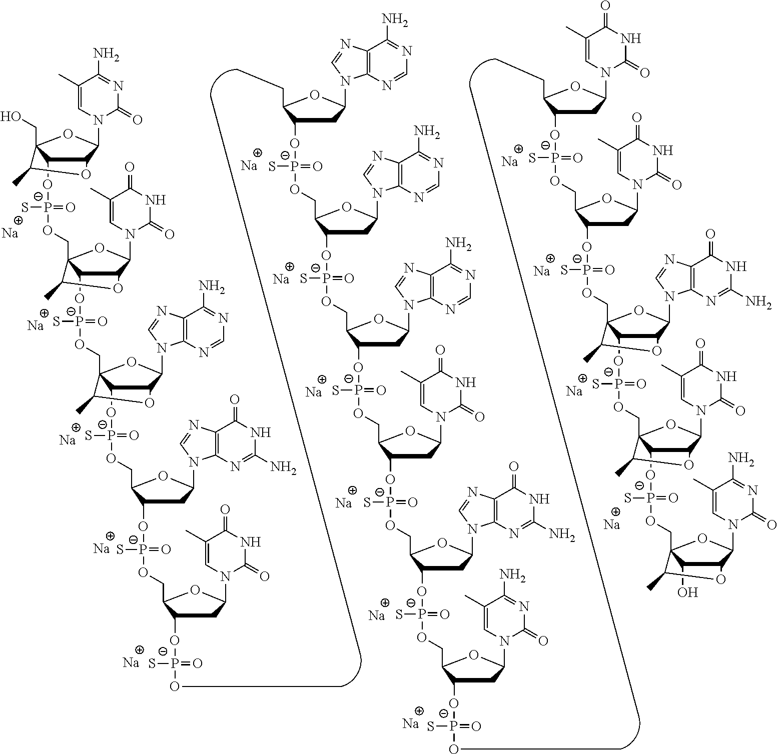

In any of the foregoing embodiments, the compound or oligonucleotide can be at least 85%, at least 90%, at least 95%, at least 98%, at least 99%, or 100% complementary to a nucleic acid encoding PNPLA3.

In any of the foregoing embodiments, the compound can be single-stranded. In certain embodiments, the compound comprises deoxyribonucleotides. In certain embodiments, the compound is double-stranded. In certain embodiments, the compound is double-stranded and comprises ribonucleotides. In any of the foregoing embodiments, the compound can be an antisense compound or oligomeric compound.

In any of the foregoing embodiments, the compound can be 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides in length. In certain embodiments, the compound comprises or consists of an oligonucleotide.

In certain embodiments, a compound comprises a modified oligonucleotide described herein and a conjugate group. In certain embodiments, the conjugate group is linked to the modified oligonucleotide at the 5′ end of the modified oligonucleotide. In certain embodiments, the conjugate group is linked to the modified oligonucleotide at the 3′ end of the modified oligonucleotide. In certain embodiments, the conjugate group comprises at least one N-Acetylgalactosamine (GalNAc), at least two N-Acetylgalactosamines (GalNAcs), or at least three N-Acetylgalactosamines (GalNAcs).

In certain embodiments, compounds or compositions provided herein comprise a pharmaceutically acceptable salt of the modified oligonucleotide. In certain embodiments, the salt is a sodium salt. In certain embodiments, the salt is a potassium salt.

In certain embodiments, the compounds or compositions as described herein are active by virtue of having at least one of an in vitro IC₅₀ of less than 2 μM, less than 1.5 μM, less than 1 μM, less than 0.9 μM, less than 0.8 μM, less than 0.7 μM, less than 0.6 μM, less than 0.5 μM, less than 0.4 μM, less than 0.3 μM, less than 0.2 μM, less than 0.1 μM, less than 0.05 μM, less than 0.04 μM, less than 0.03 μM, less than 0.02 μM, or less than 0.01 μM.

In certain embodiments, the compounds or compositions as described herein are highly tolerable as demonstrated by having at least one of an increase in alanine transaminase (ALT) or aspartate transaminase (AST) value of no more than 4 fold, 3 fold, or 2 fold over control animals, or an increase in liver, spleen, or kidney weight of no more than 30%, 20%, 15%, 12%, 10%, 5%, or 2% compared to control animals. In certain embodiments, the compounds or compositions as described herein are highly tolerable as demonstrated by having no increase of ALT or AST over control animals. In certain embodiments, the compounds or compositions as described herein are highly tolerable as demonstrated by having no increase in liver, spleen, or kidney weight over control animals.

Certain embodiments provide a composition comprising the compound of any of the aforementioned embodiments or any pharmaceutically acceptable salt thereof and at least one of a pharmaceutically acceptable carrier or diluent. In certain embodiments, the composition has a viscosity less than about 40 centipoise (cP), less than about 30 centipose (cP), less than about 20 centipose (cP), less than about 15 centipose (cP), or less than about 10 centipose (cP). In certain embodiments, the composition having any of the aforementioned viscosities comprises a compound provided herein at a concentration of about 100 mg/mL, about 125 mg/mL, about 150 mg/mL, about 175 mg/mL, about 200 mg/mL, about 225 mg/mL, about 250 mg/mL, about 275 mg/mL, or about 300 mg/mL. In certain embodiments, the composition having any of the aforementioned viscosities and/or compound concentrations has a temperature of room temperature, or about 20° C., about 21° C., about 22° C., about 23° C., about 24° C., about 25° C., about 26° C., about 27° C., about 28° C., about 29° C., or about 30° C.

### Certain Indications

Certain embodiments provided herein relate to methods of inhibiting PNPLA3 expression, which can be useful for treating, preventing, or ameliorating a disease associated with PNPLA3 in an individual, by administration of a compound that targets PNPLA3. In certain embodiments, the compound can be a PNPLA3 specific inhibitor. In certain embodiments, the compound can be an antisense compound, an oligomeric compound, or an oligonucleotide targeted to PNPLA3.

Examples of diseases associated with PNPLA3 treatable, preventable, and/or ameliorable with the methods provided herein include liver disease, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. Certain compounds provided herein are directed to compounds and compositions that reduce liver damage, steatosis, liver fibrosis, liver inflammation, liver scarring or cirrhosis, liver failure, liver enlargement, elevated transaminases, or hepatic fat accumulation in an animal.

In certain embodiments, a method of treating, preventing, or ameliorating a disease associated with PNPLA3 in an individual comprises administering to the individual a compound comprising a PNPLA3 specific inhibitor, thereby treating, preventing, or ameliorating the disease. In certain embodiments, the individual is identified as having, or at risk of having, a disease associated with PNPLA3. In certain embodiments, the disease is a liver disease. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, administering the compound improves, preserves, or prevents liver damage, steatosis, liver fibrosis, cirrhosis, elevated transaminases, or hepatic fat accumulation in an animal.

In certain embodiments, a method of treating, preventing, or ameliorating liver damage, steatosis, liver fibrosis, liver inflammation, liver scarring or cirrhosis, liver failure, liver enlargement, elevated transaminases, or hepatic fat accumulation in an animal comprises administering to the individual a compound comprising a PNPLA3 specific inhibitor, thereby treating, preventing, or ameliorating liver damage, steatosis, liver fibrosis, liver inflammation, liver scarring or cirrhosis, liver failure, liver enlargement, elevated transaminases, or hepatic fat accumulation. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, administering the compound improves, preserves, or prevents liver damage, steatosis, liver fibrosis, liver inflammation, liver scarring or cirrhosis, liver failure, liver enlargement, elevated transaminases, or hepatic fat accumulation. In certain embodiments, the individual is identified as having, or at risk of having, a disease associated with PNPLA3.

In certain embodiments, a method of inhibiting expression of PNPLA3 in an individual having, or at risk of having, a disease associated with PNPLA3 comprises administering to the individual a compound comprising a PNPLA3 specific inhibitor, thereby inhibiting expression of PNPLA3 in the individual. In certain embodiments, administering the compound inhibits expression of PNPLA3 in the liver. In certain embodiments, the disease is a liver disease. In certain embodiments, the individual has, or is at risk of having, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the individual has, or is at risk of having, liver damage, steatosis, liver fibrosis, liver inflammation, liver scarring or cirrhosis, liver failure, liver enlargement, elevated transaminases, or hepatic fat accumulation. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, administering the compound improves, preserves, or prevents liver damage, steatosis, liver fibrosis, liver inflammation, liver scarring or cirrhosis, liver failure, liver enlargement, elevated transaminases, or hepatic fat accumulation.

In certain embodiments, a method of inhibiting expression of PNPLA3 in a cell comprises contacting the cell with a compound comprising a PNPLA3 specific inhibitor, thereby inhibiting expression of PNPLA3 in the cell. In certain embodiments, the cell is a hepatocyte. In certain embodiments, the cell is in the liver. In certain embodiments, the cell is in the liver of an individual who has, or is at risk of having, liver damage, steatosis, liver fibrosis, liver inflammation, liver scarring or cirrhosis, liver failure, liver enlargement, elevated transaminases, or hepatic fat accumulation. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound.

In certain embodiments, a method of reducing or inhibiting liver damage, steatosis, liver fibrosis, liver inflammation, liver scarring or cirrhosis, liver failure, liver enlargement, elevated transaminases, or hepatic fat accumulation in an individual having, or at risk of having, a disease associated with PNPLA3 comprises administering to the individual a compound comprising a PNPLA3 specific inhibitor, thereby reducing or inhibiting liver damage, steatosis, liver fibrosis, liver inflammation, liver scarring or cirrhosis, liver failure, liver enlargement, elevated transaminases, or hepatic fat accumulation in the individual. In certain embodiments, the individual has, or is at risk of having, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, the individual is identified as having, or at risk of having, a disease associated with PNPLA3.

Certain embodiments are drawn to a compound comprising a PNPLA3 specific inhibitor for use in treating a disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally.

Certain embodiments are drawn to a compound comprising a PNPLA3 specific inhibitor for use in reducing or inhibiting liver damage, steatosis, liver fibrosis, liver inflammation, liver scarring or cirrhosis, liver failure, liver enlargement, elevated transaminases, or hepatic fat accumulation in an individual having, or at risk of having, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound.

Certain embodiments are drawn to the use of a compound comprising a PNPLA3 specific inhibitor for the manufacture or preparation of a medicament for treating a disease associated with PNPLA3. Certain embodiments are drawn to the use of a compound comprising a PNPLA3 specific inhibitor for the preparation of a medicament for treating a disease associated with PNPLA3. In certain embodiments, the disease is a liver disease. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or an oligomeric compound.

Certain embodiments are drawn to the use of a compound comprising a PNPLA3 specific inhibitor for the manufacture or preparation of a medicament for reducing or inhibiting liver damage, steatosis, liver fibrosis, liver inflammation, liver scarring or cirrhosis, liver failure, liver enlargement, elevated transaminases, or hepatic fat accumulation in an individual having, or at risk of having, a liver disease associated with PNPLA3. In certain embodiments, the liver disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. Certain embodiments are drawn to use of a compound comprising a PNPLA3 specific inhibitor for the preparation of a medicament for treating a disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or an oligomeric compound.

In any of the foregoing methods or uses, the compound can be targeted to PNPLA3. In certain embodiments, the compound comprises or consists of a modified oligonucleotide, for example, a modified oligonucleotide 8 to 80 linked nucleosides in length, 10 to 30 linked nucleosides in length, 12 to 30 linked nucleosides in length, or 20 linked nucleosides in length. In certain embodiments, the modified oligonucleotide is at least 80%, at least 85%, at least 90%, at least 95% or 100% complementary to any of the nucleobase sequences recited in SEQ ID NOs: 1-10. In certain embodiments, the modified oligonucleotide comprises at least one modified internucleoside linkage, at least one modified sugar and/or at least one modified nucleobase.

In certain embodiments, the modified internucleoside linkage is a phosphorothioate internucleoside linkage, the modified sugar is a bicyclic sugar or a 2′-O-methoxyethyl modified sugar, and the modified nucleobase is a 5-methylcytosine. In certain embodiments, the modified oligonucleotide comprises a gap segment consisting of linked deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In any of the foregoing embodiments, the modified oligonucleotide is 12 to 30, 15 to 30, 15 to 25, 15 to 24, 16 to 24, 17 to 24, 18 to 24, 19 to 24, 20 to 24, 19 to 22, 20 to 22, 16 to 20, or 16 or 20 linked nucleosides in length. In certain embodiments, the modified oligonucleotide is at least 80%, at least 85%, at least 90%, at least 95% or 100% complementary to any of the nucleobase sequences recited in SEQ ID NOs: 1-10.

In any of the foregoing methods or uses, the compound comprises or consists of a modified oligonucleotide 16 to 30 linked nucleosides in length and having a nucleobase sequence comprising any one of SEQ ID NOs: 17-2169, wherein the modified oligonucleotide comprises:
a gap segment consisting of linked 2′-deoxynucleosides;
a 5′ wing segment consisting of linked nucleosides; and
a 3′ wing segment consisting of linked nucleosides;
   wherein the gap segment is positioned between the 5′ wing segment and the 3′ wing segment and wherein each nucleoside of each wing segment comprises a modified sugar.

In any of the foregoing methods or uses, the compound comprises or consists a modified oligonucleotide 16 linked nucleosides in length having a nucleobase sequence comprising the sequence recited in any one of SEQ ID NOs: 1089, 1757, 141, 1982, 330, 1665, 408, 830, and 899, wherein the modified oligonucleotide comprises
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine. In certain embodiments, the modified oligonucleotide is 16-30 linked nucleosides in length.

In certain embodiments, a compound comprises or consists of ION 916333 or salt thereof, having the following chemical structure (SEQ ID NO: 2173):

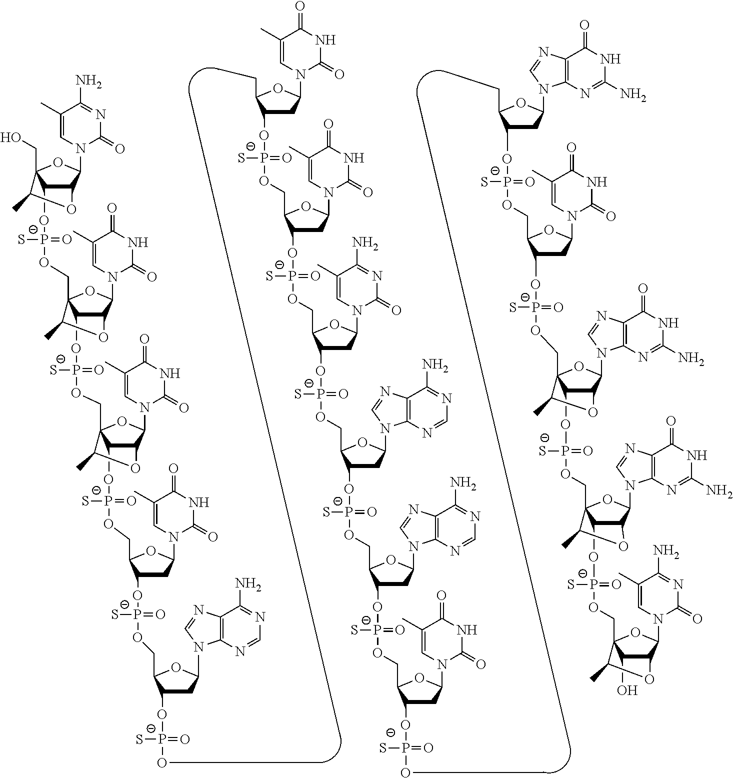

In certain embodiments, a compound comprises or consists of ION 975616 or salt thereof, having the following chemical structure (SEQ ID NO: 2174):

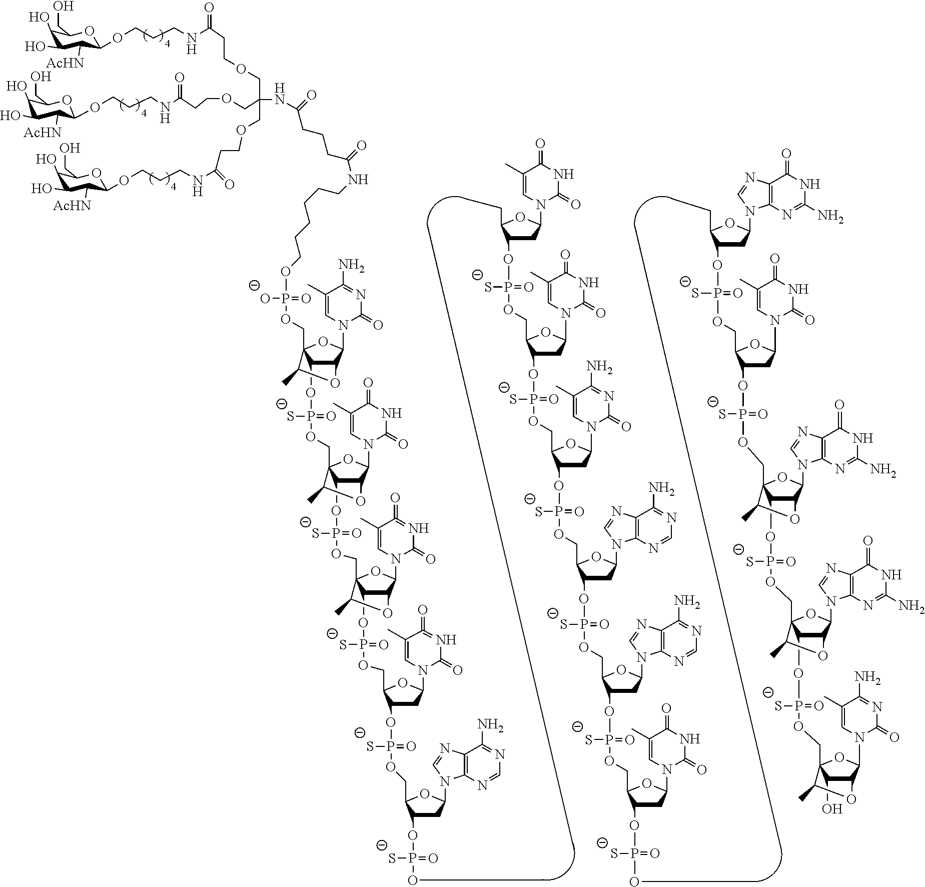

In certain embodiments, a compound comprises or consists of the sodium salt of 975616, having the following chemical structure (SEQ ID NO: 2174):

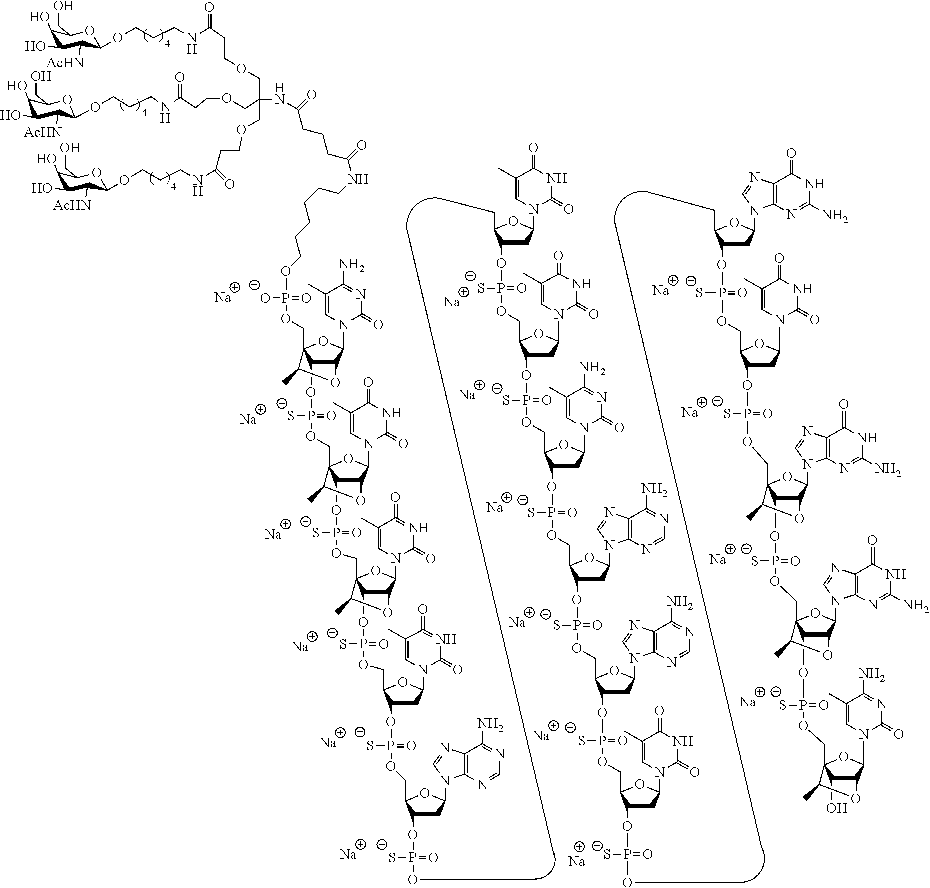

In certain embodiments, a compound comprises or consists of ION 975613 or salt thereof, having the following chemical structure (SEQ ID NO: 2176):

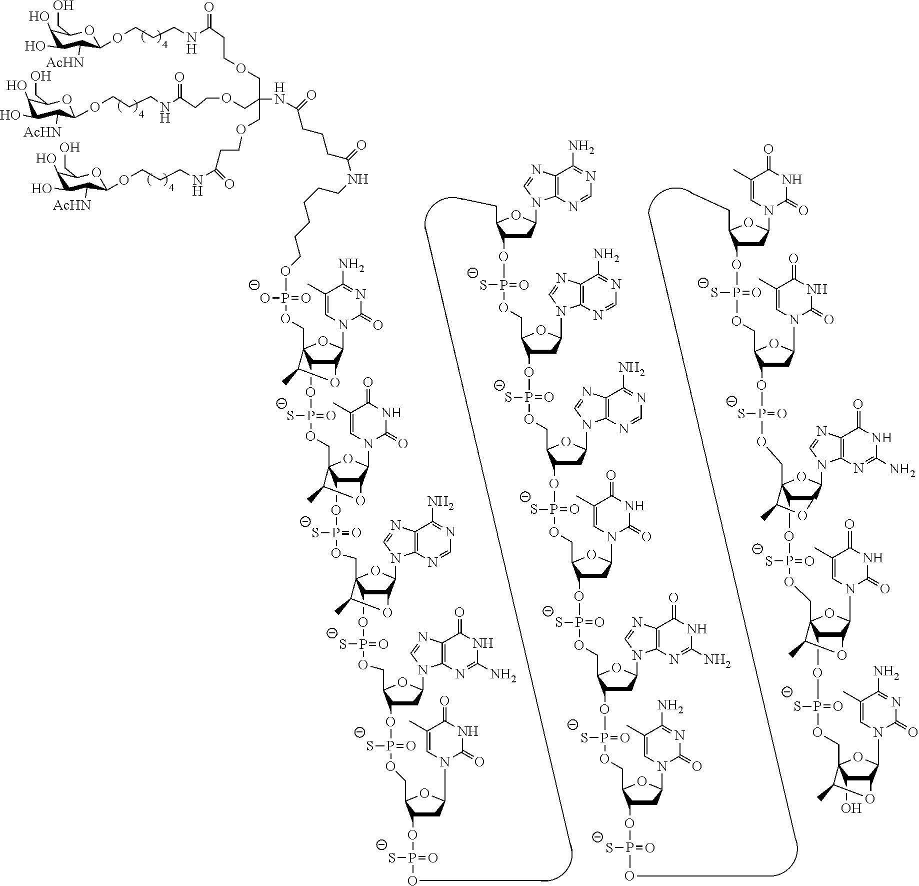

In certain embodiments, a compound comprises or consists of the sodium salt of 975613, having the following chemical structure (SEQ ID NO: 2176):

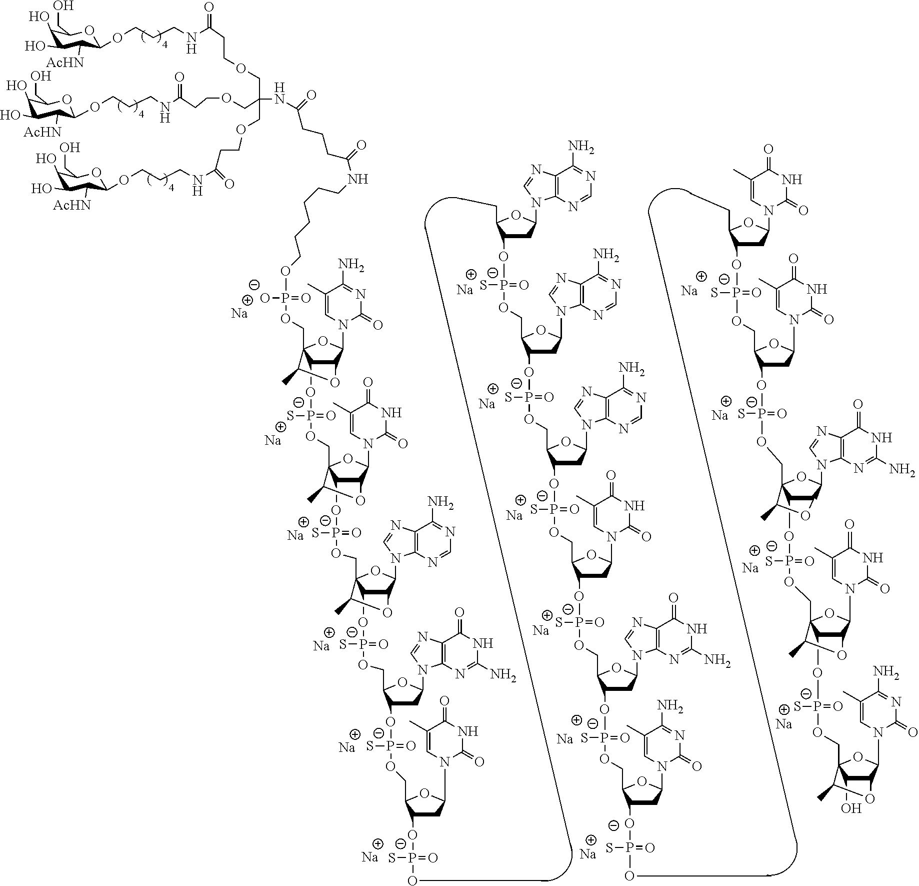

In certain embodiments, a compound comprises or consists of ION 975612 or salt thereof, having the following chemical structure (SEQ ID NO: 2178):

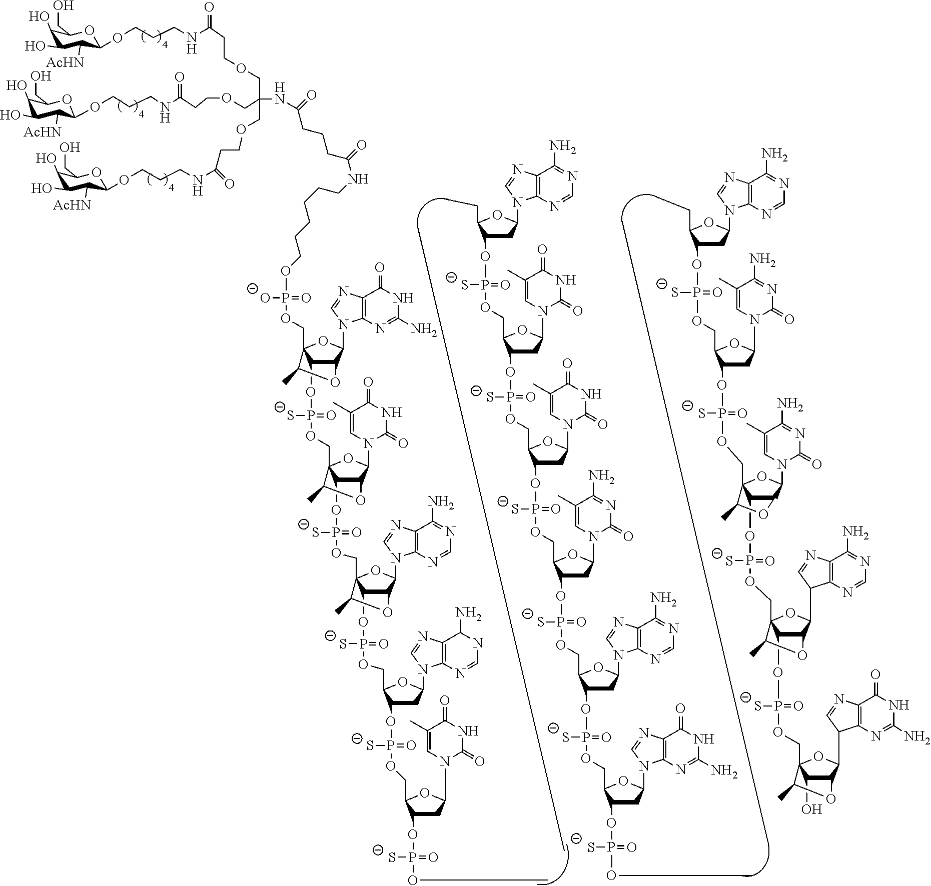

In certain embodiments, a compound comprises or consists of the sodium salt of 975612, having the following chemical structure (SEQ ID NO: 2178):

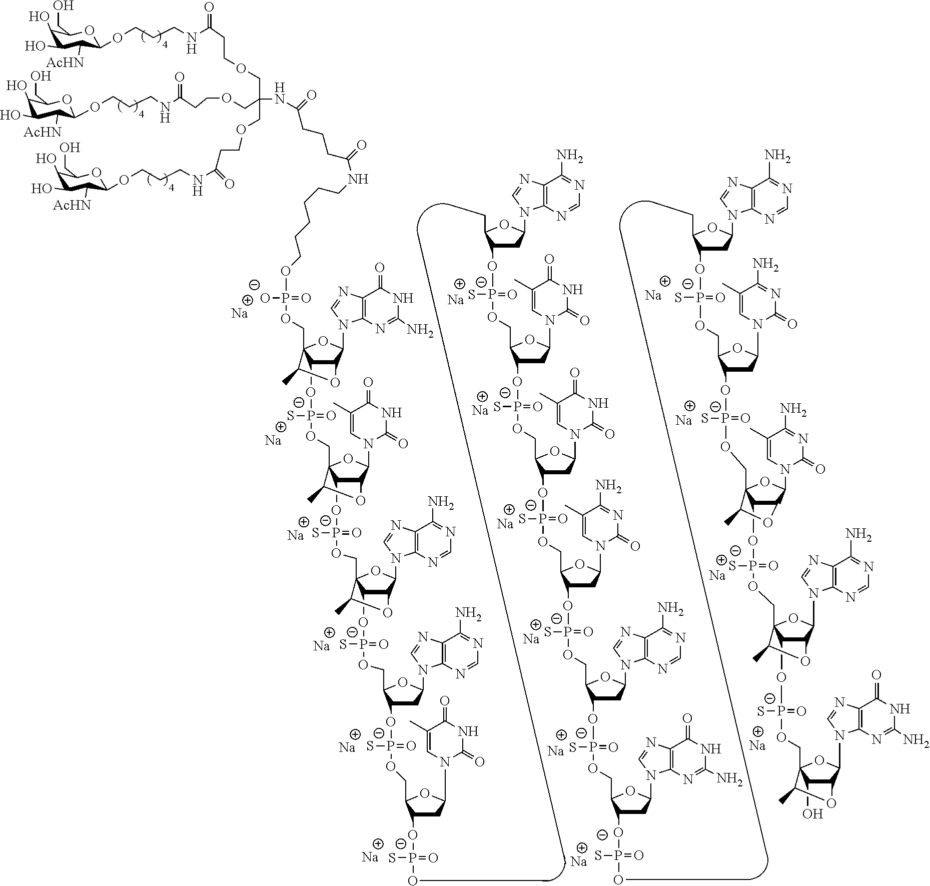

In certain embodiments, a compound comprises or consists of ION 916789 or salt thereof, having the following chemical structure (SEQ ID NO: 2177):

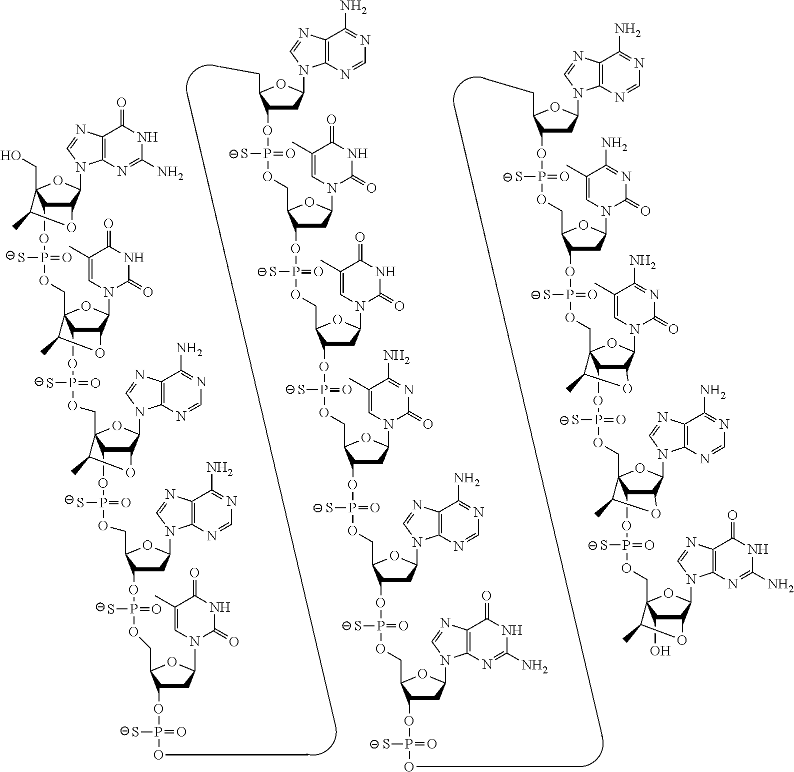

In certain embodiments, a compound comprises or consists of the sodium salt of 916789, having the following chemical structure (SEQ ID NO: 2177):

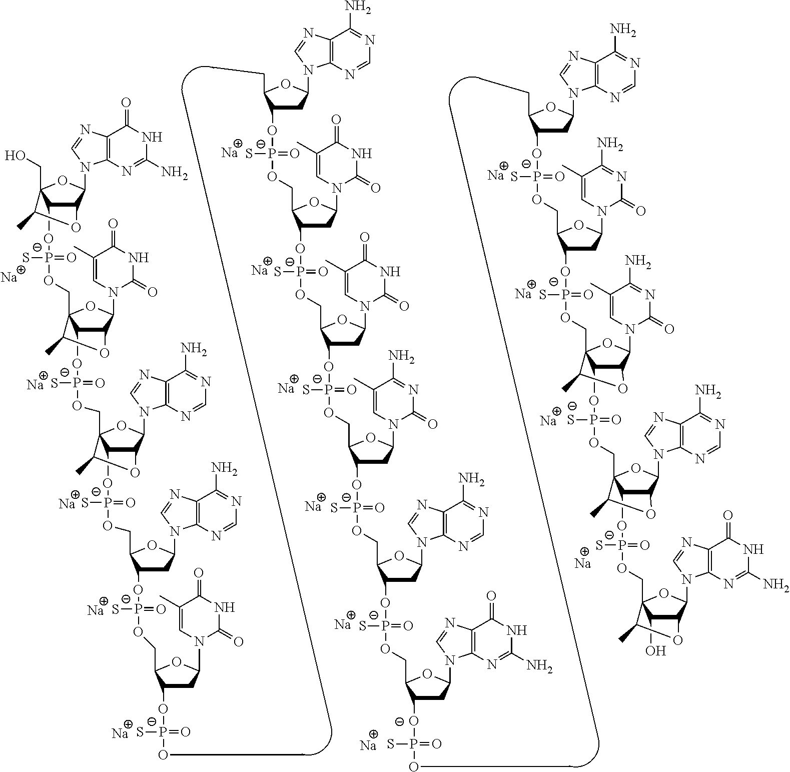

In certain embodiments, a compound comprises or consists of ION 916602 or salt thereof, having the following chemical structure (SEQ ID NO: 2175):

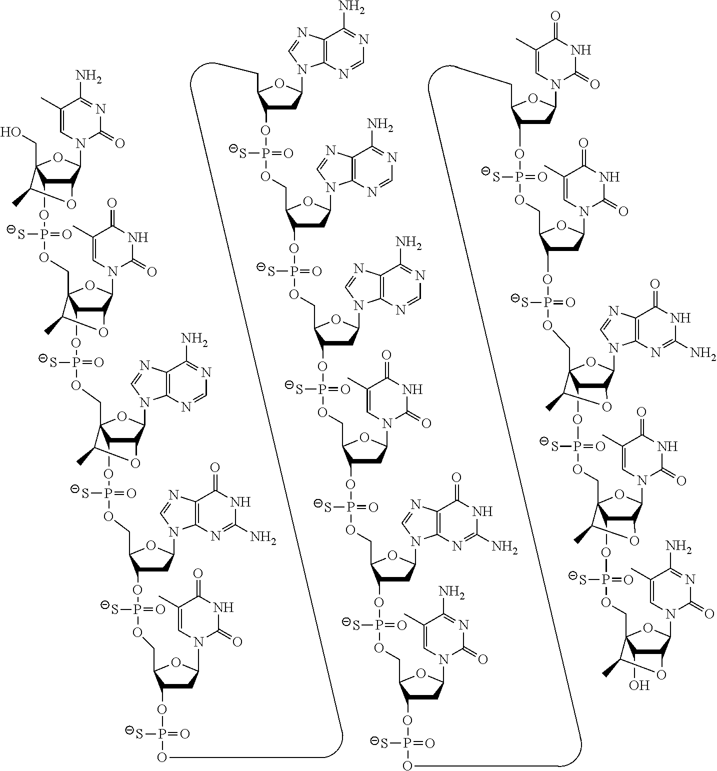

In certain embodiments, a compound comprises or consists of the sodium salt of 916602, having the following chemical structure (SEQ ID NO: 2175):

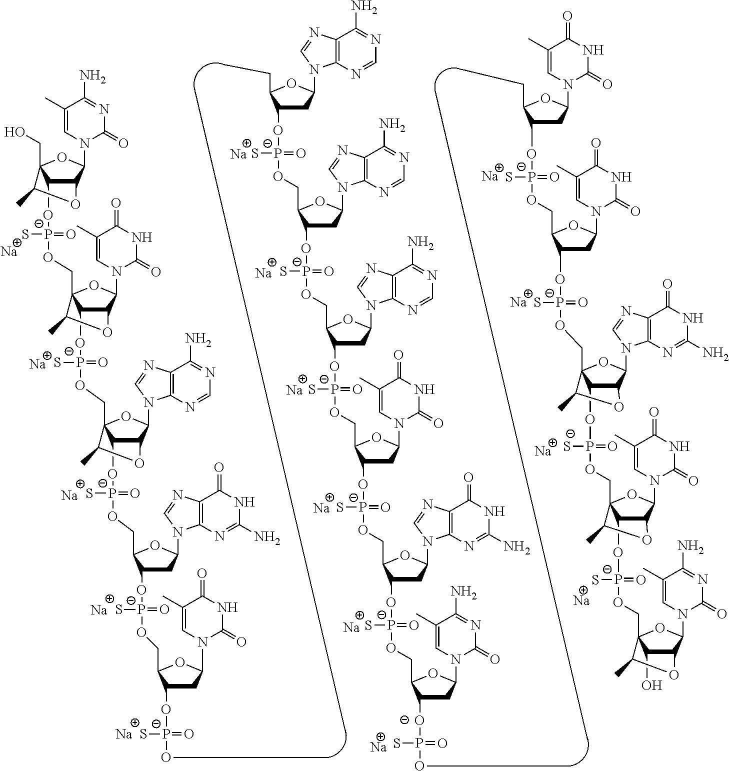

In any of the foregoing methods or uses, the compound can be administered parenterally. For example, in certain embodiments the compound can be administered through injection or infusion. Parenteral administration includes subcutaneous administration, intravenous administration, intramuscular administration, intraarterial administration, intraperitoneal administration, or intracranial administration, e.g. intrathecal or intracerebroventricular administration.

### Certain Compounds

In certain embodiments, compounds described herein can be antisense compounds. In certain embodiments, the antisense compound comprises or consists of an oligomeric compound. In certain embodiments, the oligomeric compound comprises a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide and optionally a conjugate group. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, compounds are double-stranded. Such double-stranded compounds comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, compound comprises a conjugate group. In certain embodiments, one of the modified oligonucleotides is conjugated. In certain embodiments, both the modified oligonucleotides are conjugated. In certain embodiments, the first modified oligonucleotide is conjugated. In certain embodiments, the second modified oligonucleotide is conjugated. In certain embodiments, the first modified oligonucleotide is 16-30 linked nucleosides in length and the second modified oligonucleotide is 16-30 linked nucleosides in length. In certain embodiments, one of the modified oligonucleotides has a nucleobase sequence comprising at least 8 contiguous nucleobases of any of SEQ ID NOs: 17-2169.

In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such a double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

Examples of single-stranded and double-stranded compounds include, but are not limited to, oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 12 to 30 linked subunits in length. In certain embodiments, a compound described herein comprises an oligonucleotide 12 to 22 linked subunits in length. In certain embodiments, compound described herein comprises an oligonucleotide 14 to 30 linked subunits in length. In certain embodiments, compound described herein comprises an oligonucleotide 14 to 20 linked subunits in length. In certain embodiments, a compound described herein comprises an oligonucleotide 15 to 30 linked subunits in length. In certain embodiments, a compound described herein comprises an oligonucleotide 15 to 20 linked subunits in length. In certain embodiments, a compound described herein comprises an oligonucleotide 16 to 30 linked subunits in length. In certain embodiments, a compound described herein comprises an oligonucleotide 16 to 20 linked subunits in length. In certain embodiments, a compound described herein comprises an oligonucleotide 17 to 30 linked subunits in length. In certain embodiments, a compound described herein comprises an oligonucleotide 17 to 20 linked subunits in length. In certain embodiments, a compound described herein comprises an oligonucleotide 18 to 30 linked subunits in length. In certain embodiments, a compound described herein comprises an oligonucleotide 18 to 20 linked subunits in length. In certain embodiments, a compound described herein comprises an oligonucleotide 20 to 30 linked subunits in length. In other words, such oligonucleotides are 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, or 20 to 30 subunits in length, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, or 30 linked subunits in length, or a range defined by any two of the above values. In some embodiments the linked subunits are nucleotides, nucleosides, or nucleobases.

In certain embodiments, the compound may further comprise additional features or elements, such as a conjugate group, that are attached to the oligonucleotide. In certain embodiments, such compounds are antisense compounds. In certain embodiments, such compounds are oligomeric compounds. In embodiments where a conjugate group comprises a nucleoside (i.e. a nucleoside that links the conjugate group to the oligonucleotide), the nucleoside of the conjugate group is not counted in the length of the oligonucleotide.

In certain embodiments, compounds may be shortened or truncated. For example, a single subunit may be deleted from the 5′ end (5′ truncation), or alternatively from the 3′ end (3′ truncation). A shortened or truncated compound targeted to a PNPLA3 nucleic acid may have two subunits deleted from the 5′ end, or alternatively, may have two subunits deleted from the 3′ end of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively, to the 3′ end (3′ addition) of the compound. Alternatively, the added subunits may be dispersed throughout the compound.

It is possible to increase or decrease the length of a compound, such as an oligonucleotide, and/or introduce mismatch bases without eliminating activity (Woolf et al. Proc. Natl. Acad. Sci. USA 1992, 89:7305-7309; Gautschi et al. J. Natl. Cancer Inst. March 2001, 93:463-471; Maher and Dolnick Nuc. Acid. Res. 1998, 16:3341-3358). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence-specific RNAi, for example, short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term “RNAi” is meant to be equivalent to other terms used to describe sequence-specific RNA interference, such as post transcriptional gene silencing, translational inhibition, or epigenetics.

In certain embodiments, a compound described herein can comprise any of the oligonucleotide sequences targeted to PNPLA3 described herein. In certain embodiments, the compound can be double-stranded. In certain embodiments, the compound comprises a first strand comprising at least an 8, 9, 10, 11, 12, 13, 14, 15, or 16 contiguous nucleobase portion of any one of SEQ ID NOs: 17-2169 and a second strand. In certain embodiments, the compound comprises a first strand comprising the nucleobase sequence of any one of SEQ ID NOs: 17-2169 and a second strand. In certain embodiments, the compound comprises ribonucleotides in which the first strand has uracil (U) in place of thymine (T) in any one of SEQ ID NOs: 17-2169. In certain embodiments, the compound comprises (i) a first strand comprising a nucleobase sequence complementary to the site on PNPLA3 to which any of SEQ ID NOs: 17-2169 is targeted, and (ii) a second strand. In certain embodiments, the compound comprises one or more modified nucleotides in which the 2′ position of the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains one or two capped strands, as disclosed, for example, by WO 00/63364, filed Apr. 19, 2000.

In certain embodiments, the first strand of the compound is an siRNA guide strand and the second strand of the compound is an siRNA passenger strand. In certain embodiments, the second strand of the compound is complementary to the first strand. In certain embodiments, each strand of the compound is 16, 17, 18, 19, 20, 21, 22, or 23 linked nucleosides in length. In certain embodiments, the first or second strand of the compound can comprise a conjugate group.

In certain embodiments, a compound described herein can comprise any of the oligonucleotide sequences targeted to PNPLA3 described herein. In certain embodiments, the compound is single stranded. In certain embodiments, such a compound is a single-stranded RNAi (ssRNAi) compound. In certain embodiments, the compound comprises at least an 8, 9, 10, 11, 12, 13, 14, 15, or 16 contiguous nucleobase portion of any one of SEQ ID NOs: 17-2169. In certain embodiments, the compound comprises the nucleobase sequence of any one of SEQ ID NOs: 17-2169. In certain embodiments, the compound comprises ribonucleotides in which uracil (U) is in place of thymine (T) in any one of SEQ ID NOs: 17-2169. In certain embodiments, the compound comprises a nucleobase sequence complementary to the site on PNPLA3 to which any of SEQ ID NOs: 17-2169 is targeted. In certain embodiments, the compound comprises one or more modified nucleotides in which the 2′ position in the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains a capped strand, as disclosed, for example, by WO 00/63364, filed Apr. 19, 2000. In certain embodiments, the compound consists of 16, 17, 18, 19, 20, 21, 22, or 23 linked nucleosides. In certain embodiments, the compound can comprise a conjugate group.

### Certain Mechanisms

In certain embodiments, compounds described herein comprise or consist of modified oligonucleotides. In certain embodiments, compounds described herein are antisense compounds. In certain embodiments, compounds comprise oligomeric compounds. In certain embodiments, compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

Nucleotide sequences that encode PNPLA3 include, without limitation, the following: RefSeq or GENBANK Accession Nos. NM_025225.2 (incorporated by reference, disclosed herein as SEQ ID NO: 1); GENBANK Accession No. NC_000022.11 truncated from nucleotides 43921001 to U.S. Pat. No. 43,954,500 (incorporated by reference, disclosed herein as SEQ ID NO: 2); AK123806.1 (incorporated by reference, disclosed herein as SEQ ID NO: 3); BQ686328.1 (incorporated by reference, disclosed herein as SEQ ID NO: 4); BF762711.1 (incorporated by reference, disclosed herein as SEQ ID NO: 5); DA290491.1 (incorporated by reference, disclosed herein as SEQ ID NO: 6); and the sequences listed as SEQ ID Nos. 7, 8, 9, and 10.

### Hybridization

In some embodiments, hybridization occurs between a compound disclosed herein and a PNPLA3 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with a PNPLA3 nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (mC) and guanine (G), unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a PNPLA3 nucleic acid may be tolerated provided that the compound remains able to specifically hybridize to a target nucleic acid. Moreover, a compound may hybridize over one or more segments of a PNPLA3 nucleic acid such that intervening or adjacent segments are not involved in the hybridization event (e.g., a loop structure, mismatch or hairpin structure).

In certain embodiments, the compounds provided herein, or a specified portion thereof are at least, or are up to 70%, 80%, 85%, 86%, 87%, 88%, 89%, 90%, 91%, 92%, 93%, 94%, 95%, 96%, 97%, 98%, 99%, or 100% complementary to a PNPLA3 nucleic acid, a target region, target segment, or specified portion thereof. In certain embodiments, the compounds provided herein, or a specified portion thereof, are 70% to 75%, 75% to 80%, 80% to 85%, 85% to 90%, 90% to 95%, 95% to 100%, or any number in between these ranges, complementary to a PNPLA3 nucleic acid, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid. Percent complementarity of a compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be fully complementary to a PNPLA3 nucleic acid, or a target region, or a target segment or target sequence thereof. As used herein, “fully complementary” means each nucleobase of a compound is complementary to the corresponding nucleobase of a target nucleic acid. For example, a 20 nucleobase compound is fully complementary to a target sequence that is 400 nucleobases long, so long as there is a corresponding 20 nucleobase portion of the target nucleic acid that is fully complementary to the compound. “Fully complementary” can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments, selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a PNPLA3 nucleic acid, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a PNPLA3 nucleic acid, or specified portion thereof.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the compounds, are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 17, 18, 19, 20, or more nucleobase portion of a target segment, or a range defined by any two of these values.

### Identity

The compounds provided herein may also have a defined percent identity to a particular nucleotide sequence, SEQ ID NO, or compound represented by a specific ION number, or portion thereof. In certain embodiments, compounds described herein are antisense compounds or oligomeric compounds. In certain embodiments, compounds described herein are modified oligonucleotides. As used herein, a compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of an compound is calculated according to the number of bases that have identical base pairing relative to the sequence to which it is being compared.

In certain embodiments, compounds described herein, or portions thereof, are, or are at least, 70%, 75%, 80%, 85%, 90%, 91%, 92%, 93%, 94%, 95%, 96%, 97%, 98%, 99% or 100% identical to one or more of the compounds or SEQ ID NOs, or a portion thereof, disclosed herein. In certain embodiments, compounds described herein are about 70%, 75%, 80%, 85%, 90%, 91%, 92%, 93%, 94%, 95%, 96%, 97%, 98%, or 99% identical, or any percentage between such values, to a particular nucleotide sequence, SEQ ID NO, or compound represented by a specific ION number, or portion thereof, in which the compounds comprise an oligonucleotide having one or more mismatched nucleobases. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

In certain embodiments, compounds described herein comprise or consist of antisense compounds. In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, compounds described herein comprise or consist of oligonucleotides. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Modified Nucleosides

Modified nucleosides comprise a modified sugar moiety or a modified nucleobase or both a modified sugar moiety and a modified nucleobase.

1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited, to substituents at the 2′, 4′, and/or 5′ positions. In certain embodiments, the furanosyl sugar moiety is a ribosyl sugar moiety. In certain embodiments, one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearly non-bicyclic modified sugar moieties include, but are not limited to, alkoxy (e.g., methoxy), alkyl, and those described in Manoharan et al., WO 2015/106128. Examples of 5′-substituent groups suitable for non-bicyclic modified sugar moieties include, but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃.

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2′-modified sugar moieties are referred to as 2′-substituted nucleosides or 2′-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and Re is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

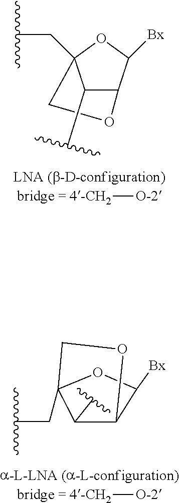

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include, but are not limited to, hexitol nucleic acid (“HNA”), anitol nucleic acid (“ANA”), manitol nucleic acid (“MNA”) (see e.g., Leumann, CJ. Bioorg. & Med. Chem. 2002, 10, 841-854), fluoro HNA:

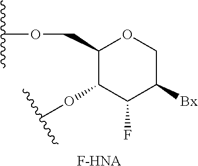

(“F-HNA”, see e.g., Swayze et al., U.S. Pat. No. 8,088,904; Swayze et al., U.S. Pat. No. 8,440,803; and Swayze et al., U.S. Pat. No. 9,005,906) F-HNA can also be referred to as a F-THP or 3′-fluoro tetrahydropyran, and nucleosides comprising additional modified THP compounds having the formula:

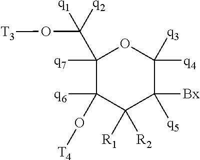

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

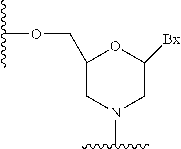

In certain embodiments, morpholinos may be modified, for example, by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378.

Many other bicyclic and tricyclic sugar and sugar surrogate ring systems are known in the art that can be used in modified nucleosides.

2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds.

In certain embodiments, compounds described herein comprise modified oligonucleotides. In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleosides that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

In certain embodiments, compounds targeted to a PNPLA3 nucleic acid comprise one or more modified nucleobases. In certain embodiments, the modified nucleobase is 5-methylcytosine. In certain embodiments, each cytosine is a 5-methylcytosine.

Modified Internucleoside Linkages

The naturally occurring internucleoside linkage of RNA and DNA is a 3′ to 5′ phosphodiester linkage. In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

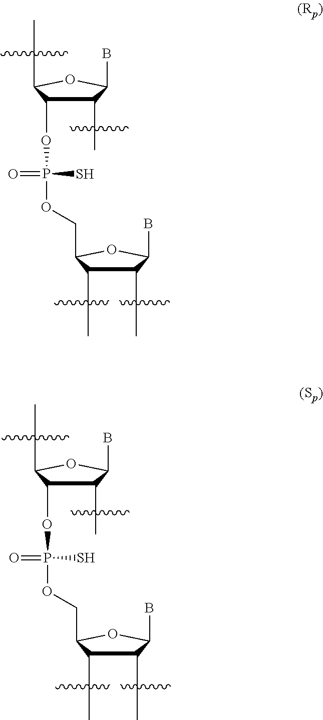

Unless otherwise indicated, chiral internucleoside linkages of modified oligonucleotides described herein can be stereorandom or in a particular stereochemical configuration.

In certain embodiments, compounds targeted to a PNPLA3 nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include, but are not limited to, phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include, but are not limited to, alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments, the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and, if it does have a gapped nucleoside motif, the wing and gap lengths may or may not be the same.

In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, oligonucleotides having a gapmer nucleoside motif comprise a linkage motif comprising all phosphorothioate linkages except for one or two methylphosphonate linkages. In certain embodiments, one methylphosphonate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments, it is desirable to decrease the number of phosphorothioate internucleoside linkages while retaining nuclease resistance. In certain embodiments, it is desirable to increase the number of phosphodiester internucleoside linkages while retaining nuclease resistance.

3. Certain Motifs

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleosides comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

a. Certain Sugar Motifs

In certain embodiments, compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include, but are not limited to, any of the sugar modifications discussed herein.

In certain embodiments, modified oligonucleotides comprise or consist of a region having a gapmer motif, which comprises two external regions or “wings” and a central or internal region or “gap”. The three regions of a gapmer motif (the 5′-wing, the gap, and the 3′-wing) form a contiguous sequence of nucleosides, wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleosides having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiments, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

b. Certain Nucleobase Motifs

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

c. Certain Internucleoside Linkage Motifs

In certain embodiments, compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

4. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are characterized by their modification, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such gapmer oligonucleotides may comprise one or more modified nucleobases independent of the gapmer pattern of the sugar modifications. Furthermore, in certain instances, an oligonucleotide is described by an overall length or range and by lengths or length ranges of two or more regions (e.g., a regions of nucleosides having specified sugar modifications). In such circumstances, it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide will be 22, which exceeds the upper limit of the overall length of the modified oligonucleotide (20). Herein, if a description of an oligonucleotide is silent with respect to one or more parameters, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

### Certain Conjugated Compounds

In certain embodiments, the compounds described herein comprise or consist of an oligonucleotide (modified or unmodified) and, optionally, one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

In certain embodiments, the oligonucleotide is modified. In certain embodiments, the oligonucleotide of a compound has a nucleobase sequence that is complementary to a target nucleic acid. In certain embodiments, oligonucleotides are complementary to a messenger RNA (mRNA). In certain embodiments, oligonucleotides are complementary to a pre-mRNA. In certain embodiments, oligonucleotides are complementary to a sense transcript.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including, but not limited to, pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

2. Conjugate linkers

Conjugate moieties are attached to oligonucleotides through conjugate linkers. In certain embodiments, a conjugate group is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units, such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include, but are not limited to, electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include, but are not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include, but are not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl.

In certain embodiments, conjugate linkers comprise 1-10 linker-nucleosides. In certain embodiments, such linker-nucleosides are modified nucleosides. In certain embodiments, such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, a compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, a compound may comprise a modified oligonucleotide consisting of 8-30 nucleosides and no conjugate group. The total number of contiguous linked nucleosides in such a compound is no more than 30. Unless otherwise indicated, conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances, compounds comprising a particular conjugate moiety are better taken up by a particular cell type, but once the compound has been taken up, it is desirable that the conjugate group be cleaved to release the unconjugated or parent oligonucleotide. Thus, certain conjugates may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

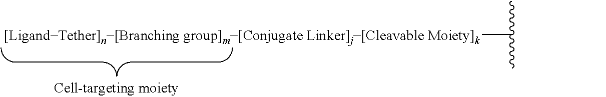

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether, and hydroxylamino groups. In certain embodiments, the branching group comprises a branched aliphatic group comprising groups selected from alkyl, amino, oxo, amide, disulfide, polyethylene glycol, ether, thioether, and hydroxylamino groups. In certain such embodiments, the branched aliphatic group comprises groups selected from alkyl, amino, oxo, amide, and ether groups. In certain such embodiments, the branched aliphatic group comprises groups selected from alkyl, amino, and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amide, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine, and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

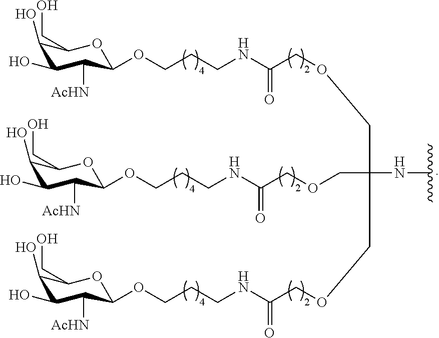

In certain embodiments, conjugate groups comprise a cell-targeting moiety having the formula:

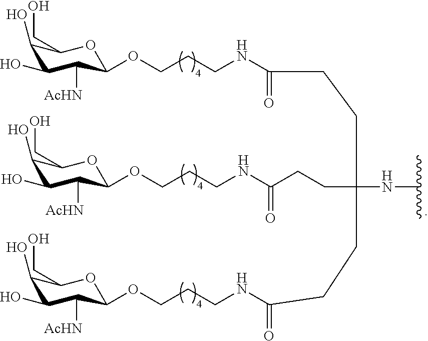

In certain embodiments, conjugate groups comprise a cell-targeting moiety having the formula:

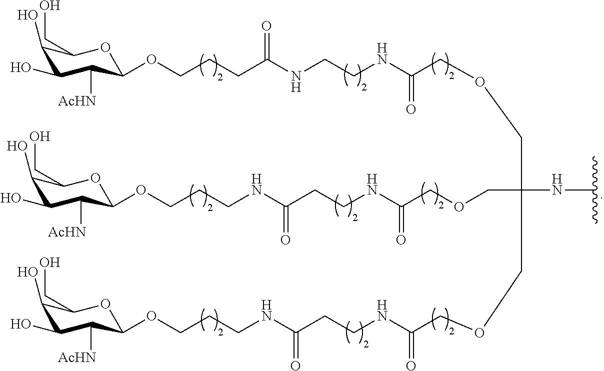

In certain embodiments, compounds described herein comprise a conjugate group described herein as “LICA-1”. LICA-1 is shown below without the optional cleavable moiety at the end of the conjugate linker:

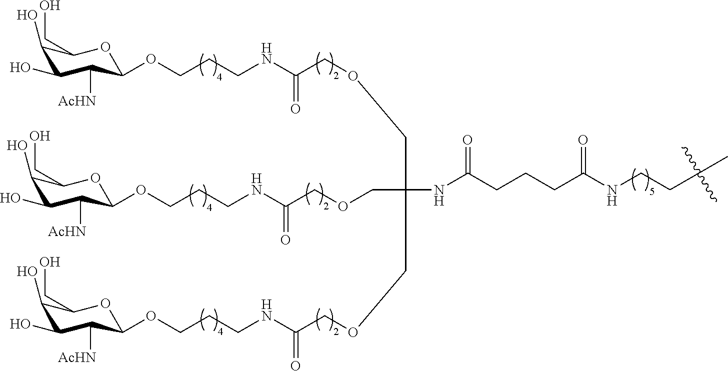

In certain embodiments, compounds described herein comprise LICA-1 and a cleavable moiety within the conjugate linker have the formula:

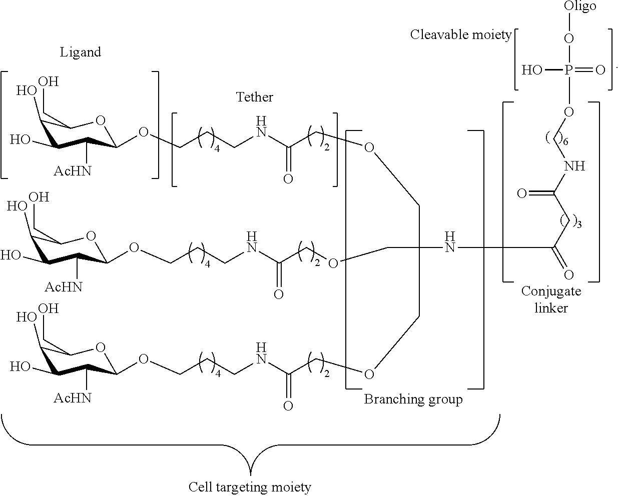

wherein ‘oligo’ is an oligonucleotide.

Representative publications that teach the preparation of certain of the above noted conjugate groups and compounds comprising conjugate groups, tethers, conjugate linkers, branching groups, ligands, cleavable moieties as well as other modifications include, without limitation, U.S. Pat. Nos. 5,994,517, 6,300,319, 6,660,720, 6,906,182, 7,262,177, 7,491,805, 8,106,022, 7,723,509, 9,127,276, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011, 19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770, each of which is incorporated by reference herein in its entirety.

In certain embodiments, compounds described herein comprise modified oligonucleotides comprising a gapmer or fully modified motif and a conjugate group comprising at least one, two, or three GalNAc ligands. In certain embodiments, compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Korilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132; each of which is incorporated by reference in its entirety.

### Compositions and Methods for Formulating Pharmaceutical Compositions

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more compounds or a salt thereof. In certain embodiments, the compounds are antisense compounds or oligomeric compounds. In certain embodiments, the compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more compound. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compounds and phosphate-buffered saline (PBS). In certain embodiments, a pharmaceutical composition consists of one or more compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

A compound described herein targeted to PNPLA3 nucleic acid can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to PNPLA3 nucleic acid and a pharmaceutically acceptable diluent. In certain embodiments, the pharmaceutically acceptable diluent is water. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound.

In certain embodiments, the compounds or compositions further comprise a pharmaceutically acceptable carrier or diluent.

### Certain Selected Compounds

Approximately 2,384 newly designed compounds of various lengths, chemistries, and motifs were tested for their effect on human PNPLA3 mRNA in vitro in several cell types (Example 1). Of 2,384 compounds tested for potency at a single dose in vitro, over 400 selected compounds were tested for dose dependent inhibition in A431 cells (Example 2). Of the over 400 compounds tested by dose response assays, the compounds were further screened for high dose tolerability in a BALB/c mouse model and 87 oligonucleotides were selected for in vivo efficacy in a PNPLA3 transgenic mouse model.

Of the 87 oligonucleotides tested in the transgenic mouse model, 23 oligonucleotides were selected to be further tested for tolerability in preclinical rodel models. In the in vivo rodent tolerability models, body weights and organ weights, liver function markers (such as alanine transaminase, aspartate transaminase and bilirubin), and kidney function markers (such as BUN and creatinine) were measured. In the CD1 mouse model and in the Sprague-Dawley rat model, ION 975591, 975605, 975612, 975613, 975616, 975617, 975735, 975736, 994282, and 994284 were found tolerable (Examples 5 and 6).

These compounds were further tested for efficacy in multi-dose assays in PNPLA3 transgenic mice (Example 7).

IONs 994284, 97605, 975616, 994282, 975613, 975617, 975735, 975736, and 975612 were tested for tolerability in cynomolgus monkeys (Example 8). Treatment with the compounds was well tolerated in the monkeys.

Accordingly, provided herein are compounds with any one or more of the improved properties. In certain embodiments, the compounds as described herein are potent and tolerable.

### EXAMPLES

The Examples below describe the screening process to identify lead compounds targeted to PNPLA3. ION 994284, 97605, 975616, 994282, 975613, 975617, 975735, 975736, and 975612 resulted in high potency and tolerability.

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to, those in the sequence listing, are intended to encompass nucleic acids containing any combination of natural or modified RNA and/or DNA, including, but not limited to, such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “ATmCGAUCG,” wherein ^{m}C indicates a cytosine base comprising a methyl group at the 5-position.

Certain compounds described herein (e.g. modified oligonucleotides) have one or more asymmetric centers and thus give rise to enantiomers, diastereomers, and other stereoisomeric configurations that may be defined, in terms of absolute stereochemistry, as (R) or (S), as a or R, such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. Likewise, all tautomeric forms of the compounds provided herein are included unless otherwise indicated. Unless otherwise indicated, oligomeric compounds and modified oligonucleotides described herein are intended to include corresponding salt forms.

Compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include, but are not limited to: 2H or ³H in place of ¹H, ¹³C or ¹⁴C in place of ¹²C, ¹⁵N in place of ¹⁴N, ¹⁷O or ¹⁸O in place of 160, and 33S, 34S, 35S, or 36S in place of 32.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Human PNPLA3 in A431 Cells

Antisense oligonucleotides were designed targeting a PNPLA3 nucleic acid and were tested for their effects on PNPLA3 mRNA in vitro. The antisense oligonucleotides were tested in a series of experiments that had similar culture conditions. The results for each experiment are presented in separate tables shown below.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human PNPLA3 mRNA, designated herein as SEQ ID NO: 1 (GENBANK Accession No. NM_025225.2) or the human PNPLA3 genomic sequence, designated herein as SEQ ID NO: 2 (GENBANK Accession No. NC_000022.11 truncated from nucleotides 43921001 to 43954500). ‘n/a’ indicates that the antisense oligonucleotide does not target that particular gene sequence with 100% complementarity.

### Study 1

Cultured A431 cells at a density of 20,000 cells per well were transfected by free uptake with 4,000 nM antisense oligonucleotide. After a treatment period of approximately 24 hours, RNA was isolated from the cells and PNPLA3 mRNA levels were measured by quantitative real-time PCR. Human primer probe set RTS36070 (forward sequence CCTTGGTATGTTCCTGCTTCA, designated herein as SEQ ID NO: 11; reverse sequence GTTGTCACTCACTCCTCCATC, designated herein as SEQ ID NO: 12; probe sequence TGGCCTTATCCCTCCTTCCTTCAGA, designated herein as SEQ ID NO: 13) was used to measure mRNA levels. PNPLA3 mRNA levels were adjusted according to total RNA content, as measured by RIBOGREEN®. Results are presented as percent inhibition of PNPLA3, relative to untreated control cells.

TABLE 1
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912709 | 27 | 42 | 2765 | 2780 | GGCATTCCCAGCGCGA | 0 | 17 |
| 912710 | 95 | 110 | 2833 | 2848 | TCCTGATCCGCAGCAG | 15 | 18 |
| 912711 | 103 | 118 | 2841 | 2856 | GGCTCGGGTCCTGATC | 0 | 19 |
| 912712 | 131 | 146 | 2869 | 2884 | GTTAGGATCTGGGTCG | 91 | 20 |
| 912713 | 164 | 179 | 2902 | 2917 | GTACATGGCGGCGGCG | 0 | 21 |
| 912714 | 183 | 198 | 2921 | 2936 | TCCAGCCGCGCTCTGC | 23 | 22 |
| 912715 | 196 | 211 | 2934 | 2949 | GCGAAGGACAAGCTCC | 60 | 23 |
| 912716 | 197 | 212 | 2935 | 2950 | CGCGAAGGACAAGCTC | 0 | 24 |
| 912717 | 272 | 287 | 3010 | 3025 | GCGGAGGAGGTGCGGG | 0 | 25 |
| 912718 | 273 | 288 | 3011 | 3026 | CGCGGAGGAGGTGCGG | 0 | 26 |
| 912719 | 274 | 289 | 3012 | 3027 | TCGCGGAGGAGGTGCG | 19 | 27 |
| 912720 | 290 | 305 | 3028 | 3043 | GAACAACATGCGCGCG | 0 | 28 |
| 912721 | 291 | 306 | 3029 | 3044 | CGAACAACATGCGCGC | 7 | 29 |
| 912722 | 292 | 307 | 3030 | 3045 | CCGAACAACATGCGCG | 21 | 30 |
| 912723 | 293 | 308 | 3031 | 3046 | GCCGAACAACATGCGC | 0 | 31 |
| 912724 | 294 | 309 | 3032 | 3047 | CGCCGAACAACATGCG | 0 | 32 |
| 912725 | 323 | 338 | 3061 | 3076 | GCCGACGCAGTGCAAC | 0 | 33 |
| 912726 | 324 | 339 | 3062 | 3077 | CGCCGACGCAGTGCAA | 0 | 34 |
| 912727 | 340 | 355 | 3078 | 3093 | GGGATACCGGAGAGGA | 43 | 35 |
| 912728 | 370 | 385 | 5944 | 5959 | TCTGAGAGGACCTGCA | 53 | 36 |
| 912729 | 375 | 390 | 5949 | 5964 | CAAGATCTGAGAGGAC | 64 | 37 |
| 912730 | 404 | 419 | 5978 | 5993 | GCCAATGTTCCGACTC | 71 | 38 |
| 912731 | 410 | 425 | 5984 | 5999 | GAAGATGCCAATGTTC | 51 | 39 |
| 912732 | 429 | 444 | 6003 | 6018 | TTAAGTTGAAGGATGG | 96 | 40 |
| 912733 | 432 | 447 | 6006 | 6021 | TGCTTAAGTTGAAGGA | 90 | 41 |
| 912734 | 478 | 493 | 6052 | 6067 | TGGACATTGGCCGGGA | 85 | 42 |
| 912735 | 479 | 494 | 6053 | 6068 | GTGGACATTGGCCGGG | 50 | 43 |
| 912736 | 484 | 499 | 6058 | 6073 | AGCTGGTGGACATTGG | 64 | 44 |
| 912737 | 528 | 543 | 6102 | 6117 | CATCAGACACTCTGGT | 5 | 45 |
| 912738 | 531 | 546 | 6105 | 6120 | CCCCATCAGACACTCT | 73 | 46 |
| 912739 | 552 | 567 | 6126 | 6141 | AGTCAGACACCAGAAC | 54 | 47 |
| 912755 | 693 | 708 | 11911 | 11926 | TGGCATCAATGAAGGG | 74 | 48 |
| 912756 | 698 | 713 | 11916 | 11931 | TGTTTTGGCATCAATG | 91 | 49 |
| 912757 | 746 | 761 | 11964 | 11979 | TTTAGGGCAGATGTCG | 89 | 50 |
| 912758 | 747 | 762 | 11965 | 11980 | CTTTAGGGCAGATGTC | 90 | 51 |
| 912759 | 795 | 810 | 12013 | 12028 | GTAGACTGAGCTTGGT | 98 | 52 |
| 912760 | 820 | 835 | 12038 | 12053 | AGGTAGAGGTTCCCTG | 0 | 53 |
| 912761 | 841 | 856 | 12059 | 12074 | GGGACAAAAGCTCTCG | 20 | 54 |
| 912762 | 873 | 888 | 13609 | 13624 | GGCATATCTCTCCCAG | 0 | 55 |
| 912763 | 874 | 889 | 13610 | 13625 | AGGCATATCTCTCCCA | 0 | 56 |
| 912764 | 886 | 901 | 13622 | 13637 | AAATATCCTCGAAGGC | 57 | 57 |
| 912765 | 888 | 903 | 13624 | 13639 | CCAAATATCCTCGAAG | 30 | 58 |
| 912766 | 889 | 904 | 13625 | 13640 | TCCAAATATCCTCGAA | 38 | 59 |
| 912767 | 894 | 909 | 13630 | 13645 | ATGCATCCAAATATCC | 58 | 60 |
| 912768 | 925 | 940 | N/A | N/A | TTGCAGATGCCCTTCT | 15 | 61 |
| 912769 | 968 | 983 | 16088 | 16103 | ATCCATCCCTTCTGAG | 34 | 62 |
| 912770 | 986 | 1001 | 16106 | 16121 | GGGCATGGCGACCTCA | 0 | 63 |
| 912771 | 1004 | 1019 | 16124 | 16139 | ACTCATGTTTGCCCAG | 67 | 64 |
| 912772 | 1068 | 1083 | 16188 | 16203 | GGTCTAGCAGCTCATC | 89 | 65 |
| 912773 | 1075 | 1090 | 16195 | 16210 | CGCAGGTGGTCTAGCA | 0 | 66 |
| 912774 | 1076 | 1091 | 16196 | 16211 | ACGCAGGTGGTCTAGC | 25 | 67 |
| 912775 | 1080 | 1095 | 16200 | 16215 | TGAGACGCAGGTGGTC | 50 | 68 |
| 912776 | 1086 | 1101 | 16206 | 16221 | GGATGCTGAGACGCAG | 67 | 69 |
| 912777 | 1172 | 1187 | 19012 | 19027 | GTATCCACCTTTGTCT | 78 | 70 |
| 912778 | 1178 | 1193 | 19018 | 19033 | GCTCATGTATCCACCT | 79 | 71 |
| 912779 | 1187 | 1202 | 19027 | 19042 | GCAAATCTTGCTCATG | 3 | 72 |
| 912780 | 1188 | 1203 | 19028 | 19043 | TGCAAATCTTGCTCAT | 13 | 73 |
| 912781 | 1189 | 1204 | 19029 | 19044 | TTGCAAATCTTGCTCA | 0 | 74 |
| 912782 | 1195 | 1210 | 19035 | 19050 | AGCAAGTTGCAAATCT | 77 | 75 |
| 912783 | 1199 | 1214 | 19039 | 19054 | GGGTAGCAAGTTGCAA | 74 | 76 |
| 912784 | 1205 | 1220 | 19045 | 19060 | CCTAATGGGTAGCAAG | 62 | 77 |
| 912785 | 1206 | 1221 | 19046 | 19061 | TCCTAATGGGTAGCAA | 79 | 78 |

TABLE 2
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912786 | 1207 | 1222 | 19047 | 19062 | ATCCTAATGGGTAGCA | 81 | 79 |
| 912787 | 1211 | 1226 | 19051 | 19066 | CATTATCCTAATGGGT | 46 | 80 |
| 912788 | 1212 | 1227 | 19052 | 19067 | ACATTATCCTAATGGG | 0 | 81 |
| 912789 | 1213 | 1228 | 19053 | 19068 | GACATTATCCTAATGG | 70 | 82 |
| 912790 | 1220 | 1235 | 19060 | 19075 | TACATAAGACATTATC | 34 | 83 |
| 912791 | 1224 | 1239 | 19064 | 19079 | GCATTACATAAGACAT | 86 | 84 |
| 912792 | 1245 | 1260 | 19085 | 19100 | CCACAGGCAGGGTACA | 76 | 85 |
| 912793 | 1246 | 1261 | 19086 | 19101 | TCCACAGGCAGGGTAC | 28 | 86 |
| 912794 | 1253 | 1268 | 19093 | 19108 | GGCAGATTCCACAGGC | 75 | 87 |
| 912795 | 1259 | 1274 | 19099 | 19114 | CGCAATGGCAGATTCC | 92 | 88 |
| 912796 | 1265 | 1280 | 19105 | 19120 | GACAATCGCAATGGCA | 64 | 89 |
| 912797 | 1266 | 1281 | 19106 | 19121 | GGACAATCGCAATGGC | 75 | 90 |
| 912798 | 1267 | 1282 | 19107 | 19122 | TGGACAATCGCAATGG | 73 | 91 |
| 912799 | 1285 | 1300 | 23690 | 23705 | AGCCATGTCACCAGTC | 67 | 92 |
| 912800 | 1289 | 1304 | 23694 | 23709 | TGGAAGCCATGTCACC | 24 | 93 |
| 912801 | 1290 | 1305 | 23695 | 23710 | CTGGAAGCCATGTCAC | 72 | 94 |
| 912802 | 1297 | 1312 | 23702 | 23717 | GGCATATCTGGAAGCC | 0 | 95 |
| 912803 | 1298 | 1313 | 23703 | 23718 | GGGCATATCTGGAAGC | 0 | 96 |
| 912804 | 1351 | 1366 | 23756 | 23771 | AGCACTCGAGTGAACA | 0 | 97 |
| 912805 | 1386 | 1401 | N/A | N/A | GCATTTGGGACCTGGA | 77 | 98 |
| 912806 | 1387 | 1402 | N/A | N/A | GGCATTTGGGACCTGG | 60 | 99 |
| 912807 | 1388 | 1403 | 25151 | 25166 | TGGCATTTGGGACCTG | 41 | 100 |
| 912808 | 1394 | 1409 | 25157 | 25172 | GCTCACTGGCATTTGG | 44 | 101 |
| 912809 | 1523 | 1538 | 25286 | 25301 | GTTCAGGCTGGACCTG | 49 | 102 |
| 912810 | 1547 | 1562 | 25310 | 25325 | AGGTACTTTATTGCCC | 11 | 103 |
| 912811 | 1550 | 1565 | 25313 | 25328 | AGCAGGTACTTTATTG | 64 | 104 |
| 912812 | 1653 | 1668 | 25416 | 25431 | AACTTTAGCACCTCTG | 91 | 105 |
| 912813 | 1655 | 1670 | 25418 | 25433 | GAAACTTTAGCACCTC | 88 | 106 |
| 912814 | 1656 | 1671 | 25419 | 25434 | GGAAACTTTAGCACCT | 53 | 107 |
| 912815 | 1669 | 1684 | 25432 | 25447 | CTGCACAAAGATGGGA | 80 | 108 |
| 912816 | 1671 | 1686 | 25434 | 25449 | AGCTGCACAAAGATGG | 45 | 109 |
| 912817 | 1685 | 1700 | 25448 | 25463 | AGCAATGCGGAGGTAG | 15 | 110 |
| 912818 | 1740 | 1755 | 25503 | 25518 | ACCAACTCAGCTCAGA | 85 | 111 |
| 912819 | 1741 | 1756 | 25504 | 25519 | AACCAACTCAGCTCAG | 79 | 112 |
| 912820 | 1757 | 1772 | 25520 | 25535 | TCCTAGCTTTTCATAA | 23 | 113 |
| 912821 | 1788 | 1803 | 25551 | 25566 | TGCTGGACCGCTGCAC | 0 | 114 |
| 912822 | 1796 | 1811 | 25559 | 25574 | GAGTTAAGTGCTGGAC | 93 | 115 |
| 912823 | 1802 | 1817 | 25565 | 25580 | GTATTAGAGTTAAGTG | 92 | 116 |
| 912824 | 1803 | 1818 | 25566 | 25581 | TGTATTAGAGTTAAGT | 79 | 117 |
| 912825 | 1806 | 1821 | 25569 | 25584 | TGATGTATTAGAGTTA | 92 | 118 |
| 912826 | 1808 | 1823 | 25571 | 25586 | GCTGATGTATTAGAGT | 80 | 119 |
| 912827 | 1821 | 1836 | 25584 | 25599 | TGAATTAACGCATGCT | 83 | 120 |
| 912828 | 1822 | 1837 | 25585 | 25600 | CTGAATTAACGCATGC | 78 | 121 |
| 912829 | 1870 | 1885 | 25633 | 25648 | AGTAAGGGACCCTCTG | 17 | 122 |
| 912830 | 1871 | 1886 | 25634 | 25649 | CAGTAAGGGACCCTCT | 28 | 123 |
| 912831 | 1872 | 1887 | 25635 | 25650 | TCAGTAAGGGACCCTC | 77 | 124 |
| 912832 | 1874 | 1889 | 25637 | 25652 | AGTCAGTAAGGGACCC | 51 | 125 |
| 912833 | 1893 | 1908 | 25656 | 25671 | ATTAATAGGGCCACGA | 80 | 126 |
| 912834 | 1895 | 1910 | 25658 | 25673 | CCATTAATAGGGCCAC | 90 | 127 |
| 912835 | 1896 | 1911 | 25659 | 25674 | ACCATTAATAGGGCCA | 81 | 128 |
| 912836 | 1906 | 1921 | 25669 | 25684 | GAACAGTCTGACCATT | 82 | 129 |
| 912837 | 1908 | 1923 | 25671 | 25686 | TGGAACAGTCTGACCA | 31 | 130 |
| 912838 | 1909 | 1924 | 25672 | 25687 | CTGGAACAGTCTGACC | 83 | 131 |
| 912839 | 1911 | 1926 | 25674 | 25689 | TGCTGGAACAGTCTGA | 72 | 132 |
| 912840 | 1916 | 1931 | 25679 | 25694 | CCTCATGCTGGAACAG | 83 | 133 |
| 912841 | 1928 | 1943 | 25691 | 25706 | TCATTCTAAGAACCTC | 96 | 134 |
| 912842 | 1945 | 1960 | 25708 | 25723 | ACCCATCCAAACACCT | 16 | 135 |
| 912843 | 1982 | 1997 | 25745 | 25760 | ACACATGGGCCAGCCT | 70 | 136 |
| 912844 | 1989 | 2004 | 25752 | 25767 | CAAGATCACACATGGG | 70 | 137 |
| 912845 | 2057 | 2072 | 25820 | 25835 | GGGACGAACTGCACCC | 0 | 138 |
| 912846 | 2098 | 2113 | 25861 | 25876 | TATCATCTTTGCAGAC | 81 | 139 |
| 912847 | 2116 | 2131 | 25879 | 25894 | GTTTTTAGTAGTCAAG | 91 | 140 |
| 912848 | 2117 | 2132 | 25880 | 25895 | CGTTTTTAGTAGTCAA | 91 | 141 |
| 912849 | 2145 | 2160 | 25908 | 25923 | TATCATCTTGTTACCC | 85 | 142 |
| 912850 | 2148 | 2163 | 25911 | 25926 | GATTATCATCTTGTTA | 70 | 143 |
| 912851 | 2150 | 2165 | 25913 | 25928 | TAGATTATCATCTTGT | 53 | 144 |
| 912852 | 2151 | 2166 | 25914 | 25929 | GTAGATTATCATCTTG | 80 | 145 |
| 912853 | 2152 | 2167 | 25915 | 25930 | AGTAGATTATCATCTT | 84 | 146 |
| 912854 | 2175 | 2190 | 25938 | 25953 | GTGAAAAAGGTGTTCT | 77 | 147 |
| 912855 | 2182 | 2197 | 25945 | 25960 | TAGTTAGGTGAAAAAG | 92 | 148 |
| 912856 | 2188 | 2203 | 25951 | 25966 | TTATTTTAGTTAGGTG | 88 | 149 |
| 912857 | 2190 | 2205 | 25953 | 25968 | CATTATTTTAGTTAGG | 86 | 150 |
| 912858 | 2273 | 2288 | 26036 | 26051 | CTACTAACATCTCACT | 55 | 151 |
| 912859 | 2274 | 2289 | 26037 | 26052 | TCTACTAACATCTCAC | 89 | 152 |
| 912860 | 2278 | 2293 | 26041 | 26056 | TTATTCTACTAACATC | 27 | 153 |
| 912861 | 2280 | 2295 | 26043 | 26058 | GCTTATTCTACTAACA | 79 | 154 |
| 912862 | 2281 | 2296 | 26044 | 26059 | GGCTTATTCTACTAAC | 81 | 155 |
| 912863 | 2632 | 2647 | 26395 | 26410 | GGTGAATGCCCTGCAC | 41 | 156 |

TABLE 3
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912864 | 2703 | 2718 | 26466 | 26481 | TTCAAGTTGTGTGCTC | 90 | 157 |
| 912865 | 2755 | 2770 | 26518 | 26533 | GGGAGAAACTCACTGA | 37 | 158 |
| 912866 | N/A | N/A | 4416 | 4431 | TGCTACTTGCCCCAGC | 2 | 159 |
| 912867 | N/A | N/A | 4421 | 4436 | CACAATGCTACTTGCC | 87 | 160 |
| 912868 | N/A | N/A | 4584 | 4599 | CCCAATGGCAGGGCTT | 58 | 161 |
| 912869 | N/A | N/A | 4592 | 4607 | TGCTCCTACCCAATGG | 46 | 162 |
| 912870 | N/A | N/A | 4766 | 4781 | GACTTTTATTGTTGCT | 95 | 163 |
| 912871 | N/A | N/A | 4883 | 4898 | TTCTATACCAGAGTGA | 89 | 164 |
| 912872 | N/A | N/A | 4884 | 4899 | TTTCTATACCAGAGTG | 89 | 165 |
| 912873 | N/A | N/A | 5405 | 5420 | GTAGATGGCCTTAATG | 83 | 166 |
| 912876 | N/A | N/A | 6155 | 6170 | TACATCCACGACTTCG | 94 | 167 |
| 912877 | N/A | N/A | 6156 | 6171 | TTACATCCACGACTTC | 76 | 168 |
| 912880 | N/A | N/A | 6606 | 6621 | GGAACATTCAGGGTTT | 13 | 169 |
| 912881 | N/A | N/A | 6834 | 6849 | ATTACTTGGGTGCAGG | 55 | 170 |
| 912884 | N/A | N/A | 6838 | 6853 | GCAGATTACTTGGGTG | 45 | 171 |
| 912885 | N/A | N/A | 6931 | 6946 | TGCAGGACAGGTTCCT | 30 | 172 |
| 912888 | N/A | N/A | 7549 | 7564 | CACACTGGGTCACCAC | 55 | 173 |
| 912889 | N/A | N/A | 7552 | 7567 | AGTCACACTGGGTCAC | 61 | 174 |
| 912928 | N/A | N/A | 12273 | 12288 | GGTATATGTTCCCAGG | 87 | 175 |
| 912929 | N/A | N/A | 12314 | 12329 | TATAACCACAGCCTGG | 29 | 176 |
| 912932 | N/A | N/A | 12321 | 12336 | CTGACTATATAACCAC | 81 | 177 |
| 912933 | N/A | N/A | 12666 | 12681 | ATCTTAGTGGCTGGGT | 91 | 178 |
| 912936 | N/A | N/A | 12767 | 12782 | CTTACTATGGTAGAGT | 88 | 179 |
| 912937 | N/A | N/A | 12768 | 12783 | TCTTACTATGGTAGAG | 74 | 180 |
| 912940 | N/A | N/A | 12835 | 12850 | TGCATTGCATAGCCTT | 97 | 181 |
| 912941 | N/A | N/A | 12836 | 12851 | TTGCATTGCATAGCCT | 96 | 182 |
| 912944 | N/A | N/A | 12907 | 12922 | TGCTTATAAAGCACAC | 61 | 183 |
| 912945 | N/A | N/A | 12988 | 13003 | GGAATAAGCCTCCACC | 14 | 184 |
| 912948 | N/A | N/A | 14055 | 14070 | GAAATCTGATTGCTTC | 59 | 185 |
| 912949 | N/A | N/A | 14393 | 14408 | TACTTATCTGCTCACT | 66 | 186 |
| 912952 | N/A | N/A | 14673 | 14688 | TCTCTTAGTGTCCCCA | 90 | 187 |
| | | | 14707 | 14722 | | | |
| 912953 | N/A | N/A | 14674 | 14689 | ATCTCTTAGTGTCCCC | 92 | 188 |
| | | | 14708 | 14723 | | | |
| 912956 | N/A | N/A | 15284 | 15299 | TCACATTCATGCTTGC | 82 | 189 |
| 912957 | N/A | N/A | 15291 | 15306 | GATAACCTCACATTCA | 0 | 190 |
| 912960 | N/A | N/A | 15712 | 15727 | GAGCTAGGTGCTTCAC | 6 | 191 |
| 912961 | N/A | N/A | 15753 | 15768 | ATAACAACTGAACCAC | 85 | 192 |
| 912964 | N/A | N/A | 15937 | 15952 | GTTATTAGCCAAATGC | 92 | 193 |
| 912965 | N/A | N/A | 16468 | 16483 | GGAGACTTGGCAAGGT | 87 | 194 |
| 912968 | N/A | N/A | 16960 | 16975 | ATTCATGACAGCCCTT | 46 | 195 |
| 912969 | N/A | N/A | 17128 | 17143 | ATCGATTTTTCAGAGT | 9 | 196 |
| 912972 | N/A | N/A | 17134 | 17149 | ACAAACATCGATTTTT | 52 | 197 |
| 912973 | N/A | N/A | 17769 | 17784 | CTCTTTAATGACCTCG | 90 | 198 |
| 912976 | N/A | N/A | 18865 | 18880 | GTCAGAGGCACTCACA | 25 | 199 |
| 912977 | N/A | N/A | 18959 | 18974 | AGCTATTATCTCCCAC | 0 | 200 |
| 912980 | N/A | N/A | 19315 | 19330 | AGTTTCTGGGCTTGCA | 90 | 201 |
| 912981 | N/A | N/A | 19382 | 19397 | GGCAATCACAAGAGAC | 73 | 202 |
| 912984 | N/A | N/A | 20286 | 20301 | AGAGGAAGCCCAATCA | 79 | 203 |
| | | | 20316 | 20331 | | | |
| 912985 | N/A | N/A | 20287 | 20302 | CAGAGGAAGCCCAATC | 93 | 204 |
| | | | 20317 | 20332 | | | |
| 912988 | N/A | N/A | 20658 | 20673 | TAGAAATTGCAGTGCC | 92 | 205 |
| 912989 | N/A | N/A | 20731 | 20746 | TCCTATCCATATATTG | 55 | 206 |
| 912992 | N/A | N/A | 21408 | 21423 | GCAATTCTAGACATGG | 88 | 207 |
| 912993 | N/A | N/A | 21558 | 21573 | AGGACTTACACCAAGA | 86 | 208 |
| 912996 | N/A | N/A | 21936 | 21951 | TTCCTAATAAGAGCCC | 24 | 209 |
| 912997 | N/A | N/A | 21946 | 21961 | GTCAAACATCTTCCTA | 66 | 210 |
| 913000 | N/A | N/A | 22077 | 22092 | AAAACTGTAGGATAGG | 47 | 211 |
| 913001 | N/A | N/A | 22162 | 22177 | GTTACATCCATAAAAC | 0 | 212 |
| 913004 | N/A | N/A | 22169 | 22184 | AGAGAATGTTACATCC | 62 | 213 |
| 913008 | N/A | N/A | 23083 | 23098 | AAAGATTAATCAGGGC | 61 | 214 |
| 913012 | N/A | N/A | 23788 | 23803 | GTATTTACCTGGAGGC | 0 | 215 |
| 913016 | N/A | N/A | 24426 | 24441 | GGCCTATGATTTTCAG | 0 | 216 |

TABLE 4
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912874 | N/A | N/A | 5869 | 5884 | ATACTTTTGGCAAGGC | 96 | 217 |
| 912875 | N/A | N/A | 5870 | 5885 | AATACTTTTGGCAAGG | 91 | 218 |
| 912878 | N/A | N/A | 6159 | 6174 | TGCTTACATCCACGAC | 12 | 219 |
| 912879 | N/A | N/A | 6296 | 6311 | CATCATGTTGGTCTCG | 54 | 220 |
| 912882 | N/A | N/A | 6835 | 6850 | GATTACTTGGGTGCAG | 39 | 221 |
| 912883 | N/A | N/A | 6837 | 6852 | CAGATTACTTGGGTGC | 69 | 222 |
| 912886 | N/A | N/A | 7083 | 7098 | TTTAATGGTGTTTTGG | 87 | 223 |
| 912887 | N/A | N/A | 7478 | 7493 | TCAAATGCCGGTATTC | 52 | 224 |
| 912890 | N/A | N/A | 7587 | 7602 | GTGAACTTCAACTTCC | 56 | 225 |
| 912930 | N/A | N/A | 12317 | 12332 | CTATATAACCACAGCC | 77 | 226 |
| 912931 | N/A | N/A | 12319 | 12334 | GACTATATAACCACAG | 92 | 227 |
| 912934 | N/A | N/A | 12670 | 12685 | AATCATCTTAGTGGCT | 91 | 228 |
| 912935 | N/A | N/A | 12765 | 12780 | TACTATGGTAGAGTGG | 80 | 229 |
| 912938 | N/A | N/A | 12786 | 12801 | GTACATGGTCTGCAAA | 84 | 230 |
| 912939 | N/A | N/A | 12787 | 12802 | TGTACATGGTCTGCAA | 57 | 231 |
| 912942 | N/A | N/A | 12843 | 12858 | GCATGCATTGCATTGC | 16 | 232 |
| 912943 | N/A | N/A | 12885 | 12900 | ACCAATCCTGTTAGAC | 93 | 233 |
| 912946 | N/A | N/A | 13557 | 13572 | GGAGACACCAAGCACC | 42 | 234 |
| 912947 | N/A | N/A | 13751 | 13766 | GCACTAAGTGTTAGAA | 79 | 235 |
| 912950 | N/A | N/A | 14396 | 14411 | GCTTACTTATCTGCTC | 0 | 236 |
| 912951 | N/A | N/A | 14501 | 14516 | GGAGATCCATCCTGCA | 0 | 237 |
| 912954 | N/A | N/A | 14675 | 14690 | CATCTCTTAGTGTCCC | 92 | 238 |
| | | | 14709 | 14724 | | | |
| 912955 | N/A | N/A | 15122 | 15137 | TCCTAATGTCCTCAAC | 9 | 239 |
| 912958 | N/A | N/A | 15293 | 15308 | AAGATAACCTCACATT | 33 | 240 |
| 912959 | N/A | N/A | 15294 | 15309 | CAAGATAACCTCACAT | 22 | 241 |
| 912962 | N/A | N/A | 15754 | 15769 | TATAACAACTGAACCA | 82 | 242 |
| 912963 | N/A | N/A | 15856 | 15871 | GCTTTAAAGCAGGACA | 8 | 243 |
| 912966 | N/A | N/A | 16774 | 16789 | AAAATTGTGGGTTTAG | 68 | 244 |
| 912967 | N/A | N/A | 16850 | 16865 | ATCATTTGGACCATAG | 81 | 245 |
| 912970 | N/A | N/A | 17130 | 17145 | ACATCGATTTTTCAGA | 83 | 246 |
| 912971 | N/A | N/A | 17133 | 17148 | CAAACATCGATTTTTC | 62 | 247 |
| 912974 | N/A | N/A | 17843 | 17858 | GCTTTACAAGCTGGTC | 0 | 248 |
| 912975 | N/A | N/A | 17879 | 17894 | ATCTATGTTCTCCTAG | 0 | 249 |
| 912978 | N/A | N/A | 19125 | 19140 | ACCTAAAATGCTCACC | 0 | 250 |
| 912979 | N/A | N/A | 19198 | 19213 | CCAGACTACATGCCAC | 79 | 251 |
| 912982 | N/A | N/A | 19446 | 19461 | TCTACTAGGCATCTCT | 63 | 252 |
| 912983 | N/A | N/A | 19447 | 19462 | TTCTACTAGGCATCTC | 42 | 253 |
| 912986 | N/A | N/A | 20288 | 20303 | TCAGAGGAAGCCCAAT | 92 | 254 |
| | | | 20318 | 20333 | | | |
| 912987 | N/A | N/A | 20656 | 20671 | GAAATTGCAGTGCCCT | 92 | 255 |
| 912990 | N/A | N/A | 21393 | 21408 | GCCAACCTATCACTGA | 60 | 256 |
| 912991 | N/A | N/A | 21400 | 21415 | AGACATGGCCAACCTA | 32 | 257 |
| 912994 | N/A | N/A | 21565 | 21580 | TGAAATAAGGACTTAC | 67 | 258 |
| 912995 | N/A | N/A | 21934 | 21949 | CCTAATAAGAGCCCCA | 31 | 259 |
| 912998 | N/A | N/A | 22041 | 22056 | GAAATCTGTCAGAGCA | 33 | 260 |
| 912999 | N/A | N/A | 22072 | 22087 | TGTAGGATAGGACTAG | 0 | 261 |
| 913002 | N/A | N/A | 22166 | 22181 | GAATGTTACATCCATA | 53 | 262 |
| 913003 | N/A | N/A | 22168 | 22183 | GAGAATGTTACATCCA | 80 | 263 |
| 913005 | N/A | N/A | 22605 | 22620 | GTGATAAATCTGCAAG | 70 | 264 |
| 913006 | N/A | N/A | 23081 | 23096 | AGATTAATCAGGGCCA | 8 | 265 |
| 913007 | N/A | N/A | 23082 | 23097 | AAGATTAATCAGGGCC | 30 | 266 |
| 913009 | N/A | N/A | 23325 | 23340 | GGTCACATGTGAGCCC | 0 | 267 |
| 913010 | N/A | N/A | 23496 | 23511 | CACTTCTGGTTCAAGA | 13 | 268 |
| 913011 | N/A | N/A | 23580 | 23595 | CCAATCTGATGACTTC | 80 | 269 |
| 913013 | N/A | N/A | 23790 | 23805 | AAGTATTTACCTGGAG | 0 | 270 |
| 913014 | N/A | N/A | 24028 | 24043 | CACTCAAAGAGACTCA | 65 | 271 |
| 913015 | N/A | N/A | 24425 | 24440 | GCCTATGATTTTCAGG | 0 | 272 |
| 913017 | N/A | N/A | 24633 | 24648 | CACTACTGCCCTCTTC | 50 | 273 |
| 913018 | N/A | N/A | 24983 | 24998 | TGCTGGGCTGATGTCA | 0 | 274 |
| 913019 | N/A | N/A | 25150 | 25165 | GGCATTTGGGACCTGA | 67 | 275 |

TABLE 5
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915343 | 1 | 16 | 2739 | 2754 | GCCCCCCTCGGACCAT | 0 | 276 |
| 915363 | 45 | 60 | 2783 | 2798 | CCTCAGTGTCTCGGCC | 0 | 277 |
| 915383 | 107 | 122 | 2845 | 2860 | AATCGGCTCGGGTCCT | 29 | 278 |
| 915403 | 190 | 205 | 2928 | 2943 | GACAAGCTCCAGCCGC | 64 | 279 |
| 915423 | 249 | 264 | 2987 | 3002 | CGCTCAGGCAGCGGGT | 0 | 280 |
| 915443 | 347 | 362 | N/A | N/A | CTCCAGCGGGATACCG | 6 | 281 |
| 915463 | 386 | 401 | 5960 | 5975 | GGCCTTCCGCACAAGA | 0 | 282 |
| 915483 | 416 | 431 | 5990 | 6005 | TGGATGGAAGATGCCA | 28 | 283 |
| 915503 | 452 | 467 | 6026 | 6041 | GAGACCCTGTCGGAGG | 45 | 284 |
| 915523 | 488 | 503 | 6062 | 6077 | GATGAGCTGGTGGACA | 70 | 285 |
| 915543 | 510 | 525 | 6084 | 6099 | GAGAGATGCCTATTTT | 92 | 286 |
| 915563 | 559 | 574 | 6133 | 6148 | GACCGAAAGTCAGACA | 7 | 287 |
| 915603 | 697 | 712 | 11915 | 11930 | GTTTTGGCATCAATGA | 94 | 288 |
| 915623 | 754 | 769 | 11972 | 11987 | GACTTGACTTTAGGGC | 98 | 289 |
| 915643 | 827 | 842 | 12045 | 12060 | CGAGAGAAGGTAGAGG | 97 | 290 |
| 915663 | 879 | 894 | 13615 | 13630 | CTCGAAGGCATATCTC | 66 | 291 |
| 915683 | 932 | 947 | 16052 | 16067 | GGGCCTGTTGCAGATG | 0 | 292 |
| 915703 | 985 | 1000 | 16105 | 16120 | GGCATGGCGACCTCAG | 6 | 293 |
| 915723 | 1037 | 1052 | 16157 | 16172 | AGCCAAGGCAGCCGAC | 0 | 294 |
| 915743 | 1132 | 1147 | 16252 | 16267 | GCGAGCCTGGGCGAGA | 0 | 295 |
| 915763 | 1177 | 1192 | 19017 | 19032 | CTCATGTATCCACCTT | 88 | 296 |
| 915783 | 1229 | 1244 | 19069 | 19084 | GGGCAGCATTACATAA | 73 | 297 |
| 915803 | 1286 | 1301 | 23691 | 23706 | AAGCCATGTCACCAGT | 34 | 298 |
| 915823 | 1348 | 1363 | 23753 | 23768 | ACTCGAGTGAACACCT | 12 | 299 |
| 915843 | 1405 | 1420 | 25168 | 25183 | GCCTGTTGGCTGCTCA | 1 | 300 |
| 915863 | 1473 | 1488 | 25236 | 25251 | CTGCTGGACAGCCCTT | 0 | 301 |
| 915883 | 1542 | 1557 | 25305 | 25320 | CTTTATTGCCCAAGAA | 72 | 302 |
| 915903 | 1601 | 1616 | 25364 | 25379 | CAGACTCTTCTCTAGT | 49 | 303 |
| 915923 | 1633 | 1648 | 25396 | 25411 | AATCTGCTAGACTCGC | 88 | 304 |
| 915943 | 1686 | 1701 | 25449 | 25464 | CAGCAATGCGGAGGTA | 80 | 305 |
| 915963 | 1768 | 1783 | 25531 | 25546 | GAAAGGTTGCTTCCTA | 84 | 306 |
| 915983 | 1789 | 1804 | 25552 | 25567 | GTGCTGGACCGCTGCA | 11 | 307 |
| 916003 | 1815 | 1830 | 25578 | 25593 | AACGCATGCTGATGTA | 69 | 308 |
| 916023 | 1848 | 1863 | 25611 | 25626 | GCTTCCTGGTGTCATT | 81 | 309 |
| 916043 | 1884 | 1899 | 25647 | 25662 | GCCACGAAACAGTCAG | 67 | 310 |
| 916063 | 1913 | 1928 | 25676 | 25691 | CATGCTGGAACAGTCT | 20 | 311 |
| 916083 | 1954 | 1969 | 25717 | 25732 | AAGGCCCCCACCCATC | 0 | 312 |
| 916103 | 1977 | 1992 | 25740 | 25755 | TGGGCCAGCCTACCCC | 0 | 313 |
| 916123 | 2026 | 2041 | 25789 | 25804 | GGAAGTGGGATCATGC | 55 | 314 |
| 916142 | 2100 | 2115 | 25863 | 25878 | GTTATCATCTTTGCAG | 57 | 315 |
| 916162 | 2139 | 2154 | 25902 | 25917 | CTTGTTACCCCCGCCA | 84 | 316 |
| 916182 | 2264 | 2279 | 26027 | 26042 | TCTCACTGATTCACAT | 83 | 317 |
| 916202 | 2624 | 2639 | 26387 | 26402 | CCCTGCACACTAGATT | 55 | 318 |
| 916222 | 2677 | 2692 | 26440 | 26455 | GAGGCGGAAGCTCCTG | 0 | 319 |
| 916242 | 2707 | 2722 | 26470 | 26485 | CAGGTTCAAGTTGTGT | 83 | 320 |
| 916282 | N/A | N/A | 4225 | 4240 | AAATGTACGGAATCTC | 79 | 321 |
| 916302 | N/A | N/A | 4822 | 4837 | GTGTAAACATTTGTCC | 74 | 322 |
| 916322 | N/A | N/A | 5414 | 5429 | AGCTTTGGTGTAGATG | 49 | 323 |
| 916342 | N/A | N/A | 5801 | 5816 | TACTATGGGAGCCACA | 42 | 324 |
| 916362 | N/A | N/A | 6866 | 6881 | TGAAATTGTAACTGCC | 70 | 325 |
| 916382 | N/A | N/A | 7492 | 7507 | TAGATCGGTGCTGTTC | 27 | 326 |
| 916402 | N/A | N/A | 7785 | 7800 | GTTATAGGCGAGAGCA | 0 | 327 |
| 916562 | N/A | N/A | 12316 | 12331 | TATATAACCACAGCCT | 58 | 328 |
| 916582 | N/A | N/A | 12932 | 12947 | ATAAGAGCTGTCTCCT | 94 | 329 |
| 916602 | N/A | N/A | 13703 | 13718 | CTAGTAAATGCTTGTC | 96 | 330 |
| 916622 | N/A | N/A | 14177 | 14192 | CTAATATTTCTACAGC | 0 | 331 |
| 916642 | N/A | N/A | 14672 | 14687 | CTCTTAGTGTCCCCAT | 95 | 332 |
| 916662 | N/A | N/A | 15542 | 15557 | TTCCATCACAAGGCCT | 50 | 333 |
| 916682 | N/A | N/A | 16317 | 16332 | TCCATAATGCACAAGA | 71 | 334 |
| 916702 | N/A | N/A | 17223 | 17238 | TGTAGCTGGTTTGTGG | 88 | 335 |
| 916722 | N/A | N/A | 18223 | 18238 | AACAGCTACATCAGGC | 44 | 336 |
| 916742 | N/A | N/A | 19249 | 19264 | GGCATTGCACATAGAC | 74 | 337 |
| 916761 | N/A | N/A | 20410 | 20425 | GTAAGCAATGCAGCCA | 88 | 338 |
| 916781 | N/A | N/A | 20659 | 20674 | TTAGAAATTGCAGTGC | 91 | 339 |
| 916801 | N/A | N/A | 20989 | 21004 | AGGTATTAAACTGCCA | 25 | 340 |
| 916821 | N/A | N/A | 21506 | 21521 | GTCCTAAGAGCACTCA | 57 | 341 |
| 916841 | N/A | N/A | 22603 | 22618 | GATAAATCTGCAAGAG | 49 | 342 |
| 916861 | N/A | N/A | 23472 | 23487 | GGGACTTACACTGAAA | 66 | 343 |
| 916881 | N/A | N/A | 24314 | 24329 | GTCAACGCAGACTGCT | 33 | 344 |

TABLE 6
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915344 | 2 | 17 | 2740 | 2755 | CGCCCCCCTCGGACCA | 0 | 345 |
| 915364 | 46 | 61 | 2784 | 2799 | GCCTCAGTGTCTCGGC | 0 | 346 |
| 915384 | 108 | 123 | 2846 | 2861 | GAATCGGCTCGGGTCC | 49 | 347 |
| 915404 | 191 | 206 | 2929 | 2944 | GGACAAGCTCCAGCCG | 9 | 348 |
| 915424 | 250 | 265 | 2988 | 3003 | TCGCTCAGGCAGCGGG | 0 | 349 |
| 915444 | 348 | 363 | N/A | N/A | GCTCCAGCGGGATACC | 0 | 350 |
| 915464 | 387 | 402 | 5961 | 5976 | TGGCCTTCCGCACAAG | 0 | 351 |
| 915484 | 428 | 443 | 6002 | 6017 | TAAGTTGAAGGATGGA | 96 | 352 |
| 915504 | 453 | 468 | 6027 | 6042 | AGAGACCCTGTCGGAG | 80 | 353 |
| 915524 | 489 | 504 | 6063 | 6078 | AGATGAGCTGGTGGAC | 81 | 354 |
| 915544 | 512 | 527 | 6086 | 6101 | AAGAGAGATGCCTATT | 77 | 355 |
| 915564 | 560 | 575 | 6134 | 6149 | GGACCGAAAGTCAGAC | 0 | 356 |
| 915604 | 700 | 715 | 11918 | 11933 | GTTGTTTTGGCATCAA | 91 | 357 |
| 915624 | 755 | 770 | 11973 | 11988 | GGACTTGACTTTAGGG | 81 | 358 |
| 915644 | 828 | 843 | 12046 | 12061 | TCGAGAGAAGGTAGAG | 24 | 359 |
| 915664 | 880 | 895 | 13616 | 13631 | CCTCGAAGGCATATCT | 41 | 360 |
| 915684 | 952 | 967 | 16072 | 16087 | GATGACTTCAGGCCTG | 0 | 361 |
| 915704 | 987 | 1002 | 16107 | 16122 | TGGGCATGGCGACCTC | 0 | 362 |
| 915724 | 1038 | 1053 | 16158 | 16173 | CAGCCAAGGCAGCCGA | 0 | 363 |
| 915744 | 1133 | 1148 | 16253 | 16268 | AGCGAGCCTGGGCGAG | 0 | 364 |
| 915764 | 1179 | 1194 | 19019 | 19034 | TGCTCATGTATCCACC | 56 | 365 |
| 915784 | 1230 | 1245 | 19070 | 19085 | AGGGCAGCATTACATA | 69 | 366 |
| 915804 | 1293 | 1308 | 23698 | 23713 | TATCTGGAAGCCATGT | 6 | 367 |
| 915824 | 1349 | 1364 | 23754 | 23769 | CACTCGAGTGAACACC | 0 | 368 |
| 915844 | 1406 | 1421 | 25169 | 25184 | GGCCTGTTGGCTGCTC | 0 | 369 |
| 915864 | 1477 | 1492 | 25240 | 25255 | GTCTCTGCTGGACAGC | 0 | 370 |
| 915884 | 1545 | 1560 | 25308 | 25323 | GTACTTTATTGCCCAA | 73 | 371 |
| 915904 | 1607 | 1622 | 25370 | 25385 | GACTCACAGACTCTTC | 92 | 372 |
| 915924 | 1634 | 1649 | 25397 | 25412 | GAATCTGCTAGACTCG | 65 | 373 |
| 915944 | 1687 | 1702 | 25450 | 25465 | ACAGCAATGCGGAGGT | 83 | 374 |
| 915964 | 1769 | 1784 | 25532 | 25547 | CGAAAGGTTGCTTCCT | 79 | 375 |
| 915984 | 1790 | 1805 | 25553 | 25568 | AGTGCTGGACCGCTGC | 38 | 376 |
| 916004 | 1816 | 1831 | 25579 | 25594 | TAACGCATGCTGATGT | 79 | 377 |
| 916024 | 1849 | 1864 | 25612 | 25627 | GGCTTCCTGGTGTCAT | 73 | 378 |
| 916044 | 1885 | 1900 | 25648 | 25663 | GGCCACGAAACAGTCA | 40 | 379 |
| 916064 | 1914 | 1929 | 25677 | 25692 | TCATGCTGGAACAGTC | 80 | 380 |
| 916084 | 1958 | 1973 | 25721 | 25736 | TCACAAGGCCCCCACC | 35 | 381 |
| 916104 | 1978 | 1993 | 25741 | 25756 | ATGGGCCAGCCTACCC | 0 | 382 |
| 916124 | 2053 | 2068 | 25816 | 25831 | CGAACTGCACCCCTTC | 38 | 383 |
| 916143 | 2101 | 2116 | 25864 | 25879 | GGTTATCATCTTTGCA | 81 | 384 |
| 916163 | 2140 | 2155 | 25903 | 25918 | TCTTGTTACCCCCGCC | 84 | 385 |
| 916183 | 2265 | 2280 | 26028 | 26043 | ATCTCACTGATTCACA | 86 | 386 |
| 916203 | 2625 | 2640 | 26388 | 26403 | GCCCTGCACACTAGAT | 65 | 387 |
| 916223 | 2678 | 2693 | 26441 | 26456 | GGAGGCGGAAGCTCCT | 0 | 388 |
| 916243 | 2709 | 2724 | 26472 | 26487 | GCCAGGTTCAAGTTGT | 62 | 389 |
| 916283 | N/A | N/A | 4226 | 4241 | CAAATGTACGGAATCT | 52 | 390 |
| 916303 | N/A | N/A | 4864 | 4879 | TACTTTAGGCTCCTGG | 90 | 391 |
| 916323 | N/A | N/A | 5422 | 5437 | AGCATTAGAGCTTTGG | 75 | 392 |
| 916343 | N/A | N/A | 5803 | 5818 | TCTACTATGGGAGCCA | 89 | 393 |
| 916363 | N/A | N/A | 6927 | 6942 | GGACAGGTTCCTTGGA | 0 | 394 |
| 916383 | N/A | N/A | 7493 | 7508 | CTAGATCGGTGCTGTT | 14 | 395 |
| 916403 | N/A | N/A | 7786 | 7801 | AGTTATAGGCGAGAGC | 0 | 396 |
| 916563 | N/A | N/A | 12318 | 12333 | ACTATATAACCACAGC | 90 | 397 |
| 916583 | N/A | N/A | 12936 | 12951 | GACAATAAGAGCTGTC | 0 | 398 |
| 916603 | N/A | N/A | 13704 | 13719 | GCTAGTAAATGCTTGT | 73 | 399 |
| 916623 | N/A | N/A | 14231 | 14246 | CCAACTTTTAGTATTA | 92 | 400 |
| 916643 | N/A | N/A | 14678 | 14693 | AGCCATCTCTTAGTGT | 50 | 401 |
| 916663 | N/A | N/A | 15566 | 15581 | TCTGATGTCGAAGAGG | 68 | 402 |
| 916683 | N/A | N/A | 16341 | 16356 | TCCCATGTGGCAGTAC | 0 | 403 |
| 916703 | N/A | N/A | 17239 | 17254 | TCCAAATGCCCAACTC | 37 | 404 |
| 916723 | N/A | N/A | 18241 | 18256 | GCAAATAATGTGCACA | 22 | 405 |
| 916743 | N/A | N/A | 19250 | 19265 | GGGCATTGCACATAGA | 59 | 406 |
| 916762 | N/A | N/A | 20413 | 20428 | GTAGTAAGCAATGCAG | 69 | 407 |
| 916782 | N/A | N/A | 20660 | 20675 | CTTAGAAATTGCAGTG | 91 | 408 |
| 916802 | N/A | N/A | 21002 | 21017 | ATTTTAACAGCTCAGG | 95 | 409 |
| 916822 | N/A | N/A | 21540 | 21555 | TATGACATTTCAGAGT | 88 | 410 |
| 916842 | N/A | N/A | 22629 | 22644 | AGTACAAGCGCAGCCT | 14 | 411 |
| 916862 | N/A | N/A | 23538 | 23553 | ACAAGGACAAGCCCAC | 37 | 412 |
| 916882 | N/A | N/A | 24339 | 24354 | GAAGTAGCGGCATCCC | 68 | 413 |

TABLE 7
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915345 | 3 | 18 | 2741 | 2756 | CCGCCCCCCTCGGACC | 0 | 414 |
| 915365 | 47 | 62 | 2785 | 2800 | TGCCTCAGTGTCTCGG | 0 | 415 |
| 915385 | 109 | 124 | 2847 | 2862 | GGAATCGGCTCGGGTC | 72 | 416 |
| 915405 | 193 | 208 | 2931 | 2946 | AAGGACAAGCTCCAGC | 41 | 417 |
| 915425 | 251 | 266 | 2989 | 3004 | CTCGCTCAGGCAGCGG | 0 | 418 |
| 915445 | 349 | 364 | N/A | N/A | TGCTCCAGCGGGATAC | 0 | 419 |
| 915465 | 388 | 403 | 5962 | 5977 | CTGGCCTTCCGCACAA | 16 | 420 |
| 915485 | 430 | 445 | 6004 | 6019 | CTTAAGTTGAAGGATG | 27 | 421 |
| 915505 | 454 | 469 | 6028 | 6043 | CAGAGACCCTGTCGGA | 72 | 422 |
| 915525 | 492 | 507 | 6066 | 6081 | CGGAGATGAGCTGGTG | 92 | 423 |
| 915545 | 513 | 528 | 6087 | 6102 | TAAGAGAGATGCCTAT | 57 | 424 |
| 915565 | 561 | 576 | 6135 | 6150 | TGGACCGAAAGTCAGA | 0 | 425 |
| 915605 | 701 | 716 | 11919 | 11934 | GGTTGTTTTGGCATCA | 97 | 426 |
| 915625 | 756 | 771 | 11974 | 11989 | TGGACTTGACTTTAGG | 93 | 427 |
| 915645 | 829 | 844 | 12047 | 12062 | CTCGAGAGAAGGTAGA | 0 | 428 |
| 915665 | 881 | 896 | 13617 | 13632 | TCCTCGAAGGCATATC | 0 | 429 |
| 915685 | 953 | 968 | 16073 | 16088 | GGATGACTTCAGGCCT | 0 | 430 |
| 915705 | 988 | 1003 | 16108 | 16123 | CTGGGCATGGCGACCT | 0 | 431 |
| 915725 | 1039 | 1054 | 16159 | 16174 | ACAGCCAAGGCAGCCG | 0 | 432 |
| 915745 | 1134 | 1149 | 16254 | 16269 | TAGCGAGCCTGGGCGA | 0 | 433 |
| 915765 | 1193 | 1208 | 19033 | 19048 | CAAGTTGCAAATCTTG | 0 | 434 |
| 915785 | 1231 | 1246 | 19071 | 19086 | CAGGGCAGCATTACAT | 74 | 435 |
| 915805 | 1300 | 1315 | 23705 | 23720 | TCGGGCATATCTGGAA | 21 | 436 |
| 915825 | 1350 | 1365 | 23755 | 23770 | GCACTCGAGTGAACAC | 0 | 437 |
| 915845 | 1407 | 1422 | 25170 | 25185 | AGGCCTGTTGGCTGCT | 0 | 438 |
| 915865 | 1480 | 1495 | 25243 | 25258 | TTGGTCTCTGCTGGAC | 21 | 439 |
| 915885 | 1546 | 1561 | 25309 | 25324 | GGTACTTTATTGCCCA | 62 | 440 |
| 915905 | 1609 | 1624 | 25372 | 25387 | GTGACTCACAGACTCT | 81 | 441 |
| 915925 | 1635 | 1650 | 25398 | 25413 | AGAATCTGCTAGACTC | 74 | 442 |
| 915945 | 1688 | 1703 | 25451 | 25466 | CACAGCAATGCGGAGG | 56 | 443 |
| 915965 | 1770 | 1785 | 25533 | 25548 | GCGAAAGGTTGCTTCC | 66 | 444 |
| 915985 | 1791 | 1806 | 25554 | 25569 | AAGTGCTGGACCGCTG | 71 | 445 |
| 916005 | 1817 | 1832 | 25580 | 25595 | TTAACGCATGCTGATG | 69 | 446 |
| 916025 | 1850 | 1865 | 25613 | 25628 | GGGCTTCCTGGTGTCA | 58 | 447 |
| 916045 | 1886 | 1901 | 25649 | 25664 | GGGCCACGAAACAGTC | 9 | 448 |
| 916065 | 1915 | 1930 | 25678 | 25693 | CTCATGCTGGAACAGT | 86 | 449 |
| 916085 | 1959 | 1974 | 25722 | 25737 | ATCACAAGGCCCCCAC | 82 | 450 |
| 916105 | 1979 | 1994 | 25742 | 25757 | CATGGGCCAGCCTACC | 0 | 451 |
| 916125 | 2054 | 2069 | 25817 | 25832 | ACGAACTGCACCCCTT | 84 | 452 |
| 916144 | 2102 | 2117 | 25865 | 25880 | AGGTTATCATCTTTGC | 90 | 453 |
| 916164 | 2141 | 2156 | 25904 | 25919 | ATCTTGTTACCCCCGC | 88 | 454 |
| 916184 | 2266 | 2281 | 26029 | 26044 | CATCTCACTGATTCAC | 91 | 455 |
| 916204 | 2626 | 2641 | 26389 | 26404 | TGCCCTGCACACTAGA | 47 | 456 |
| 916224 | 2680 | 2695 | 26443 | 26458 | GAGGAGGCGGAAGCTC | 0 | 457 |
| 916244 | 2710 | 2725 | 26473 | 26488 | AGCCAGGTTCAAGTTG | 71 | 458 |
| 916284 | N/A | N/A | 4227 | 4242 | TCAAATGTACGGAATC | 40 | 459 |
| 916304 | N/A | N/A | 4865 | 4880 | GTACTTTAGGCTCCTG | 89 | 460 |
| 916324 | N/A | N/A | 5429 | 5444 | ACATATCAGCATTAGA | 87 | 461 |
| 916344 | N/A | N/A | 5804 | 5819 | GTCTACTATGGGAGCC | 90 | 462 |
| 916364 | N/A | N/A | 6966 | 6981 | GAAGATGCATAGAGGA | 0 | 463 |
| 916384 | N/A | N/A | 7550 | 7565 | TCACACTGGGTCACCA | 43 | 464 |
| 916544 | N/A | N/A | 12135 | 12150 | GGCAATCAGGGAGGCA | 32 | 465 |
| 916564 | N/A | N/A | 12320 | 12335 | TGACTATATAACCACA | 92 | 466 |
| 916584 | N/A | N/A | 12951 | 12966 | CCCAATTGCCACTAGG | 83 | 467 |
| 916604 | N/A | N/A | 13718 | 13733 | TCTTTACCAAGACCGC | 92 | 468 |
| 916624 | N/A | N/A | 14245 | 14260 | GACAAATTCATCAACC | 87 | 469 |
| 916644 | N/A | N/A | 14778 | 14793 | CTGTATCCAAAAGGCC | 0 | 470 |
| 916664 | N/A | N/A | 15597 | 15612 | ATACATAGCAGAGCCA | 44 | 471 |
| 916684 | N/A | N/A | 16352 | 16367 | CACCCTATCGCTCCCA | 43 | 472 |
| 916704 | N/A | N/A | 17267 | 17282 | AGTTATGTCTGACTCA | 72 | 473 |
| 916724 | N/A | N/A | 18254 | 18269 | AATATACCCCACAGCA | 40 | 474 |
| 916744 | N/A | N/A | 19288 | 19303 | GTGCATGTGTGGCTTG | 82 | 475 |
| 916763 | N/A | N/A | 20414 | 20429 | TGTAGTAAGCAATGCA | 85 | 476 |
| 916783 | N/A | N/A | 20724 | 20739 | CATATATTGCGGATGA | 24 | 477 |
| 916803 | N/A | N/A | 21005 | 21020 | GTTATTTTAACAGCTC | 95 | 478 |
| 916823 | N/A | N/A | 21561 | 21576 | ATAAGGACTTACACCA | 83 | 479 |
| 916843 | N/A | N/A | 22679 | 22694 | CAGCATGCAACCACCC | 8 | 480 |
| 916863 | N/A | N/A | 23550 | 23565 | TGGGATGCTAGGACAA | 72 | 481 |
| 916883 | N/A | N/A | 24340 | 24355 | GGAAGTAGCGGCATCC | 0 | 482 |

TABLE 8
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915346 | 25 | 40 | 2763 | 2778 | CATTCCCAGCGCGACG | 0 | 483 |
| 915366 | 52 | 67 | 2790 | 2805 | TACCCTGCCTCAGTGT | 0 | 484 |
| 915386 | 112 | 127 | 2850 | 2865 | TCGGGAATCGGCTCGG | 26 | 485 |
| 915406 | 195 | 210 | 2933 | 2948 | CGAAGGACAAGCTCCA | 69 | 486 |
| 915426 | 252 | 267 | 2990 | 3005 | GCTCGCTCAGGCAGCG | 0 | 487 |
| 915446 | 350 | 365 | N/A | N/A | CTGCTCCAGCGGGATA | 0 | 488 |
| 915466 | 389 | 404 | 5963 | 5978 | CCTGGCCTTCCGCACA | 40 | 489 |
| 915486 | 431 | 446 | 6005 | 6020 | GCTTAAGTTGAAGGAT | 88 | 490 |
| 915506 | 455 | 470 | 6029 | 6044 | GCAGAGACCCTGTCGG | 32 | 491 |
| 915526 | 493 | 508 | 6067 | 6082 | CCGGAGATGAGCTGGT | 4 | 492 |
| 915546 | 514 | 529 | 6088 | 6103 | GTAAGAGAGATGCCTA | 94 | 493 |
| 915566 | 562 | 577 | 6136 | 6151 | TTGGACCGAAAGTCAG | 56 | 494 |
| 915606 | 702 | 717 | 11920 | 11935 | TGGTTGTTTTGGCATC | 99 | 495 |
| 915626 | 757 | 772 | 11975 | 11990 | GTGGACTTGACTTTAG | 89 | 496 |
| 915646 | 830 | 845 | 12048 | 12063 | TCTCGAGAGAAGGTAG | 0 | 497 |
| 915666 | 882 | 897 | 13618 | 13633 | ATCCTCGAAGGCATAT | 0 | 498 |
| 915686 | 956 | 971 | 16076 | 16091 | TGAGGATGACTTCAGG | 10 | 499 |
| 915706 | 989 | 1004 | 16109 | 16124 | GCTGGGCATGGCGACC | 10 | 500 |
| 915726 | 1064 | 1079 | 16184 | 16199 | TAGCAGCTCATCTCCC | 67 | 501 |
| 915746 | 1135 | 1150 | 16255 | 16270 | GTAGCGAGCCTGGGCG | 0 | 502 |
| 915766 | 1196 | 1211 | 19036 | 19051 | TAGCAAGTTGCAAATC | 78 | 503 |
| 915786 | 1232 | 1247 | 19072 | 19087 | ACAGGGCAGCATTACA | 87 | 504 |
| 915806 | 1302 | 1317 | 23707 | 23722 | CGTCGGGCATATCTGG | 53 | 505 |
| 915826 | 1352 | 1367 | 23757 | 23772 | CAGCACTCGAGTGAAC | 24 | 506 |
| 915846 | 1408 | 1423 | 25171 | 25186 | GAGGCCTGTTGGCTGC | 0 | 507 |
| 915866 | 1508 | 1523 | 25271 | 25286 | GAGGATGGACCGCGGG | 0 | 508 |
| 915886 | 1549 | 1564 | 25312 | 25327 | GCAGGTACTTTATTGC | 0 | 509 |
| 915906 | 1610 | 1625 | 25373 | 25388 | AGTGACTCACAGACTC | 35 | 510 |
| 915926 | 1636 | 1651 | 25399 | 25414 | AAGAATCTGCTAGACT | 69 | 511 |
| 915946 | 1689 | 1704 | 25452 | 25467 | ACACAGCAATGCGGAG | 69 | 512 |
| 915966 | 1771 | 1786 | 25534 | 25549 | GGCGAAAGGTTGCTTC | 58 | 513 |
| 915986 | 1792 | 1807 | 25555 | 25570 | TAAGTGCTGGACCGCT | 70 | 514 |
| 916006 | 1818 | 1833 | 25581 | 25596 | ATTAACGCATGCTGAT | 73 | 515 |
| 916026 | 1851 | 1866 | 25614 | 25629 | TGGGCTTCCTGGTGTC | 71 | 516 |
| 916046 | 1887 | 1902 | 25650 | 25665 | AGGGCCACGAAACAGT | 61 | 517 |
| 916066 | 1917 | 1932 | 25680 | 25695 | ACCTCATGCTGGAACA | 81 | 518 |
| 916086 | 1960 | 1975 | 25723 | 25738 | CATCACAAGGCCCCCA | 48 | 519 |
| 916106 | 1980 | 1995 | 25743 | 25758 | ACATGGGCCAGCCTAC | 54 | 520 |
| 916126 | 2055 | 2070 | 25818 | 25833 | GACGAACTGCACCCCT | 77 | 521 |
| 916145 | 2105 | 2120 | 25868 | 25883 | TCAAGGTTATCATCTT | 89 | 522 |
| 916165 | 2142 | 2157 | 25905 | 25920 | CATCTTGTTACCCCCG | 89 | 523 |
| 916185 | 2270 | 2285 | 26033 | 26048 | CTAACATCTCACTGAT | 66 | 524 |
| 916205 | 2627 | 2642 | 26390 | 26405 | ATGCCCTGCACACTAG | 62 | 525 |
| 916225 | 2681 | 2696 | 26444 | 26459 | AGAGGAGGCGGAAGCT | 25 | 526 |
| 916245 | 2711 | 2726 | 26474 | 26489 | AAGCCAGGTTCAAGTT | 83 | 527 |
| 916285 | N/A | N/A | 4240 | 4255 | ATTAGGACAAGATTCA | 75 | 528 |
| 916305 | N/A | N/A | 4866 | 4881 | TGTACTTTAGGCTCCT | 93 | 529 |
| 916325 | N/A | N/A | 5430 | 5445 | AACATATCAGCATTAG | 85 | 530 |
| 916345 | N/A | N/A | 5839 | 5854 | CAAGGATGCCACCAAC | 84 | 531 |
| 916365 | N/A | N/A | 6974 | 6989 | TCATTATGGAAGATGC | 0 | 532 |
| 916385 | N/A | N/A | 7602 | 7617 | TTAACAACCCTGTCAG | 1 | 533 |
| 916545 | N/A | N/A | 12151 | 12166 | GTAACTGGTAGCTCCT | 93 | 534 |
| 916565 | N/A | N/A | 12338 | 12353 | ACCCATACTGCACCCC | 79 | 535 |
| 916585 | N/A | N/A | 12957 | 12972 | GCCTATCCCAATTGCC | 70 | 536 |
| 916605 | N/A | N/A | 13719 | 13734 | GTCTTTACCAAGACCG | 23 | 537 |
| 916625 | N/A | N/A | 14248 | 14263 | AACGACAAATTCATCA | 84 | 538 |
| 916645 | N/A | N/A | 14788 | 14803 | TGCAATCCCCCTGTAT | 17 | 539 |
| 916665 | N/A | N/A | 15598 | 15613 | AATACATAGCAGAGCC | 68 | 540 |
| 916685 | N/A | N/A | 16366 | 16381 | TGTCATGGTTGCCTCA | 70 | 541 |
| 916705 | N/A | N/A | 17273 | 17288 | ATAAGGAGTTATGTCT | 80 | 542 |
| 916725 | N/A | N/A | 18255 | 18270 | GAATATACCCCACAGC | 58 | 543 |
| 916745 | N/A | N/A | 19295 | 19310 | GTTACAGGTGCATGTG | 75 | 544 |
| 916764 | N/A | N/A | 20435 | 20450 | AGTCATCTGGAGTCAC | 69 | 545 |
| 916784 | N/A | N/A | 20756 | 20771 | TCAGACAACCCACTGA | 24 | 546 |
| 916804 | N/A | N/A | 21046 | 21061 | AGGAATCTGAATCCTA | 0 | 547 |
| 916824 | N/A | N/A | 21640 | 21655 | GATAATTTCCTAGAGC | 29 | 548 |
| 916844 | N/A | N/A | 22699 | 22714 | GAAATAAGTGCTCAGG | 73 | 549 |
| 916864 | N/A | N/A | 23582 | 23597 | CTCCAATCTGATGACT | 53 | 550 |
| 916884 | N/A | N/A | 24347 | 24362 | GAATTCAGGAAGTAGC | 50 | 551 |

TABLE 9
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915347 | 26 | 41 | 2764 | 2779 | GCATTCCCAGCGCGAC | 0 | 552 |
| 915367 | 58 | 73 | 2796 | 2811 | GCTCTCTACCCTGCCT | 9 | 553 |
| 915387 | 113 | 128 | 2851 | 2866 | ATCGGGAATCGGCTCG | 28 | 554 |
| 915407 | 198 | 213 | 2936 | 2951 | CCGCGAAGGACAAGCT | 0 | 555 |
| 915427 | 253 | 268 | 2991 | 3006 | TGCTCGCTCAGGCAGC | 0 | 556 |
| 915447 | 351 | 366 | N/A | N/A | TCTGCTCCAGCGGGAT | 1 | 557 |
| 915467 | 391 | 406 | 5965 | 5980 | CTCCTGGCCTTCCGCA | 29 | 558 |
| 915487 | 433 | 448 | 6007 | 6022 | TTGCTTAAGTTGAAGG | 94 | 559 |
| 915507 | 456 | 471 | 6030 | 6045 | TGCAGAGACCCTGTCG | 31 | 560 |
| 915527 | 494 | 509 | 6068 | 6083 | GCCGGAGATGAGCTGG | 0 | 561 |
| 915547 | 515 | 530 | 6089 | 6104 | GGTAAGAGAGATGCCT | 0 | 562 |
| 915567 | 563 | 578 | 6137 | 6152 | TTTGGACCGAAAGTCA | 0 | 563 |
| 915607 | 703 | 718 | 11921 | 11936 | ATGGTTGTTTTGGCAT | 35 | 564 |
| 915627 | 758 | 773 | 11976 | 11991 | CGTGGACTTGACTTTA | 85 | 565 |
| 915647 | 831 | 846 | 12049 | 12064 | CTCTCGAGAGAAGGTA | 7 | 566 |
| 915667 | 883 | 898 | 13619 | 13634 | TATCCTCGAAGGCATA | 0 | 567 |
| 915687 | 959 | 974 | 16079 | 16094 | TTCTGAGGATGACTTC | 39 | 568 |
| 915707 | 996 | 1011 | 16116 | 16131 | TTGCCCAGCTGGGCAT | 0 | 569 |
| 915727 | 1065 | 1080 | 16185 | 16200 | CTAGCAGCTCATCTCC | 58 | 570 |
| 915747 | 1136 | 1151 | 16256 | 16271 | TGTAGCGAGCCTGGGC | 16 | 571 |
| 915767 | 1197 | 1212 | 19037 | 19052 | GTAGCAAGTTGCAAAT | 80 | 572 |
| 915787 | 1233 | 1248 | 19073 | 19088 | TACAGGGCAGCATTAC | 71 | 573 |
| 915807 | 1316 | 1331 | 23721 | 23736 | CAACCACAGGACATCG | 0 | 574 |
| 915827 | 1353 | 1368 | 23758 | 23773 | TCAGCACTCGAGTGAA | 0 | 575 |
| 915847 | 1409 | 1424 | 25172 | 25187 | GGAGGCCTGTTGGCTG | 0 | 576 |
| 915867 | 1509 | 1524 | 25272 | 25287 | TGAGGATGGACCGCGG | 14 | 577 |
| 915887 | 1553 | 1568 | 25316 | 25331 | ACCAGCAGGTACTTTA | 29 | 578 |
| 915907 | 1611 | 1626 | 25374 | 25389 | AAGTGACTCACAGACT | 29 | 579 |
| 915927 | 1637 | 1652 | 25400 | 25415 | AAAGAATCTGCTAGAC | 60 | 580 |
| 915947 | 1690 | 1705 | 25453 | 25468 | TACACAGCAATGCGGA | 69 | 581 |
| 915967 | 1772 | 1787 | 25535 | 25550 | AGGCGAAAGGTTGCTT | 0 | 582 |
| 915987 | 1793 | 1808 | 25556 | 25571 | TTAAGTGCTGGACCGC | 82 | 583 |
| 916007 | 1819 | 1834 | 25582 | 25597 | AATTAACGCATGCTGA | 61 | 584 |
| 916027 | 1864 | 1879 | 25627 | 25642 | GGACCCTCTGCACTGG | 43 | 585 |
| 916047 | 1888 | 1903 | 25651 | 25666 | TAGGGCCACGAAACAG | 80 | 586 |
| 916067 | 1918 | 1933 | 25681 | 25696 | AACCTCATGCTGGAAC | 72 | 587 |
| 916087 | 1961 | 1976 | 25724 | 25739 | CCATCACAAGGCCCCC | 63 | 588 |
| 916107 | 1981 | 1996 | 25744 | 25759 | CACATGGGCCAGCCTA | 74 | 589 |
| 916127 | 2056 | 2071 | 25819 | 25834 | GGACGAACTGCACCCC | 5 | 590 |
| 916146 | 2106 | 2121 | 25869 | 25884 | GTCAAGGTTATCATCT | 88 | 591 |
| 916166 | 2143 | 2158 | 25906 | 25921 | TCATCTTGTTACCCCC | 90 | 592 |
| 916186 | 2272 | 2287 | 26035 | 26050 | TACTAACATCTCACTG | 1 | 593 |
| 916206 | 2628 | 2643 | 26391 | 26406 | AATGCCCTGCACACTA | 56 | 594 |
| 916226 | 2682 | 2697 | 26445 | 26460 | GAGAGGAGGCGGAAGC | 10 | 595 |
| 916246 | 2712 | 2727 | 26475 | 26490 | TAAGCCAGGTTCAAGT | 81 | 596 |
| 916286 | N/A | N/A | 4244 | 4259 | TTTCATTAGGACAAGA | 61 | 597 |
| 916306 | N/A | N/A | 4867 | 4882 | GTGTACTTTAGGCTCC | 97 | 598 |
| 916326 | N/A | N/A | 5431 | 5446 | GAACATATCAGCATTA | 52 | 599 |
| 916346 | N/A | N/A | 5872 | 5887 | GTAATACTTTTGGCAA | 75 | 600 |
| 916366 | N/A | N/A | 7069 | 7084 | GGTATTACAAATTATC | 10 | 601 |
| 916386 | N/A | N/A | 7603 | 7618 | CTTAACAACCCTGTCA | 0 | 602 |
| 916546 | N/A | N/A | 12152 | 12167 | AGTAACTGGTAGCTCC | 88 | 603 |
| 916566 | N/A | N/A | 12343 | 12358 | CTAATACCCATACTGC | 84 | 604 |
| 916586 | N/A | N/A | 12966 | 12981 | AACTTTGCAGCCTATC | 85 | 605 |
| 916606 | N/A | N/A | 13739 | 13754 | AGAACTAAGGCAAATC | 85 | 606 |
| 916626 | N/A | N/A | 14257 | 14272 | GTCTTGGCCAACGACA | 0 | 607 |
| 916646 | N/A | N/A | 14793 | 14808 | CAGGATGCAATCCCCC | 45 | 608 |
| 916666 | N/A | N/A | 15601 | 15616 | GCCAATACATAGCAGA | 75 | 609 |
| 916686 | N/A | N/A | 16630 | 16645 | GTCCATGAAATCCAGG | 0 | 610 |
| 916706 | N/A | N/A | 17293 | 17308 | TCTCTTAGGGCACCTC | 87 | 611 |
| 916726 | N/A | N/A | 18256 | 18271 | TGAATATACCCCACAG | 24 | 612 |
| 916746 | N/A | N/A | 19337 | 19352 | AGCTCTAGGAGTCCCC | 63 | 613 |
| 916765 | N/A | N/A | 20513 | 20528 | CCAGATTGAGTCTCCT | 91 | 614 |
| 916785 | N/A | N/A | 20775 | 20790 | AATCAAGTGCCCTCCA | 73 | 615 |
| 916805 | N/A | N/A | 21211 | 21226 | TGTAGCTGTGTGGTGG | 85 | 616 |
| 916825 | N/A | N/A | 21760 | 21775 | TACCATGATCAGGTCA | 0 | 617 |
| 916845 | N/A | N/A | 22713 | 22728 | GTAAAGATGTGAGTGA | 85 | 618 |
| 916865 | N/A | N/A | 23606 | 23621 | GTTTACAAAAGCTGCC | 17 | 619 |
| 916885 | N/A | N/A | 24375 | 24390 | TGAACTCCGGCTCAGT | 0 | 620 |

TABLE 10
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915348 | 28 | 43 | 2766 | 2781 | GGGCATTCCCAGCGCG | 0 | 621 |
| 915368 | 59 | 74 | 2797 | 2812 | CGCTCTCTACCCTGCC | 0 | 622 |
| 915388 | 114 | 129 | 2852 | 2867 | GATCGGGAATCGGCTC | 32 | 623 |
| 915408 | 199 | 214 | 2937 | 2952 | CCCGCGAAGGACAAGC | 6 | 624 |
| 915428 | 275 | 290 | 3013 | 3028 | GTCGCGGAGGAGGTGC | 0 | 625 |
| 915448 | 352 | 367 | N/A | N/A | GTCTGCTCCAGCGGGA | 4 | 626 |
| 915468 | 392 | 407 | 5966 | 5981 | ACTCCTGGCCTTCCGC | 87 | 627 |
| 915488 | 434 | 449 | 6008 | 6023 | CTTGCTTAAGTTGAAG | 0 | 628 |
| 915508 | 457 | 472 | 6031 | 6046 | TTGCAGAGACCCTGTC | 63 | 629 |
| 915528 | 495 | 510 | 6069 | 6084 | TGCCGGAGATGAGCTG | 0 | 630 |
| 915548 | 516 | 531 | 6090 | 6105 | TGGTAAGAGAGATGCC | 18 | 631 |
| 915568 | 564 | 579 | 6138 | 6153 | CTTTGGACCGAAAGTC | 10 | 632 |
| 915608 | 704 | 719 | 11922 | 11937 | GATGGTTGTTTTGGCA | 98 | 633 |
| 915628 | 772 | 787 | 11990 | 12005 | ACATGAAGAAAGTTCG | 41 | 634 |
| 915648 | 832 | 847 | 12050 | 12065 | GCTCTCGAGAGAAGGT | 55 | 635 |
| 915668 | 884 | 899 | 13620 | 13635 | ATATCCTCGAAGGCAT | 33 | 636 |
| 915688 | 962 | 977 | 16082 | 16097 | CCCTTCTGAGGATGAC | 11 | 637 |
| 915708 | 998 | 1013 | 16118 | 16133 | GTTTGCCCAGCTGGGC | 0 | 638 |
| 915728 | 1067 | 1082 | 16187 | 16202 | GTCTAGCAGCTCATCT | 68 | 639 |
| 915748 | 1137 | 1152 | 16257 | 16272 | CTGTAGCGAGCCTGGG | 0 | 640 |
| 915768 | 1198 | 1213 | 19038 | 19053 | GGTAGCAAGTTGCAAA | 90 | 641 |
| 915788 | 1234 | 1249 | 19074 | 19089 | GTACAGGGCAGCATTA | 69 | 642 |
| 915808 | 1317 | 1332 | 23722 | 23737 | GCAACCACAGGACATC | 51 | 643 |
| 915828 | 1354 | 1369 | 23759 | 23774 | ATCAGCACTCGAGTGA | 0 | 644 |
| 915848 | 1410 | 1425 | 25173 | 25188 | GGGAGGCCTGTTGGCT | 17 | 645 |
| 915868 | 1510 | 1525 | 25273 | 25288 | CTGAGGATGGACCGCG | 53 | 646 |
| 915888 | 1554 | 1569 | 25317 | 25332 | CACCAGCAGGTACTTT | 0 | 647 |
| 915908 | 1612 | 1627 | 25375 | 25390 | CAAGTGACTCACAGAC | 91 | 648 |
| 915928 | 1639 | 1654 | 25402 | 25417 | TGAAAGAATCTGCTAG | 59 | 649 |
| 915948 | 1691 | 1706 | 25454 | 25469 | CTACACAGCAATGCGG | 20 | 650 |
| 915968 | 1773 | 1788 | 25536 | 25551 | CAGGCGAAAGGTTGCT | 60 | 651 |
| 915988 | 1794 | 1809 | 25557 | 25572 | GTTAAGTGCTGGACCG | 86 | 652 |
| 916008 | 1820 | 1835 | 25583 | 25598 | GAATTAACGCATGCTG | 88 | 653 |
| 916028 | 1865 | 1880 | 25628 | 25643 | GGGACCCTCTGCACTG | 0 | 654 |
| 916048 | 1889 | 1904 | 25652 | 25667 | ATAGGGCCACGAAACA | 75 | 655 |
| 916068 | 1919 | 1934 | 25682 | 25697 | GAACCTCATGCTGGAA | 72 | 656 |
| 916088 | 1962 | 1977 | 25725 | 25740 | CCCATCACAAGGCCCC | 37 | 657 |
| 916108 | 1984 | 1999 | 25747 | 25762 | TCACACATGGGCCAGC | 84 | 658 |
| 916128 | 2079 | 2094 | 25842 | 25857 | CTGACAGGCAGTGTCG | 10 | 659 |
| 916147 | 2107 | 2122 | 25870 | 25885 | AGTCAAGGTTATCATC | 81 | 660 |
| 916167 | 2144 | 2159 | 25907 | 25922 | ATCATCTTGTTACCCC | 88 | 661 |
| 916187 | 2276 | 2291 | 26039 | 26054 | ATTCTACTAACATCTC | 90 | 662 |
| 916207 | 2629 | 2644 | 26392 | 26407 | GAATGCCCTGCACACT | 72 | 663 |
| 916227 | 2691 | 2706 | 26454 | 26469 | GCTCCAGTGGAGAGGA | 14 | 664 |
| 916247 | 2713 | 2728 | 26476 | 26491 | ATAAGCCAGGTTCAAG | 88 | 665 |
| 916287 | N/A | N/A | 4308 | 4323 | GTGAGAAACAAACCCT | 92 | 666 |
| 916307 | N/A | N/A | 4882 | 4897 | TCTATACCAGAGTGAG | 84 | 667 |
| 916327 | N/A | N/A | 5514 | 5529 | AGGAATGAGTCTCCCA | 17 | 668 |
| 916347 | N/A | N/A | 5873 | 5888 | GGTAATACTTTTGGCA | 70 | 669 |
| 916367 | N/A | N/A | 7106 | 7121 | CGCTTATGAAAGCATC | 0 | 670 |
| 916387 | N/A | N/A | 7605 | 7620 | CCCTTAACAACCCTGT | 28 | 671 |
| 916547 | N/A | N/A | 12167 | 12182 | TTTGATTGTGCAGACA | 98 | 672 |
| 916567 | N/A | N/A | 12345 | 12360 | TCCTAATACCCATACT | 74 | 673 |
| 916587 | N/A | N/A | 12969 | 12984 | ACAAACTTTGCAGCCT | 95 | 674 |
| 916607 | N/A | N/A | 13742 | 13757 | GTTAGAACTAAGGCAA | 94 | 675 |
| 916627 | N/A | N/A | 14301 | 14316 | GAGCAGATAAATACAC | 91 | 676 |
| 916647 | N/A | N/A | 14892 | 14907 | TGGTATCTCGCTTCCT | 0 | 677 |
| 916667 | N/A | N/A | 15613 | 15628 | TAAAGCCACGCAGCCA | 46 | 678 |
| 916687 | N/A | N/A | 16656 | 16671 | CCAGATGCAGGACCCC | 0 | 679 |
| 916707 | N/A | N/A | 17326 | 17341 | AAACTAATGCACCTGG | 43 | 680 |
| 916727 | N/A | N/A | 18257 | 18272 | CTGAATATACCCCACA | 75 | 681 |
| 916747 | N/A | N/A | 19360 | 19375 | AGCTGCTATGTGAGGC | 12 | 682 |
| 916766 | N/A | N/A | 20520 | 20535 | TCAGTAACCAGATTGA | 25 | 683 |
| 916786 | N/A | N/A | 20778 | 20793 | TTTAATCAAGTGCCCT | 81 | 684 |
| 916806 | N/A | N/A | 21216 | 21231 | CAGGATGTAGCTGTGT | 84 | 685 |
| 916826 | N/A | N/A | 21887 | 21902 | TAAGATCCCATCTTAC | 13 | 686 |
| 916846 | N/A | N/A | 22739 | 22754 | AAAGTAAACACCCACC | 42 | 687 |
| 916866 | N/A | N/A | 23625 | 23640 | GCTTACAACACTACCC | 57 | 688 |
| 916886 | N/A | N/A | 24393 | 24408 | GTAATGGGAGCCAGGC | 38 | 689 |

TABLE 11
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915349 | 29 | 44 | 2767 | 2782 | AGGGCATTCCCAGCGC | 0 | 690 |
| 915369 | 60 | 75 | 2798 | 2813 | GCGCTCTCTACCCTGC | 23 | 691 |
| 915389 | 115 | 130 | 2853 | 2868 | GGATCGGGAATCGGCT | 54 | 692 |
| 915409 | 200 | 215 | 2938 | 2953 | GCCCGCGAAGGACAAG | 32 | 693 |
| 915429 | 276 | 291 | 3014 | 3029 | CGTCGCGGAGGAGGTG | 0 | 694 |
| 915449 | 364 | 379 | 5938 | 5953 | AGGACCTGCAGAGTCT | 21 | 695 |
| 915469 | 394 | 409 | 5968 | 5983 | CGACTCCTGGCCTTCC | 59 | 696 |
| 915489 | 435 | 450 | 6009 | 6024 | ACTTGCTTAAGTTGAA | 86 | 697 |
| 915509 | 466 | 481 | 6040 | 6055 | GGGAGGCATTTGCAGA | 57 | 698 |
| 915529 | 496 | 511 | 6070 | 6085 | TTGCCGGAGATGAGCT | 40 | 699 |
| 915549 | 518 | 533 | 6092 | 6107 | TCTGGTAAGAGAGATG | 61 | 700 |
| 915569 | 565 | 580 | 6139 | 6154 | TCTTTGGACCGAAAGT | 9 | 701 |
| 915609 | 705 | 720 | 11923 | 11938 | TGATGGTTGTTTTGGC | 99 | 702 |
| 915629 | 776 | 791 | 11994 | 12009 | GTCCACATGAAGAAAG | 32 | 703 |
| 915649 | 833 | 848 | 12051 | 12066 | AGCTCTCGAGAGAAGG | 36 | 704 |
| 915669 | 885 | 900 | 13621 | 13636 | AATATCCTCGAAGGCA | 55 | 705 |
| 915689 | 969 | 984 | 16089 | 16104 | GATCCATCCCTTCTGA | 20 | 706 |
| 915709 | 999 | 1014 | 16119 | 16134 | TGTTTGCCCAGCTGGG | 5 | 707 |
| 915729 | 1077 | 1092 | 16197 | 16212 | GACGCAGGTGGTCTAG | 0 | 708 |
| 915749 | 1138 | 1153 | N/A | N/A | GCTGTAGCGAGCCTGG | 71 | 709 |
| 915769 | 1200 | 1215 | 19040 | 19055 | TGGGTAGCAAGTTGCA | 81 | 710 |
| 915789 | 1235 | 1250 | 19075 | 19090 | GGTACAGGGCAGCATT | 88 | 711 |
| 915809 | 1318 | 1333 | 23723 | 23738 | TGCAACCACAGGACAT | 40 | 712 |
| 915829 | 1355 | 1370 | 23760 | 23775 | CATCAGCACTCGAGTG | 0 | 713 |
| 915849 | 1424 | 1439 | 25187 | 25202 | CTCAGGTGTGCATGGG | 61 | 714 |
| 915869 | 1511 | 1526 | 25274 | 25289 | CCTGAGGATGGACCGC | 70 | 715 |
| 915889 | 1556 | 1571 | 25319 | 25334 | AGCACCAGCAGGTACT | 35 | 716 |
| 915909 | 1613 | 1628 | 25376 | 25391 | TCAAGTGACTCACAGA | 84 | 717 |
| 915929 | 1645 | 1660 | 25408 | 25423 | CACCTCTGAAAGAATC | 89 | 718 |
| 915949 | 1692 | 1707 | 25455 | 25470 | ACTACACAGCAATGCG | 33 | 719 |
| 915969 | 1774 | 1789 | 25537 | 25552 | ACAGGCGAAAGGTTGC | 88 | 720 |
| 915989 | 1795 | 1810 | 25558 | 25573 | AGTTAAGTGCTGGACC | 84 | 721 |
| 916009 | 1823 | 1838 | 25586 | 25601 | GCTGAATTAACGCATG | 67 | 722 |
| 916029 | 1866 | 1881 | 25629 | 25644 | AGGGACCCTCTGCACT | 15 | 723 |
| 916049 | 1890 | 1905 | 25653 | 25668 | AATAGGGCCACGAAAC | 52 | 724 |
| 916069 | 1920 | 1935 | 25683 | 25698 | AGAACCTCATGCTGGA | 85 | 725 |
| 916089 | 1963 | 1978 | 25726 | 25741 | CCCCATCACAAGGCCC | 20 | 726 |
| 916109 | 1985 | 2000 | 25748 | 25763 | ATCACACATGGGCCAG | 72 | 727 |
| 916129 | 2080 | 2095 | 25843 | 25858 | CCTGACAGGCAGTGTC | 15 | 728 |
| 916148 | 2108 | 2123 | 25871 | 25886 | TAGTCAAGGTTATCAT | 87 | 729 |
| 916168 | 2146 | 2161 | 25909 | 25924 | TTATCATCTTGTTACC | 82 | 730 |
| 916188 | 2279 | 2294 | 26042 | 26057 | CTTATTCTACTAACAT | 87 | 731 |
| 916208 | 2630 | 2645 | 26393 | 26408 | TGAATGCCCTGCACAC | 68 | 732 |
| 916228 | 2692 | 2707 | 26455 | 26470 | TGCTCCAGTGGAGAGG | 80 | 733 |
| 916248 | 2726 | 2741 | 26489 | 26504 | GTCCCTGCAGAAAATA | 0 | 734 |
| 916288 | N/A | N/A | 4337 | 4352 | AGCATACCACACCCCA | 75 | 735 |
| 916308 | N/A | N/A | 5086 | 5101 | GGACATGCTCAGCAGC | 68 | 736 |
| 916328 | N/A | N/A | 5533 | 5548 | TGCTGTAGGCCTCAGC | 0 | 737 |
| 916348 | N/A | N/A | 5874 | 5889 | TGGTAATACTTTTGGC | 86 | 738 |
| 916368 | N/A | N/A | 7132 | 7147 | GTAAATGGAGTCCTTC | 80 | 739 |
| 916388 | N/A | N/A | 7612 | 7627 | CATAATCCCCTTAACA | 32 | 740 |
| 916548 | N/A | N/A | 12195 | 12210 | TTAACCATCAAGGACA | 77 | 741 |
| 916568 | N/A | N/A | 12665 | 12680 | TCTTAGTGGCTGGGTA | 85 | 742 |
| 916588 | N/A | N/A | 12973 | 12988 | CCTAACAAACTTTGCA | 32 | 743 |
| 916608 | N/A | N/A | 13749 | 13764 | ACTAAGTGTTAGAACT | 76 | 744 |
| 916628 | N/A | N/A | 14338 | 14353 | CTGCAGTATCCCTAGC | 0 | 745 |
| 916648 | N/A | N/A | 15012 | 15027 | TCCCATCGGTCATTTC | 45 | 746 |
| 916668 | N/A | N/A | 15682 | 15697 | GAAACCACTATCATCA | 62 | 747 |
| 916688 | N/A | N/A | 16671 | 16686 | GTAATAGGCCAAGTCC | 0 | 748 |
| 916708 | N/A | N/A | 17327 | 17342 | CAAACTAATGCACCTG | 66 | 749 |
| 916728 | N/A | N/A | 18332 | 18347 | CCAATATCATAGCTGA | 85 | 750 |
| 916748 | N/A | N/A | 19376 | 19391 | CACAAGAGACTGGACC | 64 | 751 |
| 916767 | N/A | N/A | 20551 | 20566 | TACTATGGGATGAGTA | 0 | 752 |
| 916787 | N/A | N/A | 20779 | 20794 | TTTTAATCAAGTGCCC | 38 | 753 |
| 916807 | N/A | N/A | 21218 | 21233 | GGCAGGATGTAGCTGT | 63 | 754 |
| 916827 | N/A | N/A | 21947 | 21962 | AGTCAAACATCTTCCT | 50 | 755 |
| 916847 | N/A | N/A | 22759 | 22774 | CAGACTAACTTACTAA | 77 | 756 |
| 916867 | N/A | N/A | 23626 | 23641 | AGCTTACAACACTACC | 13 | 757 |
| 916887 | N/A | N/A | 24505 | 24520 | ATGCTACGGGCTCTCA | 0 | 758 |

TABLE 12
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915350 | 30 | 45 | 2768 | 2783 | CAGGGCATTCCCAGCG | 0 | 759 |
| 915370 | 82 | 97 | 2820 | 2835 | CAGCTCCGCCCGGCGC | 14 | 760 |
| 915390 | 130 | 145 | 2868 | 2883 | TTAGGATCTGGGTCGG | 88 | 761 |
| 915410 | 201 | 216 | 2939 | 2954 | AGCCCGCGAAGGACAA | 0 | 762 |
| 915430 | 295 | 310 | 3033 | 3048 | GCGCCGAACAACATGC | 0 | 763 |
| 915450 | 366 | 381 | 5940 | 5955 | AGAGGACCTGCAGAGT | 83 | 764 |
| 915470 | 395 | 410 | 5969 | 5984 | CCGACTCCTGGCCTTC | 68 | 765 |
| 915490 | 436 | 451 | 6010 | 6025 | AACTTGCTTAAGTTGA | 41 | 766 |
| 915510 | 467 | 482 | 6041 | 6056 | CGGGAGGCATTTGCAG | 44 | 767 |
| 915530 | 497 | 512 | 6071 | 6086 | TTTGCCGGAGATGAGC | 92 | 768 |
| 915550 | 519 | 534 | 6093 | 6108 | CTCTGGTAAGAGAGAT | 20 | 769 |
| 915570 | 566 | 581 | 6140 | 6155 | GTCTTTGGACCGAAAG | 19 | 770 |
| 915590 | 627 | 642 | 7857 | 7872 | GAGGGATAAGGCCACT | 83 | 771 |
| 915610 | 706 | 721 | 11924 | 11939 | GTGATGGTTGTTTTGG | 97 | 772 |
| 915630 | 782 | 797 | 12000 | 12015 | GGTGATGTCCACATGA | 87 | 773 |
| 915650 | 834 | 849 | 12052 | 12067 | AAGCTCTCGAGAGAAG | 44 | 774 |
| 915670 | 887 | 902 | 13623 | 13638 | CAAATATCCTCGAAGG | 0 | 775 |
| 915690 | 970 | 985 | 16090 | 16105 | GGATCCATCCCTTCTG | 0 | 776 |
| 915710 | 1003 | 1018 | 16123 | 16138 | CTCATGTTTGCCCAGC | 68 | 777 |
| 915730 | 1078 | 1093 | 16198 | 16213 | AGACGCAGGTGGTCTA | 0 | 778 |
| 915750 | 1139 | 1154 | N/A | N/A | TGCTGTAGCGAGCCTG | 56 | 779 |
| 915770 | 1201 | 1216 | 19041 | 19056 | ATGGGTAGCAAGTTGC | 79 | 780 |
| 915790 | 1247 | 1262 | 19087 | 19102 | TTCCACAGGCAGGGTA | 48 | 781 |
| 915810 | 1320 | 1335 | 23725 | 23740 | ACTGCAACCACAGGAC | 22 | 782 |
| 915830 | 1356 | 1371 | 23761 | 23776 | ACATCAGCACTCGAGT | 0 | 783 |
| 915850 | 1427 | 1442 | 25190 | 25205 | CTGCTCAGGTGTGCAT | 10 | 784 |
| 915870 | 1512 | 1527 | 25275 | 25290 | ACCTGAGGATGGACCG | 69 | 785 |
| 915890 | 1557 | 1572 | 25320 | 25335 | CAGCACCAGCAGGTAC | 62 | 786 |
| 915910 | 1617 | 1632 | 25380 | 25395 | CTCCTCAAGTGACTCA | 83 | 787 |
| 915930 | 1648 | 1663 | 25411 | 25426 | TAGCACCTCTGAAAGA | 55 | 788 |
| 915950 | 1693 | 1708 | 25456 | 25471 | CACTACACAGCAATGC | 74 | 789 |
| 915970 | 1775 | 1790 | 25538 | 25553 | CACAGGCGAAAGGTTG | 72 | 790 |
| 915990 | 1797 | 1812 | 25560 | 25575 | AGAGTTAAGTGCTGGA | 92 | 791 |
| 916010 | 1824 | 1839 | 25587 | 25602 | AGCTGAATTAACGCAT | 0 | 792 |
| 916030 | 1867 | 1882 | 25630 | 25645 | AAGGGACCCTCTGCAC | 38 | 793 |
| 916050 | 1891 | 1906 | 25654 | 25669 | TAATAGGGCCACGAAA | 53 | 794 |
| 916070 | 1921 | 1936 | 25684 | 25699 | AAGAACCTCATGCTGG | 24 | 795 |
| 916090 | 1964 | 1979 | 25727 | 25742 | CCCCCATCACAAGGCC | 24 | 796 |
| 916110 | 1986 | 2001 | 25749 | 25764 | GATCACACATGGGCCA | 0 | 797 |
| 916130 | 2081 | 2096 | 25844 | 25859 | ACCTGACAGGCAGTGT | 54 | 798 |
| 916149 | 2109 | 2124 | 25872 | 25887 | GTAGTCAAGGTTATCA | 87 | 799 |
| 916169 | 2154 | 2169 | 25917 | 25932 | TAAGTAGATTATCATC | 79 | 800 |
| 916189 | 2282 | 2297 | 26045 | 26060 | AGGCTTATTCTACTAA | 85 | 801 |
| 916209 | 2631 | 2646 | 26394 | 26409 | GTGAATGCCCTGCACA | 59 | 802 |
| 916229 | 2693 | 2708 | 26456 | 26471 | GTGCTCCAGTGGAGAG | 54 | 803 |
| 916249 | 2727 | 2742 | 26490 | 26505 | GGTCCCTGCAGAAAAT | 38 | 804 |
| 916289 | N/A | N/A | 4338 | 4353 | AAGCATACCACACCCC | 79 | 805 |
| 916309 | N/A | N/A | 5278 | 5293 | AATCTTGGGATGCACA | 95 | 806 |
| 916329 | N/A | N/A | 5569 | 5584 | CATCATGGCTTCCAGT | 79 | 807 |
| 916349 | N/A | N/A | 5879 | 5894 | TGGGATGGTAATACTT | 0 | 808 |
| 916369 | N/A | N/A | 7134 | 7149 | AAGTAAATGGAGTCCT | 5 | 809 |
| 916389 | N/A | N/A | 7615 | 7630 | TTGCATAATCCCCTTA | 33 | 810 |
| 916409 | N/A | N/A | 8165 | 8180 | TTAACTAGATCACTGA | 58 | 811 |
| 916429 | N/A | N/A | 9109 | 9124 | TCCTAATGCGAGTCCC | 86 | 812 |
| 916449 | N/A | N/A | 9522 | 9537 | TGCTGCTGGGTGCACT | 45 | 813 |
| 916469 | N/A | N/A | 10199 | 10214 | GGTGATGACACAGCAT | 94 | 814 |
| 916489 | N/A | N/A | 10382 | 10397 | GCCATGTACAACTTTT | 52 | 815 |
| 916509 | N/A | N/A | 11152 | 11167 | TACAATTTGGACAGAG | 71 | 816 |
| 916529 | N/A | N/A | 11546 | 11561 | ACCTATAGGAGTGCCC | 35 | 817 |
| 916549 | N/A | N/A | 12204 | 12219 | TTATTTCCGTTAACCA | 97 | 818 |
| 916569 | N/A | N/A | 12672 | 12687 | AGAATCATCTTAGTGG | 94 | 819 |
| 916589 | N/A | N/A | 12989 | 13004 | CGGAATAAGCCTCCAC | 0 | 820 |
| 916609 | N/A | N/A | 13752 | 13767 | GGCACTAAGTGTTAGA | 57 | 821 |
| 916629 | N/A | N/A | 14375 | 14390 | TCTCACAAGGCTGGCA | 84 | 822 |
| 916649 | N/A | N/A | 15137 | 15152 | GCCATACCGGCTCCCT | 30 | 823 |
| 916669 | N/A | N/A | 15691 | 15706 | GGCCTTACAGAAACCA | 15 | 824 |
| 916689 | N/A | N/A | 16672 | 16687 | AGTAATAGGCCAAGTC | 16 | 825 |
| 916709 | N/A | N/A | 17328 | 17343 | ACAAACTAATGCACCT | 42 | 826 |
| 916729 | N/A | N/A | 18333 | 18348 | TCCAATATCATAGCTG | 32 | 827 |
| 916749 | N/A | N/A | 19445 | 19460 | CTACTAGGCATCTCTA | 32 | 828 |
| 916768 | N/A | N/A | 20553 | 20568 | CTTACTATGGGATGAG | 83 | 829 |
| 916788 | N/A | N/A | 20808 | 20823 | TAATATTCAGACCAGG | 94 | 830 |
| 916808 | N/A | N/A | 21252 | 21267 | CCATGCATGGCACAGT | 4 | 831 |
| 916828 | N/A | N/A | 21968 | 21983 | AGACAGGAATCCAACC | 0 | 832 |
| 916848 | N/A | N/A | 22767 | 22782 | GGACATGACAGACTAA | 96 | 833 |
| 916868 | N/A | N/A | 23637 | 23652 | GCAGACACAACAGCTT | 40 | 834 |
| 916888 | N/A | N/A | 24507 | 24522 | CCATGCTACGGGCTCT | 0 | 835 |

TABLE 13
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915351 | 33 | 48 | 2771 | 2786 | GGCCAGGGCATTCCCA | 0 | 836 |
| 915371 | 83 | 98 | 2821 | 2836 | GCAGCTCCGCCCGGCG | 2 | 837 |
| 915391 | 132 | 147 | 2870 | 2885 | GGTTAGGATCTGGGTC | 54 | 838 |
| 915411 | 222 | 237 | 2960 | 2975 | GGTAGAAGCCCAGGAA | 57 | 839 |
| 915431 | 321 | 336 | 3059 | 3074 | CGACGCAGTGCAACGC | 49 | 840 |
| 915451 | 368 | 383 | 5942 | 5957 | TGAGAGGACCTGCAGA | 0 | 841 |
| 915471 | 400 | 415 | 5974 | 5989 | ATGTTCCGACTCCTGG | 82 | 842 |
| 915491 | 437 | 452 | 6011 | 6026 | GAACTTGCTTAAGTTG | 23 | 843 |
| 915511 | 468 | 483 | 6042 | 6057 | CCGGGAGGCATTTGCA | 0 | 844 |
| 915531 | 498 | 513 | 6072 | 6087 | TTTTGCCGGAGATGAG | 84 | 845 |
| 915551 | 520 | 535 | 6094 | 6109 | ACTCTGGTAAGAGAGA | 5 | 846 |
| 915571 | 567 | 582 | 6141 | 6156 | CGTCTTTGGACCGAAA | 64 | 847 |
| 915611 | 708 | 723 | 11926 | 11941 | CGGTGATGGTTGTTTT | 98 | 848 |
| 915631 | 783 | 798 | 12001 | 12016 | TGGTGATGTCCACATG | 0 | 849 |
| 915651 | 835 | 850 | 12053 | 12068 | AAAGCTCTCGAGAGAA | 35 | 850 |
| 915671 | 890 | 905 | 13626 | 13641 | ATCCAAATATCCTCGA | 42 | 851 |
| 915691 | 971 | 986 | 16091 | 16106 | AGGATCCATCCCTTCT | 0 | 852 |
| 915711 | 1005 | 1020 | 16125 | 16140 | GACTCATGTTTGCCCA | 73 | 853 |
| 915731 | 1079 | 1094 | 16199 | 16214 | GAGACGCAGGTGGTCT | 0 | 854 |
| 915751 | 1140 | 1155 | N/A | N/A | GTGCTGTAGCGAGCCT | 0 | 855 |
| 915771 | 1202 | 1217 | 19042 | 19057 | AATGGGTAGCAAGTTG | 80 | 856 |
| 915791 | 1248 | 1263 | 19088 | 19103 | ATTCCACAGGCAGGGT | 37 | 857 |

| | | | | | | | |
|---|---|---|---|---|---|---|---|
| 915811 | 1327 | 1342 | 23732 | 23747 | GTCACCCACTGCAACC | 52 | 858 |
| 915831 | 1357 | 1372 | 23762 | 23777 | CACATCAGCACTCGAG | 29 | 859 |
| 915851 | 1429 | 1444 | 25192 | 25207 | TCCTGCTCAGGTGTGC | 2 | 860 |
| 915871 | 1513 | 1528 | 25276 | 25291 | GACCTGAGGATGGACC | 20 | 861 |
| 915891 | 1558 | 1573 | 25321 | 25336 | TCAGCACCAGCAGGTA | 68 | 862 |
| 915911 | 1620 | 1635 | 25383 | 25398 | CGCCTCCTCAAGTGAC | 48 | 863 |
| 915931 | 1652 | 1667 | 25415 | 25430 | ACTTTAGCACCTCTGA | 93 | 864 |
| 915951 | 1695 | 1710 | 25458 | 25473 | GTCACTACACAGCAAT | 84 | 865 |
| 915971 | 1776 | 1791 | 25539 | 25554 | GCACAGGCGAAAGGTT | 74 | 866 |
| 915991 | 1798 | 1813 | 25561 | 25576 | TAGAGTTAAGTGCTGG | 84 | 867 |
| 916011 | 1825 | 1840 | 25588 | 25603 | CAGCTGAATTAACGCA | 0 | 868 |
| 916031 | 1868 | 1883 | 25631 | 25646 | TAAGGGACCCTCTGCA | 54 | 869 |
| 916051 | 1892 | 1907 | 25655 | 25670 | TTAATAGGGCCACGAA | 75 | 870 |
| 916071 | 1922 | 1937 | 25685 | 25700 | TAAGAACCTCATGCTG | 56 | 871 |
| 916091 | 1965 | 1980 | 25728 | 25743 | CCCCCCATCACAAGGC | 9 | 872 |
| 916111 | 1987 | 2002 | 25750 | 25765 | AGATCACACATGGGCC | 26 | 873 |
| 916131 | 2084 | 2099 | 25847 | 25862 | ACCACCTGACAGGCAG | 80 | 874 |
| 916150 | 2110 | 2125 | 25873 | 25888 | AGTAGTCAAGGTTATC | 92 | 875 |
| 916170 | 2174 | 2189 | 25937 | 25952 | TGAAAAAGGTGTTCTA | 49 | 876 |
| 916190 | 2283 | 2298 | 26046 | 26061 | AAGGCTTATTCTACTA | 79 | 877 |
| 916210 | 2633 | 2648 | 26396 | 26411 | AGGTGAATGCCCTGCA | 71 | 878 |
| 916230 | 2694 | 2709 | 26457 | 26472 | TGTGCTCCAGTGGAGA | 75 | 879 |
| 916250 | 2728 | 2743 | 26491 | 26506 | TGGTCCCTGCAGAAAA | 79 | 880 |
| 916290 | N/A | N/A | 4397 | 4412 | TGCCTACTGGCTCACA | 14 | 881 |
| 916310 | N/A | N/A | 5279 | 5294 | AAATCTTGGGATGCAC | 94 | 882 |
| 916330 | N/A | N/A | 5572 | 5587 | TGACATCATGGCTTCC | 93 | 883 |
| 916350 | N/A | N/A | 6158 | 6173 | GCTTACATCCACGACT | 0 | 884 |
| 916370 | N/A | N/A | 7135 | 7150 | CAAGTAAATGGAGTCC | 77 | 885 |
| 916390 | N/A | N/A | 7620 | 7635 | ATCTATTGCATAATCC | 86 | 886 |
| 916550 | N/A | N/A | 12205 | 12220 | TTTATTTCCGTTAACC | 96 | 887 |
| 916570 | N/A | N/A | 12694 | 12709 | TTCTTGACCGTGTTTC | 98 | 888 |
| 916590 | N/A | N/A | 12990 | 13005 | CCGGAATAAGCCTCCA | 47 | 889 |
| 916610 | N/A | N/A | 13822 | 13837 | TGTACAATGGGACGGA | 69 | 890 |
| 916630 | N/A | N/A | 14418 | 14433 | ATCGACACAGCATCAC | 92 | 891 |
| 916650 | N/A | N/A | 15138 | 15153 | TGCCATACCGGCTCCC | 0 | 892 |
| 916670 | N/A | N/A | 15758 | 15773 | GGTTTATAACAACTGA | 89 | 893 |
| 916690 | N/A | N/A | 16722 | 16737 | GCCTTGAGGTGGGTGG | 0 | 894 |
| 916710 | N/A | N/A | 17512 | 17527 | AGTCATGGGATGTGCA | 58 | 895 |
| 916730 | N/A | N/A | 18395 | 18410 | ATGTTTGGAAGTCGCC | 92 | 896 |
| 916750 | N/A | N/A | 19473 | 19488 | AAGGATCCTGCTTCTA | 9 | 897 |
| 916769 | N/A | N/A | 20554 | 20569 | GCTTACTATGGGATGA | 62 | 898 |
| 916789 | N/A | N/A | 20809 | 20824 | GTAATATTCAGACCAG | 96 | 2177 |
| 916809 | N/A | N/A | 21254 | 21269 | ATCCATGCATGGCACA | 72 | 900 |
| 916829 | N/A | N/A | 21979 | 21994 | GTCAGACACGGAGACA | 0 | 901 |
| 916849 | N/A | N/A | 23110 | 23125 | GGCTTTTGAAGGAGAG | 84 | 902 |
| 916869 | N/A | N/A | 23787 | 23802 | TATTTACCTGGAGGCG | 0 | 903 |
| 916889 | N/A | N/A | 24612 | 24627 | CAAATCGGATCTTTGC | 44 | 904 |

TABLE 14
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915352 | 34 | 49 | 2772 | 2787 | CGGCCAGGGCATTCCC | 0 | 905 |
| 915372 | 86 | 101 | 2824 | 2839 | GCAGCAGCTCCGCCCG | 48 | 906 |
| 915392 | 135 | 150 | 2873 | 2888 | GCGGGTTAGGATCTGG | 25 | 907 |
| 915412 | 225 | 240 | 2963 | 2978 | CGTGGTAGAAGCCCAG | 2 | 908 |
| 915432 | 322 | 337 | 3060 | 3075 | CCGACGCAGTGCAACG | 31 | 909 |
| 915452 | 371 | 386 | 5945 | 5960 | ATCTGAGAGGACCTGC | 72 | 910 |
| 915472 | 401 | 416 | 5975 | 5990 | AATGTTCCGACTCCTG | 77 | 911 |
| 915492 | 438 | 453 | 6012 | 6027 | GGAACTTGCTTAAGTT | 55 | 912 |
| 915512 | 469 | 484 | 6043 | 6058 | GCCGGGAGGCATTTGC | 3 | 913 |
| 915532 | 499 | 514 | 6073 | 6088 | ATTTTGCCGGAGATGA | 86 | 914 |
| 915552 | 521 | 536 | 6095 | 6110 | CACTCTGGTAAGAGAG | 8 | 915 |
| 915612 | 709 | 724 | 11927 | 11942 | ACGGTGATGGTTGTTT | 87 | 916 |
| 915632 | 787 | 802 | 12005 | 12020 | AGCTTGGTGATGTCCA | 38 | 917 |
| 915652 | 836 | 851 | 12054 | 12069 | AAAAGCTCTCGAGAGA | 0 | 918 |
| 915672 | 891 | 906 | 13627 | 13642 | CATCCAAATATCCTCG | 82 | 919 |
| 915692 | 972 | 987 | 16092 | 16107 | CAGGATCCATCCCTTC | 0 | 920 |
| 915712 | 1006 | 1021 | 16126 | 16141 | AGACTCATGTTTGCCC | 77 | 921 |
| 915732 | 1081 | 1096 | 16201 | 16216 | CTGAGACGCAGGTGGT | 87 | 922 |
| 915752 | 1141 | 1156 | N/A | N/A | AGTGCTGTAGCGAGCC | 1 | 923 |
| 915772 | 1203 | 1218 | 19043 | 19058 | TAATGGGTAGCAAGTT | 77 | 924 |
| 915792 | 1257 | 1272 | 19097 | 19112 | CAATGGCAGATTCCAC | 56 | 925 |
| 915812 | 1328 | 1343 | 23733 | 23748 | GGTCACCCACTGCAAC | 58 | 926 |
| 915832 | 1358 | 1373 | 23763 | 23778 | ACACATCAGCACTCGA | 66 | 927 |
| 915852 | 1430 | 1445 | 25193 | 25208 | GTCCTGCTCAGGTGTG | 52 | 928 |
| 915872 | 1518 | 1533 | 25281 | 25296 | GGCTGGACCTGAGGAT | 0 | 929 |
| 915892 | 1570 | 1585 | 25333 | 25348 | GTGGAGAGCCCCTCAG | 47 | 930 |
| 915912 | 1621 | 1636 | 25384 | 25399 | TCGCCTCCTCAAGTGA | 8 | 931 |
| 915932 | 1654 | 1669 | 25417 | 25432 | AAACTTTAGCACCTCT | 90 | 932 |
| 915952 | 1696 | 1711 | 25459 | 25474 | GGTCACTACACAGCAA | 82 | 933 |
| 915972 | 1777 | 1792 | 25540 | 25555 | TGCACAGGCGAAAGGT | 64 | 934 |
| 915992 | 1799 | 1814 | 25562 | 25577 | TTAGAGTTAAGTGCTG | 91 | 935 |
| 916012 | 1826 | 1841 | 25589 | 25604 | CCAGCTGAATTAACGC | 32 | 936 |
| 916032 | 1869 | 1884 | 25632 | 25647 | GTAAGGGACCCTCTGC | 73 | 937 |
| 916052 | 1894 | 1909 | 25657 | 25672 | CATTAATAGGGCCACG | 81 | 938 |
| 916072 | 1923 | 1938 | 25686 | 25701 | CTAAGAACCTCATGCT | 70 | 939 |
| 916092 | 1966 | 1981 | 25729 | 25744 | ACCCCCCATCACAAGG | 30 | 940 |
| 916112 | 1988 | 2003 | 25751 | 25766 | AAGATCACACATGGGC | 86 | 941 |
| 916132 | 2085 | 2100 | 25848 | 25863 | GACCACCTGACAGGCA | 61 | 942 |
| 916151 | 2111 | 2126 | 25874 | 25889 | TAGTAGTCAAGGTTAT | 88 | 943 |
| 916171 | 2176 | 2191 | 25939 | 25954 | GGTGAAAAAGGTGTTC | 84 | 944 |
| 916191 | 2284 | 2299 | 26047 | 26062 | TAAGGCTTATTCTACT | 76 | 945 |
| 916211 | 2634 | 2649 | 26397 | 26412 | GAGGTGAATGCCCTGC | 0 | 946 |
| 916231 | 2695 | 2710 | 26458 | 26473 | GTGTGCTCCAGTGGAG | 87 | 947 |
| 916251 | 2729 | 2744 | 26492 | 26507 | CTGGTCCCTGCAGAAA | 67 | 948 |
| 916291 | N/A | N/A | 4419 | 4434 | CAATGCTACTTGCCCC | 68 | 949 |
| 916311 | N/A | N/A | 5280 | 5295 | TAAATCTTGGGATGCA | 94 | 950 |
| 916331 | N/A | N/A | 5576 | 5591 | ACAATGACATCATGGC | 97 | 951 |
| 916351 | N/A | N/A | 6165 | 6180 | GCAAACTGCTTACATC | 0 | 952 |
| 916371 | N/A | N/A | 7172 | 7187 | GTTAGACGCGCCAGGC | 7 | 953 |
| 916391 | N/A | N/A | 7624 | 7639 | TCTCATCTATTGCATA | 0 | 954 |
| 916551 | N/A | N/A | 12206 | 12221 | TTTTATTTCCGTTAAC | 73 | 955 |
| 916571 | N/A | N/A | 12714 | 12729 | TAAACTACCGAACGCA | 96 | 956 |
| 916591 | N/A | N/A | 12991 | 13006 | CCCGGAATAAGCCTCC | 47 | 957 |
| 916611 | N/A | N/A | 13823 | 13838 | CTGTACAATGGGACGG | 23 | 958 |
| 916631 | N/A | N/A | 14422 | 14437 | TCCCATCGACACAGCA | 95 | 959 |
| 916651 | N/A | N/A | 15206 | 15221 | GGAATATTGCCAGGTA | 95 | 960 |
| 916671 | N/A | N/A | 15759 | 15774 | TGGTTTATAACAACTG | 29 | 961 |
| 916691 | N/A | N/A | 16746 | 16761 | ATTAGGAGAGGTCTCA | 55 | 962 |
| 916711 | N/A | N/A | 17602 | 17617 | CTTGATAGTGAATGTG | 90 | 963 |
| 916731 | N/A | N/A | 18859 | 18874 | GGCACTCACAAAAGCG | 10 | 964 |
| 916751 | N/A | N/A | 20182 | 20197 | CCCTATGTTCTACTTT | 54 | 965 |
| 916770 | N/A | N/A | 20572 | 20587 | CAACATCTCTAGCTGG | 82 | 966 |
| 916790 | N/A | N/A | 20810 | 20825 | GGTAATATTCAGACCA | 0 | 967 |
| 916810 | N/A | N/A | 21265 | 21280 | TGAAGCTACAGATCCA | 74 | 968 |
| 916830 | N/A | N/A | 22042 | 22057 | GGAAATCTGTCAGAGC | 18 | 969 |
| 916850 | N/A | N/A | 23142 | 23157 | GAATCTAGGAAGGCGA | 77 | 970 |
| 916870 | N/A | N/A | 23789 | 23804 | AGTATTTACCTGGAGG | 0 | 971 |
| 916890 | N/A | N/A | 24738 | 24753 | AGCCTTAGGAAGCCTC | 16 | 972 |

TABLE 15
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915353 | 35 | 50 | 2773 | 2788 | TCGGCCAGGGCATTCC | 0 | 973 |
| 915373 | 87 | 102 | 2825 | 2840 | CGCAGCAGCTCCGCCC | 0 | 974 |
| 915393 | 136 | 151 | 2874 | 2889 | CGCGGGTTAGGATCTG | 0 | 975 |
| 915413 | 239 | 254 | 2977 | 2992 | GCGGGTCGCCCCGACG | 0 | 976 |
| 915433 | 325 | 340 | 3063 | 3078 | ACGCCGACGCAGTGCA | 0 | 977 |
| 915453 | 372 | 387 | 5946 | 5961 | GATCTGAGAGGACCTG | 24 | 978 |
| 915473 | 402 | 417 | 5976 | 5991 | CAATGTTCCGACTCCT | 73 | 979 |
| 915493 | 441 | 456 | 6015 | 6030 | GGAGGAACTTGCTTAA | 87 | 980 |
| 915513 | 470 | 485 | 6044 | 6059 | GGCCGGGAGGCATTTG | 0 | 981 |
| 915533 | 500 | 515 | 6074 | 6089 | TATTTTGCCGGAGATG | 75 | 982 |
| 915553 | 522 | 537 | 6096 | 6111 | ACACTCTGGTAAGAGA | 0 | 983 |
| 915613 | 710 | 725 | 11928 | 11943 | CACGGTGATGGTTGTT | 64 | 984 |
| 915633 | 788 | 803 | 12006 | 12021 | GAGCTTGGTGATGTCC | 74 | 985 |
| 915653 | 837 | 852 | 12055 | 12070 | CAAAAGCTCTCGAGAG | 0 | 986 |
| 915673 | 892 | 907 | 13628 | 13643 | GCATCCAAATATCCTC | 81 | 987 |
| 915693 | 973 | 988 | 16093 | 16108 | TCAGGATCCATCCCTT | 10 | 988 |
| 915713 | 1007 | 1022 | 16127 | 16142 | CAGACTCATGTTTGCC | 0 | 989 |
| 915733 | 1082 | 1097 | 16202 | 16217 | GCTGAGACGCAGGTGG | 64 | 990 |
| 915753 | 1142 | 1157 | N/A | N/A | CAGTGCTGTAGCGAGC | 0 | 991 |
| 915773 | 1204 | 1219 | 19044 | 19059 | CTAATGGGTAGCAAGT | 72 | 992 |
| 915793 | 1258 | 1273 | 19098 | 19113 | GCAATGGCAGATTCCA | 57 | 993 |
| 915813 | 1329 | 1344 | 23734 | 23749 | AGGTCACCCACTGCAA | 56 | 994 |
| 915833 | 1359 | 1374 | 23764 | 23779 | GACACATCAGCACTCG | 43 | 995 |
| 915853 | 1431 | 1446 | 25194 | 25209 | AGTCCTGCTCAGGTGT | 66 | 996 |
| 915873 | 1525 | 1540 | 25288 | 25303 | AAGTTCAGGCTGGACC | 54 | 997 |
| 915893 | 1571 | 1586 | 25334 | 25349 | GGTGGAGAGCCCCTCA | 0 | 998 |
| 915913 | 1622 | 1637 | 25385 | 25400 | CTCGCCTCCTCAAGTG | 52 | 999 |
| 915933 | 1660 | 1675 | 25423 | 25438 | GATGGGAAACTTTAGC | 85 | 1000 |
| 915953 | 1697 | 1712 | 25460 | 25475 | GGGTCACTACACAGCA | 78 | 1001 |
| 915973 | 1778 | 1793 | 25541 | 25556 | CTGCACAGGCGAAAGG | 35 | 1002 |
| 915993 | 1800 | 1815 | 25563 | 25578 | ATTAGAGTTAAGTGCT | 63 | 1003 |
| 916013 | 1827 | 1842 | 25590 | 25605 | ACCAGCTGAATTAACG | 66 | 1004 |
| 916033 | 1873 | 1888 | 25636 | 25651 | GTCAGTAAGGGACCCT | 52 | 1005 |
| 916053 | 1897 | 1912 | 25660 | 25675 | GACCATTAATAGGGCC | 51 | 1006 |
| 916073 | 1924 | 1939 | 25687 | 25702 | TCTAAGAACCTCATGC | 55 | 1007 |
| 916093 | 1967 | 1982 | 25730 | 25745 | TACCCCCCATCACAAG | 15 | 1008 |
| 916113 | 1990 | 2005 | 25753 | 25768 | ACAAGATCACACATGG | 72 | 1009 |
| 916133 | 2086 | 2101 | 25849 | 25864 | AGACCACCTGACAGGC | 79 | 1010 |
| 916152 | 2112 | 2127 | 25875 | 25890 | TTAGTAGTCAAGGTTA | 84 | 1011 |
| 916172 | 2177 | 2192 | 25940 | 25955 | AGGTGAAAAAGGTGTT | 88 | 1012 |
| 916192 | 2285 | 2300 | 26048 | 26063 | TTAAGGCTTATTCTAC | 82 | 1013 |
| 916212 | 2635 | 2650 | 26398 | 26413 | TGAGGTGAATGCCCTG | 58 | 1014 |
| 916232 | 2696 | 2711 | 26459 | 26474 | TGTGTGCTCCAGTGGA | 89 | 1015 |
| 916252 | 2730 | 2745 | 26493 | 26508 | GCTGGTCCCTGCAGAA | 44 | 1016 |
| 916272 | N/A | N/A | 3328 | 3343 | GGGACGCACGAGAGTC | 0 | 1017 |
| 916292 | N/A | N/A | 4432 | 4447 | GTCAATAGCTTCACAA | 86 | 1018 |
| 916312 | N/A | N/A | 5281 | 5296 | ATAAATCTTGGGATGC | 92 | 1019 |
| 916332 | N/A | N/A | 5577 | 5592 | CACAATGACATCATGG | 95 | 1020 |
| 916352 | N/A | N/A | 6170 | 6185 | GATAAGCAAACTGCTT | 19 | 1021 |
| 916372 | N/A | N/A | 7192 | 7207 | GAGGATGCAACTGGCT | 84 | 1022 |
| 916392 | N/A | N/A | 7644 | 7659 | TCGGACTTCAGGCCCA | 0 | 1023 |
| 916552 | N/A | N/A | 12208 | 12223 | CCTTTTATTTCCGTTA | 97 | 1024 |
| 916572 | N/A | N/A | 12745 | 12760 | GCATACTAAAACCACC | 85 | 1025 |
| 916592 | N/A | N/A | 13375 | 13390 | GACTTTGCAGGCACCC | 92 | 1026 |
| 916612 | N/A | N/A | 13909 | 13924 | TGACATCCCAGTTCAA | 30 | 1027 |
| 916632 | N/A | N/A | 14427 | 14442 | TACTTTCCCATCGACA | 81 | 1028 |
| 916652 | N/A | N/A | 15207 | 15222 | AGGAATATTGCCAGGT | 88 | 1029 |
| 916672 | N/A | N/A | 15768 | 15783 | GGTTAGTGTTGGTTTA | 92 | 1030 |
| 916692 | N/A | N/A | 16790 | 16805 | CATTCGATGGAGGTTC | 58 | 1031 |
| 916712 | N/A | N/A | 17629 | 17644 | GGCGGATTTCCCCACT | 11 | 1032 |
| 916732 | N/A | N/A | 18894 | 18909 | TAAAATACGCCCGTCC | 7 | 1033 |
| 916752 | N/A | N/A | 20183 | 20198 | TCCCTATGTTCTACTT | 32 | 1034 |
| 916771 | N/A | N/A | 20574 | 20589 | ATCAACATCTCTAGCT | 46 | 1035 |
| 916791 | N/A | N/A | 20811 | 20826 | GGGTAATATTCAGACC | 43 | 1036 |
| 916811 | N/A | N/A | 21313 | 21328 | TTTACTAGAGACTCTG | 69 | 1037 |
| 916831 | N/A | N/A | 22071 | 22086 | GTAGGATAGGACTAGA | 45 | 1038 |
| 916851 | N/A | N/A | 23219 | 23234 | ATAAATGCCTGACCAC | 64 | 1039 |
| 916871 | N/A | N/A | 23861 | 23876 | TGTTTCTAGAATGTCG | 68 | 1040 |
| 916891 | N/A | N/A | 24873 | 24888 | GCCTATCAGTTTCCCC | 0 | 1041 |

TABLE 16
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915354 | 36 | 51 | 2774 | 2789 | CTCGGCCAGGGCATTC | 0 | 1042 |
| 915374 | 89 | 104 | 2827 | 2842 | TCCGCAGCAGCTCCGC | 60 | 1043 |
| 915394 | 137 | 152 | 2875 | 2890 | GCGCGGGTTAGGATCT | 0 | 1044 |
| 915414 | 240 | 255 | 2978 | 2993 | AGCGGGTCGCCCCGAC | 21 | 1045 |
| 915434 | 337 | 352 | 3075 | 3090 | ATACCGGAGAGGACGC | 85 | 1046 |
| 915454 | 374 | 389 | 5948 | 5963 | AAGATCTGAGAGGACC | 24 | 1047 |
| 915474 | 403 | 418 | 5977 | 5992 | CCAATGTTCCGACTCC | 95 | 1048 |
| 915494 | 442 | 457 | 6016 | 6031 | CGGAGGAACTTGCTTA | 93 | 1049 |
| 915514 | 471 | 486 | 6045 | 6060 | TGGCCGGGAGGCATTT | 0 | 1050 |
| 915534 | 501 | 516 | 6075 | 6090 | CTATTTTGCCGGAGAT | 87 | 1051 |
| 915554 | 523 | 538 | 6097 | 6112 | GACACTCTGGTAAGAG | 26 | 1052 |
| 915614 | 711 | 726 | 11929 | 11944 | ACACGGTGATGGTTGT | 46 | 1053 |
| 915634 | 791 | 806 | 12009 | 12024 | ACTGAGCTTGGTGATG | 87 | 1054 |
| 915654 | 838 | 853 | 12056 | 12071 | ACAAAAGCTCTCGAGA | 0 | 1055 |
| 915674 | 900 | 915 | 13636 | 13651 | ACCTGAATGCATCCAA | 93 | 1056 |
| 915694 | 974 | 989 | 16094 | 16109 | CTCAGGATCCATCCCT | 43 | 1057 |
| 915714 | 1008 | 1023 | 16128 | 16143 | CCAGACTCATGTTTGC | 0 | 1058 |
| 915734 | 1083 | 1098 | 16203 | 16218 | TGCTGAGACGCAGGTG | 50 | 1059 |
| 915754 | 1143 | 1158 | N/A | N/A | TCAGTGCTGTAGCGAG | 42 | 1060 |
| 915774 | 1208 | 1223 | 19048 | 19063 | TATCCTAATGGGTAGC | 53 | 1061 |
| 915794 | 1260 | 1275 | 19100 | 19115 | TCGCAATGGCAGATTC | 67 | 1062 |
| 915814 | 1333 | 1348 | 23738 | 23753 | TGTGAGGTCACCCACT | 0 | 1063 |
| 915834 | 1360 | 1375 | 23765 | 23780 | AGACACATCAGCACTC | 24 | 1064 |
| 915854 | 1432 | 1447 | 25195 | 25210 | CAGTCCTGCTCAGGTG | 54 | 1065 |
| 915874 | 1526 | 1541 | 25289 | 25304 | GAAGTTCAGGCTGGAC | 75 | 1066 |
| 915894 | 1572 | 1587 | 25335 | 25350 | AGGTGGAGAGCCCCTC | 0 | 1067 |
| 915914 | 1623 | 1638 | 25386 | 25401 | ACTCGCCTCCTCAAGT | 0 | 1068 |
| 915934 | 1661 | 1676 | 25424 | 25439 | AGATGGGAAACTTTAG | 84 | 1069 |
| 915954 | 1724 | 1739 | 25487 | 25502 | GGCTGGGATCCTCCAC | 24 | 1070 |
| 915974 | 1779 | 1794 | 25542 | 25557 | GCTGCACAGGCGAAAG | 56 | 1071 |
| 915994 | 1801 | 1816 | 25564 | 25579 | TATTAGAGTTAAGTGC | 75 | 1072 |
| 916014 | 1828 | 1843 | 25591 | 25606 | AACCAGCTGAATTAAC | 55 | 1073 |
| 916034 | 1875 | 1890 | 25638 | 25653 | CAGTCAGTAAGGGACC | 70 | 1074 |
| 916054 | 1898 | 1913 | 25661 | 25676 | TGACCATTAATAGGGC | 74 | 1075 |
| 916074 | 1925 | 1940 | 25688 | 25703 | TTCTAAGAACCTCATG | 22 | 1076 |
| 916094 | 1968 | 1983 | 25731 | 25746 | CTACCCCCCATCACAA | 0 | 1077 |
| 916114 | 1992 | 2007 | 25755 | 25770 | CCACAAGATCACACAT | 0 | 1078 |
| 916134 | 2087 | 2102 | 25850 | 25865 | CAGACCACCTGACAGG | 78 | 1079 |
| 916153 | 2113 | 2128 | 25876 | 25891 | TTTAGTAGTCAAGGTT | 93 | 1080 |
| 916173 | 2178 | 2193 | 25941 | 25956 | TAGGTGAAAAAGGTGT | 89 | 1081 |
| 916193 | 2306 | 2321 | 26069 | 26084 | ACCCAACCGATTTTTT | 61 | 1082 |
| 916213 | 2636 | 2651 | 26399 | 26414 | CTGAGGTGAATGCCCT | 73 | 1083 |
| 916233 | 2697 | 2712 | 26460 | 26475 | TTGTGTGCTCCAGTGG | 92 | 1084 |
| 916253 | 2746 | 2761 | 26509 | 26524 | TCACTGACCATGTGGG | 16 | 1085 |
| 916273 | N/A | N/A | 3362 | 3377 | CTTCATGCACGGGCGC | 37 | 1086 |
| 916293 | N/A | N/A | 4462 | 4477 | GCATAATCTCCTGCCT | 0 | 1087 |
| 916313 | N/A | N/A | 5284 | 5299 | GCCATAAATCTTGGGA | 37 | 1088 |
| 916333 | N/A | N/A | 5605 | 5620 | CTTTATTCAATGTGGC | 97 | 2173 |
| 916353 | N/A | N/A | 6529 | 6544 | TACAACTGCCTGTGTT | 0 | 1090 |
| 916373 | N/A | N/A | 7218 | 7233 | AAAGCTTCCGCAAACA | 51 | 1091 |
| 916393 | N/A | N/A | 7657 | 7672 | CTAACATACACCCTCG | 0 | 1092 |
| 916553 | N/A | N/A | 12225 | 12240 | AGCTTCTGGGACAAGC | 10 | 1093 |
| 916573 | N/A | N/A | 12746 | 12761 | GGCATACTAAAACCAC | 55 | 1094 |
| 916593 | N/A | N/A | 13397 | 13412 | TTGAATGTCACCCTTC | 91 | 1095 |
| 916613 | N/A | N/A | 13914 | 13929 | AGTCATGACATCCCAG | 93 | 1096 |
| 916633 | N/A | N/A | 14442 | 14457 | TCTCATTGGCACCTGT | 86 | 1097 |
| 916653 | N/A | N/A | 15252 | 15267 | CCCTATCAGATGCCCT | 81 | 1098 |
| 916673 | N/A | N/A | 15799 | 15814 | CATATCTGGTTTCATG | 0 | 1099 |
| 916693 | N/A | N/A | 16842 | 16857 | GACCATAGCACTGTCT | 0 | 1100 |
| 916713 | N/A | N/A | 17737 | 17752 | ATTAATCTGGTCATAT | 0 | 1101 |
| 916733 | N/A | N/A | 18898 | 18913 | TCCATAAAATACGCCC | 69 | 1102 |
| 916753 | N/A | N/A | 20195 | 20210 | GAAAGATGGAATTCCC | 86 | 1103 |
| 916772 | N/A | N/A | 20604 | 20619 | TACGATCATCATTATT | 91 | 1104 |
| 916792 | N/A | N/A | 20841 | 20856 | GTATTAGCTCAATATT | 0 | 1105 |
| 916812 | N/A | N/A | 21314 | 21329 | GTTTACTAGAGACTCT | 64 | 1106 |
| 916832 | N/A | N/A | 22080 | 22095 | GTAAAAACTGTAGGAT | 0 | 1107 |
| 916852 | N/A | N/A | 23220 | 23235 | GATAAATGCCTGACCA | 29 | 1108 |
| 916872 | N/A | N/A | 24011 | 24026 | CCGACGGGAAGTCTTC | 0 | 1109 |
| 916892 | N/A | N/A | 24874 | 24889 | GGCCTATCAGTTTCCC | 0 | 1110 |

TABLE 17
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915355 | 37 | 52 | 2775 | 2790 | TCTCGGCCAGGGCATT | 0 | 1111 |
| 915375 | 90 | 105 | 2828 | 2843 | ATCCGCAGCAGCTCCG | 52 | 1112 |
| 915395 | 138 | 153 | 2876 | 2891 | GGCGCGGGTTAGGATC | 0 | 1113 |
| 915415 | 241 | 256 | 2979 | 2994 | CAGCGGGTCGCCCCGA | 8 | 1114 |
| 915435 | 338 | 353 | 3076 | 3091 | GATACCGGAGAGGACG | 30 | 1115 |
| 915455 | 378 | 393 | 5952 | 5967 | GCACAAGATCTGAGAG | 72 | 1116 |
| 915475 | 405 | 420 | 5979 | 5994 | TGCCAATGTTCCGACT | 0 | 1117 |
| 915495 | 443 | 458 | 6017 | 6032 | TCGGAGGAACTTGCTT | 69 | 1118 |
| 915515 | 472 | 487 | 6046 | 6061 | TTGGCCGGGAGGCATT | 9 | 1119 |
| 915535 | 502 | 517 | 6076 | 6091 | CCTATTTTGCCGGAGA | 96 | 1120 |
| 915555 | 524 | 539 | 6098 | 6113 | AGACACTCTGGTAAGA | 2 | 1121 |
| 915615 | 712 | 727 | 11930 | 11945 | GACACGGTGATGGTTG | 32 | 1122 |
| 915635 | 792 | 807 | 12010 | 12025 | GACTGAGCTTGGTGAT | 93 | 1123 |
| 915655 | 839 | 854 | 12057 | 12072 | GACAAAAGCTCTCGAG | 40 | 1124 |
| 915675 | 901 | 916 | 13637 | 13652 | AACCTGAATGCATCCA | 92 | 1125 |
| 915695 | 975 | 990 | 16095 | 16110 | CCTCAGGATCCATCCC | 0 | 1126 |
| 915715 | 1011 | 1026 | 16131 | 16146 | AATCCAGACTCATGTT | 67 | 1127 |
| 915735 | 1088 | 1103 | 16208 | 16223 | CAGGATGCTGAGACGC | 86 | 1128 |
| 915755 | 1144 | 1159 | N/A | N/A | CTCAGTGCTGTAGCGA | 25 | 1129 |
| 915775 | 1209 | 1224 | 19049 | 19064 | TTATCCTAATGGGTAG | 23 | 1130 |
| 915795 | 1261 | 1276 | 19101 | 19116 | ATCGCAATGGCAGATT | 0 | 1131 |
| 915815 | 1337 | 1352 | 23742 | 23757 | CACCTGTGAGGTCACC | 35 | 1132 |
| 915835 | 1361 | 1376 | 23766 | 23781 | CAGACACATCAGCACT | 54 | 1133 |
| 915855 | 1433 | 1448 | 25196 | 25211 | CCAGTCCTGCTCAGGT | 23 | 1134 |
| 915875 | 1530 | 1545 | 25293 | 25308 | AGAAGAAGTTCAGGCT | 81 | 1135 |
| 915895 | 1574 | 1589 | 25337 | 25352 | AAAGGTGGAGAGCCCC | 76 | 1136 |
| 915915 | 1624 | 1639 | 25387 | 25402 | GACTCGCCTCCTCAAG | 75 | 1137 |
| 915935 | 1674 | 1689 | 25437 | 25452 | GGTAGCTGCACAAAGA | 76 | 1138 |
| 915955 | 1726 | 1741 | 25489 | 25504 | GAGGCTGGGATCCTCC | 0 | 1139 |
| 915975 | 1780 | 1795 | 25543 | 25558 | CGCTGCACAGGCGAAA | 0 | 1140 |
| 915995 | 1805 | 1820 | 25568 | 25583 | GATGTATTAGAGTTAA | 82 | 1141 |
| 916015 | 1829 | 1844 | 25592 | 25607 | CAACCAGCTGAATTAA | 59 | 1142 |
| 916035 | 1876 | 1891 | 25639 | 25654 | ACAGTCAGTAAGGGAC | 81 | 1143 |
| 916055 | 1899 | 1914 | 25662 | 25677 | CTGACCATTAATAGGG | 49 | 1144 |
| 916075 | 1929 | 1944 | 25692 | 25707 | GTCATTCTAAGAACCT | 81 | 1145 |
| 916095 | 1969 | 1984 | 25732 | 25747 | CCTACCCCCCATCACA | 21 | 1146 |
| 916115 | 1995 | 2010 | 25758 | 25773 | ACCCCACAAGATCACA | 0 | 1147 |
| 916135 | 2088 | 2103 | 25851 | 25866 | GCAGACCACCTGACAG | 44 | 1148 |
| 916154 | 2131 | 2146 | 25894 | 25909 | CCCCGCCATGGAGACG | 68 | 1149 |
| 916174 | 2180 | 2195 | 25943 | 25958 | GTTAGGTGAAAAAGGT | 90 | 1150 |
| 916194 | 2308 | 2323 | 26071 | 26086 | GCACCCAACCGATTTT | 83 | 1151 |
| 916214 | 2637 | 2652 | 26400 | 26415 | GCTGAGGTGAATGCCC | 52 | 1152 |
| 916234 | 2698 | 2713 | 26461 | 26476 | GTTGTGTGCTCCAGTG | 88 | 1153 |
| 916254 | 2747 | 2762 | 26510 | 26525 | CTCACTGACCATGTGG | 13 | 1154 |
| 916274 | N/A | N/A | 3524 | 3539 | GCAAATCGGCCCCTCG | 3 | 1155 |
| 916294 | N/A | N/A | 4463 | 4478 | GGCATAATCTCCTGCC | 0 | 1156 |
| 916314 | N/A | N/A | 5324 | 5339 | TGGCATGCAAGACCAC | 0 | 1157 |
| 916334 | N/A | N/A | 5606 | 5621 | ACTTTATTCAATGTGG | 95 | 1158 |
| 916354 | N/A | N/A | 6556 | 6571 | GTTTATGTCACTCTGG | 68 | 1159 |
| 916374 | N/A | N/A | 7245 | 7260 | GAACAGACAAGTGCTG | 38 | 1160 |
| 916394 | N/A | N/A | 7658 | 7673 | ACTAACATACACCCTC | 31 | 1161 |
| 916554 | N/A | N/A | 12249 | 12264 | ATAATCAGGGTGGTGC | 0 | 1162 |
| 916574 | N/A | N/A | 12747 | 12762 | AGGCATACTAAAACCA | 47 | 1163 |
| 916594 | N/A | N/A | 13500 | 13515 | GAATCATGCAAGCTCT | 50 | 1164 |
| 916614 | N/A | N/A | 13996 | 14011 | TAAACTAAGGGTCACA | 37 | 1165 |
| 916634 | N/A | N/A | 14497 | 14512 | ATCCATCCTGCATGAG | 76 | 1166 |
| 916654 | N/A | N/A | 15254 | 15269 | GGCCCTATCAGATGCC | 0 | 1167 |
| 916674 | N/A | N/A | 15802 | 15817 | CTACATATCTGGTTTC | 0 | 1168 |
| 916694 | N/A | N/A | 16844 | 16859 | TGGACCATAGCACTGT | 60 | 1169 |
| 916714 | N/A | N/A | 17738 | 17753 | TATTAATCTGGTCATA | 18 | 1170 |
| 916734 | N/A | N/A | 18926 | 18941 | CCACTTTACTCTGTTG | 64 | 1171 |
| 916754 | N/A | N/A | 20210 | 20225 | AACTATGCCTAGAACG | 43 | 1172 |
| 916773 | N/A | N/A | 20606 | 20621 | TTTACGATCATCATTA | 77 | 1173 |
| 916793 | N/A | N/A | 20842 | 20857 | TGTATTAGCTCAATAT | 0 | 1174 |
| 916813 | N/A | N/A | 21319 | 21334 | TGGGAGTTTACTAGAG | 66 | 1175 |
| 916833 | N/A | N/A | 22118 | 22133 | AGAGAGTACTCTTGGA | 11 | 1176 |
| 916853 | N/A | N/A | 23222 | 23237 | CTGATAAATGCCTGAC | 78 | 1177 |
| 916873 | N/A | N/A | 24038 | 24053 | ATCAATGCTGCACTCA | 88 | 1178 |
| 916893 | N/A | N/A | 24889 | 24904 | ACGAATCCCTGGAGGG | 0 | 1179 |

TABLE 18
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915356 | 38 | 53 | 2776 | 2791 | GTCTCGGCCAGGGCAT | 0 | 1180 |
| 915376 | 93 | 108 | 2831 | 2846 | CTGATCCGCAGCAGCT | 28 | 1181 |
| 915396 | 165 | 180 | 2903 | 2918 | CGTACATGGCGGCGGC | 0 | 1182 |
| 915416 | 242 | 257 | 2980 | 2995 | GCAGCGGGTCGCCCCG | 0 | 1183 |
| 915436 | 339 | 354 | 3077 | 3092 | GGATACCGGAGAGGAC | 64 | 1184 |
| 915456 | 379 | 394 | 5953 | 5968 | CGCACAAGATCTGAGA | 79 | 1185 |
| 915476 | 406 | 421 | 5980 | 5995 | ATGCCAATGTTCCGAC | 83 | 1186 |
| 915496 | 444 | 459 | 6018 | 6033 | GTCGGAGGAACTTGCT | 35 | 1187 |
| 915516 | 473 | 488 | 6047 | 6062 | ATTGGCCGGGAGGCAT | 0 | 1188 |
| 915536 | 503 | 518 | 6077 | 6092 | GCCTATTTTGCCGGAG | 77 | 1189 |
| 915556 | 525 | 540 | 6099 | 6114 | CAGACACTCTGGTAAG | 62 | 1190 |
| 915616 | 730 | 745 | 11948 | 11963 | TACTCCCCATAGAAGG | 8 | 1191 |
| 915636 | 794 | 809 | 12012 | 12027 | TAGACTGAGCTTGGTG | 90 | 1192 |
| 915656 | 840 | 855 | 12058 | 12073 | GGACAAAAGCTCTCGA | 60 | 1193 |
| 915676 | 902 | 917 | 13638 | 13653 | GAACCTGAATGCATCC | 72 | 1194 |
| 915696 | 978 | 993 | 16098 | 16113 | CGACCTCAGGATCCAT | 0 | 1195 |
| 915716 | 1012 | 1027 | 16132 | 16147 | GAATCCAGACTCATGT | 0 | 1196 |
| 915736 | 1089 | 1104 | 16209 | 16224 | GCAGGATGCTGAGACG | 55 | 1197 |
| 915756 | 1145 | 1160 | N/A | N/A | ACTCAGTGCTGTAGCG | 12 | 1198 |
| 915776 | 1210 | 1225 | 19050 | 19065 | ATTATCCTAATGGGTA | 28 | 1199 |
| 915796 | 1262 | 1277 | 19102 | 19117 | AATCGCAATGGCAGAT | 0 | 1200 |
| 915816 | 1339 | 1354 | 23744 | 23759 | AACACCTGTGAGGTCA | 56 | 1201 |
| 915836 | 1365 | 1380 | 23770 | 23785 | GGAGCAGACACATCAG | 53 | 1202 |
| 915856 | 1434 | 1449 | 25197 | 25212 | GCCAGTCCTGCTCAGG | 21 | 1203 |
| 915876 | 1531 | 1546 | 25294 | 25309 | AAGAAGAAGTTCAGGC | 85 | 1204 |
| 915896 | 1575 | 1590 | 25338 | 25353 | GAAAGGTGGAGAGCCC | 78 | 1205 |
| 915916 | 1626 | 1641 | 25389 | 25404 | TAGACTCGCCTCCTCA | 32 | 1206 |
| 915936 | 1676 | 1691 | 25439 | 25454 | GAGGTAGCTGCACAAA | 91 | 1207 |
| 915956 | 1737 | 1752 | 25500 | 25515 | AACTCAGCTCAGAGGC | 46 | 1208 |
| 915976 | 1781 | 1796 | 25544 | 25559 | CCGCTGCACAGGCGAA | 0 | 1209 |
| 915996 | 1807 | 1822 | 25570 | 25585 | CTGATGTATTAGAGTT | 93 | 1210 |
| 916016 | 1830 | 1845 | 25593 | 25608 | CCAACCAGCTGAATTA | 21 | 1211 |
| 916036 | 1877 | 1892 | 25640 | 25655 | AACAGTCAGTAAGGGA | 82 | 1212 |
| 916056 | 1900 | 1915 | 25663 | 25678 | TCTGACCATTAATAGG | 13 | 1213 |
| 916076 | 1930 | 1945 | 25693 | 25708 | TGTCATTCTAAGAACC | 40 | 1214 |
| 916096 | 1970 | 1985 | 25733 | 25748 | GCCTACCCCCCATCAC | 18 | 1215 |
| 916116 | 1996 | 2011 | 25759 | 25774 | CACCCCACAAGATCAC | 50 | 1216 |
| 916136 | 2089 | 2104 | 25852 | 25867 | TGCAGACCACCTGACA | 58 | 1217 |
| 916155 | 2132 | 2147 | 25895 | 25910 | CCCCCGCCATGGAGAC | 33 | 1218 |
| 916175 | 2224 | 2239 | 25987 | 26002 | CGCTTCCTTACATTTT | 89 | 1219 |
| 916195 | 2309 | 2324 | 26072 | 26087 | TGCACCCAACCGATTT | 64 | 1220 |
| 916215 | 2638 | 2653 | 26401 | 26416 | GGCTGAGGTGAATGCC | 0 | 1221 |
| 916235 | 2699 | 2714 | 26462 | 26477 | AGTTGTGTGCTCCAGT | 85 | 1222 |
| 916255 | 2748 | 2763 | 26511 | 26526 | ACTCACTGACCATGTG | 0 | 1223 |
| 916275 | N/A | N/A | 3555 | 3570 | GGCCAAAGCCCCACTC | 0 | 1224 |
| 916295 | N/A | N/A | 4464 | 4479 | GGGCATAATCTCCTGC | 0 | 1225 |
| 916315 | N/A | N/A | 5342 | 5357 | GGCTGATCTGCACTCT | 84 | 1226 |
| 916335 | N/A | N/A | 5626 | 5641 | TAATTCTACCTGTGTC | 92 | 1227 |
| 916355 | N/A | N/A | 6557 | 6572 | AGTTTATGTCACTCTG | 27 | 1228 |
| 916375 | N/A | N/A | 7321 | 7336 | ACACTTTGCGAAGCAC | 27 | 1229 |
| 916395 | N/A | N/A | 7660 | 7675 | GAACTAACATACACCC | 1 | 1230 |
| 916555 | N/A | N/A | 12252 | 12267 | CCCATAATCAGGGTGG | 0 | 1231 |
| 916575 | N/A | N/A | 12758 | 12773 | GTAGAGTGGTAAGGCA | 95 | 1232 |
| 916595 | N/A | N/A | 13502 | 13517 | AAGAATCATGCAAGCT | 34 | 1233 |
| 916615 | N/A | N/A | 13997 | 14012 | TTAAACTAAGGGTCAC | 65 | 1234 |
| 916635 | N/A | N/A | 14549 | 14564 | TTAATGTGGATTCACG | 76 | 1235 |
| 916655 | N/A | N/A | 15295 | 15310 | CCAAGATAACCTCACA | 64 | 1236 |
| 916675 | N/A | N/A | 15806 | 15821 | CCATCTACATATCTGG | 26 | 1237 |
| 916695 | N/A | N/A | 16854 | 16869 | CACAATCATTTGGACC | 72 | 1238 |
| 916715 | N/A | N/A | 17739 | 17754 | GTATTAATCTGGTCAT | 87 | 1239 |
| 916735 | N/A | N/A | 19113 | 19128 | CACCTCTGGACAATCG | 29 | 1240 |
| 916755 | N/A | N/A | 20212 | 20227 | CAAACTATGCCTAGAA | 70 | 1241 |
| 916774 | N/A | N/A | 20608 | 20623 | ATTTTACGATCATCAT | 91 | 1242 |
| 916794 | N/A | N/A | 20846 | 20861 | TGCCTGTATTAGCTCA | 90 | 1243 |
| 916814 | N/A | N/A | 21345 | 21360 | CACATAAAGTCAAACG | 87 | 1244 |
| 916834 | N/A | N/A | 22124 | 22139 | AGAACAAGAGAGTACT | 3 | 1245 |
| 916854 | N/A | N/A | 23250 | 23265 | CACATAAAGGACCCCC | 54 | 1246 |
| 916874 | N/A | N/A | 24126 | 24141 | CGCTATCTGACACTCC | 87 | 1247 |
| 916894 | N/A | N/A | 24896 | 24911 | TCCACCAACGAATCCC | 50 | 1248 |

TABLE 19
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915357 | 39 | 54 | 2777 | 2792 | TGTCTCGGCCAGGGCA | 0 | 1249 |
| 915377 | 94 | 109 | 2832 | 2847 | CCTGATCCGCAGCAGC | 0 | 1250 |
| 915397 | 182 | 197 | 2920 | 2935 | CCAGCCGCGCTCTGCG | 0 | 1251 |
| 915417 | 243 | 258 | 2981 | 2996 | GGCAGCGGGTCGCCCC | 0 | 1252 |
| 915437 | 341 | 356 | 3079 | 3094 | CGGGATACCGGAGAGG | 57 | 1253 |
| 915457 | 380 | 395 | 5954 | 5969 | CCGCACAAGATCTGAG | 71 | 1254 |
| 915477 | 407 | 422 | 5981 | 5996 | GATGCCAATGTTCCGA | 93 | 1255 |
| 915497 | 445 | 460 | 6019 | 6034 | TGTCGGAGGAACTTGC | 85 | 1256 |
| 915517 | 474 | 489 | 6048 | 6063 | CATTGGCCGGGAGGCA | 0 | 1257 |
| 915537 | 504 | 519 | 6078 | 6093 | TGCCTATTTTGCCGGA | 44 | 1258 |
| 915557 | 526 | 541 | 6100 | 6115 | TCAGACACTCTGGTAA | 26 | 1259 |
| 915617 | 732 | 747 | 11950 | 11965 | CGTACTCCCCATAGAA | 66 | 1260 |
| 915637 | 796 | 811 | 12014 | 12029 | CGTAGACTGAGCTTGG | 98 | 1261 |
| 915657 | 857 | 872 | 12075 | 12090 | CACCTTGAGATCCGGG | 0 | 1262 |
| 915677 | 903 | 918 | 13639 | 13654 | AGAACCTGAATGCATC | 78 | 1263 |
| 915697 | 979 | 994 | 16099 | 16114 | GCGACCTCAGGATCCA | 1 | 1264 |
| 915717 | 1031 | 1046 | 16151 | 16166 | GGCAGCCGACTCCGGG | 19 | 1265 |
| 915737 | 1109 | 1124 | 16229 | 16244 | CAGGATGCTCTCATCC | 0 | 1266 |
| 915757 | 1146 | 1161 | N/A | N/A | CACTCAGTGCTGTAGC | 33 | 1267 |
| 915777 | 1214 | 1229 | 19054 | 19069 | AGACATTATCCTAATG | 42 | 1268 |
| 915797 | 1263 | 1278 | 19103 | 19118 | CAATCGCAATGGCAGA | 49 | 1269 |
| 915817 | 1340 | 1355 | 23745 | 23760 | GAACACCTGTGAGGTC | 44 | 1270 |
| 915837 | 1398 | 1413 | 25161 | 25176 | GGCTGCTCACTGGCAT | 18 | 1271 |
| 915857 | 1435 | 1450 | 25198 | 25213 | GGCCAGTCCTGCTCAG | 0 | 1272 |
| 915877 | 1534 | 1549 | 25297 | 25312 | CCCAAGAAGAAGTTCA | 76 | 1273 |
| 915897 | 1576 | 1591 | 25339 | 25354 | GGAAAGGTGGAGAGCC | 24 | 1274 |
| 915917 | 1627 | 1642 | 25390 | 25405 | CTAGACTCGCCTCCTC | 77 | 1275 |
| 915937 | 1679 | 1694 | 25442 | 25457 | GCGGAGGTAGCTGCAC | 16 | 1276 |
| 915957 | 1738 | 1753 | 25501 | 25516 | CAACTCAGCTCAGAGG | 61 | 1277 |
| 915977 | 1782 | 1797 | 25545 | 25560 | ACCGCTGCACAGGCGA | 34 | 1278 |
| 915997 | 1809 | 1824 | 25572 | 25587 | TGCTGATGTATTAGAG | 83 | 1279 |
| 916017 | 1831 | 1846 | 25594 | 25609 | CCCAACCAGCTGAATT | 42 | 1280 |
| 916037 | 1878 | 1893 | 25641 | 25656 | AAACAGTCAGTAAGGG | 92 | 1281 |
| 916057 | 1901 | 1916 | 25664 | 25679 | GTCTGACCATTAATAG | 64 | 1282 |
| 916077 | 1931 | 1946 | 25694 | 25709 | CTGTCATTCTAAGAAC | 41 | 1283 |
| 916097 | 1971 | 1986 | 25734 | 25749 | AGCCTACCCCCCATCA | 0 | 1284 |
| 916117 | 1997 | 2012 | 25760 | 25775 | CCACCCCACAAGATCA | 0 | 1285 |
| 916137 | 2090 | 2105 | 25853 | 25868 | TTGCAGACCACCTGAC | 65 | 1286 |
| 916156 | 2133 | 2148 | 25896 | 25911 | ACCCCCGCCATGGAGA | 54 | 1287 |
| 916176 | 2225 | 2240 | 25988 | 26003 | ACGCTTCCTTACATTT | 84 | 1288 |
| 916196 | 2310 | 2325 | 26073 | 26088 | CTGCACCCAACCGATT | 58 | 1289 |
| 916216 | 2639 | 2654 | 26402 | 26417 | GGGCTGAGGTGAATGC | 46 | 1290 |
| 916236 | 2700 | 2715 | 26463 | 26478 | AAGTTGTGTGCTCCAG | 86 | 1291 |
| 916256 | 2751 | 2766 | 26514 | 26529 | GAAACTCACTGACCAT | 41 | 1292 |
| 916276 | N/A | N/A | 4068 | 4083 | GGAAACAACTTTCCTC | 0 | 1293 |
| 916296 | N/A | N/A | 4730 | 4745 | GATCATGTGGCGGTCT | 68 | 1294 |
| 916316 | N/A | N/A | 5364 | 5379 | CACTTACTGGCCTGGC | 30 | 1295 |
| 916336 | N/A | N/A | 5645 | 5660 | ATATTGGGCTCAATGA | 89 | 1296 |
| 916356 | N/A | N/A | 6575 | 6590 | ATCACTGGAGGTGTAC | 0 | 1297 |
| 916376 | N/A | N/A | 7328 | 7343 | CAGGATCACACTTTGC | 17 | 1298 |
| 916396 | N/A | N/A | 7661 | 7676 | GGAACTAACATACACC | 0 | 1299 |
| 916556 | N/A | N/A | 12272 | 12287 | GTATATGTTCCCAGGT | 81 | 1300 |
| 916576 | N/A | N/A | 12788 | 12803 | GTGTACATGGTCTGCA | 94 | 1301 |
| 916596 | N/A | N/A | 13529 | 13544 | ATCATTGGAAGACCGC | 89 | 1302 |
| 916616 | N/A | N/A | 13998 | 14013 | GTTAAACTAAGGGTCA | 85 | 1303 |
| 916636 | N/A | N/A | 14550 | 14565 | CTTAATGTGGATTCAC | 91 | 1304 |
| 916656 | N/A | N/A | 15351 | 15366 | TCCAACTTCAGGCTGA | 74 | 1305 |
| 916676 | N/A | N/A | 15819 | 15834 | AGCTTTGTGGGCTCCA | 69 | 1306 |
| 916696 | N/A | N/A | 16982 | 16997 | GTTTAATAAGGGCACC | 63 | 1307 |
| 916716 | N/A | N/A | 17740 | 17755 | CGTATTAATCTGGTCA | 93 | 1308 |
| 916736 | N/A | N/A | 19126 | 19141 | CACCTAAAATGCTCAC | 17 | 1309 |
| 916756 | N/A | N/A | 20213 | 20228 | ACAAACTATGCCTAGA | 58 | 1310 |
| 916775 | N/A | N/A | 20609 | 20624 | AATTTTACGATCATCA | 78 | 1311 |
| 916795 | N/A | N/A | 20927 | 20942 | GACAGATCAGCACTCG | 80 | 1312 |
| 916815 | N/A | N/A | 21407 | 21422 | CAATTCTAGACATGGC | 88 | 1313 |
| 916835 | N/A | N/A | 22338 | 22353 | TGCACCTACCCTTTTC | 39 | 1314 |
| 916855 | N/A | N/A | 23251 | 23266 | ACACATAAAGGACCCC | 48 | 1315 |
| 916875 | N/A | N/A | 24241 | 24256 | GCATTACCAGGCACCT | 61 | 1316 |
| 916895 | N/A | N/A | 24912 | 24927 | GACATCACAGGTGTTG | 5 | 1317 |

TABLE 20
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915358 | 40 | 55 | 2778 | 2793 | GTGTCTCGGCCAGGGC | 0 | 1318 |
| 915378 | 96 | 111 | 2834 | 2849 | GTCCTGATCCGCAGCA | 0 | 1319 |
| 915398 | 184 | 199 | 2922 | 2937 | CTCCAGCCGCGCTCTG | 14 | 1320 |
| 915418 | 244 | 259 | 2982 | 2997 | AGGCAGCGGGTCGCCC | 0 | 1321 |
| 915438 | 342 | 357 | 3080 | 3095 | GCGGGATACCGGAGAG | 44 | 1322 |
| 915458 | 381 | 396 | 5955 | 5970 | TCCGCACAAGATCTGA | 41 | 1323 |
| 915478 | 408 | 423 | 5982 | 5997 | AGATGCCAATGTTCCG | 95 | 1324 |
| 915498 | 446 | 461 | 6020 | 6035 | CTGTCGGAGGAACTTG | 40 | 1325 |
| 915518 | 475 | 490 | 6049 | 6064 | ACATTGGCCGGGAGGC | 5 | 1326 |
| 915538 | 505 | 520 | 6079 | 6094 | ATGCCTATTTTGCCGG | 61 | 1327 |
| 915558 | 527 | 542 | 6101 | 6116 | ATCAGACACTCTGGTA | 0 | 1328 |
| 915618 | 748 | 763 | 11966 | 11981 | ACTTTAGGGCAGATGT | 87 | 1329 |
| 915638 | 818 | 833 | 12036 | 12051 | GTAGAGGTTCCCTGTG | 87 | 1330 |
| 915658 | 859 | 874 | N/A | N/A | AGCACCTTGAGATCCG | 0 | 1331 |
| 915678 | 926 | 941 | N/A | N/A | GTTGCAGATGCCCTTC | 24 | 1332 |
| 915698 | 980 | 995 | 16100 | 16115 | GGCGACCTCAGGATCC | 0 | 1333 |
| 915718 | 1032 | 1047 | 16152 | 16167 | AGGCAGCCGACTCCGG | 8 | 1334 |
| 915738 | 1114 | 1129 | 16234 | 16249 | GTGTCCAGGATGCTCT | 41 | 1335 |
| 915758 | 1147 | 1162 | N/A | N/A | TCACTCAGTGCTGTAG | 59 | 1336 |
| 915778 | 1217 | 1232 | 19057 | 19072 | ATAAGACATTATCCTA | 85 | 1337 |
| 915798 | 1264 | 1279 | 19104 | 19119 | ACAATCGCAATGGCAG | 66 | 1338 |
| 915818 | 1342 | 1357 | 23747 | 23762 | GTGAACACCTGTGAGG | 58 | 1339 |
| 915838 | 1400 | 1415 | 25163 | 25178 | TTGGCTGCTCACTGGC | 79 | 1340 |
| 915858 | 1436 | 1451 | 25199 | 25214 | GGGCCAGTCCTGCTCA | 0 | 1341 |
| 915878 | 1535 | 1550 | 25298 | 25313 | GCCCAAGAAGAAGTTC | 54 | 1342 |
| 915898 | 1590 | 1605 | 25353 | 25368 | CTAGTGAAAAACTGGG | 34 | 1343 |
| 915918 | 1628 | 1643 | 25391 | 25406 | GCTAGACTCGCCTCCT | 33 | 1344 |
| 915938 | 1680 | 1695 | 25443 | 25458 | TGCGGAGGTAGCTGCA | 0 | 1345 |
| 915958 | 1756 | 1771 | 25519 | 25534 | CCTAGCTTTTCATAAA | 0 | 1346 |
| 915978 | 1783 | 1798 | 25546 | 25561 | GACCGCTGCACAGGCG | 24 | 1347 |
| 915998 | 1810 | 1825 | 25573 | 25588 | ATGCTGATGTATTAGA | 86 | 1348 |
| 916018 | 1832 | 1847 | 25595 | 25610 | TCCCAACCAGCTGAAT | 3 | 1349 |
| 916038 | 1879 | 1894 | 25642 | 25657 | GAAACAGTCAGTAAGG | 64 | 1350 |
| 916058 | 1902 | 1917 | 25665 | 25680 | AGTCTGACCATTAATA | 86 | 1351 |
| 916078 | 1933 | 1948 | 25696 | 25711 | ACCTGTCATTCTAAGA | 18 | 1352 |
| 916098 | 1972 | 1987 | 25735 | 25750 | CAGCCTACCCCCCATC | 41 | 1353 |
| 916118 | 1999 | 2014 | 25762 | 25777 | CTCCACCCCACAAGAT | 0 | 1354 |
| 916138 | 2092 | 2107 | 25855 | 25870 | CTTTGCAGACCACCTG | 65 | 1355 |
| 916157 | 2134 | 2149 | 25897 | 25912 | TACCCCCGCCATGGAG | 57 | 1356 |
| 916177 | 2237 | 2252 | 26000 | 26015 | CAACAGGTAACAACGC | 88 | 1357 |
| 916197 | 2579 | 2594 | 26342 | 26357 | GTCAGACTTTCACTCA | 81 | 1358 |
| 916217 | 2659 | 2674 | 26422 | 26437 | GTGCTTGGCTCCTGCC | 43 | 1359 |
| 916237 | 2701 | 2716 | 26464 | 26479 | CAAGTTGTGTGCTCCA | 73 | 1360 |
| 916257 | 2769 | 2784 | 26532 | 26547 | CATCGCCACACATGGG | 61 | 1361 |
| 916277 | N/A | N/A | 4105 | 4120 | AGGAAGGGTCCCAAAC | 0 | 1362 |
| 916297 | N/A | N/A | 4731 | 4746 | TGATCATGTGGCGGTC | 80 | 1363 |
| 916317 | N/A | N/A | 5391 | 5406 | TGCTATCAGGTGCAGG | 60 | 1364 |
| 916337 | N/A | N/A | 5646 | 5661 | TATATTGGGCTCAATG | 71 | 1365 |
| 916357 | N/A | N/A | 6594 | 6609 | GTTTACAAACATGGAC | 26 | 1366 |
| 916377 | N/A | N/A | 7464 | 7479 | TCATTAGCATCACCGG | 33 | 1367 |
| 916397 | N/A | N/A | 7662 | 7677 | GGGAACTAACATACAC | 0 | 1368 |
| 916557 | N/A | N/A | 12274 | 12289 | GGGTATATGTTCCCAG | 0 | 1369 |
| 916577 | N/A | N/A | 12830 | 12845 | TGCATAGCCTTCTTTC | 84 | 1370 |
| 916597 | N/A | N/A | 13530 | 13545 | CATCATTGGAAGACCG | 62 | 1371 |
| 916617 | N/A | N/A | 14016 | 14031 | TCTTTAACTTCGGCCC | 70 | 1372 |
| 916637 | N/A | N/A | 14551 | 14566 | TCTTAATGTGGATTCA | 88 | 1373 |
| 916657 | N/A | N/A | 15388 | 15403 | TCAGACAACCACAGCT | 66 | 1374 |
| 916677 | N/A | N/A | 15852 | 15867 | TAAAGCAGGACACACG | 74 | 1375 |
| 916697 | N/A | N/A | 17077 | 17092 | AGACATGTTGGTGTCT | 0 | 1376 |
| 916717 | N/A | N/A | 17788 | 17803 | CCCCAGTCTTTTATTC | 0 | 1377 |
| 916737 | N/A | N/A | 19140 | 19155 | GGAAGACACGGAGCCA | 20 | 1378 |
| 916757 | N/A | N/A | 20240 | 20255 | CCTAACTGCTGGCTCT | 85 | 1379 |
| 916776 | N/A | N/A | 20610 | 20625 | TAATTTTACGATCATC | 76 | 1380 |
| 916796 | N/A | N/A | 20939 | 20954 | CTCTTTGTAGCAGACA | 90 | 1381 |
| 916816 | N/A | N/A | 21439 | 21454 | CAATATACTGAGAGGA | 92 | 1382 |
| 916836 | N/A | N/A | 22392 | 22407 | GTAGACATCCTTCCCG | 65 | 1383 |
| 916856 | N/A | N/A | 23252 | 23267 | GACACATAAAGGACCC | 59 | 1384 |
| 916876 | N/A | N/A | 24242 | 24257 | TGCATTACCAGGCACC | 37 | 1385 |
| 916896 | N/A | N/A | 24913 | 24928 | GGACATCACAGGTGTT | 19 | 1386 |

TABLE 21
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915359 | 41 | 56 | 2779 | 2794 | AGTGTCTCGGCCAGGG | 0 | 1387 |
| 915379 | 97 | 112 | 2835 | 2850 | GGTCCTGATCCGCAGC | 0 | 1388 |
| 915399 | 185 | 200 | 2923 | 2938 | GCTCCAGCCGCGCTCT | 26 | 1389 |
| 915419 | 245 | 260 | 2983 | 2998 | CAGGCAGCGGGTCGCC | 0 | 1390 |
| 915439 | 343 | 358 | 3081 | 3096 | AGCGGGATACCGGAGA | 69 | 1391 |
| 915459 | 382 | 397 | 5956 | 5971 | TTCCGCACAAGATCTG | 71 | 1392 |
| 915479 | 409 | 424 | 5983 | 5998 | AAGATGCCAATGTTCC | 93 | 1393 |
| 915499 | 448 | 463 | 6022 | 6037 | CCCTGTCGGAGGAACT | 30 | 1394 |
| 915519 | 477 | 492 | 6051 | 6066 | GGACATTGGCCGGGAG | 88 | 1395 |
| 915539 | 506 | 521 | 6080 | 6095 | GATGCCTATTTTGCCG | 60 | 1396 |
| 915559 | 529 | 544 | 6103 | 6118 | CCATCAGACACTCTGG | 30 | 1397 |
| 915579 | 595 | 610 | 7825 | 7840 | CAGGAACATACCAAGG | 98 | 1398 |
| 915599 | 674 | 689 | 11892 | 11907 | GTTGTCACTCACTCCT | 98 | 1399 |
| 915619 | 749 | 764 | 11967 | 11982 | GACTTTAGGGCAGATG | 96 | 1400 |
| 915639 | 819 | 834 | 12037 | 12052 | GGTAGAGGTTCCCTGT | 92 | 1401 |
| 915659 | 860 | 875 | N/A | N/A | CAGCACCTTGAGATCC | 0 | 1402 |
| 915679 | 928 | 943 | N/A | N/A | CTGTTGCAGATGCCCT | 58 | 1403 |
| 915699 | 981 | 996 | 16101 | 16116 | TGGCGACCTCAGGATC | 40 | 1404 |
| 915719 | 1033 | 1048 | 16153 | 16168 | AAGGCAGCCGACTCCG | 0 | 1405 |
| 915739 | 1115 | 1130 | 16235 | 16250 | GGTGTCCAGGATGCTC | 40 | 1406 |
| 915759 | 1148 | 1163 | N/A | N/A | TTCACTCAGTGCTGTA | 29 | 1407 |
| 915779 | 1219 | 1234 | 19059 | 19074 | ACATAAGACATTATCC | 86 | 1408 |
| 915799 | 1268 | 1283 | 19108 | 19123 | CTGGACAATCGCAATG | 42 | 1409 |
| 915819 | 1344 | 1359 | 23749 | 23764 | GAGTGAACACCTGTGA | 81 | 1410 |
| 915839 | 1401 | 1416 | 25164 | 25179 | GTTGGCTGCTCACTGG | 85 | 1411 |
| 915859 | 1437 | 1452 | 25200 | 25215 | AGGGCCAGTCCTGCTC | 0 | 1412 |
| 915879 | 1538 | 1553 | 25301 | 25316 | ATTGCCCAAGAAGAAG | 54 | 1413 |
| 915899 | 1591 | 1606 | 25354 | 25369 | TCTAGTGAAAAACTGG | 0 | 1414 |
| 915919 | 1629 | 1644 | 25392 | 25407 | TGCTAGACTCGCCTCC | 72 | 1415 |
| 915939 | 1681 | 1696 | 25444 | 25459 | ATGCGGAGGTAGCTGC | 39 | 1416 |
| 915959 | 1764 | 1779 | 25527 | 25542 | GGTTGCTTCCTAGCTT | 87 | 1417 |
| 915979 | 1784 | 1799 | 25547 | 25562 | GGACCGCTGCACAGGC | 0 | 1418 |
| 915999 | 1811 | 1826 | 25574 | 25589 | CATGCTGATGTATTAG | 35 | 1419 |
| 916019 | 1833 | 1848 | 25596 | 25611 | TTCCCAACCAGCTGAA | 0 | 1420 |
| 916039 | 1880 | 1895 | 25643 | 25658 | CGAAACAGTCAGTAAG | 80 | 1421 |
| 916059 | 1905 | 1920 | 25668 | 25683 | AACAGTCTGACCATTA | 85 | 1422 |
| 916079 | 1934 | 1949 | 25697 | 25712 | CACCTGTCATTCTAAG | 45 | 1423 |
| 916099 | 1973 | 1988 | 25736 | 25751 | CCAGCCTACCCCCCAT | 53 | 1424 |
| 916119 | 2022 | 2037 | 25785 | 25800 | GTGGGATCATGCTATT | 76 | 1425 |
| 916139 | 2093 | 2108 | 25856 | 25871 | TCTTTGCAGACCACCT | 85 | 1426 |
| 916158 | 2135 | 2150 | 25898 | 25913 | TTACCCCCGCCATGGA | 0 | 1427 |
| 916178 | 2238 | 2253 | 26001 | 26016 | TCAACAGGTAACAACG | 87 | 1428 |
| 916198 | 2620 | 2635 | 26383 | 26398 | GCACACTAGATTATTT | 66 | 1429 |
| 916218 | 2673 | 2688 | 26436 | 26451 | CGGAAGCTCCTGCTGT | 27 | 1430 |
| 916238 | 2702 | 2717 | 26465 | 26480 | TCAAGTTGTGTGCTCC | 91 | 1431 |
| 916258 | 2770 | 2785 | 26533 | 26548 | TCATCGCCACACATGG | 49 | 1432 |
| 916278 | N/A | N/A | 4211 | 4226 | TCATTTCCAGGAGTAC | 75 | 1433 |
| 916298 | N/A | N/A | 4735 | 4750 | CAAATGATCATGTGGC | 93 | 1434 |
| 916318 | N/A | N/A | 5394 | 5409 | TAATGCTATCAGGTGC | 95 | 1435 |
| 916338 | N/A | N/A | 5648 | 5663 | GATATATTGGGCTCAA | 97 | 1436 |
| 916358 | N/A | N/A | 6596 | 6611 | GGGTTTACAAACATGG | 75 | 1437 |
| 916378 | N/A | N/A | 7465 | 7480 | TTCATTAGCATCACCG | 78 | 1438 |
| 916398 | N/A | N/A | 7686 | 7701 | GTTAATCCATGGGTCA | 49 | 1439 |
| 916418 | N/A | N/A | 8992 | 9007 | AGCCTAAACTTCCTCC | 63 | 1440 |
| 916438 | N/A | N/A | 9318 | 9333 | AGAAGAGCCGCCCTGC | 77 | 1441 |
| 916458 | N/A | N/A | 9795 | 9810 | GCAAGACTAGCAAGTG | 85 | 1442 |
| 916478 | N/A | N/A | 10301 | 10316 | AGCATGCGGTATGTAC | 67 | 1443 |
| 916498 | N/A | N/A | 10849 | 10864 | CACACAATTTCTAGGG | 82 | 1444 |
| 916518 | N/A | N/A | 11346 | 11361 | TTGACAATTAGAACCA | 96 | 1445 |
| 916538 | N/A | N/A | 11711 | 11726 | ACAAATCCTTACCGAG | 54 | 1446 |
| 916558 | N/A | N/A | 12285 | 12300 | GTTTTAGGTCTGGGTA | 94 | 1447 |
| 916578 | N/A | N/A | 12831 | 12846 | TTGCATAGCCTTCTTT | 93 | 1448 |
| 916598 | N/A | N/A | 13660 | 13675 | CATACATACCCTTCTC | 9 | 1449 |
| 916618 | N/A | N/A | 14025 | 14040 | CGCAGAAACTCTTTAA | 89 | 1450 |
| 916638 | N/A | N/A | 14552 | 14567 | GTCTTAATGTGGATTC | 93 | 1451 |
| 916658 | N/A | N/A | 15421 | 15436 | AGCATTGGCACACTGG | 70 | 1452 |
| 916678 | N/A | N/A | 15857 | 15872 | GGCTTTAAAGCAGGAC | 62 | 1453 |
| 916698 | N/A | N/A | 17079 | 17094 | GCAGACATGTTGGTGT | 2 | 1454 |
| 916718 | N/A | N/A | 17839 | 17854 | TACAAGCTGGTCCTTG | 0 | 1455 |
| 916738 | N/A | N/A | 19211 | 19226 | GACAATCCAGGTCCCA | 70 | 1456 |
| 916758 | N/A | N/A | 20285 | 20300 | GAGGAAGCCCAATCAA | 81 | 1457 |
| 916777 | N/A | N/A | 20611 | 20626 | CTAATTTTACGATCAT | 81 | 1458 |
| 916797 | N/A | N/A | 20984 | 20999 | TTAAACTGCCAAGTCC | 83 | 1459 |
| 916817 | N/A | N/A | 21440 | 21455 | CCAATATACTGAGAGG | 96 | 1460 |
| 916837 | N/A | N/A | 22406 | 22421 | GGTAGCACCGCCAAGT | 0 | 1461 |
| 916857 | N/A | N/A | 23301 | 23316 | CACCATGGAGAGGTCT | 0 | 1462 |
| 916877 | N/A | N/A | 24243 | 24258 | TTGCATTACCAGGCAC | 17 | 1463 |
| 916897 | N/A | N/A | 24934 | 24949 | GCTACCTGGACACCTC | 47 | 1464 |

TABLE 22
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 841947 | 2094 | 2109 | 25857 | 25872 | ATCTTTGCAGACCACC | 89 | 1464 |
| 912986 | N/A | N/A | 20288 | 20303 | TCAGAGGAAGCCCAAT | 92 | 254 |
| | | | 20318 | 20333 | | | |
| 915360 | 42 | 57 | 2780 | 2795 | CAGTGTCTCGGCCAGG | 0 | 1466 |
| 915380 | 98 | 113 | 2836 | 2851 | GGGTCCTGATCCGCAG | 0 | 1467 |
| 915400 | 186 | 201 | 2924 | 2939 | AGCTCCAGCCGCGCTC | 0 | 1468 |
| 915420 | 246 | 261 | 2984 | 2999 | TCAGGCAGCGGGTCGC | 78 | 1469 |
| 915440 | 344 | 359 | 3082 | 3097 | CAGCGGGATACCGGAG | 72 | 1470 |
| 915460 | 383 | 398 | 5957 | 5972 | CTTCCGCACAAGATCT | 0 | 1471 |
| 915480 | 411 | 426 | 5985 | 6000 | GGAAGATGCCAATGTT | 94 | 1472 |
| 915500 | 449 | 464 | 6023 | 6038 | ACCCTGTCGGAGGAAC | 40 | 1473 |
| 915520 | 480 | 495 | 6054 | 6069 | GGTGGACATTGGCCGG | 38 | 1474 |
| 915540 | 507 | 522 | 6081 | 6096 | AGATGCCTATTTTGCC | 76 | 1475 |
| 915560 | 556 | 571 | 6130 | 6145 | CGAAAGTCAGACACCA | 69 | 1476 |
| 915620 | 750 | 765 | 11968 | 11983 | TGACTTTAGGGCAGAT | 89 | 1477 |
| 915640 | 821 | 836 | 12039 | 12054 | AAGGTAGAGGTTCCCT | 10 | 1478 |
| 915660 | 875 | 890 | 13611 | 13626 | AAGGCATATCTCTCCC | 47 | 1479 |
| 915680 | 929 | 944 | N/A | N/A | CCTGTTGCAGATGCCC | 22 | 1480 |
| 915700 | 982 | 997 | 16102 | 16117 | ATGGCGACCTCAGGAT | 58 | 1481 |
| 915720 | 1034 | 1049 | 16154 | 16169 | CAAGGCAGCCGACTCC | 63 | 1482 |
| 915740 | 1121 | 1136 | 16241 | 16256 | CGAGAGGGTGTCCAGG | 0 | 1483 |
| 915760 | 1149 | 1164 | N/A | N/A | CTTCACTCAGTGCTGT | 13 | 1484 |
| 915780 | 1226 | 1241 | 19066 | 19081 | CAGCATTACATAAGAC | 94 | 1485 |
| 915800 | 1270 | 1285 | 19110 | 19125 | CTCTGGACAATCGCAA | 55 | 1486 |
| 915820 | 1345 | 1360 | 23750 | 23765 | CGAGTGAACACCTGTG | 83 | 1487 |
| 915840 | 1402 | 1417 | 25165 | 25180 | TGTTGGCTGCTCACTG | 77 | 1488 |
| 915860 | 1470 | 1485 | 25233 | 25248 | CTGGACAGCCCTTGGG | 29 | 1489 |
| 915880 | 1539 | 1554 | 25302 | 25317 | TATTGCCCAAGAAGAA | 18 | 1490 |
| 915900 | 1598 | 1613 | 25361 | 25376 | ACTCTTCTCTAGTGAA | 67 | 1491 |
| 915920 | 1630 | 1645 | 25393 | 25408 | CTGCTAGACTCGCCTC | 88 | 1492 |
| 915940 | 1682 | 1697 | 25445 | 25460 | AATGCGGAGGTAGCTG | 0 | 1493 |
| 915960 | 1765 | 1780 | 25528 | 25543 | AGGTTGCTTCCTAGCT | 55 | 1494 |
| 915980 | 1785 | 1800 | 25548 | 25563 | TGGACCGCTGCACAGG | 82 | 1495 |
| 916000 | 1812 | 1827 | 25575 | 25590 | GCATGCTGATGTATTA | 52 | 1496 |
| 916020 | 1837 | 1852 | 25600 | 25615 | TCATTTCCCAACCAGC | 94 | 1497 |
| 916040 | 1881 | 1896 | 25644 | 25659 | ACGAAACAGTCAGTAA | 79 | 1498 |
| 916060 | 1907 | 1922 | 25670 | 25685 | GGAACAGTCTGACCAT | 22 | 1499 |
| 916080 | 1936 | 1951 | 25699 | 25714 | AACACCTGTCATTCTA | 71 | 1500 |
| 916100 | 1974 | 1989 | 25737 | 25752 | GCCAGCCTACCCCCCA | 23 | 1501 |
| 916120 | 2023 | 2038 | 25786 | 25801 | AGTGGGATCATGCTAT | 0 | 1502 |
| 916159 | 2136 | 2151 | 25899 | 25914 | GTTACCCCCGCCATGG | 47 | 1503 |
| 916179 | 2239 | 2254 | 26002 | 26017 | TTCAACAGGTAACAAC | 84 | 1504 |
| 916199 | 2621 | 2636 | 26384 | 26399 | TGCACACTAGATTATT | 0 | 1505 |
| 916219 | 2674 | 2689 | 26437 | 26452 | GCGGAAGCTCCTGCTG | 6 | 1506 |
| 916239 | 2704 | 2719 | 26467 | 26482 | GTTCAAGTTGTGTGCT | 85 | 1507 |
| 916259 | 2771 | 2786 | 26534 | 26549 | CTCATCGCCACACATG | 85 | 1508 |
| 916279 | N/A | N/A | 4218 | 4233 | CGGAATCTCATTTCCA | 0 | 1509 |
| 916299 | N/A | N/A | 4736 | 4751 | GCAAATGATCATGTGG | 93 | 1510 |
| 916319 | N/A | N/A | 5396 | 5411 | CTTAATGCTATCAGGT | 83 | 1511 |
| 916339 | N/A | N/A | 5649 | 5664 | GGATATATTGGGCTCA | 96 | 1512 |
| 916359 | N/A | N/A | 6597 | 6612 | AGGGTTTACAAACATG | 32 | 1513 |
| 916379 | N/A | N/A | 7466 | 7481 | ATTCATTAGCATCACC | 52 | 1514 |
| 916399 | N/A | N/A | 7687 | 7702 | GGTTAATCCATGGGTC | 0 | 1515 |
| 916559 | N/A | N/A | 12286 | 12301 | AGTTTTAGGTCTGGGT | 89 | 1516 |
| 916579 | N/A | N/A | 12833 | 12848 | CATTGCATAGCCTTCT | 96 | 1517 |
| 916599 | N/A | N/A | 13661 | 13676 | CCATACATACCCTTCT | 21 | 1518 |
| 916619 | N/A | N/A | 14077 | 14092 | ACCCACACCTGACTGG | 16 | 1519 |
| 916639 | N/A | N/A | 14572 | 14587 | CGCTCCTACTTATCCC | 96 | 1520 |
| 916659 | N/A | N/A | 15427 | 15442 | TCTTACAGCATTGGCA | 38 | 1521 |
| 916679 | N/A | N/A | 15973 | 15988 | CATCTACCAAACTGCA | 73 | 1522 |
| 916699 | N/A | N/A | 17135 | 17150 | AACAAACATCGATTTT | 47 | 1523 |
| 916719 | N/A | N/A | 17844 | 17859 | AGCTTTACAAGCTGGT | 0 | 1524 |
| 916739 | N/A | N/A | 19213 | 19228 | ACGACAATCCAGGTCC | 14 | 1525 |
| 916778 | N/A | N/A | 20612 | 20627 | TCTAATTTTACGATCA | 92 | 1526 |
| 916798 | N/A | N/A | 20985 | 21000 | ATTAAACTGCCAAGTC | 72 | 1527 |
| 916818 | N/A | N/A | 21441 | 21456 | ACCAATATACTGAGAG | 92 | 1528 |
| 916838 | N/A | N/A | 22409 | 22424 | AGCGGTAGCACCGCCA | 0 | 1529 |
| 916858 | N/A | N/A | 23323 | 23338 | TCACATGTGAGCCCAG | 46 | 1530 |
| 916878 | N/A | N/A | 24271 | 24286 | GTACAACAGAGGGTGG | 19 | 1531 |
| 916898 | N/A | N/A | 24978 | 24993 | GGCTGATGTCACCACC | 32 | 1532 |

TABLE 23
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915361 | 43 | 58 | 2781 | 2796 | TCAGTGTCTCGGCCAG | 0 | 1533 |
| 915381 | 105 | 120 | 2843 | 2858 | TCGGCTCGGGTCCTGA | 0 | 1534 |
| 915401 | 188 | 203 | 2926 | 2941 | CAAGCTCCAGCCGCGC | 22 | 1535 |
| 915421 | 247 | 262 | 2985 | 3000 | CTCAGGCAGCGGGTCG | 48 | 1536 |
| 915441 | 345 | 360 | 3083 | 3098 | CCAGCGGGATACCGGA | 0 | 1537 |
| 915461 | 384 | 399 | 5958 | 5973 | CCTTCCGCACAAGATC | 59 | 1538 |
| 915481 | 414 | 429 | 5988 | 6003 | GATGGAAGATGCCAAT | 83 | 1539 |
| 915501 | 450 | 465 | 6024 | 6039 | GACCCTGTCGGAGGAA | 26 | 1540 |
| 915521 | 482 | 497 | 6056 | 6071 | CTGGTGGACATTGGCC | 0 | 1541 |
| 915541 | 508 | 523 | 6082 | 6097 | GAGATGCCTATTTTGC | 92 | 1542 |
| 915561 | 557 | 572 | 6131 | 6146 | CCGAAAGTCAGACACC | 0 | 1543 |
| 915601 | 691 | 706 | 11909 | 11924 | GCATCAATGAAGGGTA | 91 | 1544 |
| 915621 | 751 | 766 | 11969 | 11984 | TTGACTTTAGGGCAGA | 85 | 1545 |
| 915641 | 822 | 837 | 12040 | 12055 | GAAGGTAGAGGTTCCC | 74 | 1546 |
| 915661 | 876 | 891 | 13612 | 13627 | GAAGGCATATCTCTCC | 32 | 1547 |
| 915681 | 930 | 945 | 16050 | 16065 | GCCTGTTGCAGATGCC | 0 | 1548 |
| 915701 | 983 | 998 | 16103 | 16118 | CATGGCGACCTCAGGA | 0 | 1549 |
| 915721 | 1035 | 1050 | 16155 | 16170 | CCAAGGCAGCCGACTC | 79 | 1550 |
| 915741 | 1122 | 1137 | 16242 | 16257 | GCGAGAGGGTGTCCAG | 0 | 1551 |
| 915761 | 1150 | 1165 | 18990 | 19005 | TCTTCACTCAGTGCTG | 41 | 1552 |
| 915781 | 1227 | 1242 | 19067 | 19082 | GCAGCATTACATAAGA | 75 | 1553 |
| 915801 | 1273 | 1288 | N/A | N/A | AGTCTCTGGACAATCG | 0 | 1554 |
| 915821 | 1346 | 1361 | 23751 | 23766 | TCGAGTGAACACCTGT | 52 | 1555 |
| 915841 | 1403 | 1418 | 25166 | 25181 | CTGTTGGCTGCTCACT | 80 | 1556 |
| 915861 | 1471 | 1486 | 25234 | 25249 | GCTGGACAGCCCTTGG | 0 | 1557 |
| 915881 | 1540 | 1555 | 25303 | 25318 | TTATTGCCCAAGAAGA | 75 | 1558 |
| 915901 | 1599 | 1614 | 25362 | 25377 | GACTCTTCTCTAGTGA | 67 | 1559 |
| 915921 | 1631 | 1646 | 25394 | 25409 | TCTGCTAGACTCGCCT | 50 | 1560 |
| 915941 | 1683 | 1698 | 25446 | 25461 | CAATGCGGAGGTAGCT | 39 | 1561 |
| 915961 | 1766 | 1781 | 25529 | 25544 | AAGGTTGCTTCCTAGC | 71 | 1562 |
| 915981 | 1786 | 1801 | 25549 | 25564 | CTGGACCGCTGCACAG | 0 | 1563 |
| 916001 | 1813 | 1828 | 25576 | 25591 | CGCATGCTGATGTATT | 73 | 1564 |
| 916021 | 1840 | 1855 | 25603 | 25618 | GTGTCATTTCCCAACC | 61 | 1565 |
| 916041 | 1882 | 1897 | 25645 | 25660 | CACGAAACAGTCAGTA | 34 | 1566 |
| 916061 | 1910 | 1925 | 25673 | 25688 | GCTGGAACAGTCTGAC | 82 | 1567 |
| 916081 | 1942 | 1957 | 25705 | 25720 | CATCCAAACACCTGTC | 69 | 1568 |
| 916101 | 1975 | 1990 | 25738 | 25753 | GGCCAGCCTACCCCCC | 2 | 1569 |
| 916121 | 2024 | 2039 | 25787 | 25802 | AAGTGGGATCATGCTA | 26 | 1570 |
| 916140 | 2095 | 2110 | 25858 | 25873 | CATCTTTGCAGACCAC | 92 | 1571 |
| 916160 | 2137 | 2152 | 25900 | 25915 | TGTTACCCCCGCCATG | 51 | 1572 |
| 916180 | 2257 | 2272 | 26020 | 26035 | GATTCACATAATACAA | 87 | 1573 |
| 916200 | 2622 | 2637 | 26385 | 26400 | CTGCACACTAGATTAT | 0 | 1574 |
| 916220 | 2675 | 2690 | 26438 | 26453 | GGCGGAAGCTCCTGCT | 0 | 1575 |
| 916240 | 2705 | 2720 | 26468 | 26483 | GGTTCAAGTTGTGTGC | 69 | 1576 |
| 916260 | 2772 | 2787 | 26535 | 26550 | TCTCATCGCCACACAT | 72 | 1577 |
| 916280 | N/A | N/A | 4220 | 4235 | TACGGAATCTCATTTC | 80 | 1578 |
| 916300 | N/A | N/A | 4791 | 4806 | GGCCACCTTGGGATAC | 17 | 1579 |
| 916320 | N/A | N/A | 5398 | 5413 | GCCTTAATGCTATCAG | 67 | 1580 |
| 916340 | N/A | N/A | 5650 | 5665 | TGGATATATTGGGCTC | 97 | 1581 |
| 916360 | N/A | N/A | 6603 | 6618 | ACATTCAGGGTTTACA | 18 | 1582 |
| 916380 | N/A | N/A | 7468 | 7483 | GTATTCATTAGCATCA | 51 | 1583 |
| 916400 | N/A | N/A | 7688 | 7703 | AGGTTAATCCATGGGT | 29 | 1584 |
| 916560 | N/A | N/A | 12287 | 12302 | GAGTTTTAGGTCTGGG | 95 | 1585 |
| 916580 | N/A | N/A | 12905 | 12920 | CTTATAAAGCACACGG | 95 | 1586 |
| 916600 | N/A | N/A | 13683 | 13698 | GGGCATGGCTGATCCT | 8 | 1587 |
| 916620 | N/A | N/A | 14099 | 14114 | CAAACTTGTCTAGTGG | 67 | 1588 |
| 916640 | N/A | N/A | 14600 | 14615 | TCGCATCCATGGGTCC | 83 | 1589 |
| 916660 | N/A | N/A | 15429 | 15444 | GCTCTTACAGCATTGG | 51 | 1590 |
| 916680 | N/A | N/A | 15974 | 15989 | CCATCTACCAAACTGC | 61 | 1591 |
| 916700 | N/A | N/A | 17195 | 17210 | GACTTAGTCCGTGTTC | 49 | 1592 |
| 916720 | N/A | N/A | 17883 | 17898 | CAGCATCTATGTTCTC | 67 | 1593 |
| 916740 | N/A | N/A | 19239 | 19254 | ATAGACTGTGAGCTGT | 82 | 1594 |
| 916759 | N/A | N/A | 20354 | 20369 | GACCATTCTGCTCCCC | 20 | 1595 |
| 916779 | N/A | N/A | 20632 | 20647 | GCCCATACCTTTTATC | 47 | 1596 |
| 916799 | N/A | N/A | 20987 | 21002 | GTATTAAACTGCCAAG | 81 | 1597 |
| 916819 | N/A | N/A | 21444 | 21459 | CTAACCAATATACTGA | 73 | 1598 |
| 916839 | N/A | N/A | 22506 | 22521 | GGCTGGTGATGAAACA | 0 | 1599 |
| 916859 | N/A | N/A | 23345 | 23360 | CCTCATGGTTTGCTGT | 31 | 1600 |
| 916879 | N/A | N/A | 24273 | 24288 | CAGTACAACAGAGGGT | 81 | 1601 |
| 916899 | N/A | N/A | 25064 | 25079 | CACATTGCCGGCCAGT | 58 | 1602 |

TABLE 24
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915362 | 44 | 59 | 2782 | 2797 | CTCAGTGTCTCGGCCA | 0 | 1603 |
| 915382 | 106 | 121 | 2844 | 2859 | ATCGGCTCGGGTCCTG | 30 | 1604 |
| 915402 | 189 | 204 | 2927 | 2942 | ACAAGCTCCAGCCGCG | 24 | 1605 |
| 915422 | 248 | 263 | 2986 | 3001 | GCTCAGGCAGCGGGTC | 33 | 1606 |
| 915442 | 346 | 361 | N/A | N/A | TCCAGCGGGATACCGG | 0 | 1607 |
| 915462 | 385 | 400 | 5959 | 5974 | GCCTTCCGCACAAGAT | 60 | 1608 |
| 915482 | 415 | 430 | 5989 | 6004 | GGATGGAAGATGCCAA | 60 | 1609 |
| 915502 | 451 | 466 | 6025 | 6040 | AGACCCTGTCGGAGGA | 75 | 1610 |
| 915522 | 487 | 502 | 6061 | 6076 | ATGAGCTGGTGGACAT | 74 | 1611 |
| 915542 | 509 | 524 | 6083 | 6098 | AGAGATGCCTATTTTG | 91 | 1612 |
| 915562 | 558 | 573 | 6132 | 6147 | ACCGAAAGTCAGACAC | 0 | 1613 |
| 915602 | 692 | 707 | 11910 | 11925 | GGCATCAATGAAGGGT | 88 | 1614 |
| 915622 | 752 | 767 | 11970 | 11985 | CTTGACTTTAGGGCAG | 86 | 1615 |
| 915642 | 824 | 839 | 12042 | 12057 | GAGAAGGTAGAGGTTC | 81 | 1616 |
| 915662 | 878 | 893 | 13614 | 13629 | TCGAAGGCATATCTCT | 16 | 1617 |
| 915682 | 931 | 946 | 16051 | 16066 | GGCCTGTTGCAGATGC | 0 | 1618 |
| 915702 | 984 | 999 | 16104 | 16119 | GCATGGCGACCTCAGG | 24 | 1619 |
| 915722 | 1036 | 1051 | 16156 | 16171 | GCCAAGGCAGCCGACT | 0 | 1620 |
| 915742 | 1123 | 1138 | 16243 | 16258 | GGCGAGAGGGTGTCCA | 0 | 1621 |
| 915762 | 1173 | 1188 | 19013 | 19028 | TGTATCCACCTTTGTC | 85 | 1622 |
| 915782 | 1228 | 1243 | 19068 | 19083 | GGCAGCATTACATAAG | 73 | 1623 |
| 915802 | 1283 | 1298 | N/A | N/A | CCATGTCACCAGTCTC | 59 | 1624 |
| 915822 | 1347 | 1362 | 23752 | 23767 | CTCGAGTGAACACCTG | 0 | 1625 |
| 915842 | 1404 | 1419 | 25167 | 25182 | CCTGTTGGCTGCTCAC | 88 | 1626 |
| 915862 | 1472 | 1487 | 25235 | 25250 | TGCTGGACAGCCCTTG | 0 | 1627 |
| 915882 | 1541 | 1556 | 25304 | 25319 | TTTATTGCCCAAGAAG | 38 | 1628 |
| 915902 | 1600 | 1615 | 25363 | 25378 | AGACTCTTCTCTAGTG | 60 | 1629 |
| 915922 | 1632 | 1647 | 25395 | 25410 | ATCTGCTAGACTCGCC | 86 | 1630 |
| 915942 | 1684 | 1699 | 25447 | 25462 | GCAATGCGGAGGTAGC | 38 | 1631 |
| 915962 | 1767 | 1782 | 25530 | 25545 | AAAGGTTGCTTCCTAG | 77 | 1632 |
| 915982 | 1787 | 1802 | 25550 | 25565 | GCTGGACCGCTGCACA | 79 | 1633 |
| 916002 | 1814 | 1829 | 25577 | 25592 | ACGCATGCTGATGTAT | 72 | 1634 |
| 916022 | 1841 | 1856 | 25604 | 25619 | GGTGTCATTTCCCAAC | 80 | 1635 |
| 916042 | 1883 | 1898 | 25646 | 25661 | CCACGAAACAGTCAGT | 87 | 1636 |
| 916062 | 1912 | 1927 | 25675 | 25690 | ATGCTGGAACAGTCTG | 78 | 1637 |
| 916082 | 1943 | 1958 | 25706 | 25721 | CCATCCAAACACCTGT | 64 | 1638 |
| 916102 | 1976 | 1991 | 25739 | 25754 | GGGCCAGCCTACCCCC | 0 | 1639 |
| 916122 | 2025 | 2040 | 25788 | 25803 | GAAGTGGGATCATGCT | 71 | 1640 |
| 916141 | 2097 | 2112 | 25860 | 25875 | ATCATCTTTGCAGACC | 91 | 1641 |
| 916161 | 2138 | 2153 | 25901 | 25916 | TTGTTACCCCCGCCAT | 89 | 1642 |
| 916181 | 2259 | 2274 | 26022 | 26037 | CTGATTCACATAATAC | 91 | 1643 |
| 916201 | 2623 | 2638 | 26386 | 26401 | CCTGCACACTAGATTA | 59 | 1644 |
| 916221 | 2676 | 2691 | 26439 | 26454 | AGGCGGAAGCTCCTGC | 0 | 1645 |
| 916241 | 2706 | 2721 | 26469 | 26484 | AGGTTCAAGTTGTGTG | 87 | 1646 |
| 916281 | N/A | N/A | 4224 | 4239 | AATGTACGGAATCTCA | 83 | 1647 |
| 916301 | N/A | N/A | 4810 | 4825 | GTCCATGTGGGTGTCC | 74 | 1648 |
| 916321 | N/A | N/A | 5399 | 5414 | GGCCTTAATGCTATCA | 13 | 1649 |
| 916341 | N/A | N/A | 5711 | 5726 | TAGTATGAAATATCTC | 96 | 1650 |
| 916361 | N/A | N/A | 6862 | 6877 | ATTGTAACTGCCAGGC | 0 | 1651 |
| 916381 | N/A | N/A | 7471 | 7486 | CCGGTATTCATTAGCA | 0 | 1652 |
| 916401 | N/A | N/A | 7728 | 7743 | GAGCAGGGCAACAAAC | 22 | 1653 |
| 916561 | N/A | N/A | 12315 | 12330 | ATATAACCACAGCCTG | 54 | 1654 |
| 916581 | N/A | N/A | 12906 | 12921 | GCTTATAAAGCACACG | 94 | 1655 |
| 916601 | N/A | N/A | 13702 | 13717 | TAGTAAATGCTTGTCA | 95 | 1656 |
| 916621 | N/A | N/A | 14123 | 14138 | GGCAGAAATGTGCTCT | 60 | 1657 |
| 916641 | N/A | N/A | 14632 | 14647 | CTTCATGCCATCCTGT | 83 | 1658 |
| 916661 | N/A | N/A | 15430 | 15445 | TGCTCTTACAGCATTG | 0 | 1659 |
| 916681 | N/A | N/A | 16262 | 16277 | GGTACCTGTAGCGAGC | 0 | 1660 |
| 916701 | N/A | N/A | 17197 | 17212 | TTGACTTAGTCCGTGT | 93 | 1661 |
| 916721 | N/A | N/A | 18220 | 18235 | AGCTACATCAGGCTGG | 0 | 1662 |
| 916741 | N/A | N/A | 19244 | 19259 | TGCACATAGACTGTGA | 0 | 1663 |
| 916760 | N/A | N/A | 20373 | 20388 | GACTGCTGAGCCAAGC | 61 | 1664 |
| 916780 | N/A | N/A | 20657 | 20672 | AGAAATTGCAGTGCCC | 92 | 1665 |
| 916800 | N/A | N/A | 20988 | 21003 | GGTATTAAACTGCCAA | 0 | 1666 |
| 916820 | N/A | N/A | 21447 | 21462 | TCACTAACCAATATAC | 47 | 1667 |
| 916840 | N/A | N/A | 22602 | 22617 | ATAAATCTGCAAGAGC | 62 | 1668 |
| 916860 | N/A | N/A | 23369 | 23384 | TCTCATGGTCAAGACC | 52 | 1669 |
| 916880 | N/A | N/A | 24305 | 24320 | GACTGCTAGGCTTCAC | 54 | 1670 |
| 916900 | N/A | N/A | 25100 | 25115 | CGCTGCTGCAGTGTGC | 34 | 1671 |

Human primer probe set RTS36075 (forward sequence TGAGGCTGGAGGGAGATG, designated herein as SEQ ID NO: 14; reverse sequence GCTCATGTATCCACCTTTGTCT, designated herein as SEQ ID NO: 15; probe sequence CTAGACCACCTGCGTCTCAGCATC, designated herein as SEQ ID NO: 16) was also used to measure mRNA levels. PNPLA3 mRNA levels were adjusted according to total RNA content, as measured by RIBOGREEN®. Results are presented as percent inhibition of PNPLA3, relative to untreated control cells.

TABLE 25
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 898558 | 581 | 596 | N/A | N/A | GGCATCCACGACTTCG | 87 | 1672 |
| 912709 | 27 | 42 | 2765 | 2780 | GGCATTCCCAGCGCGA | 0 | 17 |
| 912710 | 95 | 110 | 2833 | 2848 | TCCTGATCCGCAGCAG | 0 | 18 |
| 912711 | 103 | 118 | 2841 | 2856 | GGCTCGGGTCCTGATC | 0 | 19 |
| 912712 | 131 | 146 | 2869 | 2884 | GTTAGGATCTGGGTCG | 76 | 20 |
| 912713 | 164 | 179 | 2902 | 2917 | GTACATGGCGGCGGCG | 0 | 21 |
| 912714 | 183 | 198 | 2921 | 2936 | TCCAGCCGCGCTCTGC | 29 | 22 |
| 912715 | 196 | 211 | 2934 | 2949 | GCGAAGGACAAGCTCC | 31 | 23 |
| 912716 | 197 | 212 | 2935 | 2950 | CGCGAAGGACAAGCTC | 0 | 24 |
| 912717 | 272 | 287 | 3010 | 3025 | GCGGAGGAGGTGCGGG | 0 | 25 |
| 912718 | 273 | 288 | 3011 | 3026 | CGCGGAGGAGGTGCGG | 0 | 26 |
| 912719 | 274 | 289 | 3012 | 3027 | TCGCGGAGGAGGTGCG | 16 | 27 |
| 912720 | 290 | 305 | 3028 | 3043 | GAACAACATGCGCGCG | 0 | 28 |
| 912721 | 291 | 306 | 3029 | 3044 | CGAACAACATGCGCGC | 2 | 29 |
| 912722 | 292 | 307 | 3030 | 3045 | CCGAACAACATGCGCG | 0 | 30 |
| 912723 | 293 | 308 | 3031 | 3046 | GCCGAACAACATGCGC | 0 | 31 |
| 912724 | 294 | 309 | 3032 | 3047 | CGCCGAACAACATGCG | 0 | 32 |
| 912725 | 323 | 338 | 3061 | 3076 | GCCGACGCAGTGCAAC | 0 | 33 |
| 912726 | 324 | 339 | 3062 | 3077 | CGCCGACGCAGTGCAA | 0 | 34 |
| 912727 | 340 | 355 | 3078 | 3093 | GGGATACCGGAGAGGA | 32 | 35 |
| 912728 | 370 | 385 | 5944 | 5959 | TCTGAGAGGACCTGCA | 31 | 36 |
| 912729 | 375 | 390 | 5949 | 5964 | CAAGATCTGAGAGGAC | 60 | 37 |
| 912730 | 404 | 419 | 5978 | 5993 | GCCAATGTTCCGACTC | 52 | 38 |
| 912731 | 410 | 425 | 5984 | 5999 | GAAGATGCCAATGTTC | 31 | 39 |
| 912732 | 429 | 444 | 6003 | 6018 | TTAAGTTGAAGGATGG | 93 | 40 |
| 912733 | 432 | 447 | 6006 | 6021 | TGCTTAAGTTGAAGGA | 82 | 41 |
| 912734 | 478 | 493 | 6052 | 6067 | TGGACATTGGCCGGGA | 73 | 42 |
| 912735 | 479 | 494 | 6053 | 6068 | GTGGACATTGGCCGGG | 44 | 43 |
| 912736 | 484 | 499 | 6058 | 6073 | AGCTGGTGGACATTGG | 29 | 44 |
| 912737 | 528 | 543 | 6102 | 6117 | CATCAGACACTCTGGT | 0 | 45 |
| 912738 | 531 | 546 | 6105 | 6120 | CCCCATCAGACACTCT | 55 | 46 |
| 912739 | 552 | 567 | 6126 | 6141 | AGTCAGACACCAGAAC | 23 | 47 |
| 912740 | 582 | 597 | N/A | N/A | AGGCATCCACGACTTC | 40 | 1673 |
| 912741 | 584 | 599 | N/A | N/A | CAAGGCATCCACGACT | 55 | 1674 |
| 912742 | 591 | 606 | N/A | N/A | AACATACCAAGGCATC | 59 | 1675 |
| 912743 | 593 | 608 | N/A | N/A | GGAACATACCAAGGCA | 69 | 1676 |
| 912744 | 594 | 609 | 7824 | 7839 | AGGAACATACCAAGGC | 85 | 1677 |
| 912745 | 625 | 640 | 7855 | 7870 | GGGATAAGGCCACTGT | 71 | 1678 |
| 912746 | 626 | 641 | 7856 | 7871 | AGGGATAAGGCCACTG | 12 | 1679 |
| 912747 | 630 | 645 | 7860 | 7875 | AAGGAGGGATAAGGCC | 0 | 1680 |
| 912748 | 652 | 667 | N/A | N/A | ACATATCGCACGCCTC | 35 | 1681 |
| 912749 | 653 | 668 | N/A | N/A | CACATATCGCACGCCT | 3 | 1682 |
| 912750 | 654 | 669 | N/A | N/A | CCACATATCGCACGCC | 27 | 1683 |
| 912751 | 656 | 671 | N/A | N/A | ATCCACATATCGCACG | 24 | 1684 |
| 912752 | 660 | 675 | 11878 | 11893 | CTCCATCCACATATCG | 87 | 1685 |
| 912753 | 689 | 704 | 11907 | 11922 | ATCAATGAAGGGTACG | 79 | 1686 |
| 912754 | 690 | 705 | 11908 | 11923 | CATCAATGAAGGGTAC | 63 | 1687 |
| 912755 | 693 | 708 | 11911 | 11926 | TGGCATCAATGAAGGG | 68 | 48 |
| 912756 | 698 | 713 | 11916 | 11931 | TGTTTTGGCATCAATG | 88 | 49 |
| 912757 | 746 | 761 | 11964 | 11979 | TTTAGGGCAGATGTCG | 75 | 50 |
| 912758 | 747 | 762 | 11965 | 11980 | CTTTAGGGCAGATGTC | 82 | 51 |
| 912759 | 795 | 810 | 12013 | 12028 | GTAGACTGAGCTTGGT | 96 | 52 |
| 912760 | 820 | 835 | 12038 | 12053 | AGGTAGAGGTTCCCTG | 0 | 53 |
| 912761 | 841 | 856 | 12059 | 12074 | GGGACAAAAGCTCTCG | 0 | 54 |
| 912762 | 873 | 888 | 13609 | 13624 | GGCATATCTCTCCCAG | 0 | 55 |
| 912763 | 874 | 889 | 13610 | 13625 | AGGCATATCTCTCCCA | 0 | 56 |
| 912764 | 886 | 901 | 13622 | 13637 | AAATATCCTCGAAGGC | 71 | 57 |
| 912765 | 888 | 903 | 13624 | 13639 | CCAAATATCCTCGAAG | 37 | 58 |
| 912766 | 889 | 904 | 13625 | 13640 | TCCAAATATCCTCGAA | 0 | 59 |
| 912767 | 894 | 909 | 13630 | 13645 | ATGCATCCAAATATCC | 42 | 60 |
| 912768 | 925 | 940 | N/A | N/A | TTGCAGATGCCCTTCT | 5 | 61 |
| 912769 | 968 | 983 | 16088 | 16103 | ATCCATCCCTTCTGAG | 6 | 62 |
| 912770 | 986 | 1001 | 16106 | 16121 | GGGCATGGCGACCTCA | 0 | 63 |
| 912771 | 1004 | 1019 | 16124 | 16139 | ACTCATGTTTGCCCAG | 67 | 64 |
| 912782 | 1195 | 1210 | 19035 | 19050 | AGCAAGTTGCAAATCT | 71 | 75 |
| 912783 | 1199 | 1214 | 19039 | 19054 | GGGTAGCAAGTTGCAA | 37 | 76 |
| 912784 | 1205 | 1220 | 19045 | 19060 | CCTAATGGGTAGCAAG | 25 | 77 |
| 912785 | 1206 | 1221 | 19046 | 19061 | TCCTAATGGGTAGCAA | 64 | 78 |

TABLE 26
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912786 | 1207 | 1222 | 19047 | 19062 | ATCCTAATGGGTAGCA | 65 | 79 |
| 912787 | 1211 | 1226 | 19051 | 19066 | CATTATCCTAATGGGT | 43 | 80 |
| 912788 | 1212 | 1227 | 19052 | 19067 | ACATTATCCTAATGGG | 0 | 81 |
| 912789 | 1213 | 1228 | 19053 | 19068 | GACATTATCCTAATGG | 59 | 82 |
| 912790 | 1220 | 1235 | 19060 | 19075 | TACATAAGACATTATC | 8 | 83 |
| 912791 | 1224 | 1239 | 19064 | 19079 | GCATTACATAAGACAT | 86 | 84 |
| 912792 | 1245 | 1260 | 19085 | 19100 | CCACAGGCAGGGTACA | 58 | 85 |
| 912793 | 1246 | 1261 | 19086 | 19101 | TCCACAGGCAGGGTAC | 5 | 86 |
| 912794 | 1253 | 1268 | 19093 | 19108 | GGCAGATTCCACAGGC | 68 | 87 |
| 912795 | 1259 | 1274 | 19099 | 19114 | CGCAATGGCAGATTCC | 84 | 88 |
| 912796 | 1265 | 1280 | 19105 | 19120 | GACAATCGCAATGGCA | 63 | 89 |
| 912797 | 1266 | 1281 | 19106 | 19121 | GGACAATCGCAATGGC | 54 | 90 |
| 912798 | 1267 | 1282 | 19107 | 19122 | TGGACAATCGCAATGG | 59 | 91 |
| 912799 | 1285 | 1300 | 23690 | 23705 | AGCCATGTCACCAGTC | 51 | 92 |
| 912800 | 1289 | 1304 | 23694 | 23709 | TGGAAGCCATGTCACC | 32 | 93 |
| 912801 | 1290 | 1305 | 23695 | 23710 | CTGGAAGCCATGTCAC | 44 | 94 |
| 912802 | 1297 | 1312 | 23702 | 23717 | GGCATATCTGGAAGCC | 0 | 95 |
| 912803 | 1298 | 1313 | 23703 | 23718 | GGGCATATCTGGAAGC | 0 | 96 |
| 912804 | 1351 | 1366 | 23756 | 23771 | AGCACTCGAGTGAACA | 6 | 97 |
| 912805 | 1386 | 1401 | N/A | N/A | GCATTTGGGACCTGGA | 54 | 98 |
| 912806 | 1387 | 1402 | N/A | N/A | GGCATTTGGGACCTGG | 33 | 99 |
| 912807 | 1388 | 1403 | 25151 | 25166 | TGGCATTTGGGACCTG | 0 | 100 |
| 912808 | 1394 | 1409 | 25157 | 25172 | GCTCACTGGCATTTGG | 7 | 101 |
| 912809 | 1523 | 1538 | 25286 | 25301 | GTTCAGGCTGGACCTG | 17 | 102 |
| 912810 | 1547 | 1562 | 25310 | 25325 | AGGTACTTTATTGCCC | 30 | 103 |
| 912811 | 1550 | 1565 | 25313 | 25328 | AGCAGGTACTTTATTG | 55 | 104 |
| 912812 | 1653 | 1668 | 25416 | 25431 | AACTTTAGCACCTCTG | 87 | 105 |
| 912813 | 1655 | 1670 | 25418 | 25433 | GAAACTTTAGCACCTC | 85 | 106 |
| 912814 | 1656 | 1671 | 25419 | 25434 | GGAAACTTTAGCACCT | 26 | 107 |
| 912815 | 1669 | 1684 | 25432 | 25447 | CTGCACAAAGATGGGA | 66 | 108 |
| 912816 | 1671 | 1686 | 25434 | 25449 | AGCTGCACAAAGATGG | 41 | 109 |
| 912817 | 1685 | 1700 | 25448 | 25463 | AGCAATGCGGAGGTAG | 35 | 110 |
| 912818 | 1740 | 1755 | 25503 | 25518 | ACCAACTCAGCTCAGA | 76 | 111 |
| 912819 | 1741 | 1756 | 25504 | 25519 | AACCAACTCAGCTCAG | 77 | 112 |
| 912820 | 1757 | 1772 | 25520 | 25535 | TCCTAGCTTTTCATAA | 18 | 113 |
| 912821 | 1788 | 1803 | 25551 | 25566 | TGCTGGACCGCTGCAC | 1 | 114 |
| 912822 | 1796 | 1811 | 25559 | 25574 | GAGTTAAGTGCTGGAC | 90 | 115 |
| 912823 | 1802 | 1817 | 25565 | 25580 | GTATTAGAGTTAAGTG | 86 | 116 |
| 912824 | 1803 | 1818 | 25566 | 25581 | TGTATTAGAGTTAAGT | 79 | 117 |
| 912825 | 1806 | 1821 | 25569 | 25584 | TGATGTATTAGAGTTA | 89 | 118 |
| 912826 | 1808 | 1823 | 25571 | 25586 | GCTGATGTATTAGAGT | 79 | 119 |
| 912827 | 1821 | 1836 | 25584 | 25599 | TGAATTAACGCATGCT | 73 | 120 |
| 912828 | 1822 | 1837 | 25585 | 25600 | CTGAATTAACGCATGC | 69 | 121 |
| 912829 | 1870 | 1885 | 25633 | 25648 | AGTAAGGGACCCTCTG | 0 | 122 |
| 912830 | 1871 | 1886 | 25634 | 25649 | CAGTAAGGGACCCTCT | 44 | 123 |
| 912831 | 1872 | 1887 | 25635 | 25650 | TCAGTAAGGGACCCTC | 67 | 124 |
| 912832 | 1874 | 1889 | 25637 | 25652 | AGTCAGTAAGGGACCC | 50 | 125 |
| 912833 | 1893 | 1908 | 25656 | 25671 | ATTAATAGGGCCACGA | 78 | 126 |
| 912834 | 1895 | 1910 | 25658 | 25673 | CCATTAATAGGGCCAC | 72 | 127 |
| 912835 | 1896 | 1911 | 25659 | 25674 | ACCATTAATAGGGCCA | 65 | 128 |
| 912836 | 1906 | 1921 | 25669 | 25684 | GAACAGTCTGACCATT | 82 | 129 |
| 912837 | 1908 | 1923 | 25671 | 25686 | TGGAACAGTCTGACCA | 39 | 130 |
| 912838 | 1909 | 1924 | 25672 | 25687 | CTGGAACAGTCTGACC | 84 | 131 |
| 912839 | 1911 | 1926 | 25674 | 25689 | TGCTGGAACAGTCTGA | 72 | 132 |
| 912840 | 1916 | 1931 | 25679 | 25694 | CCTCATGCTGGAACAG | 84 | 133 |
| 912841 | 1928 | 1943 | 25691 | 25706 | TCATTCTAAGAACCTC | 87 | 134 |
| 912842 | 1945 | 1960 | 25708 | 25723 | ACCCATCCAAACACCT | 18 | 135 |
| 912843 | 1982 | 1997 | 25745 | 25760 | ACACATGGGCCAGCCT | 46 | 136 |
| 912844 | 1989 | 2004 | 25752 | 25767 | CAAGATCACACATGGG | 71 | 137 |
| 912845 | 2057 | 2072 | 25820 | 25835 | GGGACGAACTGCACCC | 0 | 138 |
| 912846 | 2098 | 2113 | 25861 | 25876 | TATCATCTTTGCAGAC | 68 | 139 |
| 912847 | 2116 | 2131 | 25879 | 25894 | GTTTTTAGTAGTCAAG | 90 | 140 |
| 912848 | 2117 | 2132 | 25880 | 25895 | CGTTTTTAGTAGTCAA | 94 | 141 |
| 912849 | 2145 | 2160 | 25908 | 25923 | TATCATCTTGTTACCC | 87 | 142 |
| 912850 | 2148 | 2163 | 25911 | 25926 | GATTATCATCTTGTTA | 60 | 143 |
| 912851 | 2150 | 2165 | 25913 | 25928 | TAGATTATCATCTTGT | 50 | 144 |
| 912852 | 2151 | 2166 | 25914 | 25929 | GTAGATTATCATCTTG | 72 | 145 |
| 912853 | 2152 | 2167 | 25915 | 25930 | AGTAGATTATCATCTT | 79 | 146 |
| 912854 | 2175 | 2190 | 25938 | 25953 | GTGAAAAAGGTGTTCT | 64 | 147 |
| 912855 | 2182 | 2197 | 25945 | 25960 | TAGTTAGGTGAAAAAG | 77 | 148 |
| 912856 | 2188 | 2203 | 25951 | 25966 | TTATTTTAGTTAGGTG | 82 | 149 |
| 912857 | 2190 | 2205 | 25953 | 25968 | CATTATTTTAGTTAGG | 77 | 150 |
| 912858 | 2273 | 2288 | 26036 | 26051 | CTACTAACATCTCACT | 48 | 151 |
| 912859 | 2274 | 2289 | 26037 | 26052 | TCTACTAACATCTCAC | 91 | 152 |
| 912860 | 2278 | 2293 | 26041 | 26056 | TTATTCTACTAACATC | 37 | 153 |
| 912861 | 2280 | 2295 | 26043 | 26058 | GCTTATTCTACTAACA | 77 | 154 |
| 912862 | 2281 | 2296 | 26044 | 26059 | GGCTTATTCTACTAAC | 70 | 155 |
| 912863 | 2632 | 2647 | 26395 | 26410 | GGTGAATGCCCTGCAC | 42 | 156 |

### Study 2

Cultured A431 cells at a density of 5,000 cells per well were transfected by free uptake with 1,000 nM antisense oligonucleotide. After a treatment period of approximately 24 hours, RNA was isolated from the cells and PNPLA3 mRNA levels were measured by quantitative real-time PCR. Human primer probe set RTS36070 was used to measure mRNA levels. PNPLA3 mRNA levels were adjusted according to total RNA content, as measured by RIBOGREEN®. Results are presented as percent inhibition of PNPLA3, relative to untreated control cells.

TABLE 27
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 97 | 702 |
| 959270 | 413 | 428 | 5987 | 6002 | ATGGAAGATGCCAATG | 32 | 1688 |
| 959280 | 491 | 506 | 6065 | 6080 | GGAGATGAGCTGGTGG | 66 | 1689 |
| 959290 | 793 | 808 | 12011 | 12026 | AGACTGAGCTTGGTGA | 78 | 1690 |
| 959300 | 899 | 914 | 13635 | 13650 | CCTGAATGCATCCAAA | 69 | 1691 |
| 959310 | 1084 | 1099 | 16204 | 16219 | ATGCTGAGACGCAGGT | 0 | 1692 |
| 959320 | 1256 | 1271 | 19096 | 19111 | AATGGCAGATTCCACA | 25 | 1693 |
| 959330 | 1642 | 1657 | 25405 | 25420 | CTCTGAAAGAATCTGC | 75 | 1694 |
| 959340 | 1659 | 1674 | 25422 | 25437 | ATGGGAAACTTTAGCA | 77 | 1695 |
| 959350 | 1839 | 1854 | 25602 | 25617 | TGTCATTTCCCAACCA | 79 | 1696 |
| 959360 | 2114 | 2129 | 25877 | 25892 | TTTTAGTAGTCAAGGT | 88 | 1697 |
| 959370 | 2223 | 2238 | 25986 | 26001 | GCTTCCTTACATTTTT | 85 | 1698 |
| 959380 | 2269 | 2284 | 26032 | 26047 | TAACATCTCACTGATT | 42 | 1699 |
| 959390 | N/A | N/A | 4311 | 4326 | CTAGTGAGAAACAAAC | 0 | 1700 |
| 959400 | N/A | N/A | 4761 | 4776 | TTATTGTTGCTAAACC | 32 | 1701 |
| 959410 | N/A | N/A | 4863 | 4878 | ACTTTAGGCTCCTGGG | 60 | 1702 |
| 959420 | N/A | N/A | 5285 | 5300 | AGCCATAAATCTTGGG | 24 | 1703 |
| 959430 | N/A | N/A | 5573 | 5588 | ATGACATCATGGCTTC | 93 | 1704 |
| 959440 | N/A | N/A | 5603 | 5618 | TTATTCAATGTGGCTT | 95 | 1705 |
| 959450 | N/A | N/A | 5640 | 5655 | GGGCTCAATGAAATTA | 12 | 1706 |
| 959460 | N/A | N/A | 5713 | 5728 | CTTAGTATGAAATATC | 86 | 1707 |
| 959470 | N/A | N/A | 5808 | 5823 | TACTGTCTACTATGGG | 91 | 1708 |
| 959480 | N/A | N/A | 6157 | 6172 | CTTACATCCACGACTT | 35 | 1709 |
| 959660 | N/A | N/A | 12153 | 12168 | CAGTAACTGGTAGCTC | 74 | 1710 |
| 959670 | N/A | N/A | 12169 | 12184 | TGTTTGATTGTGCAGA | 95 | 1711 |
| 959680 | N/A | N/A | 12210 | 12225 | CGCCTTTTATTTCCGT | 92 | 1712 |
| 959690 | N/A | N/A | 12313 | 12328 | ATAACCACAGCCTGGG | 66 | 1713 |
| 959700 | N/A | N/A | 12675 | 12690 | ATAAGAATCATCTTAG | 7 | 1714 |
| 959710 | N/A | N/A | 12711 | 12726 | ACTACCGAACGCAGTT | 41 | 1715 |
| 959720 | N/A | N/A | 12757 | 12772 | TAGAGTGGTAAGGCAT | 84 | 1716 |
| 959730 | N/A | N/A | 12793 | 12808 | GGTTGGTGTACATGGT | 96 | 1717 |
| 959740 | N/A | N/A | 12880 | 12895 | TCCTGTTAGACAGCTT | 93 | 1718 |
| 959750 | N/A | N/A | 12902 | 12917 | ATAAAGCACACGGGAA | 86 | 1719 |
| 959760 | N/A | N/A | 12931 | 12946 | TAAGAGCTGTCTCCTC | 85 | 1720 |
| 959770 | N/A | N/A | 12972 | 12987 | CTAACAAACTTTGCAG | 79 | 1721 |
| 959780 | N/A | N/A | 13392 | 13407 | TGTCACCCTTCCACGG | 15 | 1722 |
| 959790 | N/A | N/A | 13526 | 13541 | ATTGGAAGACCGCAGA | 43 | 1723 |
| 959800 | N/A | N/A | 13706 | 13721 | CCGCTAGTAAATGCTT | 44 | 1724 |
| 959810 | N/A | N/A | 13737 | 13752 | AACTAAGGCAAATCTC | 77 | 1725 |
| 959820 | N/A | N/A | 13915 | 13930 | GAGTCATGACATCCCA | 89 | 1726 |
| 959830 | N/A | N/A | 14299 | 14314 | GCAGATAAATACACAT | 93 | 1727 |
| 959840 | N/A | N/A | 14424 | 14439 | TTTCCCATCGACACAG | 78 | 1728 |
| 959850 | N/A | N/A | 14571 | 14586 | GCTCCTACTTATCCCC | 76 | 1729 |
| 959860 | N/A | N/A | 15202 | 15217 | TATTGCCAGGTATCTG | 64 | 1730 |
| 959870 | N/A | N/A | 15599 | 15614 | CAATACATAGCAGAGC | 23 | 1731 |
| 959880 | N/A | N/A | 17192 | 17207 | TTAGTCCGTGTTCAGG | 90 | 1732 |
| 959890 | N/A | N/A | 17222 | 17237 | GTAGCTGGTTTGTGGG | 20 | 1733 |
| 959900 | N/A | N/A | 17295 | 17310 | CATCTCTTAGGGCACC | 79 | 1734 |
| 959910 | N/A | N/A | 18393 | 18408 | GTTTGGAAGTCGCCAT | 77 | 1735 |
| 959920 | N/A | N/A | 20284 | 20299 | AGGAAGCCCAATCAAG | 85 | 1736 |
| 959930 | N/A | N/A | 20512 | 20527 | CAGATTGAGTCTCCTG | 10 | 1737 |
| 959940 | N/A | N/A | 20607 | 20622 | TTTTACGATCATCATT | 72 | 1738 |
| 959950 | N/A | N/A | 20661 | 20676 | GCTTAGAAATTGCAGT | 75 | 1739 |
| 959960 | N/A | N/A | 20812 | 20827 | AGGGTAATATTCAGAC | 86 | 1740 |
| 959970 | N/A | N/A | 20934 | 20949 | TGTAGCAGACAGATCA | 74 | 1741 |
| 959980 | N/A | N/A | 21000 | 21015 | TTTAACAGCTCAGGTA | 66 | 1742 |
| 959990 | N/A | N/A | 21405 | 21420 | ATTCTAGACATGGCCA | 51 | 1743 |
| 960000 | N/A | N/A | 21442 | 21457 | AACCAATATACTGAGA | 71 | 1744 |
| 960010 | N/A | N/A | 21545 | 21560 | AGACATATGACATTTC | 91 | 1745 |
| 960020 | N/A | N/A | 22765 | 22780 | ACATGACAGACTAACT | 55 | 1746 |
| 960030 | N/A | N/A | 24039 | 24054 | CATCAATGCTGCACTC | 13 | 1747 |

TABLE 28
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 98 | 702 |
| 959271 | 425 | 440 | 5999 | 6014 | GTTGAAGGATGGATGG | 90 | 1748 |
| 959281 | 511 | 526 | 6085 | 6100 | AGAGAGATGCCTATTT | 73 | 1749 |
| 959291 | 813 | 828 | 12031 | 12046 | GGTTCCCTGTGCAGAG | 79 | 1750 |
| 959301 | 904 | 919 | 13640 | 13655 | AAGAACCTGAATGCAT | 48 | 1751 |
| 959311 | 1085 | 1100 | 16205 | 16220 | GATGCTGAGACGCAGG | 0 | 1752 |
| 959321 | 1602 | 1617 | 25365 | 25380 | ACAGACTCTTCTCTAG | 45 | 1753 |
| 959331 | 1643 | 1658 | 25406 | 25421 | CCTCTGAAAGAATCTG | 81 | 1754 |
| 959341 | 1673 | 1688 | 25436 | 25451 | GTAGCTGCACAAAGAT | 69 | 1755 |
| 959351 | 1842 | 1857 | 25605 | 25620 | TGGTGTCATTTCCCAA | 19 | 1756 |
| 959361 | 2115 | 2130 | 25878 | 25893 | TTTTTAGTAGTCAAGG | 91 | 1757 |
| 959371 | 2240 | 2255 | 26003 | 26018 | ATTCAACAGGTAACAA | 69 | 1758 |
| 959381 | 2271 | 2286 | 26034 | 26049 | ACTAACATCTCACTGA | 33 | 1759 |
| 959391 | N/A | N/A | 4313 | 4328 | AGCTAGTGAGAAACAA | 47 | 1760 |
| 959401 | N/A | N/A | 4764 | 4779 | CTTTTATTGTTGCTAA | 77 | 1761 |
| 959411 | N/A | N/A | 4868 | 4883 | AGTGTACTTTAGGCTC | 90 | 1762 |
| 959421 | N/A | N/A | 5286 | 5301 | CAGCCATAAATCTTGG | 0 | 1763 |
| 959431 | N/A | N/A | 5574 | 5589 | AATGACATCATGGCTT | 74 | 1764 |
| 959441 | N/A | N/A | 5604 | 5619 | TTTATTCAATGTGGCT | 96 | 1765 |
| 959451 | N/A | N/A | 5642 | 5657 | TTGGGCTCAATGAAAT | 0 | 1766 |
| 959461 | N/A | N/A | 5714 | 5729 | GCTTAGTATGAAATAT | 78 | 1767 |
| 959471 | N/A | N/A | 5809 | 5824 | GTACTGTCTACTATGG | 78 | 1768 |
| 959481 | N/A | N/A | 6160 | 6175 | CTGCTTACATCCACGA | 4 | 1769 |
| 959661 | N/A | N/A | 12154 | 12169 | ACAGTAACTGGTAGCT | 72 | 1770 |
| 959671 | N/A | N/A | 12170 | 12185 | CTGTTTGATTGTGCAG | 50 | 1771 |
| 959681 | N/A | N/A | 12211 | 12226 | GCGCCTTTTATTTCCG | 14 | 1772 |
| 959691 | N/A | N/A | 12322 | 12337 | CCTGACTATATAACCA | 56 | 1773 |
| 959701 | N/A | N/A | 12689 | 12704 | GACCGTGTTTCCAAAT | 97 | 1774 |
| 959711 | N/A | N/A | 12712 | 12727 | AACTACCGAACGCAGT | 48 | 1775 |
| 959721 | N/A | N/A | 12759 | 12774 | GGTAGAGTGGTAAGGC | 95 | 1776 |
| 959731 | N/A | N/A | 12828 | 12843 | CATAGCCTTCTTTCTT | 93 | 1777 |
| 959741 | N/A | N/A | 12882 | 12897 | AATCCTGTTAGACAGC | 90 | 1778 |
| 959751 | N/A | N/A | 12903 | 12918 | TATAAAGCACACGGGA | 82 | 1779 |
| 959761 | N/A | N/A | 12933 | 12948 | AATAAGAGCTGTCTCC | 87 | 1780 |
| 959771 | N/A | N/A | 12974 | 12989 | CCCTAACAAACTTTGC | 62 | 1781 |
| 959781 | N/A | N/A | 13394 | 13409 | AATGTCACCCTTCCAC | 90 | 1782 |
| 959791 | N/A | N/A | 13527 | 13542 | CATTGGAAGACCGCAG | 76 | 1783 |
| 959801 | N/A | N/A | 13707 | 13722 | ACCGCTAGTAAATGCT | 35 | 1784 |
| 959811 | N/A | N/A | 13740 | 13755 | TAGAACTAAGGCAAAT | 65 | 1785 |
| 959821 | N/A | N/A | 13916 | 13931 | GGAGTCATGACATCCC | 42 | 1786 |
| 959831 | N/A | N/A | 14302 | 14317 | TGAGCAGATAAATACA | 75 | 1787 |
| 959841 | N/A | N/A | 14425 | 14440 | CTTTCCCATCGACACA | 79 | 1788 |
| 959851 | N/A | N/A | 14573 | 14588 | GCGCTCCTACTTATCC | 0 | 1789 |
| 959861 | N/A | N/A | 15203 | 15218 | ATATTGCCAGGTATCT | 67 | 1790 |
| 959871 | N/A | N/A | 15763 | 15778 | GTGTTGGTTTATAACA | 13 | 1791 |
| 959881 | N/A | N/A | 17194 | 17209 | ACTTAGTCCGTGTTCA | 45 | 1792 |
| 959891 | N/A | N/A | 17224 | 17239 | CTGTAGCTGGTTTGTG | 42 | 1793 |
| 959901 | N/A | N/A | 17296 | 17311 | CCATCTCTTAGGGCAC | 53 | 1794 |
| 959911 | N/A | N/A | 18394 | 18409 | TGTTTGGAAGTCGCCA | 87 | 1795 |
| 959921 | N/A | N/A | 20289 | 20304 | ATCAGAGGAAGCCCAA | 84 | 1796 |
| 959931 | N/A | N/A | 20514 | 20529 | ACCAGATTGAGTCTCC | 91 | 1797 |
| 959941 | N/A | N/A | 20613 | 20628 | CTCTAATTTTACGATC | 70 | 1798 |
| 959951 | N/A | N/A | 20662 | 20677 | AGCTTAGAAATTGCAG | 0 | 1799 |
| 959961 | N/A | N/A | 20813 | 20828 | CAGGGTAATATTCAGA | 87 | 1800 |
| 959971 | N/A | N/A | 20936 | 20951 | TTTGTAGCAGACAGAT | 18 | 1801 |
| 959981 | N/A | N/A | 21001 | 21016 | TTTTAACAGCTCAGGT | 71 | 1802 |
| 959991 | N/A | N/A | 21406 | 21421 | AATTCTAGACATGGCC | 25 | 1803 |
| 960001 | N/A | N/A | 21443 | 21458 | TAACCAATATACTGAG | 72 | 1804 |
| 960011 | N/A | N/A | 22023 | 22038 | CGCAAAAAGACAACGA | 16 | 1805 |
| 960021 | N/A | N/A | 22766 | 22781 | GACATGACAGACTAAC | 76 | 1806 |
| 960031 | N/A | N/A | 24040 | 24055 | CCATCAATGCTGCACT | 61 | 1807 |

TABLE 29
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 97 | 702 |
| 959272 | 426 | 441 | 6000 | 6015 | AGTTGAAGGATGGATG | 76 | 1808 |
| 959282 | 517 | 532 | 6091 | 6106 | CTGGTAAGAGAGATGC | 64 | 1809 |
| 959292 | 815 | 830 | 12033 | 12048 | GAGGTTCCCTGTGCAG | 81 | 1810 |
| 959302 | 905 | 920 | 13641 | 13656 | CAAGAACCTGAATGCA | 65 | 1811 |
| 959312 | 1087 | 1102 | 16207 | 16222 | AGGATGCTGAGACGCA | 66 | 1812 |
| 959322 | 1604 | 1619 | 25367 | 25382 | TCACAGACTCTTCTCT | 50 | 1813 |
| 959332 | 1644 | 1659 | 25407 | 25422 | ACCTCTGAAAGAATCT | 69 | 1814 |
| 959342 | 1675 | 1690 | 25438 | 25453 | AGGTAGCTGCACAAAG | 78 | 1815 |
| 959352 | 1903 | 1918 | 25666 | 25681 | CAGTCTGACCATTAAT | 77 | 1816 |
| 959362 | 2173 | 2188 | 25936 | 25951 | GAAAAAGGTGTTCTAA | 85 | 1817 |
| 959372 | 2242 | 2257 | 26005 | 26020 | AAATTCAACAGGTAAC | 62 | 1818 |
| 959382 | 2275 | 2290 | 26038 | 26053 | TTCTACTAACATCTCA | 80 | 1819 |
| 959392 | N/A | N/A | 4732 | 4747 | ATGATCATGTGGCGGT | 80 | 1820 |
| 959402 | N/A | N/A | 4765 | 4780 | ACTTTTATTGTTGCTA | 86 | 1821 |
| 959412 | N/A | N/A | 4869 | 4884 | GAGTGTACTTTAGGCT | 94 | 1822 |
| 959422 | N/A | N/A | 5389 | 5404 | CTATCAGGTGCAGGAG | 93 | 1823 |
| 959432 | N/A | N/A | 5575 | 5590 | CAATGACATCATGGCT | 90 | 1824 |
| 959442 | N/A | N/A | 5607 | 5622 | TACTTTATTCAATGTG | 0 | 1825 |
| 959452 | N/A | N/A | 5643 | 5658 | ATTGGGCTCAATGAAA | 35 | 1826 |
| 959462 | N/A | N/A | 5716 | 5731 | TGGCTTAGTATGAAAT | 80 | 1827 |
| 959472 | N/A | N/A | 5864 | 5879 | TTTGGCAAGGCCAGAA | 0 | 1828 |
| 959482 | N/A | N/A | 6960 | 6975 | GCATAGAGGAAGCTCG | 32 | 1829 |
| 959662 | N/A | N/A | 12155 | 12170 | GACAGTAACTGGTAGC | 92 | 1830 |
| 959672 | N/A | N/A | 12172 | 12187 | TTCTGTTTGATTGTGC | 97 | 1831 |
| 959682 | N/A | N/A | 12280 | 12295 | AGGTCTGGGTATATGT | 93 | 1832 |
| 959692 | N/A | N/A | 12323 | 12338 | CCCTGACTATATAACC | 32 | 1833 |
| 959702 | N/A | N/A | 12691 | 12706 | TTGACCGTGTTTCCAA | 94 | 1834 |
| 959712 | N/A | N/A | 12713 | 12728 | AAACTACCGAACGCAG | 92 | 1835 |
| 959722 | N/A | N/A | 12760 | 12775 | TGGTAGAGTGGTAAGG | 94 | 1836 |
| 959732 | N/A | N/A | 12829 | 12844 | GCATAGCCTTCTTTCT | 87 | 1837 |
| 959742 | N/A | N/A | 12883 | 12898 | CAATCCTGTTAGACAG | 13 | 1838 |
| 959752 | N/A | N/A | 12904 | 12919 | TTATAAAGCACACGGG | 87 | 1839 |
| 959762 | N/A | N/A | 12934 | 12949 | CAATAAGAGCTGTCTC | 81 | 1840 |
| 959772 | N/A | N/A | 13370 | 13385 | TGCAGGCACCCCAGCA | 0 | 1841 |
| 959782 | N/A | N/A | 13395 | 13410 | GAATGTCACCCTTCCA | 90 | 1842 |
| 959792 | N/A | N/A | 13528 | 13543 | TCATTGGAAGACCGCA | 84 | 1843 |
| 959802 | N/A | N/A | 13708 | 13723 | GACCGCTAGTAAATGC | 57 | 1844 |
| 959812 | N/A | N/A | 13741 | 13756 | TTAGAACTAAGGCAAA | 78 | 1845 |
| 959822 | N/A | N/A | 13917 | 13932 | TGGAGTCATGACATCC | 0 | 1846 |
| 959832 | N/A | N/A | 14303 | 14318 | CTGAGCAGATAAATAC | 74 | 1847 |
| 959842 | N/A | N/A | 14553 | 14568 | AGTCTTAATGTGGATT | 76 | 1848 |
| 959852 | N/A | N/A | 14667 | 14682 | AGTGTCCCCATCCCCA | 54 | 1849 |
| 959862 | N/A | N/A | 15204 | 15219 | AATATTGCCAGGTATC | 56 | 1850 |
| 959872 | N/A | N/A | 15765 | 15780 | TAGTGTTGGTTTATAA | 89 | 1851 |
| 959882 | N/A | N/A | 17196 | 17211 | TGACTTAGTCCGTGTT | 43 | 1852 |
| 959892 | N/A | N/A | 17225 | 17240 | TCTGTAGCTGGTTTGT | 61 | 1853 |
| 959902 | N/A | N/A | 17298 | 17313 | TCCCATCTCTTAGGGC | 24 | 1854 |
| 959912 | N/A | N/A | 18396 | 18411 | TATGTTTGGAAGTCGC | 91 | 1855 |
| 959922 | N/A | N/A | 20290 | 20305 | AATCAGAGGAAGCCCA | 40 | 1856 |
| 959932 | N/A | N/A | 20515 | 20530 | AACCAGATTGAGTCTC | 72 | 1857 |
| 959942 | N/A | N/A | 20614 | 20629 | TCTCTAATTTTACGAT | 23 | 1858 |
| 959952 | N/A | N/A | 20663 | 20678 | CAGCTTAGAAATTGCA | 24 | 1859 |
| 959962 | N/A | N/A | 20814 | 20829 | CCAGGGTAATATTCAG | 87 | 1860 |
| 959972 | N/A | N/A | 20937 | 20952 | CTTTGTAGCAGACAGA | 50 | 1861 |
| 959982 | N/A | N/A | 21003 | 21018 | TATTTTAACAGCTCAG | 94 | 1862 |
| 959992 | N/A | N/A | 21409 | 21424 | TGCAATTCTAGACATG | 12 | 1863 |
| 960002 | N/A | N/A | 21445 | 21460 | ACTAACCAATATACTG | 55 | 1864 |
| 960012 | N/A | N/A | 22541 | 22556 | CAACAGATTACTGGAC | 28 | 1865 |
| 960022 | N/A | N/A | 22768 | 22783 | AGGACATGACAGACTA | 66 | 1866 |
| 960032 | N/A | N/A | 24041 | 24056 | ACCATCAATGCTGCAC | 79 | 1867 |

TABLE 30
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 98 | 702 |
| 959273 | 427 | 442 | 6001 | 6016 | AAGTTGAAGGATGGAT | 86 | 1868 |
| 959283 | 694 | 709 | 11912 | 11927 | TTGGCATCAATGAAGG | 73 | 1869 |
| 959293 | 816 | 831 | 12034 | 12049 | AGAGGTTCCCTGTGCA | 80 | 1870 |
| 959303 | 906 | 921 | 13642 | 13657 | CCAAGAACCTGAATGC | 65 | 1871 |
| 959313 | 1090 | 1105 | 16210 | 16225 | GGCAGGATGCTGAGAC | 43 | 1872 |
| 959323 | 1605 | 1620 | 25368 | 25383 | CTCACAGACTCTTCTC | 81 | 1873 |
| 959333 | 1646 | 1661 | 25409 | 25424 | GCACCTCTGAAAGAAT | 51 | 1874 |
| 959343 | 1677 | 1692 | 25440 | 25455 | GGAGGTAGCTGCACAA | 73 | 1875 |
| 959353 | 1904 | 1919 | 25667 | 25682 | ACAGTCTGACCATTAA | 85 | 1876 |
| 959363 | 2179 | 2194 | 25942 | 25957 | TTAGGTGAAAAAGGTG | 91 | 1877 |
| 959373 | 2258 | 2273 | 26021 | 26036 | TGATTCACATAATACA | 71 | 1878 |
| 959383 | 2277 | 2292 | 26040 | 26055 | TATTCTACTAACATCT | 38 | 1879 |
| 959393 | N/A | N/A | 4733 | 4748 | AATGATCATGTGGCGG | 93 | 1880 |
| 959403 | N/A | N/A | 4767 | 4782 | TGACTTTTATTGTTGC | 87 | 1881 |
| 959413 | N/A | N/A | 4870 | 4885 | TGAGTGTACTTTAGGC | 94 | 1882 |
| 959423 | N/A | N/A | 5392 | 5407 | ATGCTATCAGGTGCAG | 0 | 1883 |
| 959433 | N/A | N/A | 5578 | 5593 | GCACAATGACATCATG | 86 | 1884 |
| 959443 | N/A | N/A | 5608 | 5623 | TTACTTTATTCAATGT | 9 | 1885 |
| 959453 | N/A | N/A | 5644 | 5659 | TATTGGGCTCAATGAA | 80 | 1886 |
| 959463 | N/A | N/A | 5798 | 5813 | TATGGGAGCCACATGT | 4 | 1887 |
| 959473 | N/A | N/A | 5866 | 5881 | CTTTTGGCAAGGCCAG | 0 | 1888 |
| 959483 | N/A | N/A | 7199 | 7214 | TTAAACAGAGGATGCA | 31 | 1889 |
| 959663 | N/A | N/A | 12156 | 12171 | AGACAGTAACTGGTAG | 75 | 1890 |
| 959673 | N/A | N/A | 12199 | 12214 | TCCGTTAACCATCAAG | 95 | 1891 |
| 959683 | N/A | N/A | 12282 | 12297 | TTAGGTCTGGGTATAT | 94 | 1892 |
| 959693 | N/A | N/A | 12324 | 12339 | CCCCTGACTATATAAC | 0 | 1893 |
| 959703 | N/A | N/A | 12692 | 12707 | CTTGACCGTGTTTCCA | 97 | 1894 |
| 959713 | N/A | N/A | 12715 | 12730 | TTAAACTACCGAACGC | 95 | 1895 |
| 959723 | N/A | N/A | 12761 | 12776 | ATGGTAGAGTGGTAAG | 77 | 1896 |
| 959733 | N/A | N/A | 12832 | 12847 | ATTGCATAGCCTTCTT | 95 | 1897 |
| 959743 | N/A | N/A | 12884 | 12899 | CCAATCCTGTTAGACA | 83 | 1898 |
| 959753 | N/A | N/A | 12908 | 12923 | CTGCTTATAAAGCACA | 2 | 1899 |
| 959763 | N/A | N/A | 12935 | 12950 | ACAATAAGAGCTGTCT | 85 | 1900 |
| 959773 | N/A | N/A | 13372 | 13387 | TTTGCAGGCACCCCAG | 63 | 1901 |
| 959783 | N/A | N/A | 13396 | 13411 | TGAATGTCACCCTTCC | 53 | 1902 |
| 959793 | N/A | N/A | 13531 | 13546 | GCATCATTGGAAGACC | 86 | 1903 |
| 959803 | N/A | N/A | 13713 | 13728 | ACCAAGACCGCTAGTA | 38 | 1904 |
| 959813 | N/A | N/A | 13743 | 13758 | TGTTAGAACTAAGGCA | 79 | 1905 |
| 959823 | N/A | N/A | 13919 | 13934 | CCTGGAGTCATGACAT | 7 | 1906 |
| 959833 | N/A | N/A | 14304 | 14319 | TCTGAGCAGATAAATA | 39 | 1907 |
| 959843 | N/A | N/A | 14554 | 14569 | AAGTCTTAATGTGGAT | 84 | 1908 |
| 959853 | N/A | N/A | 14669 | 14684 | TTAGTGTCCCCATCCC | 78 | 1909 |
| 959863 | N/A | N/A | 15205 | 15220 | GAATATTGCCAGGTAT | 86 | 1910 |
| 959873 | N/A | N/A | 15766 | 15781 | TTAGTGTTGGTTTATA | 92 | 1911 |
| 959883 | N/A | N/A | 17198 | 17213 | TTTGACTTAGTCCGTG | 86 | 1912 |
| 959893 | N/A | N/A | 17226 | 17241 | CTCTGTAGCTGGTTTG | 82 | 1913 |
| 959903 | N/A | N/A | 17601 | 17616 | TTGATAGTGAATGTGT | 83 | 1914 |
| 959913 | N/A | N/A | 18397 | 18412 | ATATGTTTGGAAGTCG | 91 | 1915 |
| 959923 | N/A | N/A | 20291 | 20306 | CAATCAGAGGAAGCCC | 34 | 1916 |
| 959933 | N/A | N/A | 20516 | 20531 | TAACCAGATTGAGTCT | 66 | 1917 |
| 959943 | N/A | N/A | 20615 | 20630 | GTCTCTAATTTTACGA | 53 | 1918 |
| 959953 | N/A | N/A | 20664 | 20679 | ACAGCTTAGAAATTGC | 89 | 1919 |
| 959963 | N/A | N/A | 20843 | 20858 | CTGTATTAGCTCAATA | 67 | 1920 |
| 959973 | N/A | N/A | 20938 | 20953 | TCTTTGTAGCAGACAG | 84 | 1921 |
| 959983 | N/A | N/A | 21004 | 21019 | TTATTTTAACAGCTCA | 92 | 1922 |
| 959993 | N/A | N/A | 21410 | 21425 | CTGCAATTCTAGACAT | 29 | 1923 |
| 960003 | N/A | N/A | 21535 | 21550 | CATTTCAGAGTATAAG | 46 | 1924 |
| 960013 | N/A | N/A | 22708 | 22723 | GATGTGAGTGAAATAA | 69 | 1925 |
| 960023 | N/A | N/A | 22769 | 22784 | AAGGACATGACAGACT | 68 | 1926 |
| 960033 | N/A | N/A | 24043 | 24058 | CCACCATCAATGCTGC | 58 | 1927 |

TABLE 31
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 99 | 702 |
| 959284 | 695 | 710 | 11913 | 11928 | TTTGGCATCAATGAAG | 69 | 1928 |
| 959294 | 817 | 832 | 12035 | 12050 | TAGAGGTTCCCTGTGC | 83 | 1929 |
| 959314 | 1091 | 1106 | 16211 | 16226 | GGGCAGGATGCTGAGA | 22 | 1930 |
| 959324 | 1606 | 1621 | 25369 | 25384 | ACTCACAGACTCTTCT | 68 | 1931 |
| 959334 | 1647 | 1662 | 25410 | 25425 | AGCACCTCTGAAAGAA | 67 | 1932 |
| 959344 | 1678 | 1693 | 25441 | 25456 | CGGAGGTAGCTGCACA | 85 | 1933 |
| 959354 | 1926 | 1941 | 25689 | 25704 | ATTCTAAGAACCTCAT | 51 | 1934 |
| 959384 | N/A | N/A | 4303 | 4318 | AAACAAACCCTCCGTC | 10 | 1935 |
| 959394 | N/A | N/A | 4734 | 4749 | AAATGATCATGTGGCG | 94 | 1936 |
| 959404 | N/A | N/A | 4768 | 4783 | CTGACTTTTATTGTTG | 74 | 1937 |
| 959414 | N/A | N/A | 4871 | 4886 | GTGAGTGTACTTTAGG | 98 | 1938 |
| 959424 | N/A | N/A | 5393 | 5408 | AATGCTATCAGGTGCA | 31 | 1939 |
| 959434 | N/A | N/A | 5579 | 5594 | TGCACAATGACATCAT | 88 | 1940 |
| 959444 | N/A | N/A | 5621 | 5636 | CTACCTGTGTCTTTTA | 90 | 1941 |
| 959454 | N/A | N/A | 5647 | 5662 | ATATATTGGGCTCAAT | 81 | 1942 |
| 959474 | N/A | N/A | 5867 | 5882 | ACTTTTGGCAAGGCCA | 17 | 1943 |
| 959484 | N/A | N/A | 7211 | 7226 | CCGCAAACAAGGTTAA | 22 | 1944 |
| 959684 | N/A | N/A | 12283 | 12298 | TTTAGGTCTGGGTATA | 93 | 1945 |
| 959694 | N/A | N/A | 12325 | 12340 | CCCCCTGACTATATAA | 18 | 1946 |
| 959704 | N/A | N/A | 12693 | 12708 | TCTTGACCGTGTTTCC | 97 | 1947 |
| 959734 | N/A | N/A | 12834 | 12849 | GCATTGCATAGCCTTC | 96 | 1948 |
| 959744 | N/A | N/A | 12886 | 12901 | AACCAATCCTGTTAGA | 59 | 1949 |
| 959754 | N/A | N/A | 12909 | 12924 | TCTGCTTATAAAGCAC | 100 | 1950 |
| 959774 | N/A | N/A | 13373 | 13388 | CTTTGCAGGCACCCCA | 72 | 1951 |
| 959784 | N/A | N/A | 13398 | 13413 | CTTGAATGTCACCCTT | 92 | 1952 |
| 959804 | N/A | N/A | 13715 | 13730 | TTACCAAGACCGCTAG | 47 | 1953 |
| 959824 | N/A | N/A | 14228 | 14243 | ACTTTTAGTATTAAAG | 0 | 1954 |
| 959844 | N/A | N/A | 14555 | 14570 | AAAGTCTTAATGTGGA | 88 | 1955 |
| 959854 | N/A | N/A | 14670 | 14685 | CTTAGTGTCCCCATCC | 76 | 1956 |
| 959874 | N/A | N/A | 15767 | 15782 | GTTAGTGTTGGTTTAT | 95 | 1957 |
| 959884 | N/A | N/A | 17199 | 17214 | GTTTGACTTAGTCCGT | 96 | 1958 |
| 959914 | N/A | N/A | 18398 | 18413 | AATATGTTTGGAAGTC | 87 | 1959 |
| 959934 | N/A | N/A | 20518 | 20533 | AGTAACCAGATTGAGT | 96 | 1960 |
| 959954 | N/A | N/A | 20665 | 20680 | CACAGCTTAGAAATTG | 88 | 1961 |
| 959964 | N/A | N/A | 20844 | 20859 | CCTGTATTAGCTCAAT | 92 | 1962 |
| 959974 | N/A | N/A | 20940 | 20955 | CCTCTTTGTAGCAGAC | 84 | 1963 |
| 959984 | N/A | N/A | 21006 | 21021 | GGTTATTTTAACAGCT | 85 | 1964 |
| 959994 | N/A | N/A | 21412 | 21427 | ACCTGCAATTCTAGAC | 61 | 1965 |
| 960014 | N/A | N/A | 22710 | 22725 | AAGATGTGAGTGAAAT | 70 | 1966 |
| 960024 | N/A | N/A | 22770 | 22785 | AAAGGACATGACAGAC | 87 | 1967 |

TABLE 32
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 959275 | 440 | 455 | 6014 | 6029 | GAGGAACTTGCTTAAG | 81 | 1968 |
| 959285 | 696 | 711 | 11914 | 11929 | TTTTGGCATCAATGAA | 62 | 1969 |
| 959305 | 1066 | 1081 | 16186 | 16201 | TCTAGCAGCTCATCTC | 64 | 1970 |
| 959335 | 1649 | 1664 | 25412 | 25427 | TTAGCACCTCTGAAAG | 72 | 1971 |
| 959345 | 1804 | 1819 | 25567 | 25582 | ATGTATTAGAGTTAAG | 77 | 1972 |
| 959355 | 1927 | 1942 | 25690 | 25705 | CATTCTAAGAACCTCA | 71 | 1973 |
| 959365 | 2183 | 2198 | 25946 | 25961 | TTAGTTAGGTGAAAAA | 81 | 1974 |
| 959375 | 2261 | 2276 | 26024 | 26039 | CACTGATTCACATAAT | 70 | 1975 |
| 959395 | N/A | N/A | 4737 | 4752 | TGCAAATGATCATGTG | 66 | 1976 |
| 959405 | N/A | N/A | 4769 | 4784 | GCTGACTTTTATTGTT | 84 | 1977 |
| 959415 | N/A | N/A | 4872 | 4887 | AGTGAGTGTACTTTAG | 94 | 1978 |
| 959425 | N/A | N/A | 5395 | 5410 | TTAATGCTATCAGGTG | 81 | 1979 |
| 959435 | N/A | N/A | 5580 | 5595 | ATGCACAATGACATCA | 86 | 1980 |
| 959445 | N/A | N/A | 5624 | 5639 | ATTCTACCTGTGTCTT | 97 | 1981 |
| 959455 | N/A | N/A | 5651 | 5666 | TTGGATATATTGGGCT | 97 | 1982 |
| 959475 | N/A | N/A | 5868 | 5883 | TACTTTTGGCAAGGCC | 70 | 1983 |
| 959485 | N/A | N/A | 7697 | 7712 | GCACAGAGTAGGTTAA | 72 | 1984 |
| 959655 | N/A | N/A | 12146 | 12161 | TGGTAGCTCCTGGCAA | 55 | 1985 |
| 959675 | N/A | N/A | 12201 | 12216 | TTTCCGTTAACCATCA | 94 | 1986 |
| 959695 | N/A | N/A | 12667 | 12682 | CATCTTAGTGGCTGGG | 93 | 1987 |
| 959705 | N/A | N/A | 12695 | 12710 | GTTCTTGACCGTGTTT | 97 | 1988 |
| 959715 | N/A | N/A | 12717 | 12732 | GGTTAAACTACCGAAC | 11 | 1989 |
| 959725 | N/A | N/A | 12783 | 12798 | CATGGTCTGCAAATTT | 89 | 1990 |
| 959745 | N/A | N/A | 12887 | 12902 | AAACCAATCCTGTTAG | 46 | 1991 |
| 959755 | N/A | N/A | 12910 | 12925 | ATCTGCTTATAAAGCA | 42 | 1992 |
| 959775 | N/A | N/A | 13374 | 13389 | ACTTTGCAGGCACCCC | 87 | 1993 |
| 959785 | N/A | N/A | 13399 | 13414 | GCTTGAATGTCACCCT | 94 | 1994 |
| 959805 | N/A | N/A | 13716 | 13731 | TTTACCAAGACCGCTA | 86 | 1995 |
| 959825 | N/A | N/A | 14230 | 14245 | CAACTTTTAGTATTAA | 0 | 1996 |
| 959835 | N/A | N/A | 14417 | 14432 | TCGACACAGCATCACC | 62 | 1997 |
| 959855 | N/A | N/A | 14671 | 14686 | TCTTAGTGTCCCCATC | 78 | 1998 |
| 959865 | N/A | N/A | 15209 | 15224 | TTAGGAATATTGCCAG | 93 | 1999 |
| 959875 | N/A | N/A | 15769 | 15784 | GGGTTAGTGTTGGTTT | 94 | 2000 |
| 959895 | N/A | N/A | 17288 | 17303 | TAGGGCACCTCAAGAA | 0 | 2001 |
| 959915 | N/A | N/A | 18400 | 18415 | CAAATATGTTTGGAAG | 50 | 2002 |
| 959925 | N/A | N/A | 20293 | 20308 | CCCAATCAGAGGAAGC | 41 | 2003 |
| 959935 | N/A | N/A | 20599 | 20614 | TCATCATTATTACCTG | 91 | 2004 |
| 959955 | N/A | N/A | 20803 | 20818 | TTCAGACCAGGGTAAT | 91 | 2005 |
| 959965 | N/A | N/A | 20845 | 20860 | GCCTGTATTAGCTCAA | 90 | 2006 |
| 959975 | N/A | N/A | 20941 | 20956 | GCCTCTTTGTAGCAGA | 0 | 2007 |
| 959995 | N/A | N/A | 21434 | 21449 | TACTGAGAGGAAATGA | 64 | 2008 |
| 960015 | N/A | N/A | 22714 | 22729 | TGTAAAGATGTGAGTG | 78 | 2009 |
| 960025 | N/A | N/A | 22772 | 22787 | TCAAAGGACATGACAG | 80 | 2010 |
| 960037 | N/A | N/A | 22590 | 22605 | GAGCAACGAGGAAGGA | 68 | 2011 |

TABLE 33
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 98 | 702 |
| 959276 | 447 | 462 | 6021 | 6036 | CCTGTCGGAGGAACTT | 37 | 2012 |
| 959286 | 699 | 714 | 11917 | 11932 | TTGTTTTGGCATCAAT | 73 | 2013 |
| 959296 | 895 | 910 | 13631 | 13646 | AATGCATCCAAATATC | 0 | 2014 |
| 959306 | 1069 | 1084 | 16189 | 16204 | TGGTCTAGCAGCTCAT | 25 | 2015 |
| 959316 | 1221 | 1236 | 19061 | 19076 | TTACATAAGACATTAT | 35 | 2016 |
| 959326 | 1614 | 1629 | 25377 | 25392 | CTCAAGTGACTCACAG | 85 | 2017 |
| 959336 | 1650 | 1665 | 25413 | 25428 | TTTAGCACCTCTGAAA | 24 | 2018 |
| 959346 | 1834 | 1849 | 25597 | 25612 | TTTCCCAACCAGCTGA | 60 | 2019 |
| 959356 | 2096 | 2111 | 25859 | 25874 | TCATCTTTGCAGACCA | 90 | 2020 |
| 959366 | 2184 | 2199 | 25947 | 25962 | TTTAGTTAGGTGAAAA | 57 | 2021 |
| 959376 | 2262 | 2277 | 26025 | 26040 | TCACTGATTCACATAA | 86 | 2022 |
| 959386 | N/A | N/A | 4306 | 4321 | GAGAAACAAACCCTCC | 0 | 2023 |
| 959396 | N/A | N/A | 4738 | 4753 | GTGCAAATGATCATGT | 69 | 2024 |
| 959406 | N/A | N/A | 4771 | 4786 | AAGCTGACTTTTATTG | 57 | 2025 |
| 959416 | N/A | N/A | 5276 | 5291 | TCTTGGGATGCACAGG | 49 | 2026 |
| 959426 | N/A | N/A | 5397 | 5412 | CCTTAATGCTATCAGG | 0 | 2027 |
| 959436 | N/A | N/A | 5581 | 5596 | AATGCACAATGACATC | 69 | 2028 |
| 959446 | N/A | N/A | 5625 | 5640 | AATTCTACCTGTGTCT | 82 | 2029 |
| 959456 | N/A | N/A | 5652 | 5667 | TTTGGATATATTGGGC | 95 | 2030 |
| 959466 | N/A | N/A | 5802 | 5817 | CTACTATGGGAGCCAC | 64 | 2031 |
| 959476 | N/A | N/A | 5871 | 5886 | TAATACTTTTGGCAAG | 29 | 2032 |
| 959486 | N/A | N/A | 7784 | 7799 | TTATAGGCGAGAGCAC | 0 | 2033 |
| 959656 | N/A | N/A | 12147 | 12162 | CTGGTAGCTCCTGGCA | 44 | 2034 |
| 959666 | N/A | N/A | 12164 | 12179 | GATTGTGCAGACAGTA | 95 | 2035 |
| 959676 | N/A | N/A | 12202 | 12217 | ATTTCCGTTAACCATC | 93 | 2036 |
| 959686 | N/A | N/A | 12288 | 12303 | TGAGTTTTAGGTCTGG | 96 | 2037 |
| 959696 | N/A | N/A | 12669 | 12684 | ATCATCTTAGTGGCTG | 91 | 2038 |
| 959706 | N/A | N/A | 12696 | 12711 | TGTTCTTGACCGTGTT | 98 | 2039 |
| 959716 | N/A | N/A | 12719 | 12734 | AAGGTTAAACTACCGA | 6 | 2040 |
| 959726 | N/A | N/A | 12785 | 12800 | TACATGGTCTGCAAAT | 90 | 2041 |
| 959736 | N/A | N/A | 12838 | 12853 | CATTGCATTGCATAGC | 96 | 2042 |
| 959746 | N/A | N/A | 12888 | 12903 | AAAACCAATCCTGTTA | 47 | 2043 |
| 959756 | N/A | N/A | 12911 | 12926 | CATCTGCTTATAAAGC | 81 | 2044 |
| 959766 | N/A | N/A | 12967 | 12982 | AAACTTTGCAGCCTAT | 93 | 2045 |
| 959776 | N/A | N/A | 13376 | 13391 | AGACTTTGCAGGCACC | 90 | 2046 |
| 959786 | N/A | N/A | 13400 | 13415 | GGCTTGAATGTCACCC | 69 | 2047 |
| 959796 | N/A | N/A | 13697 | 13712 | AATGCTTGTCAAAAGG | 70 | 2048 |
| 959806 | N/A | N/A | 13717 | 13732 | CTTTACCAAGACCGCT | 82 | 2049 |
| 959816 | N/A | N/A | 13747 | 13762 | TAAGTGTTAGAACTAA | 30 | 2050 |
| 959826 | N/A | N/A | 14232 | 14247 | ACCAACTTTTAGTATT | 79 | 2051 |
| 959836 | N/A | N/A | 14419 | 14434 | CATCGACACAGCATCA | 59 | 2052 |
| 959846 | N/A | N/A | 14557 | 14572 | CCAAAGTCTTAATGTG | 53 | 2053 |
| 959856 | N/A | N/A | 14676 | 14691 | CCATCTCTTAGTGTCC | 88 | 2054 |
| 959866 | N/A | N/A | 15210 | 15225 | CTTAGGAATATTGCCA | 87 | 2055 |
| 959876 | N/A | N/A | 15770 | 15785 | AGGGTTAGTGTTGGTT | 89 | 2056 |
| 959886 | N/A | N/A | 17202 | 17217 | TCTGTTTGACTTAGTC | 70 | 2057 |
| 959896 | N/A | N/A | 17290 | 17305 | CTTAGGGCACCTCAAG | 30 | 2058 |
| 959906 | N/A | N/A | 17735 | 17750 | TAATCTGGTCATATGG | 43 | 2059 |
| 959916 | N/A | N/A | 18445 | 18460 | TGCTTACGGAGCATAG | 0 | 2060 |
| 959926 | N/A | N/A | 20472 | 20487 | CTCTAGACGGGAAGCT | 31 | 2061 |
| 959936 | N/A | N/A | 20601 | 20616 | GATCATCATTATTACC | 71 | 2062 |
| 959946 | N/A | N/A | 20652 | 20667 | TTGCAGTGCCCTGGCC | 31 | 2063 |
| 959956 | N/A | N/A | 20804 | 20819 | ATTCAGACCAGGGTAA | 87 | 2064 |
| 959966 | N/A | N/A | 20847 | 20862 | ATGCCTGTATTAGCTC | 65 | 2065 |
| 959976 | N/A | N/A | 20942 | 20957 | AGCCTCTTTGTAGCAG | 5 | 2066 |
| 959986 | N/A | N/A | 21008 | 21023 | GAGGTTATTTTAACAG | 69 | 2067 |
| 959996 | N/A | N/A | 21435 | 21450 | ATACTGAGAGGAAATG | 68 | 2068 |
| 960006 | N/A | N/A | 21539 | 21554 | ATGACATTTCAGAGTA | 88 | 2069 |
| 960016 | N/A | N/A | 22715 | 22730 | GTGTAAAGATGTGAGT | 84 | 2070 |
| 960026 | N/A | N/A | 24033 | 24048 | TGCTGCACTCAAAGAG | 0 | 2071 |
| 960038 | N/A | N/A | 19377 | 19392 | TCACAAGAGACTGGAC | 31 | 2072 |

TABLE 34
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 98 | 702 |
| 959277 | 481 | 496 | 6055 | 6070 | TGGTGGACATTGGCCG | 5 | 2073 |
| 959287 | 707 | 722 | 11925 | 11940 | GGTGATGGTTGTTTTG | 85 | 2074 |
| 959297 | 896 | 911 | 13632 | 13647 | GAATGCATCCAAATAT | 0 | 2075 |
| 959307 | 1070 | 1085 | 16190 | 16205 | GTGGTCTAGCAGCTCA | 66 | 2076 |
| 959317 | 1223 | 1238 | 19063 | 19078 | CATTACATAAGACATT | 46 | 2077 |
| 959327 | 1615 | 1630 | 25378 | 25393 | CCTCAAGTGACTCACA | 83 | 2078 |
| 959337 | 1651 | 1666 | 25414 | 25429 | CTTTAGCACCTCTGAA | 71 | 2079 |
| 959347 | 1835 | 1850 | 25598 | 25613 | ATTTCCCAACCAGCTG | 49 | 2080 |
| 959357 | 2099 | 2114 | 25862 | 25877 | TTATCATCTTTGCAGA | 48 | 2081 |
| 959367 | 2185 | 2200 | 25948 | 25963 | TTTTAGTTAGGTGAAA | 43 | 2082 |
| 959377 | 2263 | 2278 | 26026 | 26041 | CTCACTGATTCACATA | 79 | 2083 |
| 959387 | N/A | N/A | 4307 | 4322 | TGAGAAACAAACCCTC | 0 | 2084 |
| 959397 | N/A | N/A | 4739 | 4754 | TGTGCAAATGATCATG | 82 | 2085 |
| 959407 | N/A | N/A | 4859 | 4874 | TAGGCTCCTGGGACCT | 0 | 2086 |
| 959417 | N/A | N/A | 5277 | 5292 | ATCTTGGGATGCACAG | 89 | 2087 |
| 959427 | N/A | N/A | 5567 | 5582 | TCATGGCTTCCAGTGT | 78 | 2088 |
| 959437 | N/A | N/A | 5600 | 5615 | TTCAATGTGGCTTCTA | 96 | 2089 |
| 959447 | N/A | N/A | 5627 | 5642 | TTAATTCTACCTGTGT | 72 | 2090 |
| 959457 | N/A | N/A | 5706 | 5721 | TGAAATATCTCATTAG | 77 | 2091 |
| 959467 | N/A | N/A | 5805 | 5820 | TGTCTACTATGGGAGC | 83 | 2092 |
| 959477 | N/A | N/A | 5875 | 5890 | ATGGTAATACTTTTGG | 75 | 2093 |
| 959657 | N/A | N/A | 12148 | 12163 | ACTGGTAGCTCCTGGC | 78 | 2094 |
| 959667 | N/A | N/A | 12165 | 12180 | TGATTGTGCAGACAGT | 97 | 2095 |
| 959677 | N/A | N/A | 12203 | 12218 | TATTTCCGTTAACCAT | 91 | 2096 |
| 959687 | N/A | N/A | 12289 | 12304 | CTGAGTTTTAGGTCTG | 96 | 2097 |
| 959697 | N/A | N/A | 12671 | 12686 | GAATCATCTTAGTGGC | 94 | 2098 |
| 959707 | N/A | N/A | 12697 | 12712 | TTGTTCTTGACCGTGT | 98 | 2099 |
| 959717 | N/A | N/A | 12753 | 12768 | GTGGTAAGGCATACTA | 35 | 2100 |
| 959727 | N/A | N/A | 12789 | 12804 | GGTGTACATGGTCTGC | 97 | 2101 |
| 959737 | N/A | N/A | 12839 | 12854 | GCATTGCATTGCATAG | 92 | 2102 |
| 959747 | N/A | N/A | 12890 | 12905 | GGAAAACCAATCCTGT | 69 | 2103 |
| 959757 | N/A | N/A | 12927 | 12942 | AGCTGTCTCCTCTACT | 70 | 2104 |
| 959767 | N/A | N/A | 12968 | 12983 | CAAACTTTGCAGCCTA | 95 | 2105 |
| 959777 | N/A | N/A | 13377 | 13392 | GAGACTTTGCAGGCAC | 88 | 2106 |
| 959787 | N/A | N/A | 13402 | 13417 | TCGGCTTGAATGTCAC | 67 | 2107 |
| 959797 | N/A | N/A | 13700 | 13715 | GTAAATGCTTGTCAAA | 91 | 2108 |
| 959807 | N/A | N/A | 13720 | 13735 | AGTCTTTACCAAGACC | 0 | 2109 |
| 959817 | N/A | N/A | 13911 | 13926 | CATGACATCCCAGTTC | 29 | 2110 |
| 959827 | N/A | N/A | 14233 | 14248 | AACCAACTTTTAGTAT | 27 | 2111 |
| 959837 | N/A | N/A | 14420 | 14435 | CCATCGACACAGCATC | 89 | 2112 |
| 959847 | N/A | N/A | 14567 | 14582 | CTACTTATCCCCAAAG | 16 | 2113 |
| 959857 | N/A | N/A | 14677 | 14692 | GCCATCTCTTAGTGTC | 36 | 2114 |
| 959867 | N/A | N/A | 15211 | 15226 | CCTTAGGAATATTGCC | 75 | 2115 |
| 959877 | N/A | N/A | 15771 | 15786 | GAGGGTTAGTGTTGGT | 93 | 2116 |
| 959887 | N/A | N/A | 17218 | 17233 | CTGGTTTGTGGGTTCT | 75 | 2117 |
| 959897 | N/A | N/A | 17291 | 17306 | TCTTAGGGCACCTCAA | 53 | 2118 |
| 959907 | N/A | N/A | 17736 | 17751 | TTAATCTGGTCATATG | 0 | 2119 |
| 959917 | N/A | N/A | 18852 | 18867 | ACAAAAGCGACAAGGT | 33 | 2120 |
| 959927 | N/A | N/A | 20508 | 20523 | TTGAGTCTCCTGACCA | 65 | 2121 |
| 959937 | N/A | N/A | 20602 | 20617 | CGATCATCATTATTAC | 87 | 2122 |
| 959947 | N/A | N/A | 20653 | 20668 | ATTGCAGTGCCCTGGC | 69 | 2123 |
| 959957 | N/A | N/A | 20805 | 20820 | TATTCAGACCAGGGTA | 89 | 2124 |
| 959967 | N/A | N/A | 20848 | 20863 | GATGCCTGTATTAGCT | 72 | 2125 |
| 959977 | N/A | N/A | 20944 | 20959 | GCAGCCTCTTTGTAGC | 0 | 2126 |
| 959987 | N/A | N/A | 21010 | 21025 | CTGAGGTTATTTTAAC | 40 | 2127 |
| 959997 | N/A | N/A | 21436 | 21451 | TATACTGAGAGGAAAT | 47 | 2128 |
| 960007 | N/A | N/A | 21541 | 21556 | ATATGACATTTCAGAG | 91 | 2129 |
| 960017 | N/A | N/A | 22716 | 22731 | CGTGTAAAGATGTGAG | 87 | 2130 |
| 960027 | N/A | N/A | 24035 | 24050 | AATGCTGCACTCAAAG | 31 | 2131 |
| 960039 | N/A | N/A | 20215 | 20230 | TAACAAACTATGCCTA | 44 | 2132 |

TABLE 35
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 98 | 702 |
| 959274 | 439 | 454 | 6013 | 6028 | AGGAACTTGCTTAAGT | 73 | 2133 |
| 959284 | 695 | 710 | 11913 | 11928 | TTTGGCATCAATGAAG | 60 | 1928 |
| 959294 | 817 | 832 | 12035 | 12050 | TAGAGGTTCCCTGTGC | 77 | 1929 |
| 959304 | 1063 | 1078 | 16183 | 16198 | AGCAGCTCATCTCCCT | 62 | 2134 |
| 959314 | 1091 | 1106 | 16211 | 16226 | GGGCAGGATGCTGAGA | 13 | 1930 |
| 959324 | 1606 | 1621 | 25369 | 25384 | ACTCACAGACTCTTCT | 72 | 1931 |
| 959334 | 1647 | 1662 | 25410 | 25425 | AGCACCTCTGAAAGAA | 65 | 1932 |
| 959344 | 1678 | 1693 | 25441 | 25456 | CGGAGGTAGCTGCACA | 86 | 1933 |
| 959354 | 1926 | 1941 | 25689 | 25704 | ATTCTAAGAACCTCAT | 54 | 1934 |
| 959364 | 2181 | 2196 | 25944 | 25959 | AGTTAGGTGAAAAAGG | 92 | 2135 |
| 959374 | 2260 | 2275 | 26023 | 26038 | ACTGATTCACATAATA | 78 | 2136 |
| 959384 | N/A | N/A | 4303 | 4318 | AAACAAACCCTCCGTC | 2 | 1935 |
| 959394 | N/A | N/A | 4734 | 4749 | AAATGATCATGTGGCG | 94 | 1936 |
| 959404 | N/A | N/A | 4768 | 4783 | CTGACTTTTATTGTTG | 72 | 1937 |
| 959414 | N/A | N/A | 4871 | 4886 | GTGAGTGTACTTTAGG | 97 | 1938 |
| 959424 | N/A | N/A | 5393 | 5408 | AATGCTATCAGGTGCA | 31 | 1939 |
| 959434 | N/A | N/A | 5579 | 5594 | TGCACAATGACATCAT | 87 | 1940 |
| 959444 | N/A | N/A | 5621 | 5636 | CTACCTGTGTCTTTTA | 90 | 1941 |
| 959454 | N/A | N/A | 5647 | 5662 | ATATATTGGGCTCAAT | 80 | 1942 |
| 959464 | N/A | N/A | 5799 | 5814 | CTATGGGAGCCACATG | 24 | 2137 |
| 959474 | N/A | N/A | 5867 | 5882 | ACTTTTGGCAAGGCCA | 23 | 1943 |
| 959484 | N/A | N/A | 7211 | 7226 | CCGCAAACAAGGTTAA | 0 | 1944 |
| 959664 | N/A | N/A | 12157 | 12172 | CAGACAGTAACTGGTA | 96 | 2138 |
| 959674 | N/A | N/A | 12200 | 12215 | TTCCGTTAACCATCAA | 97 | 2139 |
| 959684 | N/A | N/A | 12283 | 12298 | TTTAGGTCTGGGTATA | 93 | 1945 |
| 959694 | N/A | N/A | 12325 | 12340 | CCCCCTGACTATATAA | 26 | 1946 |
| 959704 | N/A | N/A | 12693 | 12708 | TCTTGACCGTGTTTCC | 98 | 1947 |
| 959714 | N/A | N/A | 12716 | 12731 | GTTAAACTACCGAACG | 35 | 2140 |
| 959724 | N/A | N/A | 12763 | 12778 | CTATGGTAGAGTGGTA | 93 | 2141 |
| 959734 | N/A | N/A | 12834 | 12849 | GCATTGCATAGCCTTC | 97 | 1948 |
| 959744 | N/A | N/A | 12886 | 12901 | AACCAATCCTGTTAGA | 55 | 1949 |
| 959754 | N/A | N/A | 12909 | 12924 | TCTGCTTATAAAGCAC | 0 | 1950 |
| 959764 | N/A | N/A | 12937 | 12952 | GGACAATAAGAGCTGT | 91 | 2142 |
| 959774 | N/A | N/A | 13373 | 13388 | CTTTGCAGGCACCCCA | 72 | 1951 |
| 959784 | N/A | N/A | 13398 | 13413 | CTTGAATGTCACCCTT | 92 | 1952 |
| 959794 | N/A | N/A | 13532 | 13547 | AGCATCATTGGAAGAC | 92 | 2143 |
| 959804 | N/A | N/A | 13715 | 13730 | TTACCAAGACCGCTAG | 57 | 1953 |
| 959814 | N/A | N/A | 13744 | 13759 | GTGTTAGAACTAAGGC | 94 | 2144 |
| 959824 | N/A | N/A | 14228 | 14243 | ACTTTTAGTATTAAAG | 0 | 1954 |
| 959834 | N/A | N/A | 14306 | 14321 | TTTCTGAGCAGATAAA | 66 | 2145 |
| 959844 | N/A | N/A | 14555 | 14570 | AAAGTCTTAATGTGGA | 87 | 1955 |
| 959854 | N/A | N/A | 14670 | 14685 | CTTAGTGTCCCCATCC | 77 | 1956 |
| 959864 | N/A | N/A | 15208 | 15223 | TAGGAATATTGCCAGG | 89 | 2146 |
| 959874 | N/A | N/A | 15767 | 15782 | GTTAGTGTTGGTTTAT | 94 | 1957 |
| 959884 | N/A | N/A | 17199 | 17214 | GTTTGACTTAGTCCGT | 95 | 1958 |
| 959894 | N/A | N/A | 17228 | 17243 | AACTCTGTAGCTGGTT | 41 | 2147 |
| 959904 | N/A | N/A | 17603 | 17618 | TCTTGATAGTGAATGT | 73 | 2148 |
| 959914 | N/A | N/A | 18398 | 18413 | AATATGTTTGGAAGTC | 89 | 1959 |
| 959924 | N/A | N/A | 20292 | 20307 | CCAATCAGAGGAAGCC | 58 | 2149 |
| 959934 | N/A | N/A | 20518 | 20533 | AGTAACCAGATTGAGT | 81 | 1960 |
| 959944 | N/A | N/A | 20617 | 20632 | CTGTCTCTAATTTTAC | 75 | 2150 |
| 959954 | N/A | N/A | 20665 | 20680 | CACAGCTTAGAAATTG | 87 | 1961 |
| 959964 | N/A | N/A | 20844 | 20859 | CCTGTATTAGCTCAAT | 92 | 1962 |
| 959974 | N/A | N/A | 20940 | 20955 | CCTCTTTGTAGCAGAC | 83 | 1963 |
| 959984 | N/A | N/A | 21006 | 21021 | GGTTATTTTAACAGCT | 85 | 1964 |
| 959994 | N/A | N/A | 21412 | 21427 | ACCTGCAATTCTAGAC | 54 | 1965 |
| 960004 | N/A | N/A | 21537 | 21552 | GACATTTCAGAGTATA | 93 | 2151 |
| 960014 | N/A | N/A | 22710 | 22725 | AAGATGTGAGTGAAAT | 69 | 1966 |
| 960024 | N/A | N/A | 22770 | 22785 | AAAGGACATGACAGAC | 75 | 1967 |
| 960034 | N/A | N/A | 24613 | 24628 | GCAAATCGGATCTTTG | 32 | 2152 |

TABLE 36
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915609 | 705 | 720 | 11923 | 11938 | TGATGGTTGTTTTGGC | 98 | 702 |
| 959275 | 440 | 455 | 6014 | 6029 | GAGGAACTTGCTTAAG | 80 | 1968 |
| 959285 | 696 | 711 | 11914 | 11929 | TTTTGGCATCAATGAA | 63 | 1969 |
| 959295 | 823 | 838 | 12041 | 12056 | AGAAGGTAGAGGTTCC | 48 | 2153 |
| 959305 | 1066 | 1081 | 16186 | 16201 | TCTAGCAGCTCATCTC | 66 | 1970 |
| 959315 | 1093 | 1108 | 16213 | 16228 | CAGGGCAGGATGCTGA | 2 | 2154 |
| 959325 | 1608 | 1623 | 25371 | 25386 | TGACTCACAGACTCTT | 60 | 2155 |
| 959335 | 1649 | 1664 | 25412 | 25427 | TTAGCACCTCTGAAAG | 54 | 1971 |
| 959345 | 1804 | 1819 | 25567 | 25582 | ATGTATTAGAGTTAAG | 79 | 1972 |
| 959355 | 1927 | 1942 | 25690 | 25705 | CATTCTAAGAACCTCA | 68 | 1973 |
| 959365 | 2183 | 2198 | 25946 | 25961 | TTAGTTAGGTGAAAAA | 69 | 1974 |
| 959375 | 2261 | 2276 | 26024 | 26039 | CACTGATTCACATAAT | 73 | 1975 |
| 959385 | N/A | N/A | 4305 | 4320 | AGAAACAAACCCTCCG | 70 | 2156 |
| 959395 | N/A | N/A | 4737 | 4752 | TGCAAATGATCATGTG | 69 | 1976 |
| 959405 | N/A | N/A | 4769 | 4784 | GCTGACTTTTATTGTT | 83 | 1977 |
| 959415 | N/A | N/A | 4872 | 4887 | AGTGAGTGTACTTTAG | 94 | 1978 |
| 959425 | N/A | N/A | 5395 | 5410 | TTAATGCTATCAGGTG | 82 | 1979 |
| 959435 | N/A | N/A | 5580 | 5595 | ATGCACAATGACATCA | 84 | 1980 |
| 959445 | N/A | N/A | 5624 | 5639 | ATTCTACCTGTGTCTT | 95 | 1981 |
| 959455 | N/A | N/A | 5651 | 5666 | TTGGATATATTGGGCT | 97 | 1982 |
| 959465 | N/A | N/A | 5800 | 5815 | ACTATGGGAGCCACAT | 26 | 2157 |
| 959475 | N/A | N/A | 5868 | 5883 | TACTTTTGGCAAGGCC | 69 | 1983 |
| 959485 | N/A | N/A | 7697 | 7712 | GCACAGAGTAGGTTAA | 70 | 1984 |
| 959655 | N/A | N/A | 12146 | 12161 | TGGTAGCTCCTGGCAA | 50 | 1985 |
| 959665 | N/A | N/A | 12162 | 12177 | TTGTGCAGACAGTAAC | 87 | 2158 |
| 959675 | N/A | N/A | 12201 | 12216 | TTTCCGTTAACCATCA | 95 | 1986 |
| 959685 | N/A | N/A | 12284 | 12299 | TTTTAGGTCTGGGTAT | 81 | 2159 |
| 959695 | N/A | N/A | 12667 | 12682 | CATCTTAGTGGCTGGG | 91 | 1987 |
| 959705 | N/A | N/A | 12695 | 12710 | GTTCTTGACCGTGTTT | 97 | 1988 |
| 959715 | N/A | N/A | 12717 | 12732 | GGTTAAACTACCGAAC | 26 | 1989 |
| 959725 | N/A | N/A | 12783 | 12798 | CATGGTCTGCAAATTT | 89 | 1990 |
| 959735 | N/A | N/A | 12837 | 12852 | ATTGCATTGCATAGCC | 95 | 2160 |
| 959745 | N/A | N/A | 12887 | 12902 | AAACCAATCCTGTTAG | 54 | 1991 |
| 959755 | N/A | N/A | 12910 | 12925 | ATCTGCTTATAAAGCA | 43 | 1992 |
| 959765 | N/A | N/A | 12964 | 12979 | CTTTGCAGCCTATCCC | 95 | 2161 |
| 959775 | N/A | N/A | 13374 | 13389 | ACTTTGCAGGCACCCC | 86 | 1993 |
| 959785 | N/A | N/A | 13399 | 13414 | GCTTGAATGTCACCCT | 95 | 1994 |
| 959795 | N/A | N/A | 13534 | 13549 | TCAGCATCATTGGAAG | 60 | 2162 |
| 959805 | N/A | N/A | 13716 | 13731 | TTTACCAAGACCGCTA | 82 | 1995 |
| 959815 | N/A | N/A | 13745 | 13760 | AGTGTTAGAACTAAGG | 93 | 2163 |
| 959825 | N/A | N/A | 14230 | 14245 | CAACTTTTAGTATTAA | 9 | 1996 |
| 959835 | N/A | N/A | 14417 | 14432 | TCGACACAGCATCACC | 59 | 1997 |
| 959845 | N/A | N/A | 14556 | 14571 | CAAAGTCTTAATGTGG | 84 | 2164 |
| 959855 | N/A | N/A | 14671 | 14686 | TCTTAGTGTCCCCATC | 78 | 1998 |
| 959865 | N/A | N/A | 15209 | 15224 | TTAGGAATATTGCCAG | 91 | 1999 |
| 959875 | N/A | N/A | 15769 | 15784 | GGGTTAGTGTTGGTTT | 93 | 2000 |
| 959885 | N/A | N/A | 17200 | 17215 | TGTTTGACTTAGTCCG | 96 | 2165 |
| 959895 | N/A | N/A | 17288 | 17303 | TAGGGCACCTCAAGAA | 0 | 2001 |
| 959905 | N/A | N/A | 17734 | 17749 | AATCTGGTCATATGGT | 42 | 2166 |
| 959915 | N/A | N/A | 18400 | 18415 | CAAATATGTTTGGAAG | 55 | 2002 |
| 959925 | N/A | N/A | 20293 | 20308 | CCCAATCAGAGGAAGC | 57 | 2003 |
| 959935 | N/A | N/A | 20599 | 20614 | TCATCATTATTACCTG | 93 | 2004 |
| 959945 | N/A | N/A | 20651 | 20666 | TGCAGTGCCCTGGCCT | 40 | 2167 |
| 959955 | N/A | N/A | 20803 | 20818 | TTCAGACCAGGGTAAT | 93 | 2005 |
| 959965 | N/A | N/A | 20845 | 20860 | GCCTGTATTAGCTCAA | 88 | 2006 |
| 959975 | N/A | N/A | 20941 | 20956 | GCCTCTTTGTAGCAGA | 0 | 2007 |
| 959985 | N/A | N/A | 21007 | 21022 | AGGTTATTTTAACAGC | 94 | 2168 |
| 959995 | N/A | N/A | 21434 | 21449 | TACTGAGAGGAAATGA | 65 | 2008 |
| 960005 | N/A | N/A | 21538 | 21553 | TGACATTTCAGAGTAT | 87 | 2169 |
| 960015 | N/A | N/A | 22714 | 22729 | TGTAAAGATGTGAGTG | 75 | 2009 |
| 960025 | N/A | N/A | 22772 | 22787 | TCAAAGGACATGACAG | 78 | 2010 |
| 960037 | N/A | N/A | 22590 | 22605 | GAGCAACGAGGAAGGA | 64 | 2011 |

### Example 2: Dose-Dependent Antisense Inhibition of Human PNPLA3 in A431 Cells

Gapmers from Example 1 exhibiting significant in vitro inhibition of PNPLA3 mRNA were selected and tested at various doses in A431 cells. The antisense oligonucleotides were tested in a series of experiments that had similar culture conditions. The results for each experiment are presented in separate tables shown below. Cells were plated at a density of 10,000 cells per well and transfected free uptake with different concentrations of antisense oligonucleotide, as specified in the Tables below. After a treatment period of approximately 16 hours, RNA was isolated from the cells and PNPLA3 mRNA levels were measured by quantitative real-time PCR. Human primer probe set RTS36070 was used to measure mRNA levels. PNPLA3 mRNA levels were adjusted according to total RNA content, as measured by RIBOGREEN®. Results are presented as percent inhibition of PNPLA3, relative to untreated control cells.

The half maximal inhibitory concentration (IC₅₀) of each oligonucleotide is also presented. PNPLA3 mRNA levels were significantly reduced in a dose-dependent manner in antisense oligonucleotide treated cells.

TABLE 37
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912712 | 27 | 67 | 76 | 74 | 0.2 |
| 912732 | 54 | 78 | 88 | 87 | <0.1 |
| 912733 | 45 | 74 | 85 | 88 | <0.1 |
| 912734 | 33 | 64 | 80 | 83 | 0.1 |
| 912756 | 46 | 72 | 89 | 92 | <0.1 |
| 912757 | 31 | 62 | 78 | 86 | 0.2 |
| 912758 | 38 | 70 | 85 | 90 | 0.1 |
| 912759 | 66 | 92 | 97 | 98 | <0.1 |
| 912772 | 46 | 63 | 79 | 88 | 0.1 |
| 912795 | 40 | 64 | 83 | 84 | 0.1 |
| 912812 | 43 | 81 | 88 | 88 | <0.1 |
| 912822 | 81 | 83 | 92 | 86 | <0.1 |
| 912823 | 67 | 80 | 91 | 86 | <0.1 |
| 912825 | 58 | 80 | 86 | 88 | <0.1 |
| 912834 | 37 | 75 | 81 | 84 | 0.1 |
| 912841 | 17 | 62 | 79 | 69 | 0.3 |
| 912847 | 70 | 83 | 90 | 91 | <0.1 |
| 912848 | 80 | 89 | 90 | 90 | <0.1 |
| 912855 | 48 | 62 | 77 | 80 | 0.1 |

TABLE 38
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 68 | 94 | 94 | 98 | <0.1 |
| 912813 | 57 | 84 | 90 | 87 | <0.1 |
| 912856 | 60 | 81 | 91 | 88 | <0.1 |
| 912859 | 48 | 79 | 81 | 72 | <0.1 |
| 912864 | 60 | 88 | 90 | 90 | <0.1 |
| 912870 | 67 | 81 | 91 | 94 | <0.1 |
| 912871 | 21 | 67 | 84 | 89 | 0.2 |
| 912872 | 18 | 73 | 90 | 92 | 0.2 |
| 912876 | 43 | 70 | 87 | 92 | 0.1 |
| 912933 | 68 | 89 | 90 | 90 | <0.1 |
| 912940 | 86 | 91 | 95 | 96 | <0.1 |
| 912941 | 87 | 94 | 96 | 96 | <0.1 |
| 912952 | 68 | 85 | 90 | 91 | <0.1 |
| 912953 | 80 | 90 | 95 | 93 | <0.1 |
| 912964 | 59 | 78 | 88 | 91 | <0.1 |
| 912973 | 53 | 70 | 87 | 91 | <0.1 |
| 912980 | 54 | 77 | 84 | 88 | <0.1 |
| 912985 | 23 | 61 | 81 | 87 | 0.2 |
| 912988 | 65 | 83 | 86 | 89 | <0.1 |

TABLE 39
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 72 | 95 | 97 | 99 | <0.1 |
| 912874 | 78 | 90 | 96 | 97 | <0.1 |
| 912875 | 64 | 83 | 92 | 94 | <0.1 |
| 912886 | 49 | 78 | 85 | 92 | <0.1 |
| 912931 | 68 | 88 | 94 | 95 | <0.1 |
| 912934 | 57 | 83 | 90 | 92 | <0.1 |
| 912936 | 50 | 78 | 89 | 89 | <0.1 |
| 912938 | 57 | 73 | 85 | 87 | <0.1 |
| 912943 | 64 | 84 | 90 | 93 | <0.1 |
| 912954 | 80 | 92 | 93 | 94 | <0.1 |
| 912970 | 44 | 73 | 86 | 90 | <0.1 |
| 912986 | 56 | 78 | 91 | 92 | <0.1 |
| 912987 | 79 | 90 | 92 | 88 | <0.1 |
| 912992 | 21 | 59 | 74 | 81 | 0.3 |
| 915603 | 50 | 88 | 96 | 98 | <0.1 |
| 915623 | 81 | 96 | 98 | 98 | <0.1 |
| 915643 | 67 | 89 | 94 | 96 | <0.1 |
| 916602 | 79 | 92 | 95 | 96 | <0.1 |
| 916642 | 44 | 83 | 91 | 93 | <0.1 |

TABLE 40
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 73 | 94 | 98 | 99 | <0.1 |
| 915484 | 67 | 87 | 93 | 95 | <0.1 |
| 915543 | 34 | 69 | 87 | 90 | 0.1 |
| 915604 | 54 | 78 | 91 | 95 | <0.1 |
| 915763 | 63 | 80 | 87 | 87 | <0.1 |
| 915904 | 50 | 83 | 92 | 94 | <0.1 |
| 915923 | 63 | 74 | 82 | 87 | <0.1 |
| 916183 | 33 | 78 | 89 | 91 | 0.1 |
| 916303 | 58 | 73 | 84 | 91 | <0.1 |
| 916343 | 15 | 72 | 76 | 87 | 0.2 |
| 916563 | 46 | 74 | 90 | 95 | <0.1 |
| 916582 | 48 | 74 | 89 | 91 | <0.1 |
| 916623 | 64 | 81 | 91 | 94 | <0.1 |
| 916702 | 45 | 70 | 78 | 79 | <0.1 |
| 916761 | 46 | 75 | 85 | 88 | <0.1 |
| 916781 | 55 | 79 | 86 | 87 | <0.1 |
| 916782 | 62 | 87 | 91 | 93 | <0.1 |
| 916802 | 66 | 88 | 94 | 91 | <0.1 |
| 916822 | 29 | 72 | 83 | 87 | 0.1 |

TABLE 41
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 72 | 95 | 98 | 99 | <0.1 |
| 915525 | 51 | 76 | 88 | 84 | <0.1 |
| 915546 | 39 | 79 | 90 | 94 | 0.1 |
| 915605 | 59 | 84 | 96 | 96 | <0.1 |
| 915606 | 74 | 94 | 99 | 98 | <0.1 |
| 915625 | 72 | 82 | 91 | 95 | <0.1 |
| 915944 | 36 | 71 | 75 | 83 | 0.1 |
| 916065 | 36 | 62 | 78 | 79 | 0.1 |
| 916144 | 71 | 86 | 90 | 92 | <0.1 |
| 916163 | 36 | 67 | 81 | 74 | 0.1 |
| 916164 | 82 | 88 | 89 | 92 | <0.1 |
| 916184 | 60 | 79 | 87 | 89 | <0.1 |
| 916304 | 46 | 65 | 80 | 84 | 0.1 |
| 916324 | 57 | 77 | 87 | 92 | <0.1 |
| 916344 | 41 | 70 | 83 | 88 | 0.1 |
| 916564 | 38 | 66 | 88 | 94 | 0.1 |
| 916604 | 67 | 87 | 95 | 96 | <0.1 |
| 916624 | 43 | 59 | 79 | 87 | 0.1 |
| 916803 | 67 | 84 | 93 | 92 | <0.1 |

TABLE 42
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 70 | 94 | 98 | 99 | <0.1 |
| 915486 | 35 | 64 | 82 | 90 | 0.1 |
| 915487 | 62 | 89 | 94 | 95 | <0.1 |
| 915626 | 67 | 83 | 92 | 94 | <0.1 |
| 915786 | 65 | 84 | 88 | 88 | <0.1 |
| 916145 | 53 | 66 | 85 | 87 | <0.1 |
| 916146 | 62 | 77 | 86 | 86 | <0.1 |
| 916165 | 71 | 86 | 89 | 88 | <0.1 |
| 916166 | 71 | 83 | 87 | 88 | <0.1 |
| 916305 | 57 | 86 | 90 | 92 | <0.1 |
| 916306 | 86 | 96 | 98 | 98 | <0.1 |
| 916325 | 59 | 78 | 83 | 86 | <0.1 |
| 916345 | 21 | 47 | 67 | 73 | 0.4 |
| 916545 | 63 | 88 | 95 | 94 | <0.1 |
| 916546 | 66 | 85 | 92 | 95 | <0.1 |
| 916625 | 47 | 71 | 84 | 92 | <0.1 |
| 916706 | 22 | 65 | 80 | 85 | 0.2 |
| 916765 | 67 | 85 | 92 | 93 | <0.1 |
| 916845 | 38 | 71 | 80 | 87 | 0.1 |

TABLE 43
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 97 | 99 | 100 | 100 | <0.1 |
| 915608 | 66 | 91 | 97 | 97 | <0.1 |
| 915609 | 71 | 97 | 99 | 99 | <0.1 |
| 915627 | 0 | 26 | 53 | 62 | 1.3 |
| 915768 | 39 | 69 | 86 | 91 | 0.1 |
| 915908 | 49 | 70 | 80 | 85 | <0.1 |
| 915987 | 47 | 60 | 75 | 78 | 0.1 |
| 916008 | 45 | 69 | 84 | 83 | <0.1 |
| 916187 | 71 | 82 | 88 | 92 | <0.1 |
| 916247 | 34 | 72 | 83 | 84 | 0.1 |
| 916287 | 31 | 70 | 90 | 91 | 0.1 |
| 916547 | 79 | 93 | 97 | 97 | <0.1 |
| 916566 | 8 | 45 | 73 | 81 | 0.5 |
| 916586 | 47 | 67 | 89 | 91 | <0.1 |
| 916587 | 48 | 81 | 90 | 94 | <0.1 |
| 916606 | 18 | 64 | 87 | 90 | 0.2 |
| 916607 | 72 | 94 | 94 | 95 | <0.1 |
| 916627 | 18 | 51 | 82 | 79 | 0.3 |
| 916805 | 18 | 65 | 78 | 81 | 0.2 |

TABLE 44
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 64 | 92 | 97 | 99 | <0.1 |
| 915610 | 74 | 94 | 98 | 99 | <0.1 |
| 915789 | 35 | 72 | 82 | 85 | 0.1 |
| 915909 | 52 | 69 | 82 | 86 | <0.1 |
| 915929 | 13 | 32 | 60 | 59 | 1.0 |
| 915969 | 39 | 54 | 74 | 74 | 0.2 |
| 915989 | 46 | 67 | 81 | 86 | 0.1 |
| 916069 | 24 | 59 | 75 | 56 | 0.3 |
| 916148 | 42 | 71 | 85 | 80 | <0.1 |
| 916168 | 28 | 54 | 74 | 68 | 0.3 |
| 916188 | 22 | 42 | 72 | 70 | 0.4 |
| 916309 | 30 | 77 | 91 | 96 | 0.1 |
| 916348 | 41 | 57 | 65 | 73 | 0.1 |
| 916549 | 64 | 85 | 94 | 96 | <0.1 |
| 916568 | 54 | 66 | 81 | 87 | <0.1 |
| 916569 | 60 | 86 | 92 | 95 | <0.1 |
| 916728 | 22 | 50 | 68 | 73 | 0.4 |
| 916788 | 60 | 89 | 94 | 96 | <0.1 |
| 916848 | 49 | 75 | 89 | 95 | <0.1 |

TABLE 45
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 81 | 93 | 98 | 100 | <0.1 |
| 915390 | 9 | 40 | 67 | 77 | 0.6 |
| 915611 | 46 | 80 | 93 | 88 | <0.1 |
| 915630 | 52 | 69 | 81 | 82 | <0.1 |
| 915910 | 44 | 64 | 79 | 80 | 0.1 |
| 915931 | 83 | 88 | 89 | 86 | <0.1 |
| 916149 | 73 | 87 | 89 | 83 | <0.1 |
| 916150 | 51 | 68 | 77 | 84 | <0.1 |
| 916189 | 60 | 73 | 77 | 79 | <0.1 |
| 916310 | 45 | 77 | 88 | 95 | <0.1 |
| 916330 | 48 | 67 | 84 | 86 | <0.1 |
| 916550 | 62 | 85 | 94 | 97 | <0.1 |
| 916570 | 89 | 96 | 98 | 98 | <0.1 |
| 916629 | 26 | 53 | 73 | 86 | 0.3 |
| 916630 | 52 | 68 | 87 | 91 | <0.1 |
| 916670 | 43 | 77 | 78 | 85 | <0.1 |
| 916730 | 61 | 74 | 82 | 86 | <0.1 |
| 916768 | 35 | 57 | 67 | 72 | 0.2 |
| 916789 | 79 | 92 | 96 | 96 | <0.1 |

TABLE 46
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 76 | 94 | 96 | 99 | <0.1 |
| 915532 | 31 | 66 | 82 | 92 | 0.1 |
| 915612 | 54 | 77 | 86 | 90 | <0.1 |
| 915732 | 42 | 63 | 80 | 84 | 0.1 |
| 915932 | 45 | 71 | 88 | 89 | <0.1 |
| 915951 | 26 | 58 | 71 | 74 | 0.3 |
| 915991 | 67 | 84 | 85 | 85 | <0.1 |
| 915992 | 54 | 78 | 86 | 87 | <0.1 |
| 916112 | 35 | 67 | 76 | 78 | 0.1 |
| 916151 | 51 | 79 | 87 | 90 | <0.1 |
| 916311 | 36 | 70 | 81 | 87 | 0.1 |
| 916331 | 56 | 85 | 93 | 95 | <0.1 |
| 916332 | 82 | 91 | 94 | 96 | <0.1 |
| 916390 | 30 | 41 | 68 | 64 | 0.5 |
| 916552 | 79 | 93 | 96 | 97 | <0.1 |
| 916571 | 53 | 78 | 90 | 94 | <0.1 |
| 916631 | 48 | 77 | 86 | 90 | <0.1 |
| 916651 | 81 | 89 | 94 | 95 | <0.1 |
| 916711 | 37 | 66 | 85 | 91 | 0.1 |

TABLE 47
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 58 | 90 | 98 | 99 | <0.1 |
| 915474 | 51 | 79 | 90 | 93 | <0.1 |
| 915493 | 48 | 58 | 83 | 80 | 0.1 |
| 915494 | 46 | 73 | 86 | 90 | <0.1 |
| 915674 | 49 | 72 | 89 | 93 | <0.1 |
| 915933 | 40 | 63 | 75 | 79 | 0.1 |
| 916153 | 68 | 86 | 89 | 91 | <0.1 |
| 916172 | 85 | 89 | 87 | 87 | <0.1 |
| 916173 | 81 | 90 | 91 | 88 | <0.1 |
| 916292 | 64 | 83 | 92 | 92 | <0.1 |
| 916312 | 60 | 84 | 91 | 92 | <0.1 |
| 916333 | 75 | 92 | 96 | 96 | <0.1 |
| 916572 | 29 | 62 | 79 | 88 | 0.2 |
| 916592 | 52 | 74 | 89 | 90 | <0.1 |
| 916593 | 25 | 67 | 83 | 93 | 0.2 |
| 916613 | 46 | 75 | 89 | 92 | <0.1 |
| 916652 | 65 | 83 | 91 | 88 | <0.1 |
| 916672 | 73 | 89 | 93 | 90 | <0.1 |
| 916772 | 50 | 61 | 83 | 89 | <0.1 |

TABLE 48
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 50 | 89 | 96 | 99 | <0.1 |
| 915534 | 0 | 33 | 66 | 57 | 1.1 |
| 915535 | 51 | 81 | 92 | 96 | <0.1 |
| 915634 | 18 | 67 | 79 | 84 | 0.2 |
| 915635 | 44 | 72 | 86 | 91 | 0.1 |
| 915675 | 36 | 68 | 82 | 90 | 0.1 |
| 915735 | 45 | 68 | 73 | 84 | 0.1 |
| 915936 | 36 | 67 | 78 | 83 | 0.1 |
| 915995 | 78 | 87 | 90 | 89 | <0.1 |
| 915996 | 83 | 91 | 93 | 92 | <0.1 |
| 916174 | 80 | 84 | 86 | 81 | <0.1 |
| 916175 | 55 | 82 | 86 | 89 | <0.1 |
| 916334 | 50 | 82 | 92 | 94 | <0.1 |
| 916335 | 52 | 76 | 89 | 93 | <0.1 |
| 916575 | 62 | 88 | 93 | 93 | <0.1 |
| 916753 | 49 | 69 | 76 | 74 | <0.1 |
| 916774 | 49 | 72 | 86 | 91 | <0.1 |
| 916794 | 26 | 64 | 85 | 85 | 0.2 |
| 916873 | 16 | 49 | 72 | 82 | 0.4 |

TABLE 49
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 57 | 90 | 97 | 99 | <0.1 |
| 915477 | 47 | 65 | 88 | 93 | 0.1 |
| 915478 | 47 | 78 | 90 | 95 | <0.1 |
| 915497 | 63 | 68 | 79 | 86 | <0.1 |
| 915637 | 67 | 91 | 97 | 98 | <0.1 |
| 916037 | 15 | 47 | 70 | 61 | 0.6 |
| 916236 | 80 | 87 | 90 | 88 | <0.1 |
| 916336 | 52 | 67 | 81 | 87 | <0.1 |
| 916576 | 50 | 76 | 89 | 93 | <0.1 |
| 916596 | 55 | 82 | 93 | 94 | <0.1 |
| 916636 | 42 | 71 | 87 | 90 | 0.1 |
| 916637 | 56 | 85 | 90 | 93 | <0.1 |
| 916715 | 27 | 38 | 68 | 68 | 0.5 |
| 916716 | 35 | 77 | 89 | 93 | 0.1 |
| 916796 | 14 | 62 | 84 | 89 | 0.3 |
| 916814 | 22 | 44 | 70 | 79 | 0.4 |
| 916815 | 56 | 79 | 87 | 89 | <0.1 |
| 916816 | 33 | 72 | 83 | 93 | 0.1 |
| 916874 | 5 | 34 | 61 | 70 | 0.8 |

TABLE 50
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 56 | 91 | 97 | 100 | <0.1 |
| 915479 | 38 | 70 | 89 | 94 | 0.1 |
| 915618 | 42 | 63 | 75 | 85 | 0.1 |
| 915619 | 65 | 87 | 96 | 97 | <0.1 |
| 915638 | 31 | 64 | 80 | 82 | 0.2 |
| 915639 | 33 | 78 | 88 | 93 | 0.1 |
| 915778 | 41 | 50 | 78 | 87 | 0.2 |
| 916058 | 26 | 34 | 73 | 81 | 0.4 |
| 916177 | 38 | 55 | 83 | 82 | 0.1 |
| 916238 | 84 | 91 | 93 | 93 | <0.1 |
| 916298 | 79 | 87 | 92 | 94 | <0.1 |
| 916318 | 59 | 71 | 91 | 94 | <0.1 |
| 916338 | 71 | 91 | 94 | 92 | <0.1 |
| 916558 | 73 | 89 | 94 | 94 | <0.1 |
| 916577 | 41 | 66 | 78 | 82 | 0.1 |
| 916578 | 69 | 85 | 91 | 93 | <0.1 |
| 916638 | 46 | 84 | 90 | 92 | <0.1 |
| 916757 | 33 | 60 | 82 | 88 | 0.2 |
| 916817 | 31 | 67 | 82 | 87 | 0.1 |

TABLE 51
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 841947 | 50 | 78 | 89 | 92 | <0.1 |
| 912759 | 75 | 50 | 85 | 99 | <0.1 |
| 912986 | 54 | 78 | 90 | 95 | <0.1 |
| 915480 | 61 | 87 | 94 | 97 | <0.1 |
| 915519 | 47 | 77 | 86 | 85 | <0.1 |
| 915620 | 46 | 75 | 88 | 91 | <0.1 |
| 915780 | 24 | 76 | 92 | 94 | 0.1 |
| 915920 | 23 | 65 | 79 | 82 | 0.2 |
| 916020 | 45 | 80 | 85 | 81 | <0.1 |
| 916299 | 59 | 87 | 92 | 93 | <0.1 |
| 916339 | 88 | 95 | 97 | 97 | <0.1 |
| 916340 | 83 | 96 | 97 | 98 | <0.1 |
| 916559 | 41 | 68 | 83 | 89 | 0.1 |
| 916579 | 71 | 86 | 96 | 96 | <0.1 |
| 916580 | 59 | 90 | 95 | 96 | <0.1 |
| 916618 | 10 | 47 | 70 | 78 | 0.5 |
| 916639 | 38 | 58 | 80 | 86 | 0.1 |
| 916778 | 68 | 87 | 92 | 94 | <0.1 |
| 916818 | 60 | 77 | 90 | 91 | <0.1 |

TABLE 52
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 60 | 0 | 85 | 99 | 0.3 |
| 915541 | 48 | 71 | 89 | 91 | <0.1 |
| 915542 | 50 | 72 | 86 | 93 | <0.1 |
| 915601 | 8 | 53 | 84 | 84 | 0.3 |
| 915602 | 1 | 56 | 77 | 91 | 0.4 |
| 915621 | 21 | 54 | 75 | 80 | 0.3 |
| 915622 | 0 | 44 | 73 | 84 | 0.5 |
| 915922 | 27 | 64 | 79 | 85 | 0.2 |
| 916042 | 6 | 57 | 89 | 88 | 0.3 |
| 916140 | 43 | 82 | 90 | 89 | <0.1 |
| 916141 | 72 | 88 | 93 | 91 | <0.1 |
| 916180 | 33 | 62 | 69 | 83 | 0.2 |
| 916181 | 53 | 80 | 89 | 92 | <0.1 |
| 916341 | 0 | 78 | 94 | 94 | 0.3 |
| 916560 | 72 | 91 | 95 | 94 | <0.1 |
| 916581 | 38 | 76 | 91 | 91 | 0.1 |
| 916601 | 44 | 80 | 88 | 90 | <0.1 |
| 916701 | 61 | 83 | 91 | 93 | <0.1 |
| 916780 | 75 | 91 | 93 | 94 | <0.1 |

TABLE 53
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 52 | 86 | 96 | 99 | <0.01 |
| 959430 | 42 | 76 | 90 | 95 | <0.01 |
| 959440 | 39 | 73 | 92 | 98 | 0.02 |
| 959470 | 46 | 73 | 89 | 94 | <0.01 |
| 959670 | 52 | 90 | 96 | 98 | <0.01 |
| 959680 | 50 | 75 | 91 | 96 | <0.01 |
| 959730 | 83 | 96 | 98 | 98 | <0.01 |
| 959740 | 50 | 70 | 90 | 96 | <0.01 |
| 959820 | 40 | 69 | 85 | 92 | 0.02 |
| 959830 | 46 | 69 | 93 | 97 | 0.02 |
| 959880 | 34 | 62 | 85 | 93 | 0.03 |
| 960010 | 48 | 78 | 92 | 95 | <0.01 |

TABLE 54
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 53 | 87 | 98 | 99 | <0.01 |
| 959271 | 55 | 74 | 91 | 91 | <0.01 |
| 959360 | 7 | 43 | 79 | 85 | 0.1 |
| 959361 | 60 | 87 | 93 | 94 | <0.01 |
| 959411 | 56 | 76 | 91 | 94 | <0.01 |
| 959441 | 50 | 81 | 93 | 97 | <0.01 |
| 959460 | 0 | 29 | 75 | 90 | 0.2 |
| 959701 | 62 | 91 | 97 | 98 | <0.01 |
| 959721 | 80 | 94 | 97 | 97 | <0.01 |
| 959731 | 25 | 64 | 82 | 91 | 0.05 |
| 959741 | 41 | 65 | 83 | 91 | 0.02 |
| 959750 | 0 | 26 | 65 | 87 | 0.2 |
| 959761 | 28 | 60 | 84 | 91 | 0.05 |
| 959781 | 39 | 58 | 75 | 87 | 0.04 |
| 959911 | 20 | 54 | 78 | 90 | 0.1 |
| 959921 | 37 | 61 | 83 | 91 | 0.03 |
| 959931 | 48 | 72 | 89 | 92 | <0.01 |
| 959960 | 11 | 51 | 79 | 90 | 0.1 |
| 959961 | 38 | 64 | 85 | 92 | 0.03 |

TABLE 55
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 11 | 71 | 93 | 98 | 0.1 |
| 959412 | 52 | 77 | 90 | 94 | <0.01 |
| 959413 | 34 | 82 | 95 | 97 | 0.02 |
| 959422 | 15 | 50 | 80 | 87 | 0.1 |
| 959432 | 33 | 60 | 86 | 95 | 0.04 |
| 959662 | 0 | 53 | 84 | 92 | 0.1 |
| 959672 | 54 | 85 | 95 | 97 | <0.01 |
| 959673 | 18 | 62 | 88 | 95 | 0.1 |
| 959682 | 46 | 77 | 90 | 91 | <0.01 |
| 959702 | 39 | 71 | 91 | 96 | 0.02 |
| 959703 | 81 | 96 | 99 | 99 | <0.01 |
| 959712 | 4 | 30 | 75 | 92 | 0.1 |
| 959713 | 0 | 53 | 86 | 96 | 0.1 |
| 959722 | 33 | 80 | 90 | 94 | 0.02 |
| 959733 | 31 | 68 | 92 | 96 | 0.03 |
| 959782 | 35 | 63 | 86 | 94 | 0.03 |
| 959872 | 29 | 64 | 77 | 89 | 0.04 |
| 959912 | 25 | 69 | 89 | 92 | 0.04 |
| 959982 | 21 | 61 | 83 | 91 | 0.1 |

TABLE 56
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 2 | 73 | 93 | 98 | 0.1 |
| 959363 | 49 | 82 | 91 | 91 | <0.01 |
| 959393 | 38 | 71 | 87 | 95 | 0.02 |
| 959394 | 27 | 73 | 91 | 97 | 0.03 |
| 959414 | 69 | 94 | 98 | 99 | <0.01 |
| 959664 | 51 | 77 | 95 | 98 | <0.01 |
| 959674 | 43 | 74 | 95 | 98 | 0.02 |
| 959683 | 14 | 71 | 90 | 96 | 0.05 |
| 959704 | 57 | 92 | 98 | 99 | <0.01 |
| 959724 | 0 | 68 | 90 | 95 | 0.1 |
| 959734 | 71 | 93 | 98 | 98 | <0.01 |
| 959814 | 24 | 76 | 90 | 95 | 0.03 |
| 959873 | 38 | 53 | 83 | 90 | 0.04 |
| 959874 | 51 | 82 | 95 | 97 | <0.01 |
| 959884 | 44 | 77 | 94 | 97 | <0.01 |
| 959913 | 18 | 50 | 85 | 92 | 0.1 |
| 959953 | 6 | 51 | 85 | 92 | 0.1 |
| 959983 | 22 | 54 | 81 | 92 | 0.1 |
| 960004 | 10 | 71 | 92 | 96 | 0.1 |

TABLE 57
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 12 | 69 | 93 | 98 | 0.1 |
| 959364 | 32 | 72 | 89 | 91 | 0.03 |
| 959415 | 21 | 70 | 91 | 96 | 0.04 |
| 959444 | 6 | 47 | 82 | 91 | 0.1 |
| 959445 | 32 | 70 | 92 | 97 | 0.03 |
| 959455 | 61 | 87 | 95 | 97 | <0.01 |
| 959675 | 20 | 56 | 80 | 94 | 0.1 |
| 959684 | 8 | 47 | 83 | 86 | 0.1 |
| 959705 | 77 | 95 | 98 | 99 | <0.01 |
| 959735 | 12 | 67 | 90 | 95 | 0.1 |
| 959764 | 4 | 32 | 80 | 92 | 0.1 |
| 959765 | 1 | 59 | 88 | 93 | 0.1 |
| 959784 | 3 | 35 | 75 | 90 | 0.1 |
| 959785 | 27 | 72 | 92 | 96 | 0.03 |
| 959794 | 0 | 0 | 53 | 83 | 0.3 |
| 959864 | 26 | 61 | 84 | 91 | 0.05 |
| 959885 | 49 | 81 | 95 | 96 | <0.01 |
| 959914 | 7 | 43 | 76 | 89 | 0.1 |
| 959964 | 17 | 55 | 83 | 91 | 0.1 |

TABLE 58
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 0 | 73 | 95 | 97 | 0.1 |
| 959456 | 66 | 90 | 97 | 98 | <0.01 |
| 959666 | 29 | 60 | 89 | 97 | 0.04 |
| 959676 | 15 | 44 | 81 | 93 | 0.1 |
| 959686 | 71 | 92 | 97 | 97 | <0.01 |
| 959695 | 40 | 75 | 91 | 93 | 0.02 |
| 959696 | 21 | 81 | 90 | 92 | 0.03 |
| 959706 | 81 | 95 | 98 | 98 | <0.01 |
| 959725 | 8 | 55 | 76 | 84 | 0.1 |
| 959726 | 0 | 59 | 88 | 91 | 0.1 |
| 959736 | 46 | 84 | 94 | 98 | <0.01 |
| 959766 | 22 | 57 | 83 | 94 | 0.1 |
| 959776 | 1 | 53 | 87 | 93 | 0.1 |
| 959815 | 31 | 67 | 89 | 91 | 0.03 |
| 959865 | 6 | 49 | 84 | 91 | 0.1 |
| 959875 | 34 | 74 | 91 | 92 | 0.02 |
| 959935 | 22 | 55 | 84 | 94 | 0.1 |
| 959955 | 0 | 55 | 83 | 89 | 0.1 |
| 959985 | 29 | 71 | 88 | 93 | 0.03 |

TABLE 59
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 37 | 80 | 96 | 99 | 0.02 |
| 959356 | 40 | 71 | 87 | 88 | 0.02 |
| 959417 | 25 | 58 | 83 | 92 | 0.1 |
| 959437 | 65 | 88 | 94 | 95 | <0.01 |
| 959667 | 37 | 69 | 90 | 95 | 0.02 |
| 959677 | 29 | 56 | 82 | 92 | 0.05 |
| 959687 | 51 | 79 | 93 | 97 | <0.01 |
| 959697 | 51 | 75 | 93 | 95 | <0.01 |
| 959707 | 81 | 94 | 98 | 98 | <0.01 |
| 959727 | 71 | 92 | 96 | 96 | <0.01 |
| 959737 | 45 | 75 | 89 | 94 | <0.01 |
| 959767 | 47 | 76 | 93 | 96 | <0.01 |
| 959797 | 32 | 59 | 87 | 94 | 0.04 |
| 959856 | 13 | 35 | 67 | 80 | 0.1 |
| 959876 | 38 | 75 | 89 | 90 | 0.02 |
| 959877 | 40 | 81 | 89 | 94 | <0.01 |
| 959956 | 25 | 25 | 66 | 85 | 0.1 |
| 960006 | 13 | 40 | 68 | 83 | 0.1 |
| 960007 | 24 | 59 | 88 | 91 | 0.05 |

### Example 3: Tolerability of Modified Oligonucleotides Targeting Human PNPLA3 in BALB/c Mice

BALB/c mice are a multipurpose mouse model frequently utilized for safety and efficacy testing. The mice were treated with antisense oligonucleotides selected from studies described above and evaluated for changes in the levels of various plasma chemistry markers.

Ionis oligonucleotides selected from the studies above were conjugated with 3′-THA-C₆-GalNAc₃-(3R,5S)-5-(hydroxymethyl) pyrrolidin-3-ol phosphate endcap (henceforth referred to as 3′-THA).

### Treatment

Groups of 6- to 7-week-old male mice were injected subcutaneously once with 200 mg/kg of modified oligonucleotides. One group of male BALB/c mice was injected with PBS. Mice were euthanized 72-96 hours after the single dose and plasma was harvested for further analysis.

To evaluate the effect of modified oligonucleotides on liver function, plasma levels of transaminases were measured using an automated clinical chemistry analyzer (Beckman Coulter AU480, Brea, CA). Modified oligonucleotides that caused changes in the levels of transaminases outside the expected range for antisense oligonucleotides were excluded in further studies. The oligonucleotides which were considered tolerable in this study and were selected for further evaluation are presented in the Table below. ‘Parent Oligo’ indicates the Ionis oligonucleotide that has been described in the studies above and that was conjugated with 3′-THA and tested in this study.

TABLE 60
Antisense oligonucleotides in BALB/c mouse study

| Compound ID | Parent oligo ID |
|---|---|
| 975746 | 916339 |
| 975747 | 912941 |
| 975748 | 916306 |
| 975755 | 916332 |
| 975760 | 912848 |
| 975764 | 916298 |
| 975766 | 916552 |
| 975767 | 916789 |
| 975768 | 916602 |
| 975770 | 912874 |
| 975771 | 916333 |
| 975772 | 916780 |
| 975775 | 916672 |
| 975777 | 916558 |
| 975780 | 916607 |
| 975783 | 916338 |
| 975788 | 912847 |
| 975790 | 916778 |
| 975792 | 912870 |
| 975794 | 916802 |
| 975797 | 916637 |
| 975799 | 912732 |
| 975800 | 912733 |
| 975803 | 912813 |
| 975804 | 912823 |
| 975805 | 912834 |
| 975806 | 912855 |
| 975807 | 912856 |
| 975808 | 912864 |
| 975809 | 912871 |
| 975810 | 912872 |
| 975811 | 912875 |
| 975813 | 912931 |
| 975814 | 912934 |
| 975815 | 912936 |
| 975816 | 912938 |
| 975817 | 912943 |
| 975820 | 912988 |
| 975822 | 915486 |
| 975829 | 915619 |
| 975836 | 915780 |
| 975840 | 915989 |
| 975844 | 916151 |
| 975849 | 916292 |
| 975850 | 916299 |
| 975851 | 916303 |
| 975852 | 916309 |
| 975853 | 916310 |
| 975854 | 916312 |
| 975855 | 916318 |
| 975856 | 916324 |
| 975857 | 916331 |
| 975858 | 916334 |
| 975859 | 916335 |
| 975860 | 916336 |
| 975861 | 916549 |
| 975862 | 916550 |
| 975864 | 916563 |
| 975865 | 916564 |
| 975866 | 916568 |
| 975868 | 916571 |
| 975869 | 916575 |
| 975870 | 916580 |
| 975871 | 916581 |
| 975873 | 916586 |
| 975875 | 916601 |
| 975878 | 916624 |
| 975879 | 916625 |
| 975880 | 916636 |
| 975881 | 916638 |
| 975883 | 916670 |
| 975886 | 916711 |
| 975887 | 916716 |
| 975888 | 916774 |
| 975889 | 916781 |
| 975890 | 916782 |
| 975891 | 916788 |
| 975893 | 916815 |
| 975894 | 916816 |
| 975895 | 916817 |
| 975896 | 916818 |
| 975897 | 916822 |
| 975898 | 916845 |
| 994288 | 959455 |
| 994289 | 960010 |
| 994290 | 959361 |
| 994291 | 959271 |

### Example 4: Effect of antisense inhibition of PNPLA3 in transgenic mouse model

A PNPLA3 transgenic mouse model from wild-type C57BL/6 generated by the University of California, Irvine was used. The mouse model comprises a genomic construct containing the entire PNPLA3 gene fosmid, generously provided by the University of Washington. The efficacy of Ionis oligonucleotides was evaluated in this model.

### Treatment

Transgenic mice were maintained on a 12-hour light/dark cycle and were fed ad libitum normal Purina mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

The hPNPLA3 Tg mice were divided into groups of 2 mice each. Groups received subcutaneous injections of Ionis oligonucleotide at a dose of 2.5 mg/kg on days 1 and 8. One group of 4 mice received subcutaneous injections of PBS on days 1 and 8. The saline-injected group served as the control group to which oligonucleotide-treated groups were compared.

### RNA Analysis

On day 10, RNA was extracted from liver for real-time PCR analysis of measurement of mRNA expression of PNPLA3. Primer probe sets RTS36070 and RTS36075 were both used to measure PNPLA3 mRNA levels. Results are presented as percent change of mRNA, relative to PBS control, normalized with RIBOGREEN®. As presented in the Table below, treatment with Ionis antisense oligonucleotides resulted in significant reduction of PNPLA3 mRNA in comparison to the PBS control. ‘0’ indicates that the oligonucleotides did not inhibit mRNA expression.

TABLE 61
Percent inhibition of PNPLA3 mRNA in the transgenic
mice liver relative to the PBS control

| | Inhibition (%) | Inhibition (%) |
|---|---|---|
| | measured with | measured with |
| Compound ID | RTS36070 | RTS36075 |
| 975746 | 99 | 99 |
| 975747 | 99 | 99 |
| 975748 | 98 | 98 |
| 975755 | 99 | 99 |
| 975760 | 96 | 97 |
| 975764 | 75 | 83 |
| 975766 | 99 | 99 |
| 975767 | 98 | 98 |
| 975768 | 98 | 98 |
| 975770 | 97 | 97 |
| 975771 | 98 | 99 |
| 975772 | 96 | 96 |
| 975775 | 90 | 91 |
| 975777 | 85 | 89 |
| 975780 | 44 | 63 |
| 975783 | 87 | 90 |
| 975788 | 0 | 26 |
| 975790 | 0 | 0 |
| 975792 | 9 | 34 |
| 975794 | 44 | 50 |
| 975797 | 0 | 0 |
| 975799 | 0 | 0 |
| 975800 | 0 | 5 |
| 975803 | 68 | 68 |
| 975804 | 11 | 38 |
| 975805 | 0 | 0 |
| 975806 | 0 | 0 |
| 975807 | 0 | 0 |
| 975808 | 47 | 58 |
| 975809 | 0 | 19 |
| 975810 | 12 | 22 |
| 975811 | 19 | 32 |
| 975813 | 36 | 39 |
| 975814 | 48 | 54 |
| 975815 | 78 | 77 |
| 975816 | 56 | 56 |
| 975817 | 84 | 86 |
| 975820 | 35 | 45 |
| 975822 | 0 | 0 |
| 975829 | 98 | 98 |
| 975836 | 85 | 91 |
| 975840 | 19 | 44 |
| 975844 | 21 | 31 |
| 975849 | 88 | 89 |
| 975850 | 41 | 48 |
| 975851 | 5 | 18 |
| 975852 | 24 | 41 |
| 975853 | 0 | 0 |
| 975854 | 0 | 0 |
| 975855 | 0 | 0 |
| 975856 | 45 | 31 |
| 975857 | 73 | 67 |
| 975858 | 58 | 40 |
| 975860 | 92 | 92 |
| 975861 | 66 | 49 |
| 975862 | 46 | 36 |
| 975864 | 16 | 21 |
| 975865 | 0 | 0 |
| 975866 | 40 | 41 |
| 975868 | 56 | 48 |
| 975869 | 30 | 19 |
| 975870 | 0 | 14 |
| 975871 | 0 | 0 |
| 975875 | 75 | 73 |
| 975878 | 18 | 12 |
| 975879 | 7 | 0 |
| 975880 | 0 | 0 |
| 975881 | 54 | 54 |
| 975883 | 77 | 80 |
| 975886 | 18 | 28 |
| 975887 | 49 | 57 |
| 975888 | 10 | 9 |
| 975889 | 90 | 91 |
| 975890 | 96 | 98 |
| 975891 | 97 | 98 |
| 975893 | 95 | 95 |
| 975894 | 85 | 87 |
| 975895 | 89 | 89 |
| 975896 | 91 | 89 |
| 975898 | 94 | 95 |
| 975897 | 96 | 97 |
| 975873 | 99 | 99 |
| 994288 | 99 | 99 |
| 994289 | 98 | 99 |
| 994290 | 98 | 99 |
| 994291 | 95 | 95 |
| 975859 | 95 | 96 |

### Example 5: Tolerability of Modified Oligonucleotides Targeting Human PNPLA3 in CD1 Mice

CD1 ® mice (Charles River, MA) are a multipurpose mice model, frequently utilized for safety and efficacy testing. The mice were treated with Ionis antisense oligonucleotides selected from studies described above and evaluated for changes in the levels of various plasma chemistry markers.

Ionis oligonucleotides selected from the studies above were conjugated with 5′-Trishexylamino-(THA)-C₆GalNAC3 endcap (henceforth referred to as 5′-THA). The Ionis oligonucleotides tested are presented in the Table below. ‘Unconjugated parent ION No.’ refers to the Ionis oligonucleotide described in the in vitro studies above with the same sequence. ‘3’-THA counterpart ION No.′ refers to the 3′-THA conjugated oligonucleotide with the same sequence and evaluated in the mice studies above.

TABLE 62
5′-THA oligonucleotides tested in CD1 mice tolerability study

| | Unconjugated | 3′-THA | | SEQ |
|---|---|---|---|---|
| Compound | parent ION | counterpart | | ID |
| ID | No. | ION No | Sequence | NO |
| 975591 | 916339 | 975746 | GGATATATTGGGCTCA | 1512 |
| 975592 | 912941 | 975747 | TTGCATTGCATAGCCT | 182 |
| 975593 | 916306 | 975748 | GTGTACTTTAGGCTCC | 598 |
| 975600 | 916332 | 975755 | CACAATGACATCATGG | 1020 |
| 975605 | 912848 | 975760 | CGTTTTTAGTAGTCAA | 141 |
| 975611 | 916552 | 975766 | CCTTTTATTTCCGTTA | 1024 |
| 975612 | 916789 | 975767 | GTAATATTCAGACCAG | 2178 |
| 975613 | 916602 | 975768 | CTAGTAAATGCTTGTC | 2176 |
| 975615 | 912874 | 975770 | ATACTTTTGGCAAGGC | 217 |
| 975616 | 916333 | 975771 | CTTTATTCAATGTGGC | 2174 |
| 975617 | 916780 | 975772 | AGAAATTGCAGTGCCC | 1665 |
| 975674 | 915619 | 975829 | GACTTTAGGGCAGATG | 1400 |
| 975704 | 916335 | 975859 | TAATTCTACCTGTGTC | 1227 |
| 975718 | 916586 | 975873 | AACTTTGCAGCCTATC | 605 |
| 975735 | 916782 | 975890 | CTTAGAAATTGCAGTG | 408 |
| 975736 | 916788 | 975891 | TAATATTCAGACCAGG | 830 |
| 975738 | 916815 | 975893 | CAATTCTAGACATGGC | 1313 |
| 975742 | 916822 | 975897 | TATGACATTTCAGAGT | 410 |
| 975743 | 916845 | 975898 | GTAAAGATGTGAGTGA | 618 |
| 994282 | 959455 | 994288 | TTGGATATATTGGGCT | 1982 |
| 994283 | 960010 | 994289 | AGACATATGACATTTC | 1745 |
| 994284 | 959361 | 994290 | TTTTTAGTAGTCAAGG | 1757 |
| 994285 | 959271 | 994291 | GTTGAAGGATGGATGG | 1748 |

### Treatment

Groups of four CD1 mice each were weekly injected subcutaneously with 15 mg/kg of Ionis oligonucleotides for 6 weeks, with one loading dose at day 4 (total 8 doses). One group of male CD1 mice was injected subcutaneously for 6 weeks with PBS. Mice were euthanized 48 hours after the last dose, and organs and plasma were harvested for further analysis.

### Plasma Chemistry Markers

To evaluate the effect of Ionis oligonucleotides on liver and kidney function, plasma levels of transaminases (ALT and AST), albumin, total bilirubin, and creatinine were measured at week 3 using an automated clinical chemistry analyzer (Beckman Coulter AU480, Brea, CA). The results are presented in the Table below. Ionis oligonucleotides that caused changes in the levels of any of the liver or kidney function markers outside the expected range for antisense oligonucleotides were excluded in further studies.

TABLE 63
Plasma chemistry marker levels in CD1 mice at week 3

| | | | | Total | |
|---|---|---|---|---|---|
| | Albumin | ALT | AST | bilirubin | Creatinine |
| | (g/dL) | (IU/L) | (IU/L) | (mg/dL) | (mg/dL) |
| PBS | 2.9 | 31 | 64 | 0.4 | 0.1 |
| 975611 | 2.7 | 640 | 385 | 0.3 | 0.1 |
| 994282 | 2.4 | 76 | 83 | 0.3 | 0.1 |
| 975592 | 3.0 | 786 | 942 | 0.5 | 0.1 |
| 975600 | 2.7 | 334 | 431 | 0.3 | 0.1 |
| 975591 | 2.6 | 62 | 115 | 0.4 | 0.1 |
| 975718 | 2.4 | 1717 | 2183 | 1.2 | 0.1 |
| 994284 | 2.7 | 41 | 97 | 0.3 | 0.1 |
| 994283 | 2.8 | 216 | 154 | 0.3 | 0.1 |
| 975616 | 3.0 | 69 | 137 | 0.3 | 0.1 |
| 975612 | 2.7 | 47 | 218 | 0.4 | 0.1 |
| 975674 | 2.9 | 134 | 114 | 0.4 | 0.1 |
| 975613 | 2.8 | 60 | 277 | 0.3 | 0.1 |
| 975593 | 2.7 | 429 | 405 | 0.4 | 0.1 |
| 975736 | 2.9 | 46 | 63 | 0.2 | 0.2 |
| 975735 | 2.5 | 46 | 79 | 0.2 | 0.1 |
| 975742 | 2.6 | 152 | 96 | 0.2 | 0.1 |
| 975615 | 2.9 | 207 | 189 | 0.4 | 0.1 |
| 975617 | 2.9 | 65 | 70 | 0.3 | 0.1 |
| 975605 | 2.9 | 67 | 92 | 0.3 | 0.1 |
| 975704 | 2.4 | 33 | 61 | 0.2 | 0.1 |
| 975738 | 2.6 | 43 | 67 | 0.2 | 0.1 |
| 975743 | 2.9 | 119 | 126 | 0.4 | 0.1 |
| 994285 | 2.8 | 400 | 353 | 0.2 | 0.1 |

### Hematology Assays

Blood obtained from selected mouse groups at week 6 were sent to IDEXX BioResearch for measurement of platelet count. The results are presented in the tables below. Ionis oligonucleotides that caused changes in the platelet count outside the expected range for antisense oligonucleotides were excluded in further studies.

TABLE 64
Platelet count in CD1 mice

| | Platelet (×10³/μL) |
|---|---|
| PBS | 1067 |
| 975605 | 1202 |
| 975612 | 1200 |
| 975613 | 1417 |
| 975616 | 1178 |
| 975617 | 922 |
| 975674 | 618 |
| 975591 | 941 |
| 975743 | 1127 |
| 994282 | 1384 |
| 994284 | 1255 |
| 975704 | 939 |
| 975735 | 1039 |
| 975736 | 1116 |
| 975738 | 1126 |
| 975742 | 808 |

### Example 6: Tolerability of Modified Oligonucleotides Targeting Human PNPLA3 in Sprague-Dawley Rats

Sprague-Dawley rats are a multipurpose model used for safety and efficacy evaluations. The rats were treated with Ionis antisense oligonucleotides from the studies described in the Examples above and evaluated for changes in the levels of various plasma chemistry markers.

### Treatment

Male Sprague-Dawley rats were maintained on a 12-hour light/dark cycle and fed ad libitum with Purina normal rat chow, diet 5001. Groups of 4 Sprague-Dawley rats each were weekly injected subcutaneously with 15 mg/kg of Ionis oligonucleotide for 6 weeks, with one loading dose on day 4 (total 8 doses). Forty eight hours after the last dose, rats were euthanized and organs and plasma were harvested for further analysis.

### Plasma Chemistry Markers

To evaluate the effect of Ionis oligonucleotides on hepatic function, plasma levels of transaminases were measured using an automated clinical chemistry analyzer (Beckman Coulter AU480, Brea, CA). Plasma levels of ALT (alanine transaminase) and AST (aspartate transaminase) were measured and the results are presented in the Table below expressed in IU/L. Plasma levels of bilirubin, creatinine, albumin, and BUN were also measured using the same clinical chemistry analyzer and the results are also presented in the Table below expressed in mg/dL. Ionis oligonucleotides that caused changes in the levels of any markers of liver function outside the expected range for antisense oligonucleotides were excluded in further studies.

TABLE 65
Plasma chemistry markers in Sprague-Dawley rats

| | | | | Total | | |
|---|---|---|---|---|---|---|
| | Albumin | ALT | AST | bilirubin | Creatinine | BUN |
| | (g/dL) | (IU/L) | (IU/L) | (mg/dL) | (mg/dL) | (mg/dL) |
| PBS | 3 | 35 | 81 | 0.2 | 0.2 | 12 |
| 975591 | 3 | 57 | 161 | 0.2 | 0.3 | 14 |
| 975605 | 4 | 62 | 176 | 0.3 | 0.2 | 14 |
| 975612 | 3 | 106 | 153 | 0.2 | 0.3 | 13 |
| 975613 | 3 | 32 | 94 | 0.2 | 0.2 | 12 |
| 975616 | 4 | 31 | 106 | 0.2 | 0.3 | 13 |
| 975617 | 3 | 49 | 263 | 0.2 | 0.2 | 12 |
| 975735 | 3 | 44 | 128 | 0.2 | 0.2 | 14 |
| 975736 | 3 | 73 | 293 | 0.3 | 0.3 | 14 |
| 994282 | 3 | 41 | 135 | 0.1 | 0.3 | 12 |
| 994284 | 3 | 32 | 95 | 0.1 | 0.2 | 13 |

### Kidney Function

To evaluate the effect of Ionis oligonucleotides on kidney function, urinary levels of protein and creatinine were measured using an automated clinical chemistry analyzer (Beckman Coulter AU480, Brea, CA). The ratios of total protein to creatinine are presented in the Table below. Ionis oligonucleotides that caused changes in the levels of the ratio outside the expected range for antisense oligonucleotides were excluded in further studies.

TABLE 66
Total protein to creatinine ratio in Sprague-Dawley rats

| | |
|---|---|
| PBS | 1.5 |
| 975591 | 2.0 |
| 975605 | 1.6 |
| 975612 | 1.9 |
| 975613 | 2.3 |
| 975616 | 2.0 |
| 975617 | 1.4 |
| 975735 | 2.2 |
| 975736 | 1.1 |
| 994282 | 2.1 |
| 994284 | 2.1 |

### Organ Weights

Liver, heart, spleen and kidney weights were measured at the end of the study, and are presented in the Table below. Ionis oligonucleotides that caused any changes in organ weights outside the expected range for antisense oligonucleotides were excluded from further studies.

TABLE 67
Organ weights (g)

| | Liver | Kidney | Spleen |
|---|---|---|---|
| Saline | 16 | 3 | 1 |
| 975591 | 16 | 4 | 1 |
| 975605 | 21 | 3 | 1 |
| 975612 | 12 | 3 | 1 |
| 975613 | 16 | 3 | 1 |
| 975616 | 15 | 3 | 1 |
| 975617 | 19 | 4 | 2 |
| 975735 | 14 | 4 | 1 |
| 975736 | 15 | 3 | 1 |
| 994282 | 14 | 3 | 1 |
| 994284 | 15 | 3 | 1 |

### Example 7: Effect of Antisense Inhibition of PNPLA3 in Transgenic Mouse Model

Ionis oligonucleotides were tested in a multi-dose assay in the hPNPLA3 Tg model.

### Treatment

Transgenic mice were maintained on a 12-hour light/dark cycle and were fed ad libitum normal Purina mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

### Study 1

The hPNPLA3 Tg mice were divided into groups of 4 mice each. Groups received subcutaneous injections of Ionis oligonucleotide at a weekly dose of 5 mg/kg, 1 mg/kg, or 0.25 mg/kg administered on days 1, 5, 8, 15, and 23. One group of 4 mice received subcutaneous injections of PBS on days 1, 5, 8, 15, and 23. The saline-injected group served as the control group to which oligonucleotide-treated groups were compared.

### RNA Analysis

On day 26, RNA was extracted from liver for real-time PCR analysis of measurement of mRNA expression of PNPLA3. Primer probe sets RTS36070 and RTS36075 were both used to measure PNPLA3 mRNA levels. Results are presented as percent change of mRNA, relative to PBS control, normalized with RIBOGREEN®. As presented in the Table below, treatment with Ionis antisense oligonucleotides resulted in significant dose-dependent reduction of PNPLA3 mRNA in comparison to the PBS control.

TABLE 68
Percent inhibition of PNPLA3 mRNA in the transgenic
mice liver relative to the PBS control

| | | Inhibition | Inhibition | |
|---|---|---|---|---|
| | | measured by | measured by | EC₅₀ |
| | mg/kg/wk | RTS36070 | RTS36075 | (μg/g) |
| 975605 | 5 | 93 | 90 | 2.2 |
| | 1 | 66 | 57 | |
| | 0.25 | 45 | 46 | |
| 975612 | 5 | 98 | 99 | 3.1 |
| | 1 | 89 | 88 | |
| | 0.25 | 34 | 44 | |
| 975613 | 5 | 98 | 97 | 1.0 |
| | 1 | 87 | 85 | |
| | 0.25 | 58 | 56 | |
| 975616 | 5 | 93 | 93 | 0.5 |
| | 1 | 85 | 87 | |
| | 0.25 | 60 | 63 | |
| 975617 | 5 | 97 | 97 | 0.3 |
| | 1 | 76 | 78 | |
| | 0.25 | 55 | 53 | |
| 975735 | 5 | 97 | 98 | 1.5 |
| | 1 | 74 | 75 | |
| | 0.25 | 29 | 33 | |
| 975736 | 5 | 98 | 98 | 0.9 |
| | 1 | 73 | 71 | |
| | 0.25 | 44 | 45 | |
| 994282 | 5 | 98 | 98 | 0.2 |
| | 1 | 91 | 80 | |
| | 0.25 | 62 | 58 | |
| 994284 | 5 | 99 | 100 | 0.3 |
| | 1 | 89 | 88 | |
| | 0.25 | 53 | 47 | |

### Study 2

The hPNPLA3 Tg mice were divided into groups of 4 mice each. Groups received subcutaneous injections of Ionis oligonucleotide at a weekly dose of 5 mg/kg, 2.5 mg/kg, 1 mg/kg, 0.5 mg/kg, or 0.25 mg/kg administered on days 1, 5, 8, 15, and 23. One group of 4 mice received subcutaneous injections of PBS on days 1, 5, 8, 15, and 23. The saline-injected group served as the control group to which oligonucleotide-treated groups were compared.

### RNA Analysis

On day 26, RNA was extracted from liver for real-time PCR analysis of measurement of mRNA expression of PNPLA3. Primer probe sets RTS36070 and RTS36075 were both used to measure PNPLA3 mRNA levels. Results are presented as percent change of mRNA, relative to PBS control, normalized with RIBOGREEN®. As presented in the Table below, treatment with Ionis antisense oligonucleotides resulted in significant dose-dependent reduction of PNPLA3 mRNA in comparison to the PBS control.

TABLE 69
Percent inhibition of PNPLA3 mRNA in the transgenic
mice liver relative to the PBS control

| | | Inhibition | Inhibition | | |
|---|---|---|---|---|---|
| | | measured by | measured by | EC₅₀ | EC₉₀ |
| | mg/kg/wk | RTS36070 | RTS36075 | (μg/g) | (μg/g) |
| 975612 | 5 | 96 | 97 | 1.0 | 8.6 |
| | 2.5 | 98 | 98 | | |
| | 1 | 95 | 96 | | |
| | 0.5 | 82 | 83 | | |
| | 0.25 | 43 | 44 | | |
| 975613 | 5 | 99 | 99 | 0.9 | 7.7 |
| | 2.5 | 99 | 99 | | |
| | 1 | 91 | 91 | | |
| | 0.5 | 82 | 83 | | |
| | 0.25 | 69 | 74 | | |
| 975616 | 5 | 96 | 96 | 1.0 | 9.4 |
| | 2.5 | 94 | 93 | | |
| | 1 | 89 | 89 | | |
| | 0.5 | 81 | 81 | | |
| | 0.25 | 73 | 60 | | |

### Example 8: Effect of Modified Oligonucleotides Targeting Human PNPLA3 in Cynomolgus Monkeys

Cynomolgus monkeys were treated with Ionis antisense oligonucleotides selected from studies described in the Examples above. Antisense oligonucleotide tolerability was evaluated.

### Treatment

Prior to the study, the monkeys were kept in quarantine during which the animals were observed daily for general health. The monkeys were 2-4 years old and weighed 2-4 kg. Nine groups of 5 randomly assigned male cynomolgus monkeys each were injected subcutaneously with Ionis oligonucleotide or PBS in a clock-wise rotation between four different sites on the back. The monkeys were dosed twice per week (days 1, 5, 9, and 14) for the first 2 weeks, and then subsequently once a week for 10 weeks with 10 mg/kg of Ionis oligonucleotide on days 21, 28, 35, 42, 49, 56, 63, 70, 77, and 84. A control group of 5 cynomolgus monkeys was injected with PBS in a similar manner and served as the control group.

During the study period, the monkeys were observed twice daily for signs of illness or distress. Any animal experiencing more than momentary or slight pain or distress due to the treatment, injury or illness was treated by the veterinary staff with approved analgesics or agents to relieve the pain after consultation with the Study Director. Any animal in poor health or in a possible moribund condition was identified for further monitoring and possible euthanasia. Scheduled euthanasia of the animals was conducted on day 86 approximately 48 hours after the last dose by exsanguination while under deep anesthesia. The protocols described in the Example were approved by the Institutional Animal Care and Use Committee (IACUC).

### Body and Organ Weight Measurements

To evaluate the effect of Ionis oligonucleotides on the overall health of the animals, body and organ weights were measured. Body weights and organ weights were measured on day 86 and the data is presented in the Table below. The results indicate that effect of treatment with antisense oligonucleotides on body and organ weights was within the expected range for antisense oligonucleotides. Specifically, treatment with ION 945616 was well tolerated in terms of the body and organ weights of the monkeys.

TABLE 70
Final body and organ weights in cynomolgus monkey

| | | | | Liver with |
|---|---|---|---|---|
| | Body Wt | Spleen | Kidney | gallbladder |
| | (kg) | (g) | (g) | (g) |
| PBS Control | 2797 | 2.6 | 13.1 | 53 |
| 994284 | 2789 | 3.3 | 14.7 | 69 |
| 975605 | 2685 | 4.1 | 12.2 | 58 |
| 975616 | 2868 | 3.1 | 12.9 | 63 |
| 994282 | 2782 | 4.4 | 12.1 | 62 |
| 975613 | 2704 | 3.0 | 13.5 | 60 |
| 975617 | 2761 | 3.8 | 14.1 | 61 |
| 975735 | 2765 | 4.1 | 15.5 | 67 |
| 975736 | 2844 | 3.0 | 14.1 | 66 |
| 975612 | 2711 | 2.8 | 13.2 | 60 |

### Liver Function

To evaluate the effect of Ionis oligonucleotides on hepatic function, blood samples were collected from all the study groups on day 86. The monkeys were fasted overnight prior to blood collection. Blood was collected in tubes without anticoagulant for serum separation. The tubes were kept at room temperature for a minimum of 90 minutes and then centrifuged at 3000 rpm for 10 minutes to obtain serum. Levels of various liver function markers were measured using a Toshiba 200FR NEO chemistry analyzer (Toshiba Co., Japan). Plasma levels of ALT and AST were measured and the results are presented in the Table below, expressed in IU/L. Bilirubin, a liver function marker, was similarly measured and is presented in the Table below, expressed in mg/dL. The results indicate that antisense oligonucleotides had no effect on liver function outside the expected range for antisense oligonucleotides.

TABLE 71
Liver function markers in cynomolgus monkey plasma

| | ALT | AST | Bilirubin | Albumin |
|---|---|---|---|---|
| | (IU/L) | (IU/L) | (mg/dL) | (g/dL) |
| PBS Control | 38 | 55 | 0.2 | 4.3 |
| 994284 | 64 | 48 | 0.2 | 3.7 |
| 975605 | 48 | 54 | 0.3 | 4.0 |
| 975616 | 54 | 57 | 0.3 | 3.9 |
| 994282 | 89 | 53 | 0.3 | 4.0 |
| 975613 | 60 | 71 | 0.4 | 4.0 |
| 975617 | 65 | 61 | 0.3 | 4.0 |
| 975735 | 59 | 79 | 0.3 | 4.1 |
| 975736 | 70 | 56 | 0.3 | 3.9 |
| 975612 | 61 | 66 | 0.3 | 3.9 |

### Kidney Function

To evaluate the effect of Ionis oligonucleotides on kidney function, blood samples were collected from all the study groups on day 86. The monkeys were fasted overnight prior to blood collection. Blood was collected in tubes without anticoagulant for serum separation. The tubes were kept at room temperature for a minimum of 90 minutes and then centrifuged at 3000 rpm for 10 minutes to obtain serum. Levels of BUN and creatinine were measured using a Toshiba 200FR NEO chemistry analyzer (Toshiba Co., Japan). Results are presented in the Table below, expressed in mg/dL.

The plasma chemistry data indicate that most of the Ionis oligonucleotides did not have any effect on the kidney function outside the expected range for antisense oligonucleotides.

TABLE 72
Plasma BUN and creatinine levels
(mg/dL) in cynomolgus monkeys

| | BUN | Creatinine |
|---|---|---|
| PBS Control | 23 | 0.8 |
| 994284 | 24 | 0.8 |
| 975605 | 27 | 0.7 |
| 975616 | 21 | 0.8 |
| 994282 | 24 | 0.8 |
| 975613 | 23 | 0.9 |
| 975617 | 21 | 0.7 |
| 975735 | 20 | 0.8 |
| 975736 | 23 | 0.8 |
| 975612 | 20 | 0.8 |

### Hematology

To evaluate any effect of Ionis oligonucleotides in cynomolgus monkeys on hematologic parameters, blood samples of approximately 0.5 mL of blood was collected from each of the available study animals on day 86. The samples were collected in tubes containing K₂-EDTA. Samples were analyzed for red blood cell (RBC) count, white blood cells (WBC) count, individual white blood cell counts, such as that of monocytes, neutrophils, lymphocytes, as well as for platelet count, hemoglobin content and hematocrit, using an ADVIA2120i hematology analyzer (Siemens, USA).

The data indicate the oligonucleotides did not cause any changes in hematologic parameters outside the expected range for antisense oligonucleotides at this dose.

TABLE 73
Blood cell counts in cynomolgus monkeys

| | RBC | Platelets | WBC | Neutrophils | Lymphocytes | Monocytes |
|---|---|---|---|---|---|---|
| | (×10⁶/μL) | (×10³/μL) | (×10³/μL) | (×10³/μL) | (×10³/μL) | (×10³/μL) |
| PBS Control | 6.0 | 342 | 12 | 3.2 | 7.8 | 0.3 |
| 994284 | 6.0 | 410 | 10 | 2.7 | 6.7 | 0.3 |
| 975605 | 5.8 | 326 | 10 | 4.8 | 4.5 | 0.4 |
| 975616 | 6.0 | 362 | 10 | 3.4 | 5.8 | 0.3 |
| 994282 | 5.8 | 359 | 10 | 3.9 | 5.5 | 0.3 |
| 975613 | 5.5 | 327 | 8 | 2.6 | 5.5 | 0.2 |
| 975617 | 6.1 | 358 | 10 | 3.1 | 6.4 | 0.3 |
| 975735 | 5.9 | 241 | 13 | 5.4 | 6.6 | 0.4 |
| 975736 | 5.8 | 360 | 10 | 3.5 | 6.4 | 0.2 |
| 975612 | 6.2 | 421 | 11 | 5.1 | 5.7 | 0.2 |

TABLE 74
Hematologic parameters in cynomolgus monkeys

| | Hemoglobin | HCT |
|---|---|---|
| | (g/dL) | (%) |
| PBS Control | 14 | 49 |
| 994284 | 14 | 48 |
| 975605 | 14 | 46 |
| 975616 | 14 | 49 |
| 994282 | 14 | 47 |
| 975613 | 13 | 46 |
| 975617 | 14 | 49 |
| 975735 | 14 | 48 |
| 975736 | 14 | 48 |
| 975612 | 14 | 49 |

### Pro-Inflammatory Proteins Analysis

To evaluate any inflammatory effect of Ionis oligonucleotides in cynomolgus monkeys, blood samples were taken for analysis. The monkeys were fasted overnight prior to blood collection. Approximately 1.5 mL of blood was collected from each animal and put into tubes without anticoagulant for serum separation. The tubes were kept at room temperature for a minimum of 90 min and then centrifuged at 3,000 rpm for 10 min at room temperature to obtain serum. C-reactive protein (CRP), which is synthesized in the liver and which serves as a marker of inflammation, and complement C3 were measured using a Toshiba 200FR NEO chemistry analyzer (Toshiba Co., Japan).

### Example 9: Measurement of Viscosity of Antisense Oligonucleotides Targeting Human PNPLA3

The viscosity of select antisense oligonucleotides from the studies described above was measured with the aim of screening out antisense oligonucleotides which have a viscosity of more than 40 centipoise (cP). Oligonucleotides having a viscosity greater than 40 cP would have less than optimal viscosity.

Oligonucleotides (32-35 mg) were weighed into a glass vial, 120 μL of water was added and the antisense oligonucleotide was dissolved into solution by heating the vial at 50° C. Part (75 μL) of the pre-heated sample was pipetted to a micro-viscometer (Cambridge). The temperature of the micro-viscometer was set to 25° C. and the viscosity of the sample was measured. Another part (20 μL) of the pre-heated sample was pipetted into 10 mL of water for UV reading at 260 nM at 85° C. (Cary UV instrument). The results are presented in the Table below, where the concentration of each antisense oligonucleotide was 200 mg/ml, and indicate that most of the antisense oligonucleotides solutions are optimal in their viscosity under the criterion stated above.

TABLE 75
Viscosity of antisense oligonucleotides at 200 mg/mL

| | Viscosity (cP) |
|---|---|
| 994284 | 21 |
| 975605 | 19 |
| 975616 | 20 |
| 994282 | 30 |
| 975613 | 24 |
| 975617 | 22 |
| 975735 | 15 |
| 975736 | 49 |
| 975612 | 25 |

### Example 10: Design of Oligonucleotides at the Site of ION 975616

Additional antisense oligonucleotides were designed targeting a PNPLA3 nucleic acid that overlap the target site of ION 916333, which is the unconjugated version of ION 975616, and with different chemical modifications and motifs.

The newly designed chimeric antisense oligonucleotides in the Tables below were designed as 3-10-3 cEt gapmers or deoxy, MOE, and cEt oligonucleotides. The 3-10-3 cEt gapmers are 16 nucleosides in length, wherein the central gap segment comprises of ten 2′-deoxynucleosides and is flanked by wing segments on the 5′ direction and the 3′ direction comprising three nucleosides. Each nucleoside in the 5′ wing segment and each nucleoside in the 3′ wing segment has a cEt sugar modification. The intermucleoside linkages throughout each gapmer are phosphorothioate (P═S) linkages. All cytosine residues throughout each gapmer are 5-methylcytosines. The deoxy, MOE and (S)-cEt oligonucleotides are 16 nucleosides in length wherein the nucleoside have either a MOE sugar modification, an (S)-cEt sugar modification, or a deoxy modification. The ‘Chemistry’ column describes the sugar modifications of each oligonucleotide. ‘k’ indicates an (S)-cEt sugar modification; ‘d’ indicates deoxyribose; the number after the ‘d’ indicates the number of deoxyribose; and ‘e’ indicates a MOE modification. The intermucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence (SEQ ID NO: 2).

TABLE 76
Modified oligonucleotides targeting human PNPLA3

| Start | Stop | | Compound | | SEQ ID |
|---|---|---|---|---|---|
| Site | Site | Sequence | Number | Chemistry | NO |
| 5599 | 5614 | TCAATGTGGCTTCTAG | 995553 | kkk-d10-kkk | 2170 |
| 5600 | 5615 | TTCAATGTGGCTTCTA | 959437 | kkk-d10-kkk | 2089 |
| 5601 | 5616 | ATTCAATGTGGCTTCT | 959438 | kkk-d10-kkk | 2171 |
| 5602 | 5617 | TATTCAATGTGGCTTC | 959439 | kkk-d10-kkk | 2172 |
| 5603 | 5618 | TTATTCAATGTGGCTT | 959440 | kkk-d10-kkk | 1705 |
| 5603 | 5618 | TTATTCAATGTGGCTT | 995696 | k-d10-kekek | 1705 |
| 5603 | 5618 | TTATTCAATGTGGCTT | 995906 | kk-d9-eeekk | 1705 |
| 5603 | 5618 | TTATTCAATGTGGCTT | 996116 | kk-d9-ekeke | 1705 |
| 5604 | 5619 | TTTATTCAATGTGGCT | 959441 | kkk-d10-kkk | 1765 |
| 5604 | 5619 | TTTATTCAATGTGGCT | 995697 | k-d10-kekek | 1765 |
| 5604 | 5619 | TTTATTCAATGTGGCT | 995907 | kk-d9-eeekk | 1765 |
| 5604 | 5619 | TTTATTCAATGTGGCT | 996117 | kk-d9-ekeke | 1765 |
| 5605 | 5620 | CTTTATTCAATGTGGC | 916333 | kkk-d10-kkk | 2173 |
| 5605 | 5620 | CTTTATTCAATGTGGC | 995698 | k-d10-kekek | 1089 |
| 5605 | 5620 | CTTTATTCAATGTGGC | 995908 | kk-d9-eeekk | 1089 |
| 5605 | 5620 | CTTTATTCAATGTGGC | 996118 | kk-d9-ekeke | 1089 |
| 5605 | 5620 | CTTTATTCAATGTGGC | 996277 | kek-d9-eekk | 1089 |
| 5606 | 5621 | ACTTTATTCAATGTGG | 916334 | kkk-d10-kkk | 1158 |
| 5606 | 5621 | ACTTTATTCAATGTGG | 995699 | k-d10-kekek | 1158 |
| 5606 | 5621 | ACTTTATTCAATGTGG | 995909 | kk-d9-eeekk | 1158 |
| 5606 | 5621 | ACTTTATTCAATGTGG | 996119 | kk-d9-ekeke | 1158 |
| 5607 | 5622 | TACTTTATTCAATGTG | 959442 | kkk-d10-kkk | 1825 |
| 5608 | 5623 | TTACTTTATTCAATGT | 959443 | kkk-d10-kkk | 1885 |

The oligonucleotides were tested in a series of experiments. Cultured A-431 cells at a density of 10,000 cells per well were treated using free uptake with modified oligonucleotides diluted to different concentrations. After a treatment period of approximately 48 hours, PNPLA3 mRNA levels were measured as previously described using the Human PNPLA3 primer-probe set RTS36070. PNPLA3 mRNA levels were adjusted according to total RNA content, as measured by RIBOGREEN®. The IC₅₀ ratios of the assays are presented in the tables below, which is the ratio of the IC₅₀ of a benchmark oligonucleotide to the IC₅₀ of the oligonucleotide. Hence, a bigger value of the ratio indicates that the oligonucleotide is more active than the benchmark.

TABLE XX
Efficacy of modified oligonucleotides targeting
human PNPLA3

| Start | Stop | Compound | | IC₅₀ |
|---|---|---|---|---|
| Site | Site | Number | Chemistry | ratio |
| 5600 | 5615 | 959437 | kkk-d10-kkk | 1.42 |
| 5601 | 5616 | 959438 | kkk-d10-kkk | 0.49 |
| 5602 | 5617 | 959439 | kkk-d10-kkk | 0.36 |
| 5603 | 5618 | 959440 | kkk-d10-kkk | 0.55 |
| 5603 | 5618 | 995906 | kk-d9-eeekk | 1.42 |
| 5604 | 5619 | 959441 | kkk-d10-kkk | 1.66 |
| 5605 | 5620 | 916333 | kkk-d10-kkk | 1.96 |
| 5605 | 5620 | 995908 | kk-d9-eeekk | 0.70 |
| 5606 | 5621 | 916334 | kkk-d10-kkk | 0.95 |
| 5606 | 5621 | 995909 | kk-d9-eeekk | 1.47 |

1-41. (canceled)

42. A method of treating, preventing, or ameliorating a disease associated with PNPLA3 in an individual comprising administering to the individual a compound, wherein the anionic form of the compound has the following formula (SEQ ID NO: 1089): 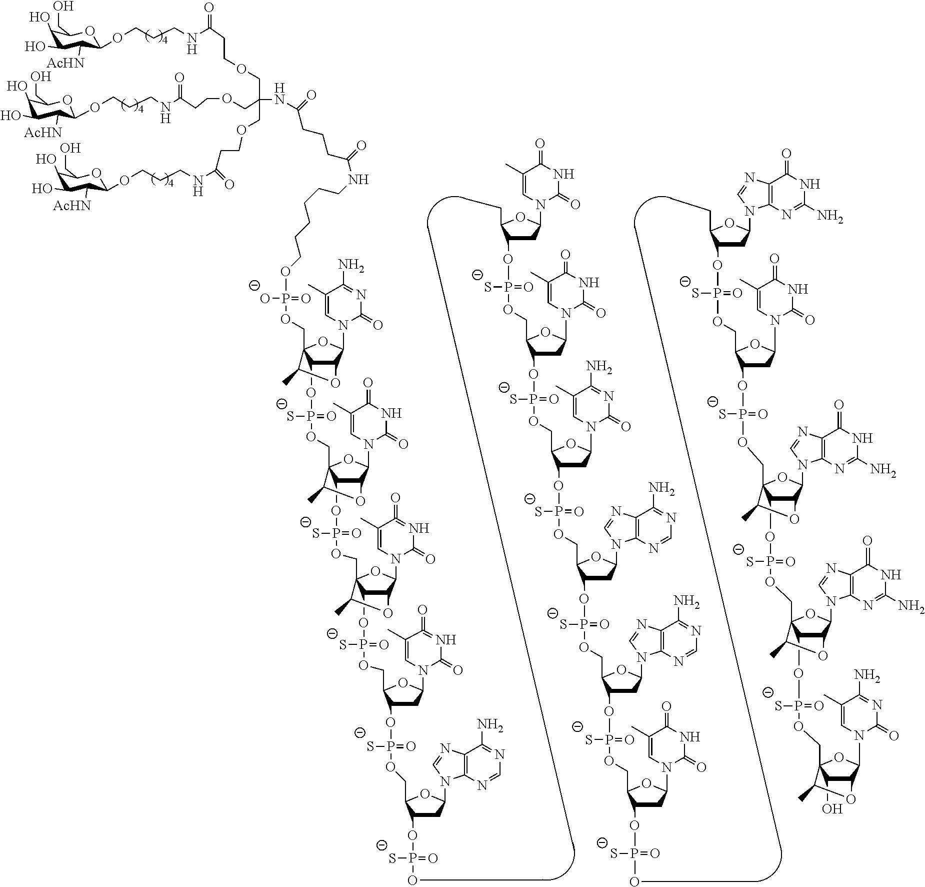
or a pharmaceutically acceptable salt thereof, thereby treating, preventing, or ameliorating the disease.

43. (canceled)

44. (canceled)

45. The method of claim 42, wherein the disease is liver disease, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

46. The method of claim 42, wherein administering the compound inhibits or reduces or improves liver damage, steatosis, liver fibrosis, liver inflammation, liver scarring or cirrhosis, liver failure, liver enlargement, elevated transaminases, or hepatic fat accumulation in the individual.

47. A method of inhibiting expression of PNPLA3 in a cell comprising contacting the cell with a compound, wherein the anionic form of the compound has the following formula (SEQ ID NO: 1089): 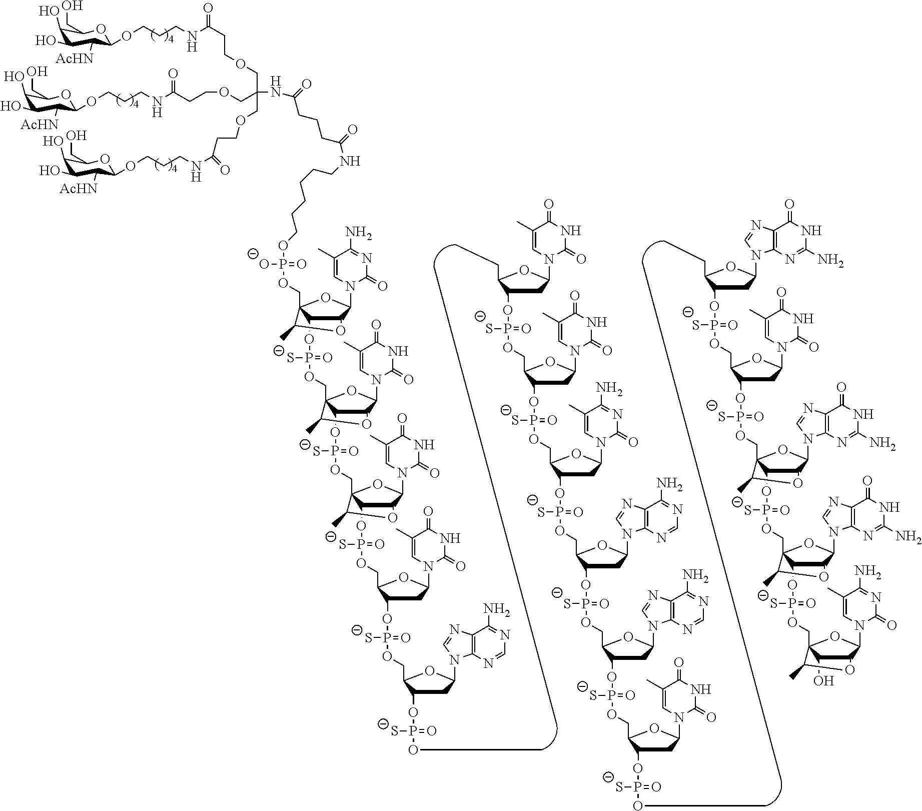
or a pharmaceutically acceptable salt thereof, thereby inhibiting expression of PNPLA3 in the cell.

48. The method of claim 47, wherein the cell is in the liver of an individual.

49. The method of claim 48, wherein the individual has, or is at risk of having, liver disease, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

50. A method of reducing or inhibiting liver damage, steatosis, liver fibrosis, liver inflammation, liver scarring or cirrhosis, liver failure, liver enlargement, elevated transaminases, or hepatic fat accumulation in an individual, comprising administering a compound, wherein the anionic form of the compound has the following formula (SEQ ID NO: 1089): 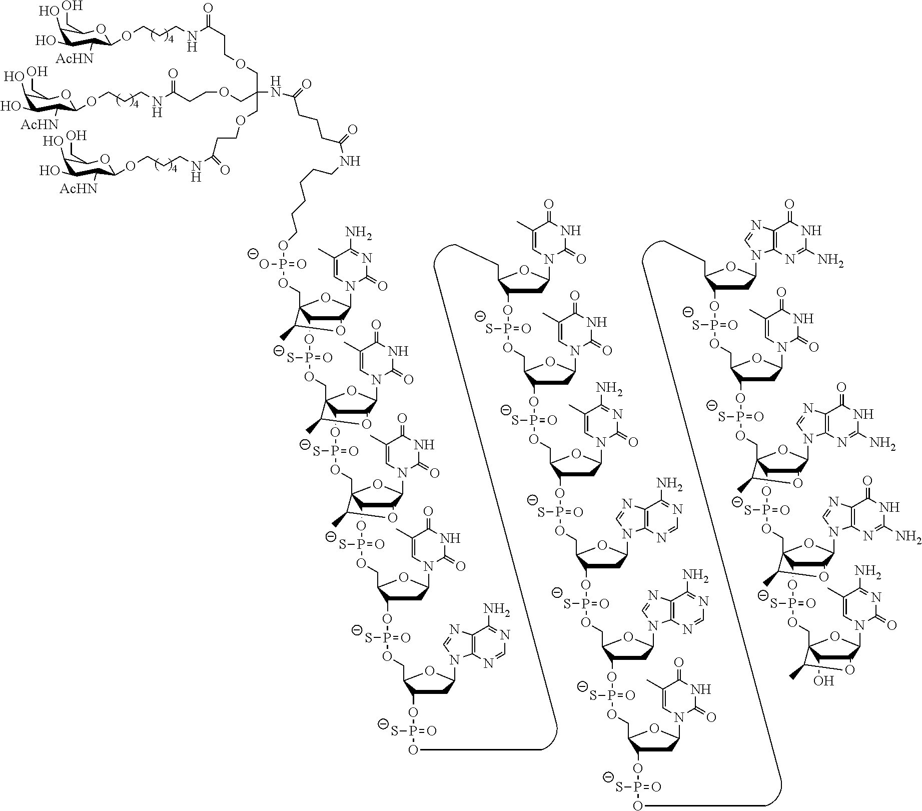
or a pharmaceutically acceptable salt thereof, to the individual, thereby reducing or inhibiting liver damage, steatosis, liver fibrosis, liver inflammation, liver scarring or cirrhosis, liver failure, liver enlargement, elevated transaminases, or hepatic fat accumulation in the individual.

51. The method of claim 50, wherein the individual has, or is at risk of having, liver disease, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

52-62. (canceled)
